(19) United States
(12) Patent Application Publication
(10) Pub. No.: US 2022/0030868 A1
(43) Pub. Date: Feb. 3, 2022

(54) FUNGICIDAL HALOMETHYL KETONES AND HYDRATES

(71) Applicant: FMC CORPORATION, Philadelphia, PA (US)

(72) Inventors: ROBERT JAMES PASTERIS, NEWARK, DE (US); SRINIVAS CHITTABOINA, BIBIPET (IN); RAVISEKHARA P. REDDY, SECUNDERABAD (IN); TRAVIS CHANDLER MCMAHON, MIDDLETOWN, DE (US); HENGBIN WANG, NEWARK, DE (US); ALVIN DONALD CREWS, Jr., VOORHEES, NJ (US); LIANA HIE, WILMINGTON, DE (US); EARL WILLIAM REED, SMYRNA, DE (US)

(21) Appl. No.: 17/276,211

(22) PCT Filed: Sep. 12, 2019
(86) PCT No.: PCT/US2019/050750

§ 371 Date: Mar. 15, 2021

Related U.S. Application Data
(60) Provisional application No. 62/731,190, filed on Sep. 14, 2018.
(60) Provisional application No. 62/754,912, filed on Nov. 2, 2018.
(60) Provisional application No. 62/885,488, filed on Aug. 12, 2019.

(51) Int. Cl. A01N 43/50; A01N 43/78; A01N 25/30

(52) U.S. Cl. CPC A01N 43/50; A01N 25/30; A01N 43/78

(57) ABSTRACT

Disclosed are compounds of Formulae 1 and 10 including all geometric and stereoisomers, tautomers, N oxides, and salts thereof, wherein E, L, J, A, T, R1, R2a, R2b, X, Y, R6a, R6b and R29 are as defined in the disclosure. Also disclosed are compositions containing the compounds of Formula 1 and methods for controlling plant disease caused by a fungal pathogen comprising applying an effective amount of a compound or a composition of the invention.

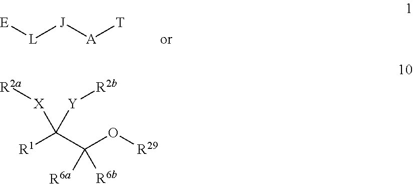

### FIELD OF THE INVENTION

This invention relates to certain halomethyl ketones and hydrates, their N-oxides, salts and compositions, and methods of using them as fungicides.

### BACKGROUND OF THE INVENTION

The control of plant diseases caused by fungal plant pathogens is extremely important in achieving high crop efficiency. Plant disease damage to ornamental, vegetable, field, cereal and fruit crops can cause significant reduction in productivity and thereby result in increased costs to the consumer. Many products are commercially available for these purposes, but the need continues for new compounds which are more effective, less costly, less toxic, environmentally safer or have different sites of action.

PCT Patent Publications WO 2018/080859, WO 2018/118781, WO 2018/187553 and WO 2019/010192 discloses trifluoromethyl-oxadiazole derivatives and their use as fungicides.

### SUMMARY OF THE INVENTION

This invention is directed to compounds of Formula 1 (including all stereoisomers), tautomers, N-oxides, and salts thereof, agricultural compositions containing them and their use as fungicides:

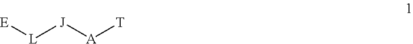

wherein

T is selected from the group consisting of:

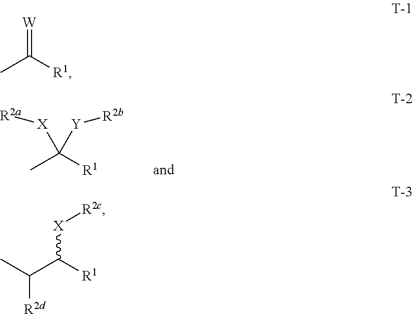

wherein the bond extending to the left is attached to A;
R¹ is CF₃, CHF₂, CCl₃, CHCl₂, CF₂Cl, CFCl₂ or CHFCl;
W is O, S or NR³;
R³ is H, cyano, nitro, C(═O)OH, benzyl, C₁-C₄ alkyl, C₂-C₄ alkylcarbonyl, C₂-C₄ haloalkylcarbonyl, OR^{3a} or NR^{3b}R^{3c};
R^{3a} is H, benzyl, C₁-C₄ alkyl, C₂-C₄ alkylcarbonyl or C₂-C₄ haloalkylcarbonyl;
R^{3b} is H, C₁-C₄ alkyl, C₂-C₄ alkylcarbonyl or C₂-C₄ haloalkylcarbonyl;
R^{3c} is H or C₁-C₄ alkyl; or
R^{3b} and R^{3c} are taken together to form a 4- to 6-membered fully saturated heterocyclic ring, each ring containing ring members, in addition to the connecting nitrogen atom, selected from carbon atoms and up to 2 heteroatoms independently selected from up to 2 O, up to 2 S and up to 2 N atoms, each ring optionally substituted with up to 2 methyl groups;
X is O, S or NR^{5a};
Y is O, S or NR^{5b};
R^{5a} and R^{5b} are each independently H, hydroxy or C₁-C₄ alkyl;
R^{2a} and R^{2b} are each independently H, C₁-C₄ alkyl, C₂-C₄ alkenyl, (CR^{4a}R^{4b})ₚ—OH, (CR^{4a}R^{4b})ₚ—SH, (CR^{4a}R^{4b})ₚ—Cl or (CR^{4a}R^{4b})ₚ—Br; or
R^{2a} and R^{2b} are taken together with the atoms X and Y to which they are attached to form a 5- to 7-membered saturated ring containing ring members, in addition to the atoms X and Y, selected from carbon atoms, wherein up to 2 carbon atom ring members are independently selected from C(═O) and C(═S), the ring optionally substituted with up to 2 substituents independently selected from halogen, cyano, C₁-C₂ alkyl, C₁-C₂ haloalkyl, C₁-C₂ alkoxy and C₁-C₂ haloalkoxy on carbon atom ring members;
R^{2c} is C₁-C₄ alkyl, C₁-C₄ haloalkyl, C₂-C₄ alkenyl, C₂-C₄ haloalkenyl, C₂-C₄ alkynyl or C₂-C₄ haloalkynyl, each optionally substituted with up 2 substituents independently selected from cyano, hydroxy, SC≡N and C₁-C₂ alkoxy;
R^{2d} is H, cyano, halogen or C₁-C₄ alkyl;
each R^{4a} and R^{4b} is independently H or C₁-C₄ alkyl;
p is 2 or 3;
when T is T-1 or T-2, then A is A¹-A²-CR^{6a}R^{6b}, wherein A¹ is connected to J, and CR^{6a}R^{6b} is connected to T;
when T is T-3, then A is A¹-A², wherein A¹ is connected to J, and A² is connected to T;
A¹ is CR^{6c}R^{6d}, N(R^{7a}), O or S;
A² is a direct bond, CR^{6e}R^{6f}, N(R^{7b}), O or S;
R^{6a}, R^{6b}, R^{6c}, R^{6d}, R^{6e} and R^{6f} are each independently H, cyano, hydroxy, halogen or C₁-C₄ alkyl;
R^{7a} and R^{7b} are each independently H, C(═O)H, C₁-C₄ alkyl or C₂-C₄ alkylcarbonyl;
J is selected from the group consisting of:

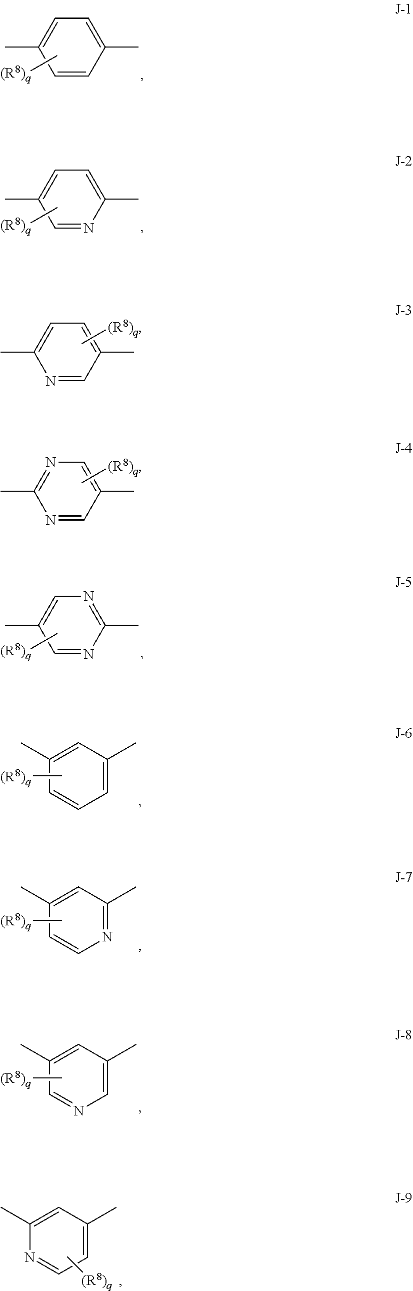

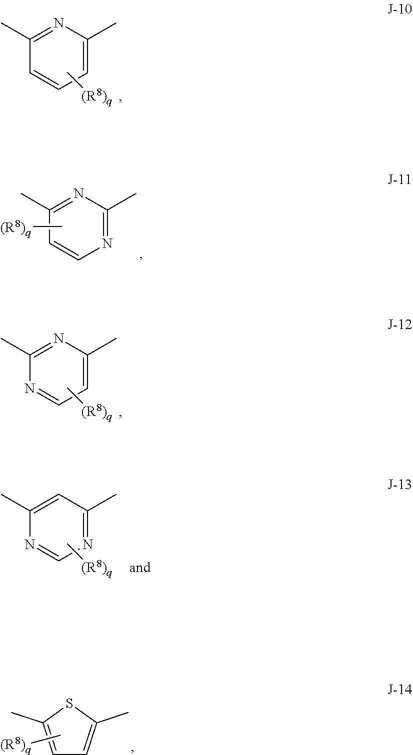

wherein the bond extending to the left is attached to L, and the bond extending to the right is attached to A;
each R⁸ is independently F, Cl, methyl or methoxy;
q is 0, 1 or 2;
L is (CR^{9a}R^{9b})ₙ;
each R^{9a} and R^{9b} is independently H, halogen, cyano, hydroxy, nitro, C₁-C₃ alkyl, C₁-C₃ haloalkyl, C₁-C₃ alkoxy or C₁-C₃ haloalkoxy;
n is 0, 1, 2 or 3;
E is E¹ or E²;
E¹ is amino, cyano, hydroxy, nitro, CH(═O), C(═O)OH, C(═O)NH₂, C(═S)NH₂, OC(═O)NH₂, OC(═S)NH₂, NHC(═O)NH₂, NHC(═S)NH₂, SC≡N, —CH═NNHC(═O)OC₁-C₆ alkyl or —N(OCH₃)C(═O)C₁-C₆ alkyl; or
E¹ is C₁-C₆ alkoxy, C₂-C₆ alkenyloxy, C₂-C₆ alkynyloxy, C₁-C₆ alkylthio, C₂-C₆ alkenylthio, C₂-C₆ alkynylthio, C₁-C₆ alkylsulfinyl, C₂-C₆ alkenylsulfinyl, C₂-C₆ alkynylsulfinyl, C₁-C₆ alkylsulfonyl, C₂-C₆ alkenylsulfonyl, C₂-C₆ alkynylsulfonyl, C₁-C₆ alkylsulfonylamino, C₂-C₆ alkenylsulfonylamino, C₂-C₆ alkynylsulfonylamino, C₁-C₆ alkylaminosulfonyl, C₂-C₆ dialkylaminosulfonyl, C₂-C₆ alkenylaminosulfonyl, C₂-C₆ alkynylaminosulfonyl, C₁-C₆ alkylaminosulfonylamino, C₂-C₆ alkenylaminosulfonylamino, C₂-C₆ alkynylaminosulfonylamino, C₂-C₆ alkylcarbonyl, C₃-C₆ alkenylcarbonyl, C₃-C₆ alkynylcarbonyl, C₂-C₆ alkylaminocarbonyl, C₃-C₆ alkenylaminocarbonyl, C₃-C₆ alkynylaminocarbonyl, C₂-C₆ alkylcarbonylamino, C₃-C₆ alkenylcarbonylamino, C₃-C₆ alkynylcarbonylamino, C₂-C₆ alkylaminocarbonylamino, C₃-C₆ alkenylaminocarbonylamino, C₃-C₆ alkynylaminocarbonylamino, C₂-C₆ alkylcarbonyloxy, C₃-C₆ alkenylcarbonyloxy, C₃-C₆ alkynylcarbonyloxy, C₂-C₆ alkoxycarbonyl, C₃-C₆ alkenyloxycarbonyl, C₃-C₆ alkynyloxycarbonyl, C₂-C₆ alkylaminocarbonyloxy, C₃-C₆ alkenylaminocarbonyloxy, C₃-C₆ alkynylaminocarbonyloxy, C₂-C₆ alkoxycarbonylamino, C₃-C₆ alkenyloxy carbonylamino, C₃-C₆ alkynyloxy carbonylamino, C₂-C₆ alkylamino(thiocarbonyl)oxy, C₃-C₆ alkenylamino(thiocarbonyl)oxy, C₃-C₆ alkynylamino(thiocarbonyl)oxy, C₂-C₆) alkoxy(thiocarbonyl)amino, C₃-C₆ alkenyloxy(thiocarbonyl)amino, C₃-C₆ alkynyloxy(thiocarbonyl)amino, C₂-C₆ alkyl(thiocarbonyl), C₂-C₆ (alkylthio)carbonyl, C₃-C₆ alkenyl(thiocarbonyl), C₃-C₆ (alkenylthio)carbonyl, C₃-C₆ alkynyl(thiocarbonyl), C₃-C₆ (alkynylthio)carbonyl, C₂-C₆ alkylamino(thiocarbonyl), C₃-C₆ alkenylamino(thiocarbonyl), C₃-C₆ alkynylamino(thiocarbonyl), C₂-C₆, alkyl(thiocarbonyl)amino, C₂-C₆ (alkylthio)carbonylamino, C₃-C₆ alkenyl(thiocarbonyl)amino, C₃-C₆ (alkenylthio)carbonylamino, C₃-C₆ alkynyl(thiocarbonyl)amino, C₃-C₆ (alkynylthio)carbonylamino, C₂-C₆ alkylamino(thiocarbonyl)amino, C₃-C₆ alkenylamino(thiocarbonyl)amino or C₃-C₆ alkynylamino(thiocarbonyl)amino, wherein each carbon atom is optionally substituted with up to 1 substituent selected from R^{10a} and up to 3 substituents independently selected from R^{10b};
R^{10a} is phenyl optionally substituted with up to 3 substituents independently selected from R^{11a}; or a 5- to 6-membered heterocyclic ring containing ring members selected from carbon atoms and 1 to 4 heteroatoms independently selected from up to 2 O, up to 2 S and up to 4 N atoms, wherein up to 3 carbon atom ring members are independently selected from C(═O) and C(═S), and sulfur atom ring members are independently S(═O)ᵤ(═NR¹²)ᵥ, each ring optionally substituted with up to 3 substituents independently selected from R^{11a} on carbon atom ring members and R^{11b} on nitrogen atom ring members;
each R^{10b} is independently amino, cyano, halogen, hydroxy, nitro, SC≡N, —SH, C₁-C₄ alkyl, C₁-C₄ haloalkyl, C₃-C₆ cycloalkyl, C₃-C₆ halocycloalkyl, C₁-C₄ alkoxy, C₁-C₄ haloalkoxy, C₁-C₄ alkylthio, C₁-C₄ alkylsulfinyl, C₁-C₄ alkylsulfonyl, C₁-C₄ haloalkylsulfonyl, C₁-C₄ alkylamino, C₂-C₄ dialkylamino, C₂-C₄ alkylcarbonyl, C₂-C₄ haloalkylcarbonyl, C₂-C₅ alkoxycarbonyl, C₂-C₅ haloalkoxycarbonyl, C₂-C₅ alkylaminocarbonyl or C₃-C₅ dialkylaminocarbonyl;
each R^{11a} is independently halogen, hydroxy, cyano, amino, nitro, C₁-C₄ alkyl, C₁-C₄ haloalkyl, C₂-C₄ alkenyl, C₂-C₄ alkynyl, C₁-C₄ hydroxyalkyl, C₃-C₆ cycloalkyl, C₄-C₇ cycloalkylalkyl, C₁-C₄ alkoxy, C₁-C₄ haloalkoxy, C₂-C₄ alkenyloxy, C₂-C₄ alkynyloxy, C₂-C₄ alkoxyalkyl, C₂-C₆ alkylcarbonyloxy, C₁-C₄ alkylthio, C₁-C₄ haloalkylthio, C₂-C₆ alkylcarbonylthio, C₁-C₄ alkylsulfinyl, C₁-C₄ haloalkylsulfinyl, C₁-C₄ alkylsulfonyl, C₁-C₄ haloalkylsulfonyl, C₁-C₄ alkylsulfonyloxy, C₁-C₄ alkylamino, C₂-C₈ dialkylamino, C₃-C₆ cycloalkylamino, C₂-C₄ alkylcarbonyl, C₃-C₅ alkenylcarbonyl, C₃-C₅ alkynylcarbonyl, C₄-C₇ cycloalkylcarbonyl, C₅-C₈ cycloalkylalkylcarbonyl, C₂-C₆ alkoxycarbonyl, C₃-C₇ alkenyloxycarbonyl, C₃-C₇ alkynyloxycarbonyl, C₄-C₇ cycloalkoxylcarbonyl, C₅-C₈ cycloalkylalkoxylcarbonyl, C₂-C₆ alkylaminocarbonyl, C₃-C₆ alkenylaminocarbonyl, C₃-C₆ alkynylaminocarbonyl, C₄-C₇ cycloalkylaminocarbonyl, C₅-C₈ cycloalkylalkylaminocarbonyl, C₃-C₈ dialkylaminocarbonyl or C₃-C₆ trialkylsilyl; each R^{11b} is independently C(═O)H, C₁-C₃ alkyl, C₁-C₃ alkoxy, C₂-C₃ alkylcarbonyl or C₂-C₃ alkoxycarbonyl;
each R¹² is independently H, cyano, C₁-C₃ alkyl or C₁-C₃ haloalkyl;
each u and v are independently 0, 1 or 2, provided that the sum of u and v are 0, 1 or 2;
E² is G-Z, wherein Z is attached to L;
G is phenyl optionally substituted with up to 3 substituents independently selected from R¹³; or
G is a 5- to 6-membered heteroaromatic ring, each ring containing ring members selected from carbon atoms and 1 to 4 heteroatoms independently selected from up to 2 O, up to 2 S and up to 4 N atoms, each ring optionally substituted with up to 3 substituents independently selected from R¹³; or
G is a 3- to 7-membered nonaromatic ring or an 8- to 11-membered bicyclic ring system, each ring or ring system containing ring members selected from carbon atoms and optionally up to 4 heteroatoms independently selected from up to 2 O, up to 2 S and up to 4 N atoms, wherein up to 2 ring members are independently selected from C(═O), C(═S), S(═O) and S(═O)₂, each ring or ring system optionally substituted with up to 3 substituents independently selected from R¹³;
each R¹³ is independently cyano, halogen, hydroxy, nitro, —SH, SF₅, CH(═O), C(═O)OH, NR^{14a}R^{14b}C(═O)NR^{14a}R^{14b}, C(═O)C(═O)NR^{14a}R^{14b}, C(═S)NR^{14a}R^{14b}, C(R¹⁵)═NR¹⁶, N═CR¹⁷NR^{18a}R^{18b} or —U—V-Q; or C₁-C₆ alkyl, C₂-C₆ alkenyl, C₂-C₆ alkynyl, C₃-C₇ cycloalkyl, C₃-C₇ cycloalkenyl, C₁-C₆ alkoxy, C₂-C₆ alkenyloxy, C₂-C₆ alkynyloxy, C₃-C₇ cycloalkoxy, C₁-C₀ alkylthio, C₁-C₀ alkylsulfinyl, C₁-C₆ alkylsulfonyl, C₁-C₆ alkylaminosulfinyl, C₂-C₆ dialkylaminosulfinyl, C₁-C₀ alkylsulfonyloxy, C₁-C₆ alkylsulfonylamino, C₂-C₆ alkylcarbonyl, C₄-C₇ cycloalkylcarbonyl, C₂-C₆ alkoxy carbonyl, C₃-C₆ alkenyloxy carbonyl, C₃-C₆ alkynyloxy carbonyl, C₄-C₇ cycloalkoxy carbonyl, C₃-C₆ alkoxy carbonylcarbonyl, C₂-C₆ alkylcarbonyloxy, C₄-C₇ cycloalkylcarbonyloxy, C₂-C₆ alkoxycarbonyloxy, C₄-C₇ cycloalkoxycarbonyloxy, C₂-C₆ alkylaminocarbonyloxy, C₄-C₇ cycloalkylaminocarbonyloxy, C₂-C₆ alkylcarbonylamino, C₄-C₇ cycloalkylcarbonylamino, C₂-C₆ alkoxycarbonylamino, C₄-C₇ cycloalkoxycarbonylamino, C₂-C₆ alkylaminocarbonylamino, C₄-C₇ cycloalkylaminocarbonylamino or C₂-C₆ dialkoxyphosphinyl, each optionally substituted with up to 3 substituents independently selected from R¹⁹;
each R^{14a} is independently H, cyano, hydroxy, C₁-C₄ alkyl, C₁-C₄ haloalkyl, C₂-C₄ alkenyl, C₂-C₄ haloalkenyl, C₂-C₄ alkynyl, C₂-C₄ haloalkynyl, C₁-C₅ alkoxy, C₂-C₄ alkoxyalkyl, C₁-C₄ alkylsulfonyl, C₁-C₄ haloalkylsulfonyl, C₂-C₄ alkylthioalkyl, C₂-C₄ alkylsulfinylalkyl, C₂-C₄ alkylsulfonylalkyl, C₂-C₄ alkylcarbonyl, C₂-C₄ haloalkylcarbonyl, C₄-C₇ cycloalkylcarbonyl, C₂-C₅ alkoxy carbonyl, C₃-C₅ alkoxycarbonylalkyl, C₂-C₅ alkylaminocarbonyl or C₃-C₅ dialkylaminocarbonyl;
each R^{14b} is independently H, C₁-C₆ alkyl, C₁-C₆ haloalkyl, C₂-C₆ alkenyl, C₂-C₆ haloalkenyl, C₂-C₆ alkynyl, C₂-C₆ haloalkynyl, C₁-C₀ hydroxyalkyl, C₂-C₆ cyanoalkyl, C₃-C₈ cycloalkyl, C₃-C₈ halocycloalkyl, C₃-C₈ cycloalkenyl, C₃-C₈ halocycloalkenyl, C₄-C₁₀ alkylcycloalkyl, C₄-C₁₀ cycloalkylalkyl, C₄-C₁₀ halocycloalkylalkyl, C₆-C₁₄ cycloalkylcycloalkyl, C₅-C₁₀ alkylcycloalkylalkyl, C₂-C₆ alkoxyalkyl, C₂-C₆ haloalkoxyalkyl, C₄-C₁₀ cycloalkoxyalkyl, C₃-C₈ alkoxyalkoxyalkyl, C₂-C₆ alkylthioalkyl, C₂-C₆ alkylsulfinylalkyl, C₂-C₆ alkylsulfonylalkyl, C₂-C₆ alkylaminoalkyl, C₂-C₆ haloalkylaminoalkyl, C₃-C₈ dialkylaminoalkyl or C₄-C₁₀ cycloalkylaminoalkyl, each optionally substituted with up to 1 substituent selected from cyano, hydroxy, nitro, C₂-C₄ alkylcarbonyl, C₂-C₄ alkoxycarbonyl, C₃-C₁₅ trialkylsilyl and C₃-C₁₅ halotrialkylsilyl; or
R^{14a} and R^{14b} are taken together to form a 4- to 6-membered fully saturated heterocyclic ring, each ring containing ring members, in addition to the connecting nitrogen atom, selected from carbon atoms and up to 2 heteroatoms independently selected from up to 2 O, up to 2 S and up to 2 N atoms, each ring optionally substituted with up to 3 substituents independently selected from halogen and C₁-C₃ alkyl;
each R¹⁵ is independently H, cyano, halogen, methyl, methoxy, methylthio or methoxy carbonyl;
each R¹⁶ is independently hydroxy or NR^{20a}R^{20b}; or C₁-C₄ alkoxy, C₂-C₄ alkenyloxy, C₂-C₄ alkynyloxy, C₂-C₄ alkylcarbonyloxy, C₂-C₅ alkoxycarbonyloxy, C₂-C₅ alkylaminocarbonyloxy or C₃-C₅ dialkylaminocarbonyloxy, each optionally substituted with up to 1 substituent selected from cyano, halogen, hydroxy and C(═O)OH;
each R¹⁷ is independently H, methyl, methoxy or methylthio;
each R^{18a} and R^{18b} is independently H or C₁-C₄ alkyl; or
R^{18a} and R^{18b} are taken together to form a 5- to 6-membered fully saturated heterocyclic ring, each ring containing ring members, in addition to the connecting nitrogen atom, selected from carbon atoms and up to 2 heteroatoms independently selected from up to 2 O, up to 2 S and up to 2 N atoms, each ring optionally substituted with up to 2 methyl groups;
each R¹⁹ is independently amino, cyano, halogen, hydroxy, nitro, —SH, C₁-C₄ alkyl, C₁-C₄ haloalkyl, C₃-C₆ cycloalkyl, C₃-C₆ halocycloalkyl, C₁-C₄ alkoxy, C₁-C₄ haloalkoxy, C₂-C₄ alkoxyalkoxy, C₁-C₄ alkylthio, C₁-C₄ alkylsulfinyl, C₁-C₄ alkylsulfonyl, C₁-C₄ haloalkylsulfonyl, C₂-C₄ alkylcarbonyl, C₂-C₄ haloalkylcarbonyl, C₂-C₅ alkoxycarbonyl, C₁-C₆ alkylamino, C₂-C₆ dialkylamino, C₂-C₅ alkylaminocarbonyl, C₃-C₅ dialkylaminocarbonyl, C₃-C₅ alkylthioalkylcarbonyl, C₃-C₁₅ trialkylsily, C₃-C₁₅ halotrialkylsilyl, C(R²¹)═NOR²² or C(R²³)═NR²⁴;
each U is independently a direct bond, C(═O)O, C(═O)N(R²⁵) or C(═S)N(R²⁶), wherein the atom to the left is connected to G, and the atom to the right is connected to V;
each V is independently a direct bond; or C₁-C₆ alkylene, C₂-C₆ alkenylene, C₃-C₆ alkynylene, C₃-C₆ cycloalkylene or C₃-C₆ cycloalkenylene, wherein up to 1 carbon atom is C(═O), each optionally substituted with up to 3 substituents independently selected from halogen, cyano, nitro, hydroxy, C₁-C₂ alkyl, C₁-C₂ haloalkyl, C₁-C₂ alkoxy and C₁-C₂ haloalkoxy;
each Q is independently phenyl or phenoxy, each optionally substituted with up to 2 substituents independently selected from R²⁷; or
each Q is independently a 5- to 6-membered heteroaromatic ring, each ring containing ring members selected from carbon atoms and 1 to 4 heteroatoms independently selected from up to 2 O, up to 2 S and up to 4 N atoms, each ring optionally substituted with up to 2 substituents independently selected from R²⁷; or
each Q is independently a 3- to 7-membered nonaromatic heterocyclic ring, each ring containing ring members selected from carbon atoms and 1 to 4 heteroatoms independently selected from up to 2 O, up to 2 S and up to 4 N atoms, wherein up to 2 ring members are independently selected from C(═O), C(═S), S(═O) and S(═O)₂, each ring optionally substituted with up to 2 substituents independently selected from R²⁷;
each R^{20a} is independently H, C₁-C₄ alkyl or C₂-C₄ alkylcarbonyl;
each R^{20b} is independently H, cyano, C₁-C₅ alkyl, C₂-C₅ alkylcarbonyl, C₂-C₅ haloalkylcarbonyl, C₄-C₇ cycloalkylcarbonyl, C₂-C₅ alkoxycarbonyl, C₃-C₅ alkoxycarbonylalkyl, C₂-C₅ alkylaminocarbonyl or C₃-C₅ dialkylaminocarbonyl; or
R^{20a} and R^{20b} are taken together to form a 5- to 6-membered fully saturated heterocyclic ring, each ring containing ring members, in addition to the connecting nitrogen atom, selected from carbon atoms and up to 2 heteroatoms independently selected from up to 2 O, up to 2 S and up to 2 N atoms, each ring optionally substituted with up to 2 methyl groups;
each R²¹ and R²³ is independently H, cyano, halogen, C₁-C₃ alkyl, C₁-C₃ haloalkyl, C₃-C₆ cycloalkyl or C₁-C₃ alkoxy; or phenyl optionally substituted with up to 2 substituents independently selected from halogen and C₁-C₃ alkyl;
each R²² is independently H, C₁-C₅ alkyl, C₁-C₅ haloalkyl, C₂-C₅ alkenyl, C₂-C₅ haloalkenyl, C₂-C₅ alkynyl, C₃-C₆ cycloalkyl, C₃-C₆ halocycloalkyl, C₂-C₅ alkylcarbonyl or C₂-C₅ alkoxy carbonyl; or
each R²² is phenyl optionally substituted with up to 2 substituents independently selected halogen and C₁-C₃ alkyl; or a 5- to 6-membered fully saturated heterocyclic ring, each ring containing ring members selected from carbon atoms and up to 2 heteroatoms independently selected from up to 2 O, up to 2 S and up to 2 N atoms, each ring optionally substituted with up to 2 substituents independently selected from halogen and C₁-C₃ alkyl;
each R²⁴ is independently H, cyano, C₁-C₃ alkyl, C₁-C₃ haloalkyl, C₁-C₄ alkoxy, C₂-C₄ alkylcarbonyl or C₂-C₄ alkoxy carbonyl;
each R²⁵ and R²⁶ is independently H, cyano, hydroxy, C₁-C₄ alkyl, C₁-C₄ haloalkyl, C₂-C₄ alkylcarbonyl, C₂-C₄ haloalkylcarbonyl, C₂-C₄ alkoxycarbonyl or C₂-C₄ haloalkoxy carbonyl;
each R²⁷ is independently halogen, cyano, hydroxy, nitro, C₁-C₄ alkyl, C₁-C₄ haloalkyl, C₂-C₄ alkenyl, C₁-C₄ alkoxy, C₂-C₄ alkylcarbonyl or C₂-C₄ alkoxycarbonyl; Z is a direct bond, O, S(═O)ₘ, N(R²⁸), C(═O), C(═O)N(R²⁸), NR²⁸C(═O), N(R²⁸)C(═O)N(R²⁸), N(R²⁸)C(═S)N(R²⁸), OC(═O)N(R²⁸), N(R²⁸)C(═O)O, S(O)₂N(R²⁸), N(R²⁸)S(═O)₂ or N(R²⁸)S(O)₂N(R²⁸), wherein the atom to the right is connected to L;
each R²⁸ is independently H, C₁-C₃ alkyl, C₁-C₃ alkoxy, C₂-C₃ alkylcarbonyl or C₂-C₃ alkoxycarbonyl; and
m is 0, 1 or 2;
provided that:
(a) when A¹ is N(R^{7a}), O or S, then A² is a direct bond or CR^{6e}R^{6f}; and
(b) when A² is N(R^{7b}), O or S; then A¹ is CR^{6c}R^{6d}.

More particularly, this invention pertains to a compound of Formula 1 (including all stereoisomers), tautomers, a tautomer, an N-oxide or a salt thereof.

This invention also relates to a fungicidal composition comprising (a) a compound of the invention (i.e. in a fungicidally effective amount); and (b) at least one additional component selected from the group consisting of surfactants, solid diluents and liquid diluents.

This invention also relates to a fungicidal composition comprising (a) a compound of the invention; and (b) at least one other fungicide (e.g., at least one other fungicide having a different site of action).

This invention further relates to a method for controlling plant diseases caused by fungal plant pathogens comprising applying to the plant or portion thereof, or to the plant seed, a fungicidally effective amount of a compound of the invention (e.g., as a composition described herein).

This invention also relates to a composition comprising a compound of Formula 1, an N-oxide, or a salt thereof, and at least one invertebrate pest control compound or agent.

The invention also relates to compounds of Formula 10 (including all stereoisomers), N-oxides, and salts thereof:

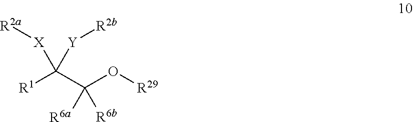

wherein
R²⁹ is S(═O)₂R³⁰;
R³⁰ is C₁-C₄ alkyl, C₁-C₄ haloalkyl, phenyl, 4-methylphenyl 4-bromophenyl or 4-nitrophenyl; and
R¹, R^{2a}, R^{2b}, X, Y, R^{6a} and R^{6b} are as defined above for above for Formula 1.
   Compounds of Formula 10 can be used as process intermediates to prepare compounds of Formula 1.

### DETAILS OF THE INVENTION

As used herein, the terms “comprises,” “comprising,” “includes,” “including,” “has,” “having,” “contains,” “containing,” “characterized by” or any other variation thereof, are intended to cover a non-exclusive inclusion, subject to any limitation explicitly indicated. For example, a composition, mixture, process, method, article, or apparatus that comprises a list of elements is not necessarily limited to only those elements but may include other elements not expressly listed or inherent to such composition, mixture, process, method, article, or apparatus.

The transitional phrase “consisting of” excludes any element, step, or ingredient not specified. If in the claim, such would close the claim to the inclusion of materials other than those recited except for impurities ordinarily associated therewith. When the phrase “consisting of” appears in a clause of the body of a claim, rather than immediately following the preamble, it limits only the element set forth in that clause; other elements are not excluded from the claim as a whole.

The transitional phrase “consisting essentially of” is used to define a composition, method or apparatus that includes materials, steps, features, components, or elements, in addition to those literally disclosed, provided that these additional materials, steps, features, components, or elements do not materially affect the basic and novel characteristic(s) of the claimed invention.

The term “consisting essentially of” occupies a middle ground between “comprising” and “consisting of”.

Where applicants have defined an invention or a portion thereof with an open-ended term such as “comprising,” it should be readily understood that (unless otherwise stated) the description should be interpreted to also describe such an invention using the terms “consisting essentially of” or “consisting of.”

Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

As referred to in the present disclosure and claims, “plant” includes members of Kingdom Plantae, particularly seed plants (Spermatopsida), at all life stages, including young plants (e.g., germinating seeds developing into seedlings) and mature, reproductive stages (e.g., plants producing flowers and seeds). Portions of plants include geotropic members typically growing beneath the surface of the growing medium (e.g., soil), such as roots, tubers, bulbs and corms, and also members growing above the growing medium, such as foliage (including stems and leaves), flowers, fruits and seeds.

As referred to herein, the term “seedling”, used either alone or in a combination of words means a young plant developing from the embryo of a seed.

As referred to herein, the term “broadleaf” used either alone or in words such as “broadleaf crop” means dicot or dicotyledon, a term used to describe a group of angiosperms characterized by embryos having two cotyledons.

As referred to in this disclosure, the terms “fungal pathogen” and “fungal plant pathogen” include pathogens in the Ascomycota, Basidiomycota and Zygomycota phyla, and the fungal-like Oomycota class that are the causal agents of a broad spectrum of plant diseases of economic importance, affecting ornamental, turf, vegetable, field, cereal and fruit crops. In the context of this disclosure, “protecting a plant from disease” or “control of a plant disease” includes preventative action (interruption of the fungal cycle of infection, colonization, symptom development and spore production) and/or curative action (inhibition of colonization of plant host tissues).

As used herein, the term “mode of action” (MO A) is as define by the Fungicide Resistance Action Committee (FRAC), and is used to distinguish fungicides according to their biochemical mode of action in the biosynthetic pathways of plant pathogens, and their resistance risk. FRAC-defined modes of actions include (A) nucleic acid synthesis, (B) mitosis and cell division, (C) respiration, (D) amino acid and protein synthesis, (E) signal transduction, (F) lipid synthesis and membrane integrity, (G) sterol biosynthesis in membranes, (H) cell wall biosynthesis, (I) melanin synthesis in cell wall, (P) host plant defense induction, (U) unknown mode of action, (NC) not classified, (M) multi-site contact activity and (BM) biologicals with multiple modes of action. Each mode of action (i.e. letters A through BM) contain one or more subgroups (e.g., A includes subgroups A1, A2, A3 and A4) based either on individual validated target sites of action, or in cases where the precise target site is unknown, based on cross resistance profiles within a group or in relation to other groups. Each of these subgroups (e.g., A1, A2, A3 and A4) is assigned a FRAC code (a number and/or letter). For example, the FRAC code for subgroup A1 is 4. Additional information on target sites and FRAC codes can be obtained from publicly available databases maintained, for example, by FRAC.

As used herein, the term “cross resistance” refers to the phenomenon that occurs when a pathogen develops resistance to one fungicide and simultaneously becomes resistant to one or more other fungicides. These other fungicides are typically, but not always, in the same chemical class or have the same target site of action, or can be detoxified by the same mechanism.

Generally when a molecular fragment (i.e. radical) is denoted by a series of atom symbols (e.g., C, H, N, O and S) the implicit point or points of attachment will be easily recognized by those skilled in the art. In some instances herein, particularly when alternative points of attachment are possible, the point or points of attachment may be explicitly indicated by a hyphen (“-”). The dotted line in rings depicted in the present description (e.g., the rings G-44, G-45, G-48 and G-49 shown in Exhibit A) represents that the bond indicated can be a single bond or double bond.

In the above recitations, the term “alkyl”, used either alone or in compound words such as “alkylthio” or “haloalkyl” includes straight-chain and branched alkyl, such as, methyl, ethyl, n-propyl, i-propyl, and the different butyl, pentyl and hexyl isomers. “Alkenyl” includes straight-chain and branched alkenes such as ethenyl, 1-propenyl, 2-propenyl, and the different butenyl, pentenyl and hexenyl isomers. “Alkenyl” also includes polyenes such as 1,2-propadienyl and 2,4-hexadienyl. “Alkynyl” includes straight-chain and branched alkynes such as ethynyl, 1-propynyl, 2-propynyl, and the different butynyl, pentynyl and hexynyl isomers. “Alkynyl” can also include moieties comprised of multiple triple bonds such as 2,5-hexadiynyl. “Alkylene” denotes a straight-chain or branched alkanediyl. Examples of “alkylene” include CH₂, CH₂CH₂, CH(CH₃), CH₂CH₂CH₂, CH₂CH(CH₃), and the different butylene isomers. “Alkenylene” denotes a straight-chain or branched alkenediyl containing one olefinic bond. Examples of “alkenylene” include CH═CH, CH₂CH═CH, CH═C(CH₃) and the different butenylene isomers. “Alkynylene” denotes a straight-chain or branched alkynediyl containing one triple bond. Examples of “alkynylene” include CH₂CAC, CACCH₂, and the different butynylene, pentynylene or hexynylene isomers. The term “cycloalkylene” denotes a cycloalkanediyl ring. Examples of “cycloalkylene” include cyclobutanediyl, cyclopentanediyl and cyclohexanediyl. The term “cycloalkenylene” denotes a cycloalkenediyl ring containing one olefinic bond. Examples of “cycloalkenylene” include cyclopropenediyl and cyclopentenediyl.

“Alkoxy” includes, for example, methoxy, ethoxy, n-propyloxy, i-propyloxy, and the different butoxy, pentoxy and hexyloxy isomers. “Alkenyloxy” includes straight-chain and branched alkenyl attached to and linked through an oxygen atom. Examples of “alkenyloxy” include H₂C═CHCH₂O and CH₃CH═CHCH₂O. “Alkynyloxy” includes straight-chain and branched alkynyl attached to and linked through an oxygen atom. Examples of “alkynyloxy” include HC═CCH₂O and CH₃C═CCH₂O.

The term “alkylthio” includes straight-chain and branched alkylthio moieties such as methylthio, ethylthio, and the different propylthio and butylthio isomers. “Alkylsulfinyl” includes both enantiomers of an alkylsulfinyl group. Examples of “alkylsulfinyl” include CH₃S(═O), CH₃CH₂S(═O), CH₃CH₂CH₂S(═O), (CH₃)₂CHS(═O), and the different butylsulfinyl isomers. Examples of “alkylsulfonyl” include CH₃S(═O)₂, CH₃CH₂S(═O)₂, CH₃CH₂CH₂S(═O)₂, (CH₃)₂CHS(═O)₂, and the different butylsulfonyl isomers. “Alkenylthio” includes straight-chain and branched alkenyl attached to and linked through a sulfur atom. Examples of “alkenylthio” include H₂C═CHCH₂S and CH₃CH═CHCH₂S. “Alkenylsulfinyl” includes both enantiomers of an alkenylsulfinyl group. Examples of “alkenylsulfinyl” include H₂C═CHCH₂S(═O), CH₃CH═CHCH₂S(═O), (CH₃)₂C═CHCH₂S(═O), and the different butynylsulfinyl isomers. Examples of “alkenylsulfonyl” include CH₃CH═CHS(═O)₂, (CH₃)₂C═CHCH₂S(═O)₂, and the different butynylsulfonyl isomers. “Alkynylthio” includes straight-chain and branched alkynyl attached to and linked through a sulfur atom. Examples of “alkynylthio” include HC≡CCH₂S and CH₃C≡CCH₂S. “Alkynylsulfinyl” includes both enantiomers of an alkynylsulfinyl group. Examples of “alkynylsulfinyl” include HC≡CCH₂S(═O), CH₃C═CCH₂S(═O), and the different butenylsulfinyl isomers. Examples of “alkynylsulfonyl” include CH₃C═CS(═O)₂, CH₃C═CCH₂S(═O)₂, and the different butenylsulfonyl isomers.

“Alkylthioalkyl” denotes alkylthio substitution on alkyl. Examples of “alkylthioalkyl” include CH₃SCH₂, CH₃SCH₂CH₂, CH₃CH₂SCH₂, CH₃CH₂CH₂SCH₂ and CH₃CH₂SCH₂CH₂; “alkylsulfinylalkyl” and “alkylsulfonylalkyl” include the corresponding sulfoxides and sulfones, respectively.

“(Alkylthio)carbonyl” denotes a straight-chain or branched alkylthio group bonded to a C(═O) moiety. Examples of “(alkylthio)carbonyl” include CH₃SC(═O), CH₃CH₂CH₂SC(═O) and (CH₃)₂CHSC(═O). The terms “(alkenylthio)carbonyl” and “(alkynylthio)carbonyl” are likewise defined. Examples of “(alkenylthio)carbonyl” include H₂C═CHCH₂SC(═O) and CH₃CH₂CH═CHSC(═O). Examples of “(alkynylthio)carbonyl” include HC≡CCH₂SC(═O) and CH₃C≡CCH₂SC(═O).

“Alkyl(thiocarbonyl)” denotes a straight-chain or branched alkyl group bonded to a C(═S) moiety. Examples of “alkyl(thiocarbonyl)” include CH₃CH₂C(═S), CH₃CH₂CH₂C(═S) and (CH₃)₂CHCH₂C(═S). The terms “alkenyl(thiocarbonyl)” and “alkynyl(thiocarbonyl)” are likewise defined. Examples of “alkenyl(thiocarbonyl)” include H₂C═CHCH₂CH₂C(═S) and CH₃CH₂CH═CHC(═S). Examples of “alkynyl(thiocarbonyl)” include HC═CCH₂SC(═O) and CH₃C═CCH₂C(═S).

“Alkylamino(thiocarbonyl)” denotes a straight-chain or branched alkylamino group bonded to a C(═S) moiety. Examples of “alkylamino(thiocarbonyl)” include CH₃NHC(═S), CH₃CH₂CH₂NHC(═S) and (CH₃)₂CHNHC(═S). The terms “alkenylamino(thiocarbonyl)” and “alkynylamino(thiocarbonyl)” are likewise defined. Examples of “alkenylamino(thiocarbonyl)” include H₂C═CHCH₂CH₂NHC(═S) and CH₃CH₂CH═CHNHC(═S). Examples of “alkynylamino(thiocarbonyl)” include HC≡CCH₂CH₂NHC(═S) and CH₃C≡CCH₂NHC(═S).

“(Alkylthio)carbonylamino” denotes a straight-chain or branched alkylthio group bonded to a C(═O)NH moiety. Examples of “(alkylthio)carbonylamino” include CH₃CH₂SC(═O)NH, CH₃CH₂CH₂SC(═O)NH and (CH₃)₂CHSC(═O)NH. The terms “(alkenylthio)carbonylamino” and “(alkynylthio)carbonylamino” are likewise defined. Examples of “(alkenylthio)carbonylamino include H₂C═CHCH₂SC(═O)NH and CH₃CH═CHSC(═O)NH.

Examples of “(alkynylthio)carbonylamino” include HC≡CCH₂CH₂SC(═O)NH and CH₃C≡CCH₂CH₂SC(═O)NH.

“Alkylamino” includes an NH radical substituted with a straight-chain or branched alkyl group. Examples of “alkylamino” include CH₃CH₂NH, CH₃CH₂CH₂NH, and (CH₃)₂CHCH₂NH. Examples of “dialkylamino” include (CH₃)₂N, (CH₃CH₂CH₂)₂N and CH₃CH₂(CH₃)N. “Alkylaminoalkyl” denotes alkylamino substitution on alkyl. Examples of “alkylammoalkyl” include CH₃NHCH₂, CH₃NHCH₂CH₂, CH₃CH₂NHCH₂, CH₃CH₂CH₂CH₂NHCH₂ and CH₃CH₂NHCH₂CH₂.

“Alkylcarbonyl” denotes a straight-chain or branched alkyl group bonded to a C(═O) moiety. Examples of “alkylcarbonyl” include CH₃C(═O), CH₃CH₂CH₂C(═O) and (CH₃)₂CHC(═O). The terms “alkenylcarbonyl” and “alkynylcarbonyl” are likewise defined. Examples of “alkenylcarbonyl” include H₂C═CHCH₂C(═O) and CH₃CH₂CH═CHC(═O). Examples of “alkynylcarbonyl” include HC≡CCH₂C(═O) and CH₃C≡CCH₂C(═O). “Alkoxycarbonyl” includes a C(═O) moiety substituted with a straight-chain or branched alkoxy group. Examples of “alkoxycarbonyl” include CH₃OC(═O), CH₃CH₂C(═O), CH₃CH₂CH₂OC(═O), (CH₃)₂CHOC(═O), and the different butoxy- and pentoxycarbonyl isomers. The terms “alkenyloxycarbonyl” and “alkynyloxycarbonyl” are likewise defined. Examples of “alkenyloxycarbonyl” include H₂C═CHCH₂OC(═O) and CH₃CH₂CH═CHOC(═O). Examples of “alkynyloxycarbonyl” include HC≡CCH₂OC(═O) and CH₃C≡CCH₂OC(═O).

“Alkylaminocarbonyl” denotes a straight-chain or branched alkyl group bonded to a NHC(═O) moiety. Examples of “alkylaminocarbonyl” include CH₃NHC(═O), CH₃CH₂NHC(═O), CH₃CH₂CH₂NHC(═O), (CH₃)₂CHNHC(═O), and the different butylamino- and pentylaminocarbonyl isomers. The terms “alkenylaminocarbonyl” and “alkynylaminocarbonyl” are likewise defined. Examples of “alkenylaminocarbonyl” include H₂C═CHCH₂NHC(═O) and (CH₃)₂C═CHCH₂NHC(═O). Examples of “alkynylaminocarbonyl” include CH₃C≡CNHC(═O) and CH₃C≡CCH₂NHC(═O). Examples of “dialkylaminocarbonyl” include (CH₃)₂N(═O), (CH₃CH₂)₂NC(═O), CH₃CH₂(CH₃)NC(═O), (CH₃)₂CH(CH₃)NC(═O) and CH₃CH₂CH₂(CH₃)NC(═O).

The term “alkylcarbonylamino” denotes a straight-chain or branched alkyl group bonded to a C(═O)NH moiety. Examples of “alkylcarbonylamino” include CH₃CH₂C(═O)NH and CH₃CH₂CH₂C(═O)NH. The terms “alkenylcarbonylamino” and “alkynylcarbonylamino” are likewise defined. Examples of “alkenylcarbonylamino” include H₂C═CHCH₂C(═O)NH and (CH₃)₂C═CHCH₂C(═O)NH. Examples of “alkynylcarbonylamino” include CH₃C≡CCH(CH₃)C(═O)NH and HC≡CCH₂CH₂C(═O)NH. The term “alkoxycarbonylamino” denotes alkoxy bonded to a C(═O)NH moiety. Examples of “alkoxycarbonylamino” include CH₃OC(═O)NH and CH₃CH₂OC(═O)NH.

The term “alkylaminocarbonylamino” denotes a straight-chain or branched alkyl group bonded to a NHC(═O)NH moiety. Examples of “alkylaminocarbonylamino” include CH₃CH₂NHC(═O)NH and (CH₃CH₂)₂CH₂NHC(═O)NH. The terms “alkenylaminocarbonylamino” and “alkynylaminocarbonylamino” are likewise defined. Examples of “alkenylaminocarbonylamino” include H₂C═CHCH₂NHC(═O)NH and (CH₃)₂C═CHCH₂NHC(═O)NH. Examples of “alkynylaminocarbonylamino” include CH₃C≡CCH(CH₃)NHC(═O)NH and HC≡CCH₂CH₂NHC(═O)NH.

“Alkylsulfonylamino” denotes an NH radical substituted with alkylsulfonyl. Examples of “alkylsulfonylamino” include CH₃CH₂S(═O)₂NH and (CH₃)₂CHS(═O)₂NH. The terms “alkenylsulfonylamino” and “alkynylsulfonylamino” are likewise defined. Examples of “alkenylsulfonylammo” include H₂C═CHCH₂CH₂S(═O)₂NH and (CH₃)₂C═CHCH₂S(═O)₂NH. Examples of “alkynylsulfonylamino” include CH₃C═CCH(CH₃)S(═O)₂NH and HC≡CCH₂CH₂S(═O)₂NH. The term “alkylsulfonyloxy” denotes an alkylsulfonyl group bonded to an oxygen atom. Examples of “alkylsulfonyloxy” include CH₃S(═O)₂O, CH₃CH₂S(═O)₂O, CH₃CH₂CH₂S(═O)₂O, (CH₃)₂CHS(═O)₂O, and the different butylsulfonyloxy, pentylsulfonyloxy and hexylsulfonyloxy isomers.

“Alkylaminosulfonyl” denotes a straight-chain or branched alkyl group bonded to a NHS(═O)₂ moiety. Examples of “alkylaminosulfonyl” include CH₃CH₂NHS(═O)₂ and (CH₃)₂CHNHS(═O)₂. The terms “alkenylaminosulfonyl” and “alkynylaminosulfonyl” are likewise defined. Examples of “alkenylaminosulfonyl” include H₂C═CHCH₂CH₂NHS(═O)₂ and (CH₃)₂C═CHCH₂NHS(═O)₂. Examples of “alkynylaminosulfonyl” include CH₃C≡CCH(CH₃)NHS(═O)₂ and HC≡CCH₂CH₂NHS(═O)₂.

“Alkylaminosulfonylamino” denotes a straight-chain or branched alkyl group bonded to a NHS(═O)₂NH moiety. Examples of “alkylaminosulfonylamino” include CH₃CH₂NHS(═O)₂NH and (CH₃)₂CHNHS(═O)₂NH. The terms “alkenylaminosulfonylamino” and “alkynylaminosulfonylamino” are likewise defined. Examples of “alkenylaminosulfonylamino” include H₂C═CHCH₂CH₂NHS(═O)₂NH and (CH₃)₂C═CHCH₂NHS(═O)₂NH. Examples of “alkynylaminosulfonylamino” include CH₃C≡CCH(CH₃)NHS(═O)₂NH and HC≡CCH₂CH₂NHS(═O)₂NH.

“Alkoxyalkyl” denotes alkoxy substitution on alkyl. Examples of “alkoxyalkyl” include CH₃OCH₂, CH₃OCH₂CH₂, CH₃CH₂OCH₂, CH₃CH₂CH₂OCH₂ and CH₃CH₂OCH₂CH₂.

“Alkoxyalkoxy” denotes alkoxy substitution on another alkoxy moiety. “Alkoxyalkoxyalkyl” denotes alkoxyalkoxy substitution on alkyl. Examples of “alkoxyalkoxyalkyl” include CH₃OCH₂OCH₂, CH₃OCH₂OCH₂CH₂ and CH₃CH₂OCH₂OCH₂.

The term “alkylcarbonyloxy” denotes a straight-chain or branched alkyl bonded to a C(═O)O moiety. Examples of “alkylcarbonyloxy” include CH₃CH₂C(═O)O and (CH₃)₂CHC(═O)O. The terms “alkenylcarbonyloxy” and “alkynylcarbonyloxy” are likewise defined. Examples of “alkenylcarbonyloxy” include H₂C═CHCH₂CH₂C(═O)O and (CH₃)₂C═CHCH₂C(═O)O. Examples of “alkynylcarbonyloxy” include CH₃C≡CCH(CH₃)C(═O)O and HC≡CCH₂CH₂C(═O)O. The term “alkoxycarbonyloxy” denotes a straight-chain or branched alkoxy bonded to a C(═O)O moiety. Examples of “alkoxycarbonyloxy” include CH₃CH₂CH₂OC(═O)O and (CH₃)₂CHOC(═O)O. The term “alkoxycarbonylalkyl” denotes alkoxycarbonyl substitution on alkyl. Examples of “alkoxycarbonylalkyl” include CH₃CH₂OC(═O)CH₂, (CH₃)₂CHOC(═O)CH₂ and CH₃OC(═O)CH₂CH₂. The term “alkylaminocarbonyloxy” denotes a straight-chain or branched alkylaminocarbonyl attached to and linked through an oxygen atom. Examples of “alkylaminocarbonyloxy” include (CH₃)₂CHCH₂NHC(═C))C) and CH₃CH₂NHC(═C))C). The terms “alkenylaminocarbonyloxy” and “alkynylaminocarbonyloxy” are likewise defined.

The term “alkylcarbonylthio” denotes a straight-chain or branched alkyl group bonded to a C(═O)S moiety. Examples of “alkylcarbonylthio” include CH₃CH₂C(═O)S and CH₃CH₂CH₂C(═O)S.

The term “cycloalkylalkyl” denotes cycloalkyl substitution on an alkyl moiety. Examples of “cycloalkylalkyl” include cyclopropylmethyl, cyclopentylethyl, and other cycloalkyl moieties bonded to a straight-chain or branched alkyl group. The term “alkylcycloalkyl” denotes alkyl substitution on a cycloalkyl moiety and includes, for example, ethylcyclopropyl, i-propylcyclobutyl, methylcyclopentyl and methylcyclohexyl. “Alkylcycloalkylalkyl” denotes alkylcycloalkyl substitution on alkyl. Examples of “alkylcycloalkylalkyl” include methylcyclohexylmethyl and ethylcycloproylmethyl. “Cycloalkenyl” includes groups such as cyclopentenyl and cyclohexenyl as well as groups with more than one double bond such as 1,3- or 1,4-cyclohexadienyl. The term “cycloalkylcycloalkyl” denotes cycloalkyl substitution on another cycloalkyl ring, wherein each cycloalkyl ring independently has from 3 to 7 carbon atom ring members. Examples of cycloalkylcycloalkyl include cyclopropylcyclopropyl (such as 1,1′-bicyclopropyl-1-yl, 1,1′-bicyclopropyl-2-yl), cyclohexylcyclopentyl (such as 4-cyclopentylcyclohexyl) and cyclohexylcyclohexyl (such as 1,1′-bicyclohexyl-1-yl), and the different cis- and trans-cycloalkylcycloalkyl isomers, (such as (1R,2S)-1,1′-bicyclopropyl-2-yl and (1R,2R)-1,1′-bicyclopropyl-2-yl).

The term “cycloalkoxy” denotes cycloalkyl attached to and linked through an oxygen atom including, for example, cyclopentyloxy and cyclohexyloxy. The term “cycloalkoxyalkyl” denotes cycloalkoxy substitution on an alkyl moiety. Examples of “cycloalkoxyalkyl” include cyclopropyloxymethyl, cyclopentyloxyethyl, and other cycloalkoxy groups bonded to a straight-chain or branched alkyl moiety.

The term “cycloalkylaminoalkyl” denotes cycloalkylamino substitution on an alkyl group. Examples of “cycloalkylaminoalkyl” include cyclopropylaminomethyl, cyclopentylaminoethyl, and other cycloalkylamino moieties bonded to a straight-chain or branched alkyl group.

“Cycloalkylcarbonyl” denotes cycloalkyl bonded to a C(═O) group including, for example, cyclopropylcarbonyl and cyclopentylcarbonyl. “Cycloalkylcarbonyloxy” denotes cycloalkylcarbonyl attached to and linked through an oxygen atom. Examples of “cycloalkylcarbonyloxy” include cyclohexylcarbonyloxy and cyclopentylcarbonyloxy. The term “cycloalkoxycarbonyl” means cycloalkoxy bonded to a C(═O) group, for example, cyclopropyloxycarbonyl and cyclopentyloxycarbonyl. “Cycloalkylaminocarbonylamino” denotes cycloalkylamino bonded to a C(═O)NH group, for example, cyclopentylaminocarbonylamino and cyclohexylaminocarbonylamino.

The term “halogen”, either alone or in compound words such as “haloalkyl”, or when used in descriptions such as “alkyl substituted with halogen” includes fluorine, chlorine, bromine or iodine. Further, when used in compound words such as “haloalkyl”, or when used in descriptions such as “alkyl substituted with halogen” said alkyl may be partially or fully substituted with halogen atoms which may be the same or different. Examples of “haloalkyl” or “alkyl substituted with halogen” include CF₃, ClCH₂, CF₃CH₂ and CF₃CCl₂. The terms “haloalkenyl”, “haloalkynyl” “haloalkoxy”, “haloalkylsulfonyl”, “halocycloalkyl”, and the like, are defined analogously to the term “haloalkyl”. Examples of “haloalkenyl” include Cl₂C═CHCH₂ and CF₃CH₂CH═CHCH₂. Examples of “haloalkynyl” include HC≡CCHCl, CF₃C≡C, CCl₃C≡C and FCH₂C≡CCH₂. Examples of “haloalkoxy” include CF₃O, CCl₃CH₂O, F₂CHCH₂CH₂O and CF₃CH₂O. Examples of “haloalkylsulfonyl” include CF₃S(═O)₂, CCl₃S(═O)₂, CF₃CH₂S(═O)₂ and CF₃CF₂S(═O)₂. Examples of “halocycloalkyl” include 2-chlorocyclopropyl, 2-fluorocyclobutyl, 3-bromocyclopentyl and 4-chorocyclohexyl.

“Cyanoalkyl” denotes an alkyl group substituted with one cyano group. Examples of “cyanoalkyl” include NCCH₂, NCCH₂CH₂ and CH₃CH(CN)CH₂. “Hydroxyalkyl” denotes an alkyl group substituted with one hydroxy group. Examples of “hydroxyalkyl” include HOCH₂CH₂, CH₃CH₂(OH)CH and HOCH₂CH₂CH₂CH₂.

“Trialkylsilyl” includes 3 branched and/or straight-chain alkyl radicals attached to and linked through a silicon atom, such as trimethylsilyl, triethylsilyl and tert-butyl dimethylsilyl.

The total number of carbon atoms in a substituent group is indicated by the “Cᵢ-Cⱼ” prefix where i and j are numbers from 1 to 15. For example, C₁-C₄ alkylsulfonyl designates methylsulfonyl through butylsulfonyl; C₂ alkoxyalkyl designates CH₃OCH₂; C₃ alkoxyalkyl designates, for example, CH₃CH(OCH₃), CH₃OCH₂CH₂ or CH₃CH₂OCH₂; and C₄ alkoxyalkyl designates the various isomers of an alkyl group substituted with an alkoxy group containing a total of four carbon atoms, examples including CH₃CH₂CH₂OCH₂ and CH₃CH₂OCH₂CH₂.

The term “unsubstituted” in connection with a group such as a ring or ring system means the group does not have any substituents other than its one or more attachments to the remainder of Formula 1. The term “optionally substituted” means that the number of substituents can be zero. Unless otherwise indicated, optionally substituted groups may be substituted with as many optional substituents as can be accommodated by replacing a hydrogen atom with a non-hydrogen substituent on any available carbon or nitrogen atom. Commonly, the number of optional substituents (when present) ranges from 1 to 3. As used herein, the term “optionally substituted” is used interchangeably with the phrase “substituted or unsubstituted” or with the term “(un)substituted.”

The number of optional substituents may be restricted by an expressed limitation. For example, the phrase “optionally substituted with up to 3 substituents independently selected from R¹³” means that 0, 1, 2 or 3 substituents can be present (if the number of potential connection points allows). When a range specified for the number of substituents (e.g., x being an integer from 0 to 3 in Exhibit A) exceeds the number of positions available for substituents on a ring (e.g., 1 position available for (R¹³)ₓ on G-7 in Exhibit A), the actual higher end of the range is recognized to be the number of available positions.

When a compound is substituted with a substituent bearing a subscript that indicates the number of said substituents can vary (e.g., (R¹³)ₓ in Exhibit A wherein x is 1 to 3), then said substituents are independently selected from the group of defined substituents, unless otherwise indicated. When a variable group is shown to be optionally attached to a position, for example (R¹³)ₓ in Exhibit A wherein x may be 0, then hydrogen may be at the position even if not recited in the definition of the variable group.

Naming of substituents in the present disclosure uses recognized terminology providing conciseness in precisely conveying to those skilled in the art the chemical structure. For sake of conciseness, locant descriptors may be omitted.

Unless otherwise indicated, a “ring” or “ring system” as a component of Formula 1 (e.g., G) is carbocyclic or heterocyclic. The term “ring system” denotes two or more connected rings. The term “spirocyclic ring system” denotes a ring system consisting of two rings connected at a single atom (so the rings have a single atom in common). The term “bicyclic ring system” denotes a ring system consisting of two rings sharing two or more common atoms. In a “fused bicyclic ring system” the common atoms are adjacent, and therefore the rings share two adjacent atoms and a bond connecting them.

The term “ring member” refers to an atom (e.g., C, O, N or S) or other moiety (e.g., C(═O), C(═S), S(═O) and S(═O)₂) forming the backbone of a ring or ring system. The term “aromatic” indicates that each of the ring atoms is essentially in the same plane and has a p-orbital perpendicular to the ring plane, and that (4n+2) π electrons, where n is a positive integer, are associated with the ring to comply with Hückel's rule

The term “carbocyclic ring” denotes a ring wherein the atoms forming the ring backbone are selected only from carbon. Unless otherwise indicated, a carbocyclic ring can be a saturated, partially unsaturated, or fully unsaturated ring. When a fully unsaturated carbocyclic ring satisfies Hückel's rule, then said ring is also called an “aromatic ring”. “Saturated carbocyclic” refers to a ring having a backbone consisting of carbon atoms linked to one another by single bonds; unless otherwise specified, the remaining carbon valences are occupied by hydrogen atoms.

As used herein, the term “partially unsaturated ring” or “partially unsaturated heterocycle” refers to a ring which contains unsaturated ring atoms and one or more double bonds but is not aromatic.

The terms “heterocyclic ring” or “heterocycle” denotes a ring wherein at least one of the atoms forming the ring backbone is other than carbon. Unless otherwise indicated, a heterocyclic ring can be a saturated, partially unsaturated, or fully unsaturated ring. When a fully unsaturated heterocyclic ring satisfies Hückel's rule, then said ring is also called a “heteroaromatic ring” or aromatic heterocyclic ring. “Saturated heterocyclic ring” refers to a heterocyclic ring containing only single bonds between ring members.

Unless otherwise indicated, heterocyclic rings and ring systems are attached to the remainder of Formula 1 through any available carbon or nitrogen atom by replacement of a hydrogen on said carbon or nitrogen atom.

Compounds of this invention can exist as one or more stereoisomers. Stereoisomers are isomers of identical constitution but differing in the arrangement of their atoms in space and include enantiomers, diastereomers, cis- and trans-isomers (also known as geometric isomers) and atropisomers. Atropisomers result from restricted rotation about single bonds where the rotational barrier is high enough to permit isolation of the isomeric species. One skilled in the art will appreciate that one stereoisomer may be more active and/or may exhibit beneficial effects when enriched relative to the other stereoisomer(s) or when separated from the other stereoisomer(s). Additionally, the skilled artisan knows how to separate, enrich, and/or to selectively prepare said stereoisomers. For a comprehensive discussion of all aspects of stereoisomerism, see Ernest L. Eliel and Samuel H. Wilen, Stereochemistry of Organic Compounds, John Wiley & Sons, 1994.

Compounds of this invention may be present as a mixture of stereoisomers, individual stereoisomers, or as an optically active form. For example, when T is T-3, then Formula 1 compounds contain at least one double bond and the configuration of substituents about that double bond can be (Z) or (E) (cis or trans), or a mixture thereof. In the context of the present disclosure and claims, a wavy bond (e.g., as shown in the T-3 moiety in the Summary of the Invention) indicates a single bond which is linked to an adjacent double bond wherein the geometry about the adjacent double bond is either (Z)-configuration (syn-isomer or cis-isomer) or (E)-configuration (anti-isomer or trans-isomer), or a mixture thereof. That is, a wavy bond represents an unspecified (Z)- or (E)- (cis- or trans-) isomer, or mixture thereof. In addition, the compounds of the present invention can contain one or more chiral centers and therefore exist in enantiomeric and diastereomeric forms. Unless the structural formula or the language of this application specifically designate a particular cis- or trans-isomer, or a configuration of a chiral center, the scope of the present invention is intended to cover all such isomers per se, as well as mixtures of cis- and trans-isomers, mixtures of diastereomers and racemic mixtures of enantiomers (optical isomers) as well.

This invention also includes compounds of Formula 1 wherein one stereoisomer is enriched relative to the other stereoisomer(s). Of note are compounds of Formula 1 wherein T is T-3 and the substituents attached to the double bond in the T-3 moiety are in a predominately (Z)-configuration, or predominately an (E)-configuration. The ratio of the (Z)- to (E)-isomers in any compounds of Formula 1, whether produced stereoselectivity or non-stereoselectivity, may take on a broad range of values. For example, compounds of Formula 1 may comprise from about 10 to 90 percent by weight of the (Z)-isomer to about 90 to 10 percent by weight of the (E)-isomer. In other embodiments, Formula 1 compounds may contain from about 15 to 85 percent by weight of the (Z)-isomer and about 85 to 15 percent by weight of the (E)-isomer; in another embodiment, the mixture contains about 25 to 75 percent by weight of the (Z)-isomer and about 75 to 25 percent by weight of the (E)-isomer; in another embodiment, the mixture contains about 35 to 65 percent by weight of the (Z)-isomer and about 65 to 35 percent by weight of the (E)-isomer; in another embodiment, the mixture contains about 45 to 55 percent by weight of the (Z)-isomer and about 55 to 45 percent by weight of the (E)-isomer. These percentages by weight are based on the total weight of the composition, and it will be understood that the sum of the weight percent of the (Z)-isomer and the (E)-isomer is 100 weight percent. In other words, compounds of Formula 1 might contain 65 percent by weight of the (Z)-isomer and 35 percent by weight of the (E)-isomer, or vice versa.

In addition, this invention includes compounds that are enriched compared to the racemic mixture in an enantiomer of Formula 1. Also included are the essentially pure enantiomers of compounds of Formula 1. When enantiomerically enriched, one enantiomer is present in greater amounts than the other, and the extent of enrichment can be defined by an expression of enantiomeric excess (“ee”), which is defined as (2x−1) 100%, where x is the mole fraction of the dominant enantiomer in the mixture (e.g., an ee of 20% corresponds to a 60:40 ratio of enantiomers).

Preferably the compositions of this invention have at least a 50% enantiomeric excess; more preferably at least a 75% enantiomeric excess; still more preferably at least a 90% enantiomeric excess; and the most preferably at least a 94% enantiomeric excess of the more active isomer. Of particular note are enantiomerically pure embodiments of the more active isomer.

Compounds of this invention can exist as one or more conformational isomers due to restricted rotation about an amide bond (e.g., C(═O)—N) in Formula 1. This invention comprises mixtures of conformational isomers. In addition, this invention includes compounds that are enriched in one conformer relative to others.

This invention comprises all stereoisomers, conformational isomers and mixtures thereof in all proportions as well as isotopic forms such as deuterated compounds.

One skilled in the art will appreciate that not all nitrogen containing heterocycles can form N-oxides since the nitrogen requires an available lone pair for oxidation to the oxide; one skilled in the art will recognize those nitrogen-containing heterocycles which can form N-oxides. One skilled in the art will also recognize that tertiary amines can form N-oxides. Synthetic methods for the preparation of N-oxides of heterocycles and tertiary amines are very well known by one skilled in the art including the oxidation of heterocycles and tertiary amines with peroxy acids such as peracetic and m-chloroperbenzoic acid (MCPBA), hydrogen peroxide, alkyl hydroperoxides such as t-butyl hydroperoxide, sodium perborate, and dioxiranes such as dimethyldioxirane. These methods for the preparation of N-oxides have been extensively described and reviewed in the literature, see for example: T. L. Gilchrist in Comprehensive Organic Synthesis, vol. 7, pp 748-750, S. V. Ley, Ed., Pergamon Press; M. Tisler and B. Stanovnik in Comprehensive Heterocyclic Chemistry, vol. 3, pp 18-20, A. J. Boulton and A. McKillop, Eds., Pergamon Press; M. R. Grimmett and B. R. T. Keene in Advances in Heterocyclic Chemistry, vol. 43, pp 149-161, A. R. Katritzky, Ed., Academic Press; M. Tisler and B. Stanovnik in Advances in Heterocyclic Chemistry, vol. 9, pp 285-291, A. R. Katritzky and A. J. Boulton, Eds., Academic Press; and G. W. H. Cheeseman and E. S. G. Werstiuk in Advances in Heterocyclic Chemistry, vol. 22, pp 390-392, A. R. Katritzky and A. J. Boulton, Eds., Academic Press.

One skilled in the art recognizes that because in the environment and under physiological conditions salts of chemical compounds are in equilibrium with their corresponding nonsalt forms, salts share the biological utility of the nonsalt forms. Thus a wide variety of salts of the compounds of Formula 1 are useful for control of plant diseases caused by fungal plant pathogens (i.e. are agriculturally suitable). The salts of the compounds of Formula 1 include acid-addition salts with inorganic or organic acids such as hydrobromic, hydrochloric, nitric, phosphoric, sulfuric, acetic, butyric, fumaric, lactic, maleic, malonic, oxalic, propionic, salicylic, tartaric, 4-toluenesulfonic or valeric acids. When a compound of Formula 1 contains an acidic moiety such as a carboxylic acid, salts also include those formed with organic or inorganic bases such as pyridine, triethylamine or ammonia, or amides, hydrides, hydroxides or carbonates of sodium, potassium, lithium, calcium, magnesium or barium. Accordingly, the present invention comprises compounds selected from Formula 1, N-oxides, and agriculturally suitable salts, and solvates thereof.

Compounds selected from Formula 1, stereoisomers, tautomers, N-oxides, and salts thereof, typically exist in more than one form, and Formula 1 thus includes all crystalline and non-crystalline forms of the compounds that Formula 1 represents. Non-crystalline forms include embodiments which are solids such as waxes and gums as well as embodiments which are liquids such as solutions and melts. Crystalline forms include embodiments which represent essentially a single crystal type and embodiments which represent a mixture of polymorphs (i.e. different crystalline types). The term “polymorph” refers to a particular crystalline form of a chemical compound that can crystallize in different crystalline forms, these forms having different arrangements and/or conformations of the molecules in the crystal lattice. Although polymorphs can have the same chemical composition, they can also differ in composition due to the presence or absence of co-crystallized water or other molecules, which can be weakly or strongly bound in the lattice. Polymorphs can differ in such chemical, physical and biological properties as crystal shape, density, hardness, color, chemical stability, melting point, hygroscopicity, suspensibility, dissolution rate and biological availability. One skilled in the art will appreciate that a polymorph of a compound represented by Formula 1 can exhibit beneficial effects (e.g., suitability for preparation of useful formulations, improved biological performance) relative to another polymorph or a mixture of polymorphs of the same compound represented by Formula 1. Preparation and isolation of a particular polymorph of a compound represented by Formula 1 can be achieved by methods known to those skilled in the art including, for example, crystallization using selected solvents and temperatures. For a comprehensive discussion of polymorphism see R. Hilfiker, Ed., Polymorphism in the Pharmaceutical Industry, Wiley-VCH, Weinheim, 2006.

One skilled in the art recognizes that compounds of Formula 1 can exist as mixtures of ketonic and solvated forms (e.g., hemiketals, ketals and hydrates) and each are independently interconvertible and agriculturally active. For example, ketones of Formula 1¹ (i.e. compounds of Formula 1 wherein T is T-1) may exist in equilibrium with their corresponding hydrates of Formula 1² (i.e. compounds of Formula 1 wherein T is T-2, and R^{2a}X and R^{2b}Y are both OH). In cases where the ketone group is in close proximity to an electron-withdrawing group, such as when R¹ is a trifluoromethyl group, the equilibrium typically favors the hydrate form.

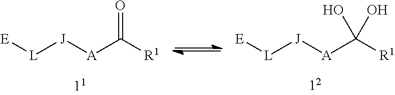

This invention comprises all ketonic and solvated forms of Formula 1 compounds, and mixtures thereof in all proportions. Unless otherwise indicated, reference to a compound by one tautomer description is to be considered to include all tautomers.

Additionally, some of the unsaturated rings and ring systems depicted in Exhibit A can have an arrangement of single and double bonds between ring members different from that depicted. Such differing arrangements of bonds for a particular arrangement of ring atoms correspond to different tautomers. For these unsaturated rings and ring systems, the particular tautomer depicted is to be considered representative of all the tautomers possible for the arrangement of ring atoms shown.

Embodiments of the present invention as described in the Summary of the Invention include those described below. In the following Embodiments, Formula 1 includes stereoisomers, N-oxides, and salts thereof, and reference to “a compound of Formula 1” includes the definitions of substituents specified in the Summary of the Invention unless further defined in the Embodiments.
Embodiment 1. A compound of Formula 1 wherein T is T-1.
Embodiment 2. A compound of Formula 1 wherein T is T-2. Embodiments. A compound of Formula 1 wherein T is T-3.
Embodiment 3a. A compound of Formula 1 wherein T is T-2 or T-3.
Embodiment 4. A compound of Formula 1 or any one of Embodiments 1 through 3a wherein R¹ is CF₃, CHF₂, CCl₃, CF₂Cl or CFCl₂.
Embodiment 5. A compound of Embodiment 4 wherein R¹ is CF₃, CCl₃ or CF₂Cl.
Embodiment 6. A compound of Embodiment 5 wherein R¹ is CF₃.
Embodiment 7. A compound of Formula 1 or any one of Embodiments 1 through 6 wherein W is O or S.
Embodiment 8. A compound of Embodiment 7 wherein W is O.
Embodiment 9. A compound of Formula 1 or any one of Embodiments 1 through 6 wherein W is NR³.
Embodiment 10. A compound of Formula 1 or Embodiments 1 and 9 wherein R³ is H, cyano, C(═O)OH, C₁-C₂ alkyl, C₂-C₃ alkylcarbonyl, C₂-C₃ haloalkylcarbonyl, OR^{3a} or NR^{3b}R^{3c}.
Embodiment 11. A compound of Embodiment 10 wherein R³ is H, cyano, C₁-C₂ alkyl or OR^{3a}.
Embodiment 12. A compound of Embodiment 11 wherein R³ is H, cyano or OR^{3a}.
Embodiment 13. A compound of Formula 1 or any one of Embodiments 1 through 12 wherein R^{3a} is H, C₁-C₂ alkyl, C₂-C₃ alkylcarbonyl or C₂-C₃ haloalkylcarbonyl.
Embodiment 14. A compound of Embodiment 13 wherein R^{3a} is H.
Embodiment 15. A compound of Formula 1 or any one of Embodiments 1 and 14 wherein when R^{3b} is separate (i.e. not taken together with R^{3c} to form a ring), then R^{3b} is H, C₁-C₃ alkyl, C₂-C₃ alkylcarbonyl or C₂-C₃ haloalkylcarbonyl.
Embodiment 16. A compound of Embodiment 15 wherein R^{3b} is H or methyl.
Embodiment 17. A compound of Formula 1 or any one of Embodiments 1 and 16 wherein when R^{3c} is separate (i.e. not taken together with R^{3b} to form a ring), then R^{3c} is H or C₁-C₂ alkyl.
Embodiment 18. A compound of Embodiment 17 wherein R^{3c} is H or methyl.
Embodiment 19. A compound of Formula 1 or any one of Embodiments 1 through 18 wherein X is O or NR^{5a}.
Embodiment 20. A compound of Formula 1 or any one of Embodiments 1 through 18 wherein X is O, S, NH or NOH.
Embodiment 20a. A compound of Embodiment 20 wherein X is O or NOH.
Embodiment 21. A compound of Embodiment 20 wherein X is O.
Embodiment 22. A compound of Formula 1 or any one of Embodiments 1 through 21 wherein Y is O or NR^{5b}.
Embodiment 23. A compound of Formula 1 or any one of Embodiments 1 through 21 wherein Y is O, S, NH or NOH.
Embodiment 23a. A compound of Embodiment 23 wherein Y is O or NOH.
Embodiment 24. A compound of Embodiment 22 wherein Y is O.
Embodiment 25. A compound of Formula 1 or any one of Embodiments 1 and 24 wherein R^{5a} and R^{5b} are each independently H, hydroxy or C₁-C₂ alkyl.
Embodiment 26. A compound of Embodiment 25 wherein R^{5a} and R^{5b} are each independently H, hydroxy or methyl.
Embodiment 27. A compound of Formula 1 or any one of Embodiments 1 through 26 wherein when R^{2a} and R^{2b} are separate (i.e. not taken together to form a ring), then R^{2a} and R^{2b} are each independently H, C₁-C₃ alkyl, C₂-C₃ alkenyl, (CR^{4a}R^{4b})ₚ—OH, (CR^{4a}R^{4b})ₚ—Cl or (CR^{4a}R^{4b})ₚ—Br.
Embodiment 28. A compound of Embodiment 27 wherein R^{2a} and R^{2b} are each independently H, C₁-C₃ alkyl, (CR^{4a}R^{4b})ₚ—Cl or (CR^{4a}R^{4b})ₚ—Br.
Embodiment 29. A compound of Embodiment 28 wherein R^{2a} and R^{2b} are each independently H, methyl, (CR^{4a}R^{4b})ₚ—Cl or (CR^{4a}R^{4b})ₚ—Br.
Embodiment 30. A compound of Embodiment 28 wherein R^{2a} and R^{2b} are each independently H or C₁-C₃ alkyl.
Embodiment 31. A compound of Embodiment 30 wherein R^{2a} and R^{2b} are each independently H or C₁-C₂ alkyl.
Embodiment 32. A compound of Embodiment 31 wherein R^{2a} and R^{2b} are each independently H or methyl.
Embodiment 33. A compound of Embodiment 32 wherein R^{2a} and R^{2b} are each H.
Embodiment 34. A compound of Formula 1 or any one of Embodiments 1 through 33 wherein when R^{2a} and R^{2b} are separate (i.e. not taken together to form a ring), then one of R^{2a} and R^{2b} is (CR^{4a}R^{4b})ₚ—OH, (CR^{4a}R^{4b})ₚ—SH, (CR^{4a}R^{4b})ₚ—Cl or (CR^{4a}R^{4b})ₚ—Br, and the other is H.
Embodiment 35. A compound of Embodiment 34 wherein one of R^{2a} and R^{2b} is (CR^{4a}R^{4b})ₚ—Cl or (CR^{4a}R^{4b})ₚ—Br, and the other is H.
Embodiment 36. A compound of Formula 1 or any one of Embodiments 1 through 35 wherein R^{2a} and R^{2b} are each independently H, methyl, (CR^{4a}R^{4b})ₚ—OH, (CR^{4a}R^{4b})ₚ—Cl or (CR^{4a}R^{4b})ₚ—Br; or R^{2a} and R^{2b} are taken together with the atoms X and Y to which they are attached to form a 5- to 6-membered saturated ring containing ring members, in addition to the atoms X and Y, selected from carbon atoms, wherein up to 2 carbon atom ring members are independently selected from C(═O) and C(═S), the ring optionally substituted with up to 2 substituents independently selected from halogen, cyano, C₁-C₂ alkyl, C₁-C₂ haloalkyl, C₁-C₂ alkoxy and C₁-C₂ haloalkoxy on carbon atom ring members.
Embodiment 37. A compound of Embodiment 36 wherein R^{2a} and R^{2b} are each independently H or methyl; or R^{2a} and R^{2b} are taken together with the atoms X and Y to which they are attached to form a 5- to 6-membered saturated ring containing ring members, in addition to the atoms X and Y, selected from carbon atoms, wherein up to 1 carbon atom ring member is selected from C(═O), the ring optionally substituted with up to 2 substituents independently selected from halogen, cyano, methyl, halomethyl, methoxy and halomethoxy on carbon atom ring members.
Embodiment 38. A compound of Embodiment 37 wherein R^{2a} and R^{2b} are each independently H or methyl; or R^{2a} and R^{2b} are taken together with the atoms X and Y to which they are attached to form a 5-membered saturated ring containing ring members, in addition to the atoms X and Y, selected from carbon atoms, the ring optionally substituted with up to 1 substituent selected from halogen, cyano and methyl on carbon atom ring members.
Embodiment 39. A compound of Embodiment 38 wherein R^{2a} and R^{2b} are each H; or R^{2a} and R^{2b} are taken together with the atoms X and Y to which they are attached to form a 5-membered saturated ring containing ring members, in addition to the atoms X and Y, selected from carbon atoms, the ring optionally substituted with up to 1 substituent selected methyl on a carbon atom ring member.
Embodiment 40. A compound of Embodiment 39 wherein R^{2a} and R^{2b} are each H; or R^{2a} and R^{2b} are taken together with the atoms X and Y to which they are attached to form a 5-membered saturated ring containing ring members, in addition to the atoms X and Y, selected from carbon atoms.
Embodiment 41. A compound of Formula 1 or any one of Embodiments 1 through 40 wherein when R^{2a} and R^{2b} are taken together to form a ring (i.e. not separate), then R^{2a} and R^{2b} are taken together with the atoms X and Y to which they are attached to form a 5- to 6-membered saturated ring containing ring members, in addition to the atoms X and Y, selected from carbon atoms, wherein up to 1 carbon atom ring member is selected from C(═O), the ring optionally substituted with up to 2 substituents independently selected from halogen, cyano, methyl, halomethyl, methoxy and halomethoxy on carbon atom ring members.
Embodiment 42. A compound of Embodiment 41 wherein R^{2a} and R^{2b} are taken together with the atoms X and Y to which they are attached to form a 5-membered saturated ring containing ring members, in addition to the atoms X and Y, selected from carbon atoms, the ring optionally substituted with up to 2 substituents independently selected from halogen, cyano, methyl, halomethyl and methoxy on carbon atom ring members.
Embodiment 43. A compound of Embodiment 42 wherein R^{2a} and R^{2b} are taken together with the atoms X and Y to which they are attached to form a 5-membered saturated ring containing ring members, in addition to the atoms X and Y, selected from carbon atoms, the ring optionally substituted with up to 1 substituent selected from halogen, methyl and halomethyl on a carbon atom ring member.
Embodiment 44. A compound of Embodiment 43 wherein R^{2a} and R^{2b} are taken together with the atoms X and Y to which they are attached to form a 5-membered saturated ring containing ring members, in addition to the atoms X and Y, selected from carbon atoms.
Embodiment 45. A compound of Formula 1 or any one of Embodiments 1 through 44 wherein R^{2c} is C₁-C₃ alkyl, C₁-C₃ haloalkyl, C₂-C₃ alkenyl, C₂-C₃ haloalkenyl, C₂-C₃ alkynyl or C₂-C₃ haloalkynyl, each optionally substituted with up 1 substituent selected from cyano, hydroxy, SC≡N and C₁-C₂ alkoxy.
Embodiment 46. A compound of Embodiment 45 wherein R^{2c} is C₁-C₂ alkyl, C₁-C₂ haloalkyl, C₂-C₃ alkenyl, C₂-C₃ haloalkenyl, C₂-C₃ alkynyl or C₂-C₃ haloalkynyl, each optionally substituted with up 1 substituent selected from cyano and methoxy.
Embodiment 46a. A compound of Embodiment 46 wherein R^{2c} is C₁-C₂ alkyl, C₁-C₂ haloalkyl, C₂-C₃ alkenyl, C₂-C₃ haloalkenyl or C₂-C₃ alkynyl.
Embodiment 47. A compound of Embodiment 46a wherein R^{2c} is C₁-C₂ alkyl, C₂-C₃ alkenyl or C₂-C₃ alkynyl.
Embodiment 48. A compound of Embodiment 47 wherein R^{2c} is methyl or ethyl.
Embodiment 48a. A compound of Embodiment 48 wherein R^{2c} is ethyl.
Embodiment 49. A compound of Formula 1 or any one of Embodiments 1 through 48a wherein R^{2d} is H, cyano, halogen or C₁-C₂ alkyl.
Embodiment 49a. A compound of Embodiment 49 wherein R^{2b} is H, cyano, Cl, F or methyl.
Embodiment 50. A compound of Embodiment 49a wherein R^{2b} is H or methyl.
Embodiment 51. A compound of Embodiment 50 wherein R^{2b} is H.
Embodiment 52. A compound of Formula 1 or any one of Embodiments 1 through 51 wherein each R^{4a} and R^{4b} is independently H or C₁-C₂ alkyl.
Embodiment 53. A compound of Embodiment 52 wherein each R^{4a} and R^{4b} is independently H or methyl.
Embodiment 54. A compound of Embodiment 53 wherein each R^{4a} and R^{4b} is H.
Embodiment 55. A compound of Formula 1 or any one of Embodiments 1 through 54 wherein p is 2.
Embodiment 56. A compound of Formula 1 or any one of Embodiments 1 through 54 wherein p is 3.
Embodiment 57. A compound of Formula 1 or any one of Embodiments 1 through 56 wherein A¹ is CR^{6c}R^{6d} O or S.
Embodiment 58. A compound of Embodiment 57 wherein A¹ is CR^{6c}R^{6d} or O.
Embodiment 59. A compound of Embodiment 58 wherein A¹ is CR^{6c}R^{6d}.
Embodiment 60. A compound of Embodiment 58 wherein A¹ is O.
Embodiment 61. A compound of Formula 1 or any one of Embodiments 1 through 60 wherein A¹ is CH₂, NH, O or S.
Embodiment 62. A compound of Formula 1 or any one of Embodiments 1 through 61 wherein A¹ is N(R^{7a}).
Embodiment 63. A compound of Formula 1 or any one of Embodiments 1 through 62 wherein A² is a direct bond, CR^{6e}R^{6f}, O or S.
Embodiment 64. A compound of Embodiment 63 wherein A² is a direct bond, CR^{6e}R^{6f} or O.
Embodiment 65. A compound of Embodiment 64 wherein A² is a direct bond or O.
Embodiment 66. A compound of Embodiment 65 wherein A² is a direct bond.
Embodiment 67. A compound of Formula 1 or any one of Embodiments 1 through 66 wherein A² is a direct bond, CH₂, NH, O or S.
Embodiment 67a. A compound of Embodiment 67 wherein A² is a direct bond, CH₂ or O.
Embodiment 68. A compound of Embodiment 67a wherein A² is a direct bond or O.
Embodiment 69. A compound of Formula 1 or any one of Embodiments 1 through 68 wherein A² is N(R^{7b}).
Embodiment 70. A compound of Formula 1 or any one of Embodiments 1 through 69 wherein when A is A¹-A²-CR^{6a}R^{6b}, then A¹-A²-CR^{6a}R^{6b} is selected from OCH₂, OCH(Me), CH(OH)CH₂, CH₂CH₂, SCH₂, OCF₂ and CH₂OCH₂.
Embodiment 71. A compound of Embodiment 70 wherein A¹-A²-CR^{6a}R^{6b} is selected from OCH₂, OCH(Me) and CH₂CH₂.
Embodiment 72. A compound of Embodiment 71 wherein A¹-A²-CR^{6a}R^{6b} is selected from OCH₂ and CH₂CH₂.
Embodiment 73. A compound of Embodiment 72 wherein A¹-A²-CR^{6a}R^{6b} is OCH₂.
Embodiment 74. A compound of Formula 1 or any one of Embodiments 1 through 73 wherein when A is A¹-A², then A¹-A² is selected from O, CH₂, OCH₂ and CH₂O.
Embodiment 75. A compound of Embodiment 74 wherein A¹-A² is selected from O, CH₂ and CH₂O.
Embodiment 76. A compound of Embodiment 75 wherein A¹-A² is selected from O and CH₂.
Embodiment 77. A compound of Embodiment 76 wherein A¹-A² is O.
Embodiment 78. A compound of Formula 1 or any one of Embodiments 1 through 77 wherein R^{6a}, R^{6b}, R^{6c}, R^{6d}, R^{6e} and R^{6f} are each independently H, cyano, hydroxy, Br, Cl, F or methyl.
Embodiment 79. A compound of Embodiment 78 wherein R^{6a}, R^{6b}, R^{6c}, R^{6d}, R^{6e} and R^{6f} are each independently H, cyano hydroxy or methyl.
Embodiment 80. A compound of Embodiment 79 wherein R^{6a}, R^{6b}, R^{6c}, R^{6d}, R^{6e} and R^{6f} are each independently H or methyl.
Embodiment 81. A compound of Embodiment 80 wherein R^{6a}, R^{6b}, R^{6c}, R^{6d}, R^{6e} and R^{6f} are each H.
Embodiment 82. A compound of Formula 1 or any one of Embodiments 1 through 81 wherein R^{7a} and R^{7b} are each independently H, C₁-C₂ alkyl or C₂-C₃ alkyl carbonyl.
Embodiment 83. A compound of Embodiment 82 wherein R^{7a} and R^{7b} are each independently H or C₁-C₂ alkyl.
Embodiment 84. A compound of Embodiment 83 wherein R^{7a} and R^{7b} are each H.
Embodiment 85. A compound of Formula 1 or any one of Embodiments 1 through 84 wherein when T is T-1 or T-2, then A is A¹-A²-CH₂.
Embodiment 86. A compound of Formula 1 or any one of Embodiments 1 through 85 wherein when T is T-1 or T-2, then A is OCH₂, SCH₂, NHCH₂, CH₂CH₂, OCH₂CH₂, SCH₂CH₂, NHCH₂CH₂, CH₂OCH₂, CH₂SCH₂ or CH₂NHCH₂.
Embodiment 87. A compound of Embodiment 86 wherein when T is T-1 or T-2, then A is OCH₂, SCH₂, CH₂CH₂, OCH₂CH₂, SCH₂CH₂, CH₂OCH₂ or CH₂SCH₂.
Embodiment 88. A compound of Embodiment 87 wherein when T is T-1 or T-2, then A is OCH₂ or CH₂CH₂.
Embodiment 89. A compound of Embodiment 88 wherein when T is T-1 or T-2, then A is OCH₂.
Embodiment 90. A compound of Formula 1 or any one of Embodiments 1 through 89 wherein when T is T-3, then A is O, OCH₂, SCH₂, NHCH₂, CH₂, CH₂CH₂, CH₂O, CH₂S or CH₂NH.
Embodiment 91. A compound of Embodiment 82 wherein when T is T-3, then A is O, CH₂ or OCH₂.
Embodiment 92. A compound of Embodiment 91 wherein when T is T-3, then A is O or CH₂.
Embodiment 93. A compound of Embodiment 92 wherein when T is T-3, then A is O.
Embodiment 94. A compound of Formula 1 or any one of Embodiments 1 through 93 wherein J is J-1 through J-3, J-6 through J-10 or J-14.
Embodiment 95. A compound of Embodiment 94 wherein J is J-1, J-2, J-3, J-6 or J-14.
Embodiment 96. A compound of Embodiment 95 wherein J is J-1, J-6 or J-14.
Embodiment 97. A compound of Embodiment 96 wherein J is J-1 or J-6.
Embodiment 97a. A compound of Embodiment 96 wherein J is J-14.
Embodiment 98. A compound of Embodiment 97 wherein J is J-1.
Embodiment 99. A compound of Embodiment 97 wherein J is J-6.
Embodiment 100. A compound of Formula 1 or any one of Embodiments 1 through 99 wherein each R⁸ is independently F, Cl or methyl.
Embodiment 100a. A compound of Embodiment 100 wherein each R⁸ is independently F or Cl.
Embodiment 101. A compound of Embodiment 100 wherein each R⁸ is independently F or methyl.
Embodiment 101a. A compound of Embodiment 101 wherein each R⁸ is F.
Embodiment 102. A compound of Formula 1 or any one of Embodiments 1 through 101a wherein q is 0 or 1.
Embodiment 103. A compound of Embodiment 102 wherein q is 0.
Embodiment 103a. A compound of Embodiment 102 wherein q is 1.
Embodiment 104. A compound of Formula 1 or any one of Embodiments 1 through 103a wherein each R^{9a} and R^{9b} is independently H, halogen, C₁-C₃ alkyl, C₁-C₃ haloalkyl, C₁-C₃ alkoxy or C₁-C₃ haloalkoxy.
Embodiment 105. A compound of Embodiment 104 wherein each R^{9a} and R^{9b} is independently H, halogen, C₁-C₂ alkyl or C₁-C₂ haloalkyl.
Embodiment 106. A compound of Embodiment 105 wherein each R^{9a} and R^{9b} is independently H, halogen or methyl.
Embodiment 107. A compound of Embodiment 106 wherein each R^{9a} and R^{9b} is independently H or methyl.
Embodiment 108. A compound of Embodiment 107 wherein each R^{9a} and R^{9b} is H.
Embodiment 109. A compound of Formula 1 or any one of Embodiments 1 through 108 wherein n is 0, 1 or 2.
Embodiment 109a. A compound of Embodiment 109 wherein n is 1 or 2.
Embodiment 110. A compound of Formula 1 or any one of Embodiments 1 through 108 wherein n is 0 or 1.
Embodiment 111. A compound of Embodiments 109, 109a or 110 wherein nisi.
Embodiment 112. A compound of Embodiments 109 or 110 wherein n is 0.
Embodiment 113. A compound of Formula 1 or any one of Embodiments 1 through 112 wherein L is a direct bond, CH₂, CH(Me) or CH₂CH₂.
Embodiment 113a. A compound of Embodiment 113 wherein L is a direct bond, CH₂ or CH₂CH₂.
Embodiment 114. A compound of Embodiment 113a wherein L is a direct bond or CH₂.
Embodiment 115. A compound of Embodiment 114 wherein L is CH₂.
Embodiment 115a. A compound of Embodiment 114 wherein L is a direct bond.
Embodiment 116. A compound of Formula 1 or any one of Embodiments 1 through 115a wherein E is E¹.
Embodiment 116a. A compound of Formula 1 or any one of Embodiments 1 through 115a wherein when L is a direct bond, then E is E¹.
Embodiment 117. A compound of Formula 1 or any one of Embodiments 1 through 116a wherein E¹ is cyano, nitro, C(═O)H, C(═O)OH or SC≡N; or C₁-C₃ alkoxy, C₂-C₆ alkenyloxy, C₁-C₆ alkylsulfonyl, C₂-C₆ alkenylsulfonyl, C₂-C₆ alkynylsulfonyl, C₁-C₆ alkylsulfonylamino, C₂-C₆ alkenylsulfonylamino, C₂-C₆ alkynylsulfonylamino, C₁-C₆ alkylaminosulfonyl, C₂-C₆ dialkylaminosulfonyl, C₂-C₆ alkenylaminosulfonyl, C₂-C₆ alkylcarbonyl, C₂-C₆ alkylaminocarbonyl, C₃-C₆ alkenylaminocarbonyl, C₃-C₆ alkynylaminocarbonyl, C₂-C₆ alkoxy carbonyl, C₃-C₆ alkenyloxy carbonyl, C₃-C₆ alkynyloxy carbonyl or C₂-C₆ alkoxycarbonylamino, wherein each carbon atom is optionally substituted with up to 1 substituent selected from R^{10a} and up to 3 substituents independently selected
from R^{10b}.
Embodiment 118. A compound of Embodiment 117 wherein E¹ is cyano, nitro, C(═O)H, C(═O)OH or SC≡N; or C₁-C₆ alkoxy, C₂-C₆ alkenyloxy, C₁-C₆ alkylsulfonyl, C₁-C₆ alkylsulfonylamino, C₂-C₆ alkenylsulfonylamino, C₂-C₆ alkylcarbonyl, C₂-C₆ alkoxy carbonyl, C₃-C₆ alkenyloxy carbonyl or C₃-C₆ alkynyloxy carbonyl, wherein each carbon atom is optionally substituted with up to 1 substituent selected from R^{10a} and up to 3 substituents independently selected from R^{10b}.
Embodiment 119. A compound of Embodiment 118 wherein E¹ is C₁-C₆ alkoxy, C₁-C₆ alkylsulfonyl, C₂-C₆ alkylcarbonyl or C₂-C₆ alkoxycarbonyl, wherein each carbon atom is optionally substituted with up to 1 substituent selected from R^{10a} and up to 3 substituents independently selected from R^{10b}.
Embodiment 120. A compound of Embodiment 119 wherein E¹ is C₁-C₃ alkoxy, C₂-C₃ alkylcarbonyl or C₂-C₃ alkoxycarbonyl, wherein each carbon atom is optionally substituted with up to 1 substituent selected from R^{10a} and up to 3 substituents independently selected from R^{10b}.
Embodiment 120a. A compound of Embodiment 120 wherein E¹ is C₁-C₃ alkoxy or C₂-C₃ alkoxycarbonyl, wherein each carbon atom is optionally substituted with up to 1 substituent selected from R^{10a}.
Embodiment 121. A compound of Embodiment 120 wherein E¹ is C₁-C₂ alkoxy, wherein each carbon atom is optionally substituted with up to 1 substituent selected from R^{10a} and up to 3 substituents independently selected from R^{10b}.
Embodiment 121a. A compound of Embodiment 120 wherein E¹ is C₁-C₂ alkoxy, wherein each carbon atom is optionally substituted with up to 1 substituent selected from R^{10a}.
Embodiment 121b. A compound of Embodiment 121a wherein E¹ is methoxy optionally substituted with up to 1 substituent selected from R^{10a}.
Embodiment 121c. A compound of Embodiment 121a wherein E¹ is methoxy substituted with 1 substituent selected from R^{10a}.
Embodiment 122. A compound of Formula 1 or any one of Embodiments 1 through 121c wherein R^{10a} is phenyl optionally substituted with up to 3 substituents independently selected from R^{11a}; or a 5- to 6-membered heterocyclic ring containing ring members selected from carbon atoms and 1 to 4 heteroatoms independently selected from up to 2 O, up to 2 S and up to 4 N atoms, each ring optionally substituted with up to 3 substituents independently selected from R^{11a} on carbon atom ring members and R_{11b} on nitrogen atom ring members.
Embodiment 123. A compound of Embodiment 122 wherein R^{10a} is phenyl optionally substituted with up to 2 substituents independently selected from R^{11a}; or a 5- to 6-membered heterocyclic ring containing ring members selected from carbon atoms and 1 to 4 heteroatoms independently selected from up to 2 O, up to 2 S and up to 4 N atoms, each ring optionally substituted with up to 2 substituents independently selected from R^{11a} on carbon atom ring members and R^{1b} on nitrogen atom ring members.
Embodiment 123a. A compound of Embodiment 123 wherein R^{10a} is a 5-membered heterocyclic ring containing ring members selected from carbon atoms and 1 to 4 heteroatoms independently selected from up to 2 O and up to 3 N atoms, each ring optionally substituted with up to 2 substituents independently selected from R^{11a} on carbon atom ring members.
Embodiment 123b. A compound of Embodiment 123a wherein R^{10a} is pyrazolyl, imidazolyl or triazolyl, each optionally substituted with up to 2 substituents independently selected from R^{11a} on carbon atom ring members.
Embodiment 123c. A compound of Embodiment 123b wherein R^{10a} is pyrazolyl or imidazolyl, each optionally substituted with up to 2 substituents independently selected from R^{11a} on carbon atom ring members.
Embodiment 123d. A compound of Embodiment 123c wherein R^{10a} is pyrazolyl optionally substituted with up to 1 substituent selected from R^{11a} on a carbon atom ring member.
Embodiment 124. A compound of Formula 1 or any one of Embodiments 1 through 123c wherein each R^{10b} is independently cyano, halogen, hydroxy, C₁-C₄ alkyl, C₁-C₄ haloalkyl, C₃-C₅ cycloalkyl, C₁-C₄ alkoxy, C₁-C₄ haloalkoxy, C₁-C₄ alkylsulfonyl, C₁-C₄ haloalkylsulfonyl, C₁-C₄ alkylamino, C₂-C₄ dialkylamino, C₂-C₄ alkylcarbonyl or C₂-C₅ alkoxycarbonyl.
Embodiment 125. A compound of Embodiment 124 wherein each R^{10b} is independently halogen, hydroxy, C₁-C₄ alkyl, C₁-C₄ haloalkyl, C₁-C₄ alkoxy, C₁-C₄ haloalkoxy, C₁-C₄ alkylsulfonyl, C₂-C₄ alkylcarbonyl or C₂-C₅ alkoxycarbonyl.
Embodiment 125a. A compound of Embodiment 125 wherein each R^{10b} is independently halogen, C₁-C₂ alkyl, C₁-C₂ haloalkyl, C₁-C₂ alkoxy or C₂-C₄ alkoxycarbonyl.
Embodiment 126. A compound of Formula 1 or any one of Embodiments 1 through 125a wherein each R^{11a} is independently halogen, C₁-C₄ alkyl, C₁-C₄ haloalkyl, C₂-C₄ alkenyl, C₂-C₄ alkynyl, C₁-C₄ alkoxy, C₁-C₄ haloalkoxy, C₂-C₄ alkenyloxy, C₂-C₄ alkynyloxy, C₂-C₄ alkoxyalkyl, C₂-C₅ alkylcarbonyloxy, C₁-C₄ alkylsulfonyl, C₁-C₄ haloalkylsulfonyl, C₁-C₄ alkylsulfonyloxy, C₂-C₄ alkylcarbonyl, C₃-C₅ alkenylcarbonyl, C₃-C₅ alkynylcarbonyl, C₂-C₅ alkoxycarbonyl, C₃-C₇ alkenyloxycarbonyl, C₃-C₇ alkynyloxycarbonyl, C₂-C₆ alkylaminocarbonyl, C₃-C₅ alkenylaminocarbonyl, C₃-C₅ alkynylaminocarbonyl or C₃-C₈ dialkylaminocarbonyl.
Embodiment 127. A compound of Embodiment 126 wherein each R^{11a} is independently halogen, C₁-C₄ alkyl, C₁-C₄ haloalkyl, C₂-C₄ alkenyl, C₁-C₄ alkoxy, C₁-C₄ haloalkoxy, C₂-C₄ alkenyloxy, C₂-C₄ alkoxyalkyl, C₂-C₄ alkylcarbonyl, C₂-C₆ alkoxy carbonyl, C₃-C₇ alkenyloxy carbonyl or C₂-C₆ alkylaminocarbonyl.
Embodiment 128. A compound of Embodiment 127 wherein each R^{11a} is independently halogen, C₁-C₄ alkyl, C₁-C₄ haloalkyl, C₁-C₄ alkoxy, C₁-C₄ haloalkoxy, C₂-C₄ alkenyloxy, C₂-C₄ alkylcarbonyl, C₂-C₄ alkoxycarbonyl or C₃-C₅ alkenyloxy carbonyl.
Embodiment 128a. A compound of Embodiment 128 wherein each R^{11a} is independently halogen, C₁-C₂ alkyl, C₁-C₂ haloalkyl, C₁-C₂ alkoxy or C₂-C₃ alkoxycarbonyl.
Embodiment 128b. A compound of Embodiment 128a wherein each R^{11a} is independently methoxycarbonyl or ethoxycarbonyl.
Embodiment 128c. A compound of Embodiment 128b wherein each R^{11a} is ethoxycarbonyl.
Embodiment 129. A compound of Formula 1 or any one of Embodiments 1 through 128c wherein each R^{11b} is independently C₁-C₂ alkyl, C₁-C₂ alkoxy, C₂-C₃ alkylcarbonyl or C₂-C₃ alkoxycarbonyl.
Embodiment 130. A compound of Embodiment 129 wherein each R^{11b} is independently methyl, methoxy, methylcarbonyl or methoxycarbonyl.
Embodiment 131. A compound of Embodiment 130 wherein each R^{11b} is independently methyl or methoxy.
Embodiment 132. A compound of Formula 1 or any one of Embodiments 1 through 131 wherein E is E².
Embodiment 133. A compound of Formula 1 or any one of Embodiments 1 through 132 wherein G is phenyl optionally substituted with up to 3 substituents independently selected from R¹³; or a 5- to 6-membered heteroaromatic ring, each ring containing ring members selected from carbon atoms and 1 to 4 heteroatoms independently selected from up to 2 O, up to 2 S and up to 4 N atoms, each ring optionally substituted with up to 3 substituents independently selected from R¹³; or a 3- to 7-membered nonaromatic ring or an 8- to 11-membered bicyclic ring system, each ring or ring system containing ring members selected from carbon atoms and optionally up to 4 heteroatoms independently selected from up to 2 O, up to 2 S and up to 4 N atoms, wherein up to 2 ring members are independently selected from C(═O), S(═O) and S(═O)₂, each ring or ring system optionally substituted with up to 3 substituents independently selected from R¹³.
Embodiment 134. A compound of Embodiment 133 wherein G is selected from G-1 through G-118 as shown in Exhibit A.

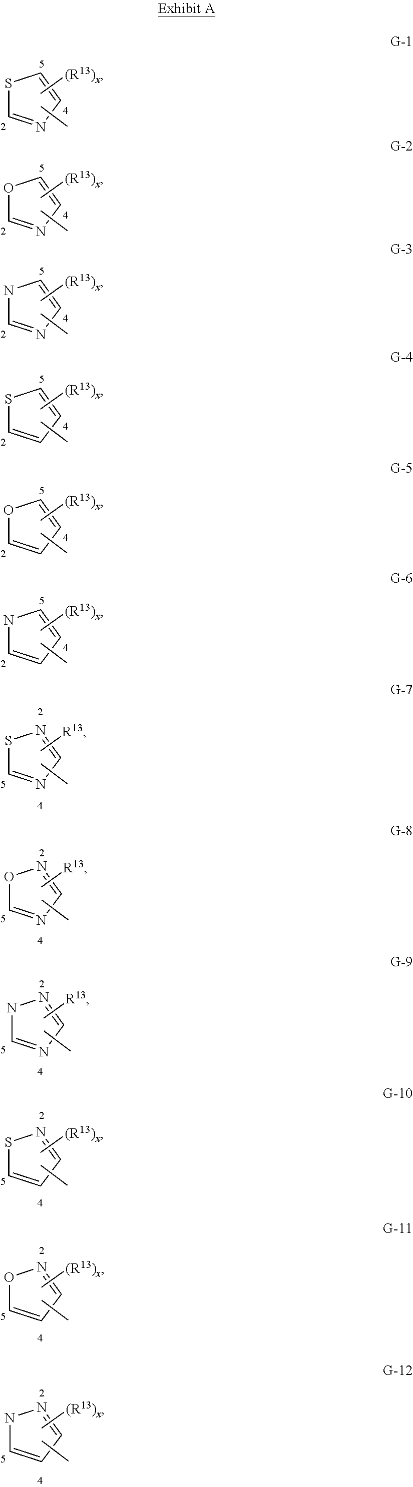

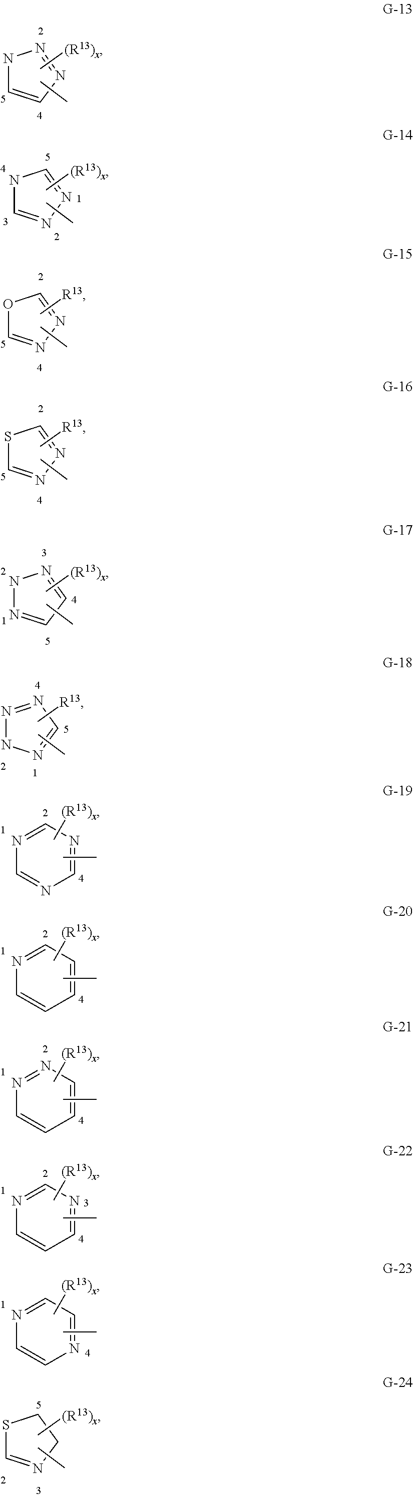

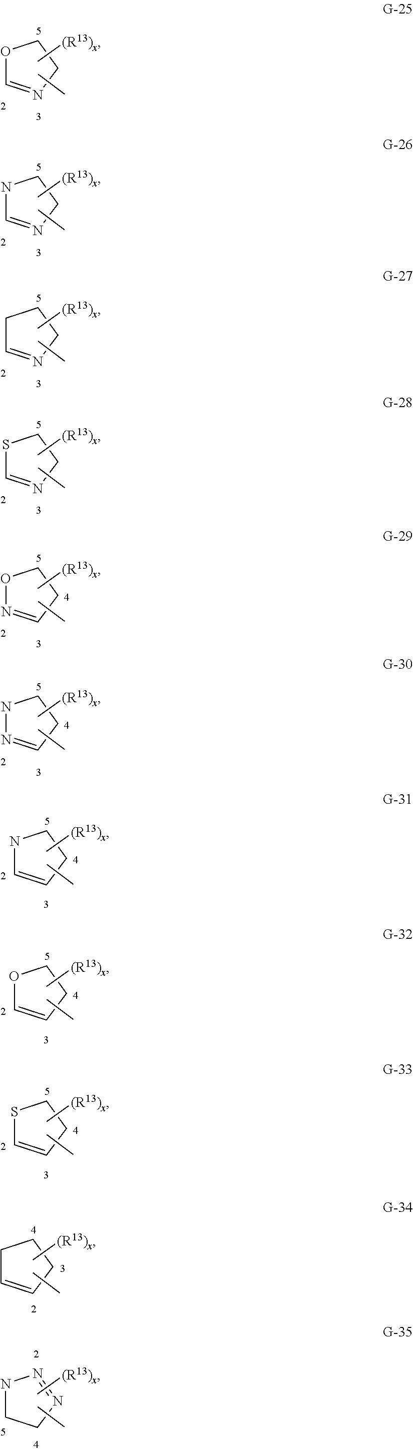

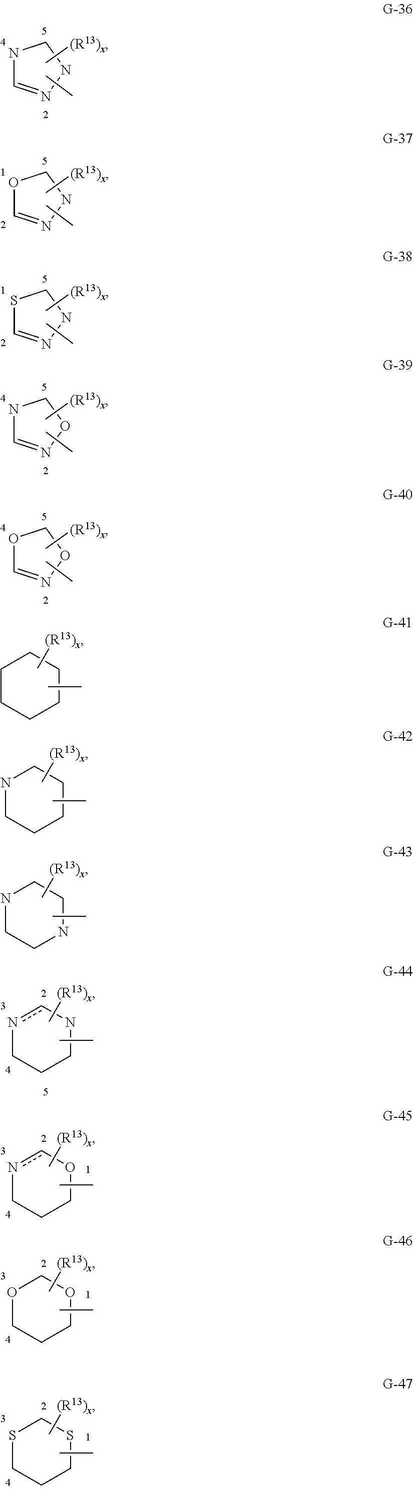

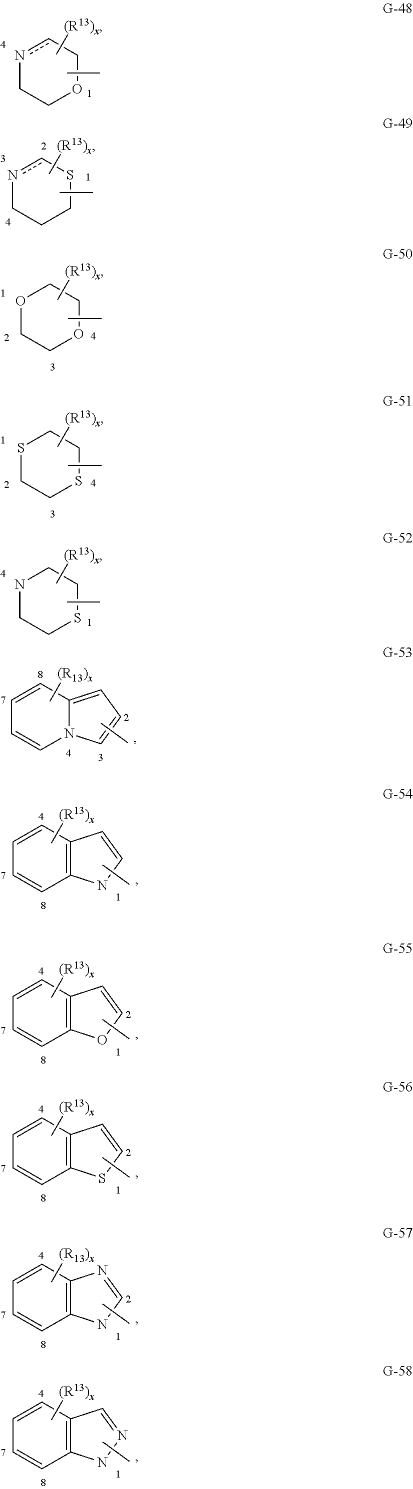

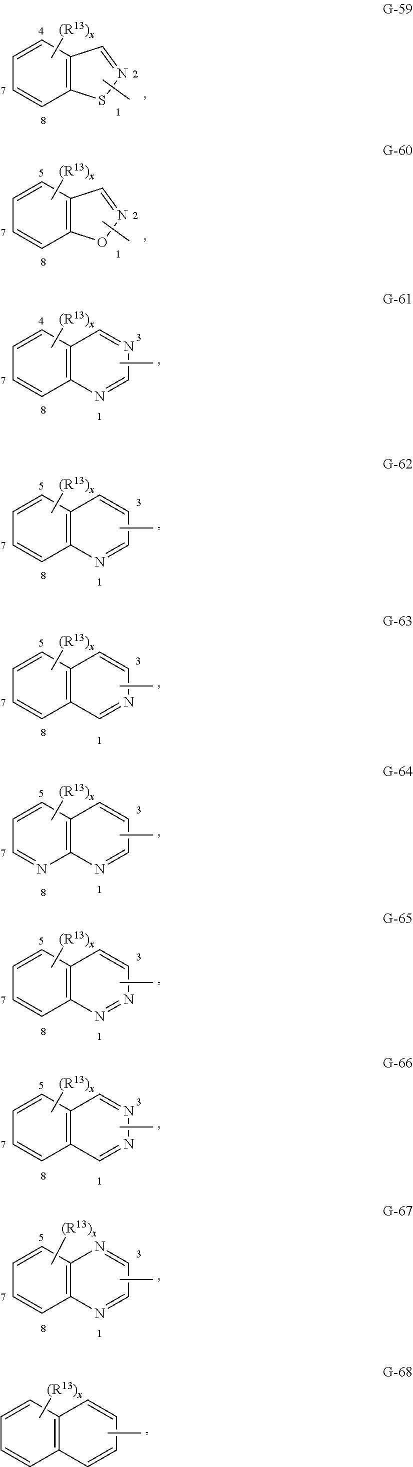

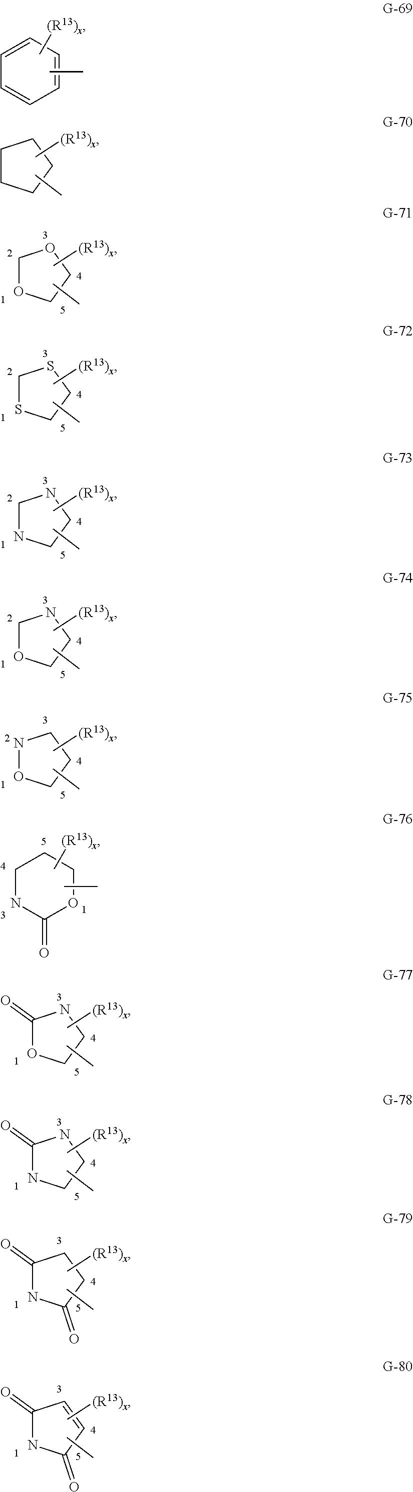

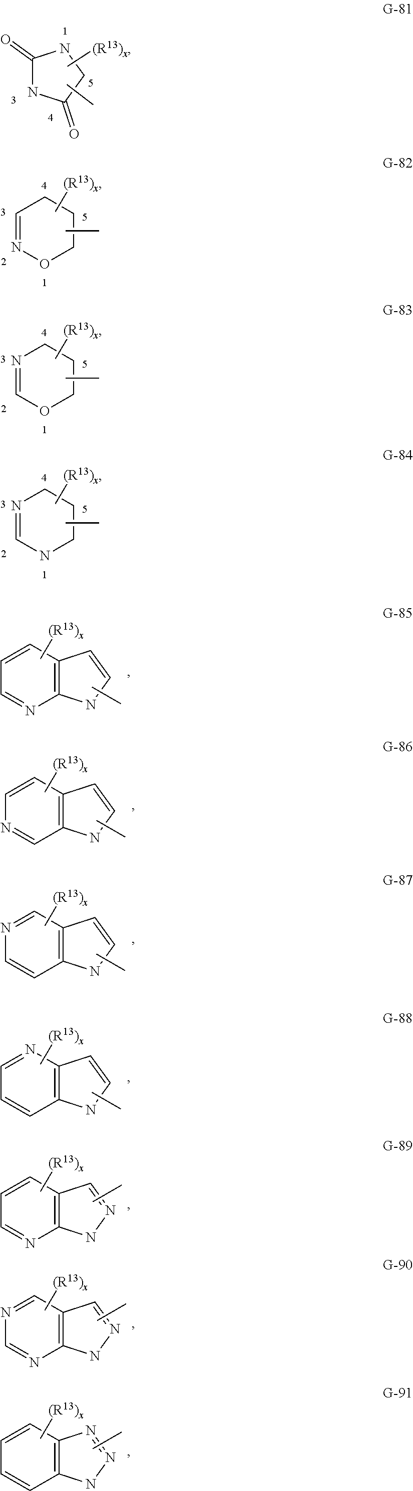

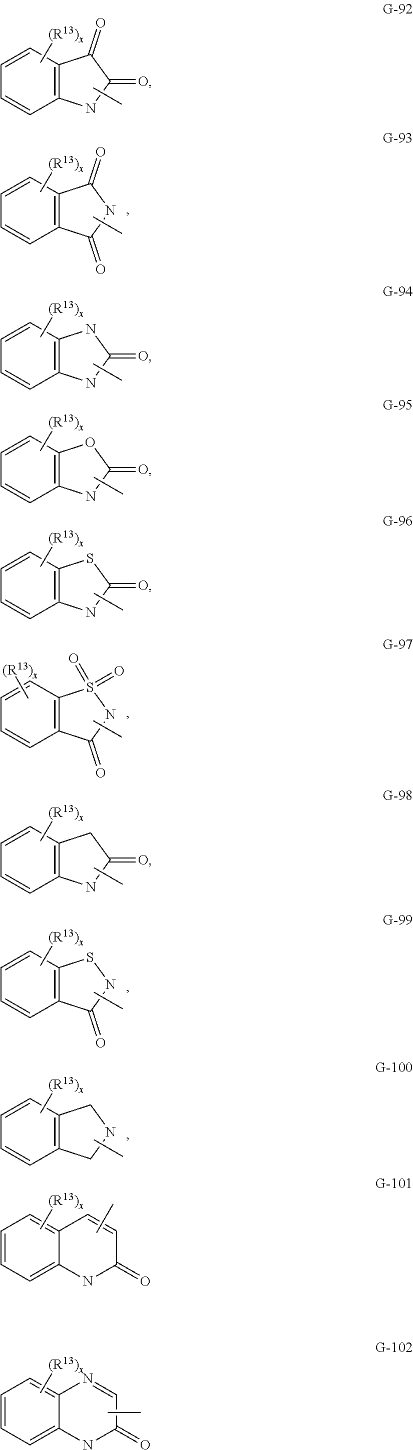

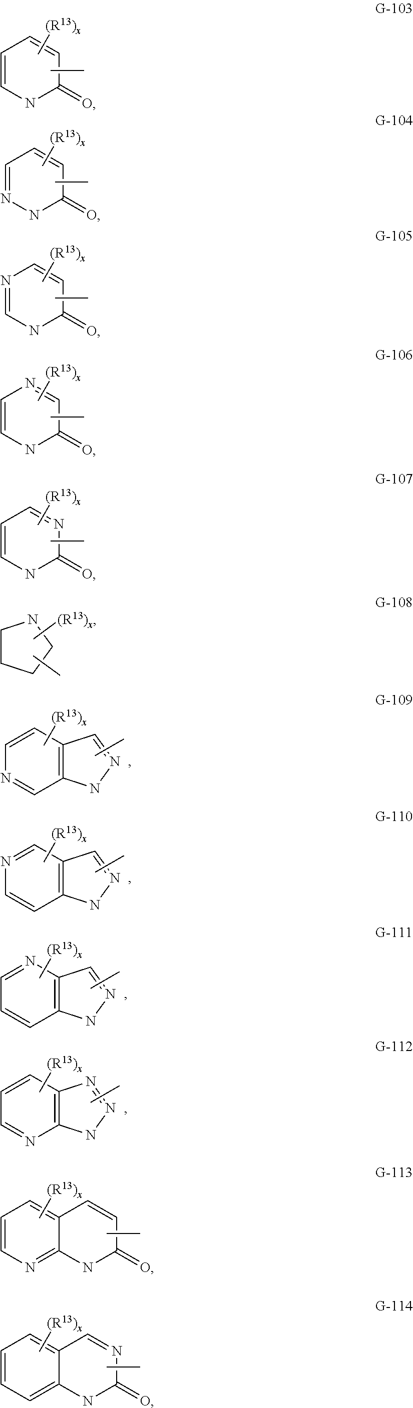

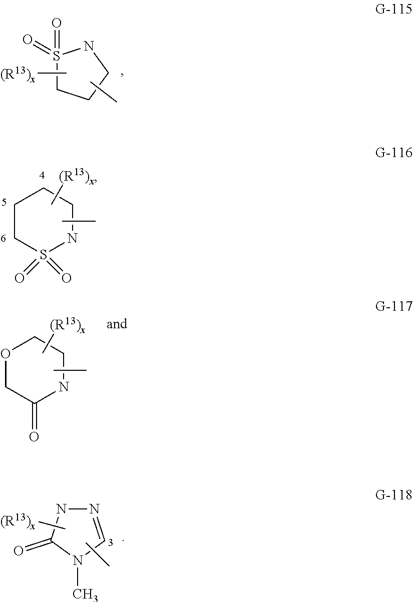

wherein the floating bond is connected to Z in Formula 1 through any available carbon or nitrogen atom of the depicted ring or ring system; and x is 0, 1, 2 or 3.
Embodiment 135. A compound of Embodiment 134 wherein G is G-1 through G-16, G-20, G-22 through G-30, G-36 through G-42, G-54 through G-60, G-85, G-86, G-108, G-110 or G-111.
Embodiment 136. A compound of Embodiment 135 wherein G is G-1 through G-16, G-22, G-24, G-25, G-26, G-28, G-29, G-30, G-36, G-37, G-38, G-41, G-42, G-54, G-57, G-58, G-59, G-60, G-85, G-86, G-108, G-110 or G-111.
Embodiment 137. A compound of Embodiment 136 wherein G is G-1 through G-13, G-22, G-24, G-25, G-26, G-28, G-29, G-41, G-42, G-54, G-57, G-58, G-59 or G-60.
Embodiment 138. A compound of Embodiment 137 wherein G is G-1, G-2, G-3, G-7, G-8, G-9, G-10, G-12, G-13, G-22, G-29, G-42, G-54 or G-58.
Embodiment 139. A compound of Embodiment 138 wherein G is G-1, G-3, G-12, G-13, G-22 or G-42.
Embodiment 140. A compound of Embodiment 139 wherein G is G-1, G-3, G-12, G-13 or G-22.
Embodiment 141. A compound of Embodiment 140 wherein G is G-1, G-3, G-12 or G-22.
Embodiment 142. A compound of Embodiment 141 wherein G is G-1 or G-12.
Embodiment 143. A compound of Embodiment 142 wherein G is G-1.
Embodiment 144. A compound of Embodiment 142 wherein G is G-12.
Embodiment 145. A compound of Embodiment 140 wherein G is G-3.
Embodiment 146. A compound of Embodiment 140 wherein G is G-22.
Embodiment 147. A compound of Embodiment 143 wherein the 2-position of G-1 is connected to Z and the 4-position is connected to R¹³.
Embodiment 148. A compound of Embodiment 143 wherein the 2-position of G-1 is connected to Z and the 5-position is connected to R¹³.
Embodiment 149. A compound of Embodiment 144 wherein the 1-position of G-12 is connected to Z and the 4-position is connected to R¹³.
Embodiment 150. A compound of Embodiment 144 wherein the 1-position of G-12 is connected to Z and the 3-position is connected to R¹³.
Embodiment 151. A compound of Embodiment 144 wherein the 1-position of G-12 is connected to Z and the 3- and 5-positions are connected to R¹³.
Embodiment 152. A compound of Embodiment 144 wherein the 1-position of G-12 is connected to Z and the 5-position is connected to R¹³.
Embodiment 153. A compound of Embodiment 145 wherein the 1-position of G-3 is connected to Z and the 4-position is connected to R¹³.
Embodiment 154. A compound of Embodiment 146 wherein the 4-position of G-22 is connected to Z and the 2-position is connected to R¹³.
Embodiment 155. A compound of any one of Embodiments 147 through 154 wherein Z is a direct bond.
Embodiment 156. A compound of any one of Embodiments 147 through 155 wherein x is 1 and R¹³ is methoxy carbonyl or ethoxy carbonyl.
Embodiment 157. A compound of any one of Embodiments 134 through 155 wherein x is 1 or 2.
Embodiment 158. A compound of Embodiment 157 wherein x is 1.
Embodiment 159. A compound of Embodiment 157 wherein x is 2.
Embodiment 160. A compound of any one of Embodiments 134 through 155 wherein x is 0.
Embodiment 161. A compound of Formula 1 or any one of Embodiments 1 through 159 wherein each R¹³ is independently cyano, halogen, NR^{14a}R^{14b}, C(═O)NR^{14a}R^{14b}, C(R¹⁵)═NR¹⁶, N═CR¹⁷NR^{18a}R^{18b} or —U—V-Q; or C₁-C₆ alkyl, C₂-C₆ alkenyl, C₂-C₆ alkynyl, C₁-C₃ alkoxy, C₂-C₆ alkenyloxy, C₂-C₆ alkynyloxy, C₁-C₆ alkylsulfonyl, C₁-C₃ alkylsulfonyloxy, C₁-C₆ alkylsulfonylamino, C₂-C₆ alkylcarbonyl, C₂-C₆ alkoxycarbonyl, C₃-C₆ alkenyloxycarbonyl, C₃-C₆ alkynyloxycarbonyl, C₄-C₇ cycloalkoxycarbonyl, C₂-C₆ alkylcarbonyloxy, C₂-C₆ alkoxycarbonyloxy, C₄-C₇ cycloalkoxycarbonyloxy, C₂-C₆ alkylaminocarbonyloxy, C₂-C₆ alkylcarbonylamino, C₂-C₆ alkoxycarbonylamino or C₂-C₆ alkylaminocarbonylamino, each optionally substituted with up to 3 substituents independently selected from R¹⁹.
Embodiment 162. A compound of Embodiment 161 wherein each R¹³ is independently cyano, halogen, C(═O)NR^{14a}R^{14b} or —U—V-Q; or C₁-C₆ alkyl, C₂-C₆ alkenyl, C₂-C₆ alkynyl, C₁-C₆ alkoxy, C₂-C₆ alkenyloxy, C₂-C₆ alkynyloxy, C₁-C₆ alkylsulfonyl, C₁-C₆ alkylsulfonyloxy, C₁-C₆ alkylsulfonylamino, C₂-C₆ alkylcarbonyl, C₂-C₆ alkoxycarbonyl, C₃-C₆ alkenyloxycarbonyl, C₃-C₆ alkynyloxycarbonyl or C₂-C₆ alkoxycarbonyloxy, each optionally substituted with up to 3 substituents independently selected from R¹⁹.
Embodiment 163. A compound of Embodiment 162 wherein each R¹³ is independently C(═O)NR^{14a}R^{14b} or —U—V-Q; or C₁-C₆ alkyl, C₂-C₆ alkenyl, C₂-C₆ alkynyl, C₁-C₆ alkoxy, C₂-C₆ alkenyloxy, C₂-C₆ alkynyloxy, C₁-C₆ alkylsulfonyl, C₁-C₆ alkylsulfonyloxy, C₁-C₆ alkylsulfonylamino, C₂-C₆ alkylcarbonyl, C₂-C₆ alkoxycarbonyl, C₃-C₆ alkenyloxycarbonyl, C₃-C₆ alkynyloxycarbonyl, C₄-C₈ cycloalkoxycarbonyl or C₂-C₆ alkoxycarbonyloxy, each optionally substituted with up to 3 substituents independently selected from R¹⁹.
Embodiment 163a. A compound of Embodiment 162 wherein each R¹³ is independently C(═O)NR^{14a}R^{14b} or —U—V-Q; or C₂-C₅ alkoxycarbonyl, C₃-C₅ alkenyloxycarbonyl, C₃-C₅ alkynyloxycarbonyl or C₄-C₅ cycloalkoxycarbonyl, each optionally substituted with up to 3 substituents independently selected from R¹⁹.
Embodiment 163b. A compound of Embodiment 163a wherein each R¹³ is independently C(═O)NR^{14a}R^{14b} or —U—V-Q; or C₂-C₅ alkoxycarbonyl, C₃-C₅ alkynyloxycarbonyl or C₄-C₆ cycloalkoxycarbonyl, each optionally substituted with up to 1 substituent selected from R¹⁹.
Embodiment 164. A compound of Embodiment 163a wherein each R¹³ is independently C(═O)NR^{14a}R^{14b} or —U—V-Q; or C₂-C₆ alkoxycarbonyl, C₃-C₅ alkenyloxycarbonyl, C₃-C₆ alkynyloxycarbonyl or C₂-C₆, alkoxycarbonyloxy, each optionally substituted with up to 3 substituents independently selected from R¹⁹.
Embodiment 164a. A compound of Embodiment 164 wherein each R¹³ is independently C(═O)NR^{14a}R^{14b} or —U—V-Q; or C₂-C₆, alkoxycarbonyl, C₃-C₆ alkenyloxycarbonyl, or C₃-C₆ alkynyloxycarbonyl, each optionally substituted with up to 3 substituents independently selected from R¹⁹.
Embodiment 165. A compound of Embodiment 164a wherein each R¹³ is independently C(═O)NR^{14a}R^{14b} or —U—V-Q; or C₂-C₅ alkoxycarbonyl, C₃-C₅ alkenyloxycarbonyl, or C₃-C₅ alkynyloxycarbonyl, each optionally substituted with up to 3 substituents independently selected from R¹⁹.
Embodiment 165a. A compound of Embodiment 165 wherein each R¹³ is independently C(═O)NR^{14a}R^{14b} or —U—V-Q; or C₂-C₅ alkoxycarbonyl or C₃-C₅ alkenyloxycarbonyl, each optionally substituted with up to 3 substituents independently selected from R¹⁹.
Embodiment 166. A compound of Embodiment 165 wherein each R¹³ is independently C₂-C₅ alkoxycarbonyl or C₃-C₅ alkenyloxycarbonyl, each optionally substituted with up to 3 substituents independently selected from R¹⁹.
Embodiment 167. A compound of Embodiment 166 wherein each R¹³ is independently C₂-C₅ alkoxycarbonyl, each optionally substituted with up to 3 substituents independently selected from R¹⁹.
Embodiment 168. A compound of Embodiment 167 wherein each R¹³ is independently methoxycarbonyl or ethoxycarbonyl, each optionally substituted with up to 3 substituents independently selected from R¹⁹.
Embodiment 169. A compound of Embodiment 168 wherein each R¹³ is independently methoxycarbonyl or ethoxycarbonyl, each optionally substituted with up to 1 substituent selected from R¹⁹.
Embodiment 170. A compound of Embodiment 169 wherein each R¹³ is independently ethoxycarbonyl optionally substituted with up to 1 substituent selected from R¹⁹.
Embodiment 171. A compound of Embodiment 169 wherein each R¹³ is independently methoxycarbonyl or ethoxycarbonyl.
Embodiment 172. A compound of Embodiment 171 wherein each R¹³ is ethoxycarbonyl.
Embodiment 173. A compound of Formula 1 or any one of Embodiments 1 through 172 wherein when each R^{14a} is separate (i.e. not taken together with R^{14b} to form a ring), then each R^{14a} is independently H, cyano, hydroxy, C₁-C₄ alkyl, C₁-C₄ haloalkyl, C₂-C₄ alkenyl, C₂-C₄ haloalkenyl, C₂-C₄ alkynyl, C₂-C₄ haloalkynyl, C₂-C₄ alkylcarbonyl, C₂-C₅ alkoxycarbonyl or C₃-C₅ dialkylaminocarbonyl.
Embodiment 174. A compound of Embodiment 173 wherein each R^{14a} is independently H, cyano, C₁-C₄ alkyl, C₁-C₄ haloalkyl, C₂-C₄ alkenyl, C₂-C₄ alkynyl, C₂-C₄ alkylcarbonyl, C₂-C₅ alkoxycarbonyl or C₃-C₅ dialkylaminocarbonyl.
Embodiment 175. A compound of Embodiment 174 wherein each R^{14a} is independently H, C₁-C₂ alkyl, C₂-C₄ alkenyl, C₂-C₄ alkynyl, C₂-C₄ alkylcarbonyl or C₂-C₄ alkoxycarbonyl.
Embodiment 176. A compound of Embodiment 175 wherein each R^{14a} is independently H or C₁-C₂ alkyl.
Embodiment 177. A compound of Embodiment 176 wherein each R^{14a} is independently H or methyl.
Embodiment 177a. A compound of Embodiment 177 wherein each R^{14a} is H.
Embodiment 178. A compound of Formula 1 or any one of Embodiments 1 through 177a wherein when each R^{14b} is separate (i.e. not taken together with R^{14a} to form a ring), then each R^{14b} is independently H, C₁-C₆ alkyl, C₁-C₆ haloalkyl, C₂-C₆ alkenyl, C₂-C₆ haloalkenyl, C₂-C₅ alkynyl, C₂-C₅ haloalkynyl, C₂-C₆ cyanoalkyl, C₃-C₈ cycloalkyl, C₃-C₈ halocycloalkyl, C₃-C₈ cycloalkenyl, C₃-C₈ halocycloalkenyl, C₄-C₁₀ alkylcycloalkyl, C₄-C₁₀ cycloalkylalkyl, C₄-C₁₀ halocycloalkylalkyl, C₂-C₆ alkoxyalkyl, C₂-C₆ haloalkoxyalkyl, C₂-C₆ alkylthioalkyl, C₂-C₅ alkylsulfonylalkyl, C₂-C₅ alkylaminoalkyl or C₃-C₈ dialkylaminoalkyl, each optionally substituted with up to 1 substituent selected from cyano, hydroxy, nitro, C₂-C₄ alkylcarbonyl or C₂-C₄ alkoxycarbonyl.
Embodiment 179. A compound of Embodiment 178 wherein each R^{14b} is independently H, C₁-C₄ alkyl, C₁-C₄ haloalkyl, C₂-C₄ alkenyl, C₂-C₄ haloalkenyl, C₂-C₄ alkynyl, C₃-C₅ cycloalkyl, C₄-C₆ cycloalkylalkyl, C₂-C₄ alkoxyalkyl, C₂-C₄ haloalkoxyalkyl, C₂-C₄ alkylaminoalkyl or C₃-C₅ dialkylaminoalkyl.
Embodiment 180. A compound of Embodiment 179 wherein each R^{14b} is independently H, C₁-C₃ alkyl, C₁-C₃ haloalkyl, C₂-C₄ alkenyl, C₂-C₄ haloalkenyl, C₃-C₅ cycloalkyl, C₄-C₀ cycloalkylalkyl or C₂-C₄ alkoxyalkyl.
Embodiment 181. A compound of Embodiment 180 wherein each R^{14b} is independently H, C₁-C₃ alkyl, C₁-C₃ haloalkyl, cyclopropylmethyl or C₂-C₄ alkoxyalkyl.
Embodiment 181a. A compound of Embodiment 181 wherein each R^{14b} is independently H, C₁-C₂ alkyl, C₁-C₂ haloalkyl or cyclopropylmethyl.
Embodiment 181b. A compound of Embodiment 181a wherein each R^{14b} is independently H, methyl or cyclopropylmethyl.
Embodiment 182. A compound of Formula 1 or any one of Embodiments 1 through 181b wherein when R^{14a} and R^{14b} are taken together to form a 4- to 6-membered fully saturated heterocyclic ring, then said ring contains ring members, in addition to the connecting nitrogen atom, selected from carbon atoms and up to 1 heteroatom selected from up to 1 O, up to 1 S and up to 1 N atom, each ring optionally substituted with up to 2 substituents independently selected from halogen or methyl.
Embodiment 183. A compound of Embodiment 182 wherein R^{14a} and R^{14b} are taken together to form an azetidinyl, morpholinyl, pyrrolidinyl, piperidinyl, piperazinyl or thiomorpholinyl ring, each ring optionally substituted with up to 2 substituents independently selected from halogen or methyl.
Embodiment 184. A compound of Embodiment 183 wherein R^{14a} and R^{14b} are taken together to form an azetidinyl or pyrrolidinyl ring, each ring optionally substituted with up to 2 substituents independently selected from halogen or methyl.
Embodiment 185. A compound of Formula 1 or any one of Embodiments 1 through 185 wherein each R¹⁵ is independently H, cyano, halogen, methyl or methoxy.
Embodiment 186. A compound of Embodiment 185 wherein each R¹⁵ is independently H or methyl.
Embodiment 187. A compound of Formula 1 or any one of Embodiments 1 through 186 wherein each R¹⁶ is independently hydroxy, NR^{20a}R^{20b}, C₁-C₂ alkoxy, C₂-C₄ alkenyloxy, C₂-C₄ alkylcarbonyloxy or C₂-C₄ alkoxycarbonyloxy.
Embodiment 188. A compound of Embodiment 187 wherein each R¹⁶ is independently hydroxy, NR^{20a}R^{20b} or C₁-C₄ alkoxy.
Embodiment 189. A compound of Embodiment 188 wherein each R¹⁶ is independently hydroxy, NR^{20a}R^{20b} or methoxy.
Embodiment 190. A compound of Embodiment 189 wherein each R¹⁶ is hydroxy.
Embodiment 191. A compound of Formula 1 or any one of Embodiments 1 through 190 wherein each R¹⁷ is independently H or methyl.
Embodiment 192. A compound of Embodiment 191 wherein each R¹⁷ is H.
Embodiment 193. A compound of Formula 1 or any one of Embodiments 1 through 192 wherein when each R^{18a} and R^{18b} is separate (i.e. not taken together to form a ring), then each R^{18a} and R^{18b} is independently H, methyl or ethyl.
Embodiment 194. A compound of Embodiment 193 wherein each R^{18a} and R^{18b} is independently H or methyl.
Embodiment 195. A compound of Formula 1 or any one of Embodiments 1 through 194 wherein when R^{18a} and R^{18b} are taken together to form a 5- to 6-membered fully saturated heterocyclic ring, then said ring contains ring members, in addition to the connecting nitrogen atom, selected from carbon atoms and up to 1 heteroatom selected from up to 1 O, up to 1 S and up to 1 N atom, each ring optionally substituted with up to 2 methyl groups.
Embodiment 196. A compound of Embodiment 195 wherein R^{18a} and R^{18b} are taken together to form an azetidinyl, morpholinyl, pyrrolidinyl, piperidinyl, piperazinyl, or thiomorpholinyl ring, each ring optionally substituted with up to 2 methyl groups.
Embodiment 197. A compound of Formula 1 or any one of Embodiments 1 through 196 wherein each R¹⁹ is independently cyano, halogen, hydroxy, C₁-C₃ alkyl, C₁-C₃ haloalkyl, C₃-C₆ cycloalkyl, C₁-C₃ alkoxy, C₁-C₃ haloalkoxy, C₂-C₃ alkoxyalkoxy, C₁-C₃ alkylthio, C₁-C₃ alkylsulfinyl, C₁-C₃ alkylsulfonyl, C₁-C₃ haloalkylsulfonyl, C₂-C₃ alkylcarbonyl, C₂-C₃ haloalkylcarbonyl, C₂-C₃ alkoxycarbonyl, C₂-C₃ alkylaminocarbonyl or C₃-C₅ dialkylaminocarbonyl.
Embodiment 198. A compound of Embodiment 197 wherein each R¹⁹ is independently cyano, halogen, hydroxy, C₁-C₂ alkyl, C₁-C₂ haloalkyl, C₃-C₆ cycloalkyl, C₁-C₂ alkoxy, C₁-C₂ haloalkoxy, C₁-C₂ alkylthio, C₁-C₂ alkylsulfonyl, C₁-C₂ haloalkylsulfonyl, C₂-C₃ alkylcarbonyl, C₂-C₃ haloalkylcarbonyl, C₂-C₃ alkoxycarbonyl or C₂-C₃ alkylaminocarbonyl.
Embodiment 199. A compound of Embodiment 197 wherein each R¹⁹ is independently cyano, halogen, C₁-C₂ alkyl, C₁-C₂ haloalkyl, C₃-C₆ cycloalkyl, C₁-C₂ alkoxy, C₁-C₂ haloalkoxy, C₂-C₃ alkylcarbonyl, C₂-C₃ haloalkylcarbonyl or C₂-C₃ alkoxycarbonyl.
Embodiment 200. A compound of Embodiment 199 wherein each R¹⁹ is independently cyano, halogen, cyclopropyl, cyclobutyl, methoxy, halomethoxy or methoxy carbonyl.
Embodiment 200a. A compound of Embodiment 200 wherein each R¹⁹ is independently cyano, halogen, cyclopropyl or methoxy.
Embodiment 200b. A compound of Embodiment 200a wherein each R¹⁹ is independently cyano, Cl, F, cyclopropyl or methoxy.
Embodiment 201. A compound of Formula 1 or any one of Embodiments 1 through 200b wherein each E1 is independently a direct bond, C(═O)O or C(═O)N(R²⁵).
Embodiment 202. A compound of Embodiment 201 wherein each E1 is independently a direct bond or C(═O)O.
Embodiment 203. A compound of Embodiment 202 wherein each E1 is C(═O)O.
Embodiment 204. A compound of Formula 1 or any one of Embodiments 1 through 203 wherein each V is independently a direct bond; or C₁-C₆ alkylene, C₂-C₆ alkenylene or C₃-C₆ alkynylene, each optionally substituted with up to 2 substituents independently selected from halogen, cyano, nitro, hydroxy, C₁-C₂ alkyl, C₁-C₂ haloalkyl, C₁-C₂ alkoxy and C₁-C₂ haloalkoxy.
Embodiment 205. A compound of Embodiment 204 wherein each V is independently a direct bond; or C₁-C₃ alkylene, each optionally substituted with up to 2 substituents independently selected from halogen, hydroxy, C₁-C₂ alkyl, C₁-C₂ alkoxy and C₁-C₂ haloalkoxy.
Embodiment 206. A compound of Embodiment 205 wherein each V is independently a direct bond or C₁-C₃ alkylene.
Embodiment 207. A compound of Embodiment 206 wherein each V is independently a direct bond or CH₂.
Embodiment 208. A compound of Embodiment 207 wherein each V is a direct bond.
Embodiment 209. A compound of Embodiment 207 wherein each V is independently C₁-C₂ alkylene.
Embodiment 210. A compound of Embodiment 209 wherein each V is CH₂.
Embodiment 211. A compound of Formula 1 or any one of Embodiments 1 through 210 wherein each Q is independently phenyl optionally substituted with up to 2 substituents independently selected from R²⁷; or a 5- to 6-membered heteroaromatic ring, each ring containing ring members selected from carbon atoms and 1 to 4 heteroatoms independently selected from up to 2 O, up to 2 S and up to 4 N atoms, each ring optionally substituted with up to 2 substituents independently selected from R²⁷; or a 3- to 6-membered nonaromatic heterocyclic ring, each ring containing ring members selected from carbon atoms and 1 to 4 heteroatoms independently selected from up to 2 O, up to 2 S and up to 4 N atoms, wherein up to 2 ring members are independently selected from C(═O), C(═S), S(═O) and S(═O)₂, each ring optionally substituted with up to 2 substituents independently selected from R²⁷.
Embodiment 212. A compound of Embodiment 210 wherein each Q is independently phenyl optionally substituted with up to 2 substituents independently selected from R²⁷; or pyridinyl, pyrazolyl, imidazolyl, triazolyl, thiazolyl, oxazolyl, isoxazolyl, thienyl, isoxazolinyl, piperidinyl, morpholinyl or piperazinyl, each optionally substituted with up to 2 substituents independently selected from R²⁷.
Embodiment 213. A compound of Embodiment 212 wherein each Q is independently phenyl optionally substituted with up to 2 substituents independently selected from R²⁷; or pyridinyl, pyrazolyl, imidazolyl, triazolyl, thiazolyl or oxazolyl, each optionally substituted with up to 2 substituents independently selected from R²⁷.
Embodiment 214. A compound of Embodiment 213 wherein each Q is independently phenyl optionally substituted with up to 2 substituents independently selected from R²⁷; or pyridinyl or pyrazolyl, each optionally substituted with up to 2 substituents independently selected from R²⁷.
Embodiment 214a. A compound of Embodiment 214 wherein each Q is independently phenyl or pyridinyl, each optionally substituted with up to 2 substituents independently selected from R²⁷.
Embodiment 214b. A compound of Embodiment 214a wherein each Q is independently phenyl optionally substituted with up to 2 substituents independently selected from R²⁷
Embodiment 215. A compound of Formula 1 or any one of Embodiments 1 through 214b wherein when each R^{20a} is separate (i.e. not taken together with R^{20b} to form a ring), then each R^{20a} is independently H, methyl or methylcarbonyl.
Embodiment 216. A compound of Formula 1 or any one of Embodiments 1 through 215 wherein when each R^{20b} is separate (i.e. not taken together with R^{20a} to form a ring), then each R^{20b} is independently H, cyano, methyl, methylcarbonyl, methoxycarbonyl, methoxycarbonylmethyl, methylaminocarbonyl or dimethylaminocarbonyl.
Embodiment 217. A compound of Formula 1 or any one of Embodiments 1 through 216 wherein when R^{20a} and R^{20b} are taken together to form a 5- to 6-membered fully saturated heterocyclic ring, then said ring contains ring members, in addition to the connecting nitrogen atom, selected from carbon atoms and up to 1 heteroatom selected from up to 1 O, up to 1 S and up to 1 N atom, each ring optionally substituted with up to 2 methyl groups.
Embodiment 218. A compound of Embodiment 217 wherein R^{20a} and R^{20b} are taken together to form an azetidinyl, morpholinyl, pyrrolidinyl, piperidinyl, piperazinyl or thiomorpholinyl ring, each ring optionally substituted with up to 2 methyl groups.
Embodiment 219. A compound of Formula 1 or any one of Embodiments 1 through 218 wherein each R²¹ and R²³ is independently H, cyano, halogen, methyl or methoxy.
Embodiment 220. A compound of Formula 1 or any one of Embodiments 1 through 219 wherein each R²² is independently H, C₁-C₃ alkyl, C₁-C₃ haloalkyl, C₂-C₃ alkylcarbonyl or C₂-C₃ alkoxycarbonyl; or phenyl optionally substituted with up to 2 substituents independently selected halogen and methyl; or a 5- to 6-membered fully saturated heterocyclic ring, each ring containing ring members selected from carbon atoms and up to 2 heteroatoms independently selected from up to 2 O, up to 2 S and up to 2 N atoms, each ring optionally substituted with up to 2 substituents independently selected from halogen and methyl.
Embodiment 221. A compound of Embodiment 220 wherein each R²² is independently H or C₁-C₂ alkyl.
Embodiment 222. A compound of Formula 1 or any one of Embodiments 1 through 221 wherein each R²⁴ is independently H, cyano or C₁-C₂ alkyl.
Embodiment 223. A compound of Formula 1 or any one of Embodiments 1 through 222 wherein each R²⁵ and R²⁶ is independently H, cyano, hydroxy, C₁-C₄ alkyl or C₁-C₄ haloalkyl.
Embodiment 224. A compound of Embodiment 223 wherein each R²⁵ and R²⁶ is independently H, cyano, hydroxy or C₁-C₂ alkyl.
Embodiment 225. A compound of Formula 1 or any one of Embodiments 1 through 224 wherein each R²⁷ is independently halogen, cyano, C₁-C₄ alkyl, C₁-C₄ haloalkyl or C₁-C₄ alkoxy.
Embodiment 226. A compound of Embodiment 225 wherein each R²⁷ is independently halogen, cyano, C₁-C₂ alkyl, C₁-C₂ haloalkyl or C₁-C₂ alkoxy.
Embodiment 227. A compound of Embodiment 226 wherein each R²⁷ is independently halogen, methyl or methoxy.
Embodiment 228. A compound of Embodiment 227 wherein each R²⁷ is independently halogen.
Embodiment 229. A compound of Formula 1 or any one of Embodiments 1 through 228 wherein Z is a direct bond, O, NH, C(═O), C(═O)NH, NHC(═O), NHC(═O)NH, OC(═O)NH, NHC(═O)O, S(═O)₂NH, NHS(═O)₂ or NHS(═O)₂NH.
Embodiment 230. A compound of Embodiment 229 wherein Z is a direct bond, O, NH, C(═O), C(═O)NH or NHC(═O).
Embodiment 231. A compound of Embodiment 230 wherein Z is a direct bond, O, NH or C(═O).
Embodiment 232. A compound of Embodiment 231 wherein Z is a direct bond.
Embodiment 233. A compound of Formula 1 or any one of Embodiments 1 through 232 wherein each R²⁸ is independently H or C₁-C₃ alkyl.
Embodiment 234. A compound of Embodiment 233 wherein each R²⁸ is independently H or methyl.
Embodiment 235. A compound of Formula 1 or any one of Embodiments 1 through 234 wherein m is 0 or 2.
Embodiment 236. A compound of Embodiment 235 wherein m is 2.

Embodiments of this invention, including Embodiments 1-236 above as well as any other embodiments described herein, can be combined in any manner, and the descriptions of variables in the embodiments pertain not only to the compounds of Formula 1 but also to the starting compounds and intermediate compounds (e.g. compounds of Formula 10) useful for preparing the compounds of Formula 1. In addition, embodiments of this invention, including Embodiments 1-236 above as well as any other embodiments described herein, and any combination thereof, pertain to the compositions and methods of the present invention.

Combinations of Embodiments 1-236 are illustrated by:

Embodiment A. A compound of Formula 1 wherein
R¹ is CF₃, CCl₃ or CF₂C₁;
W is O;
R^{5a} and R^{5b} are each independently H, hydroxy or methyl;
R^{2a} and R^{2b} are each independently H or methyl; or
R^{2a} and R^{2b} are taken together with the atoms X and Y to which they are attached to form a 5- to 6-membered saturated ring containing ring members, in addition to the atoms X and Y, selected from carbon atoms, wherein up to 1 carbon atom ring member is selected from C(═O), the ring optionally substituted with up to 2 substituents independently selected from halogen, cyano, methyl, halomethyl, methoxy and halomethoxy on carbon atom ring members;
R^{2c} is C₁-C₂ alkyl, C₂-C₃ alkenyl or C₂-C₃ alkynyl;
R^{2d} is H or methyl;
A¹ is CR^{6c}R^{6d} or O; A² is a direct bond, CR^{6e}R^{6f} or O;
R^{6a}, R^{6b}, R^{6c}, R^{6d}, R^{6e} and R^{6f} are each independently H, cyano, hydroxy, Br, Cl, F or methyl;
J is J-1, J-6 or J-14;
each R⁸ is independently F, Cl or methyl;
each R^{9a} and R^{9b} is independently H, halogen or methyl;
n is 0, 1 or 2;
E¹ is C₁-C₆ alkoxy, C₁-C₆ alkylsulfonyl, C₂-C₆ alkylcarbonyl or C₂-C₆ alkoxycarbonyl, wherein each carbon atom is optionally substituted with up to 1 substituent selected from R^{10a} and up to 3 substituents independently selected from R^{10b};
R^{10a} is phenyl optionally substituted with up to 2 substituents independently selected from R^{11a}; or a 5- to 6-membered heterocyclic ring containing ring members selected from carbon atoms and 1 to 4 heteroatoms independently selected from up to 2 O, up to 2 S and up to 4 N atoms, each ring optionally substituted with up to 2 substituents independently selected from R^{11a} on carbon atom ring members and R^{11b} on nitrogen atom ring members;
each R^{10b} is independently halogen, hydroxy, C₁-C₄ alkyl, C₁-C₄ haloalkyl, C₁-C₄ alkoxy, C₁-C₄ haloalkoxy, C₁-C₄ alkylsulfonyl, C₂-C₄ alkylcarbonyl or C₂-C₅ alkoxycarbonyl;
each R^{11a} is independently halogen, C₁-C₂ alkyl, C₁-C₂ haloalkyl, C₁-C₂ alkoxy or C₂-C₃ alkoxycarbonyl;
each R^{11b} is independently methyl, methoxy, methylcarbonyl or methoxycarbonyl;
G is selected from the group consisting of:

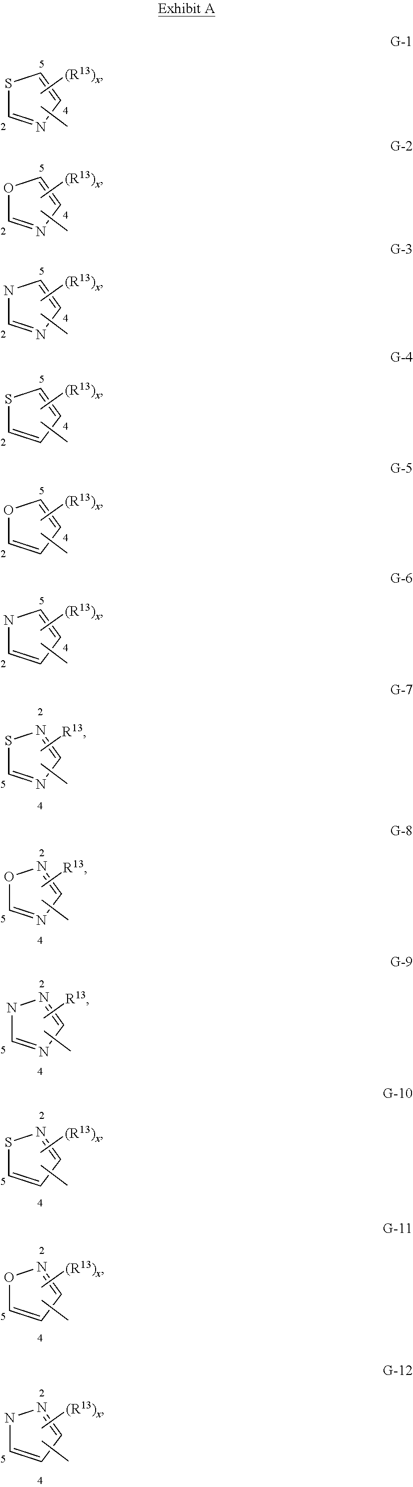

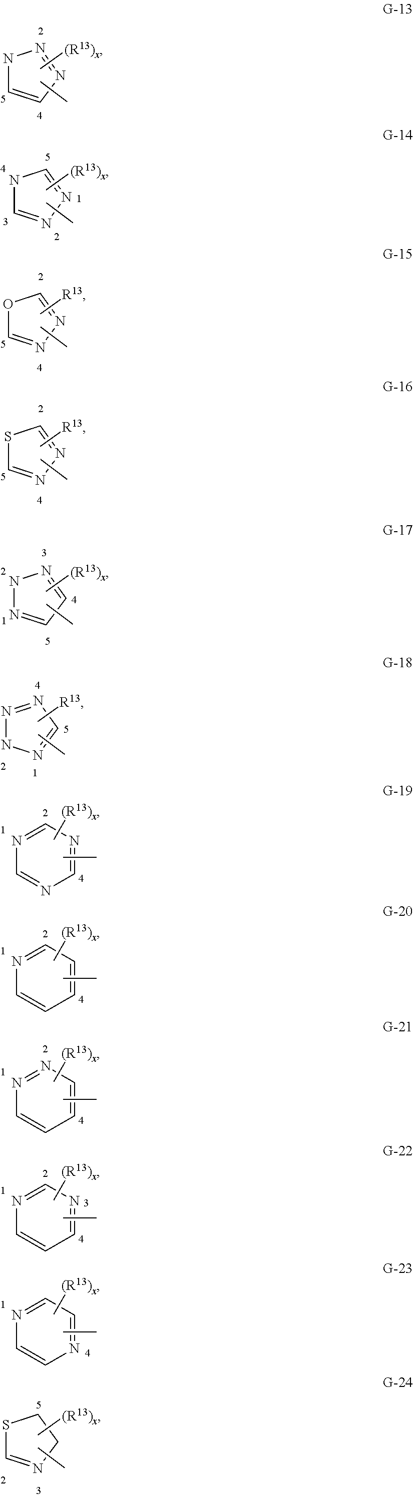

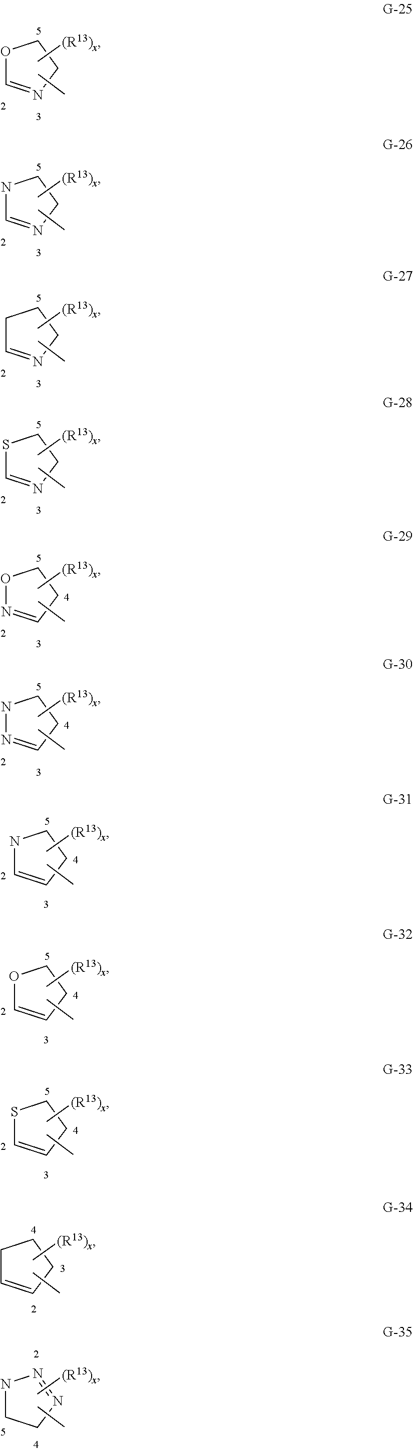

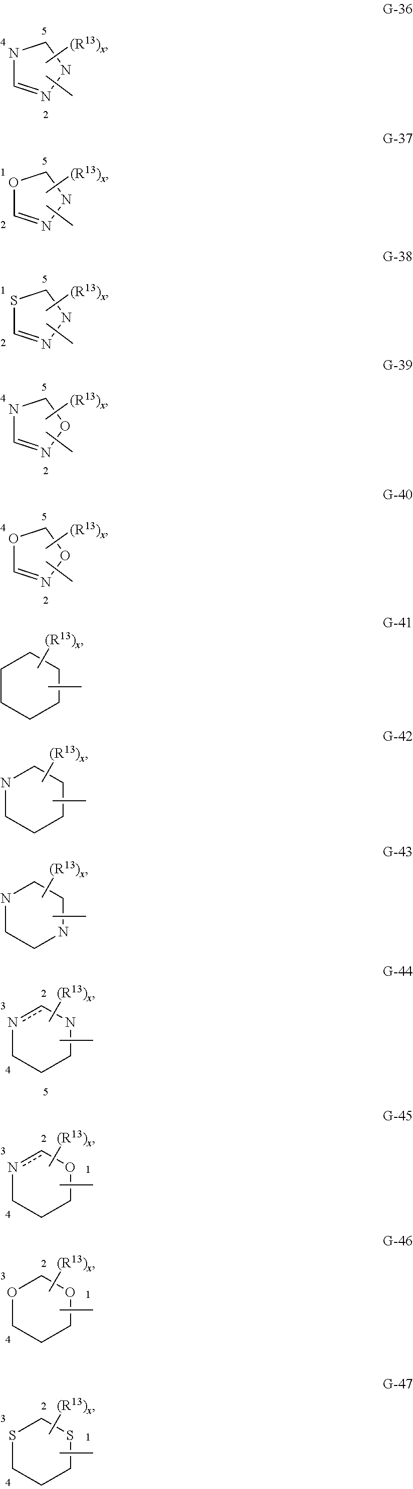

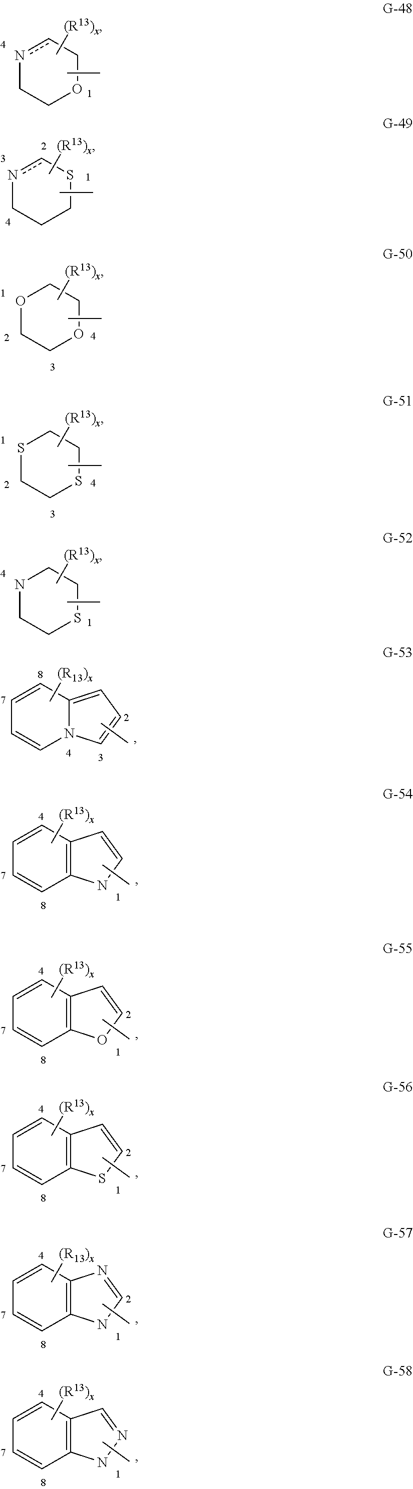

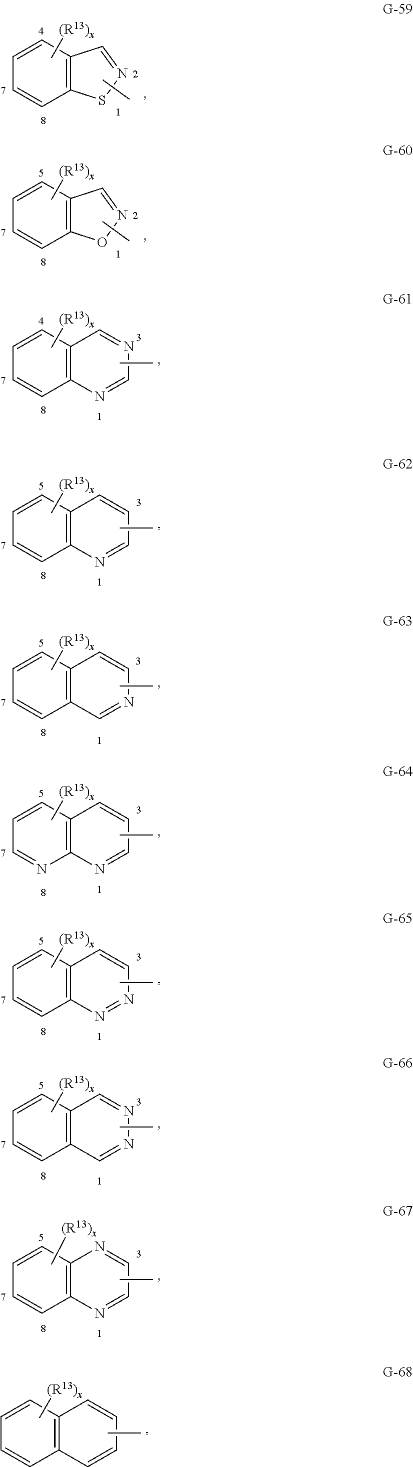

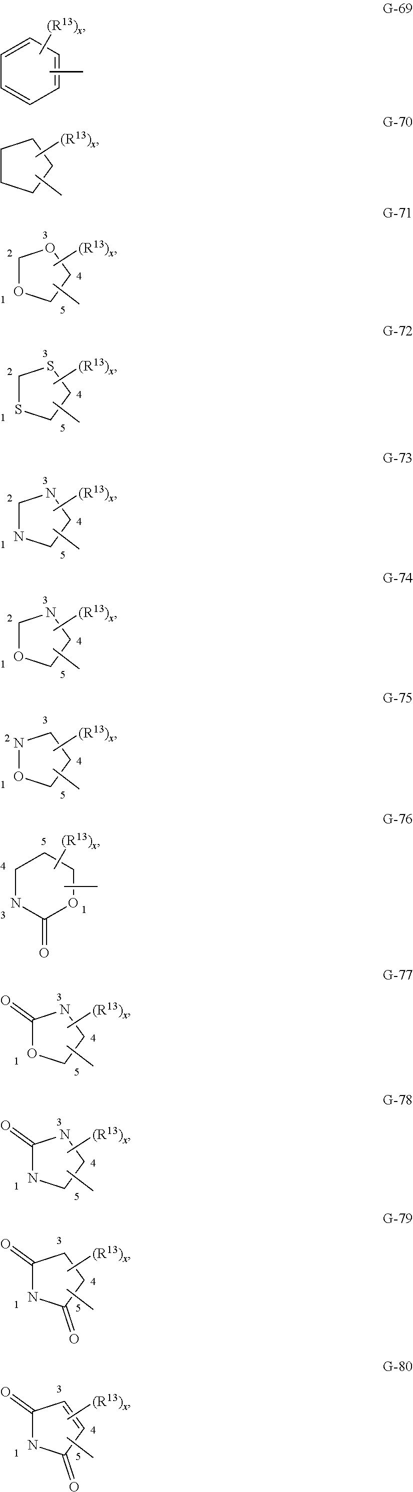

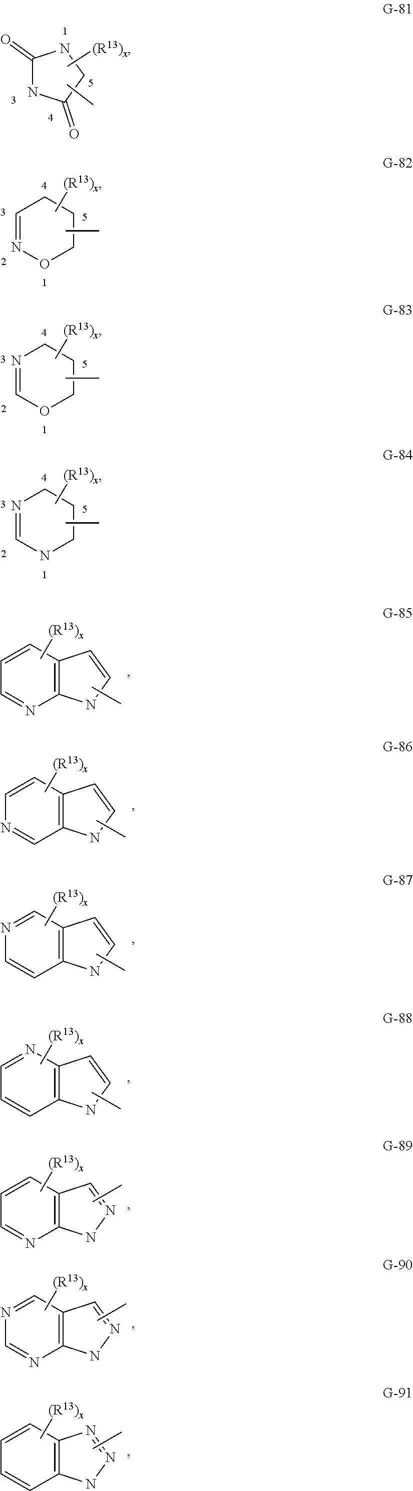

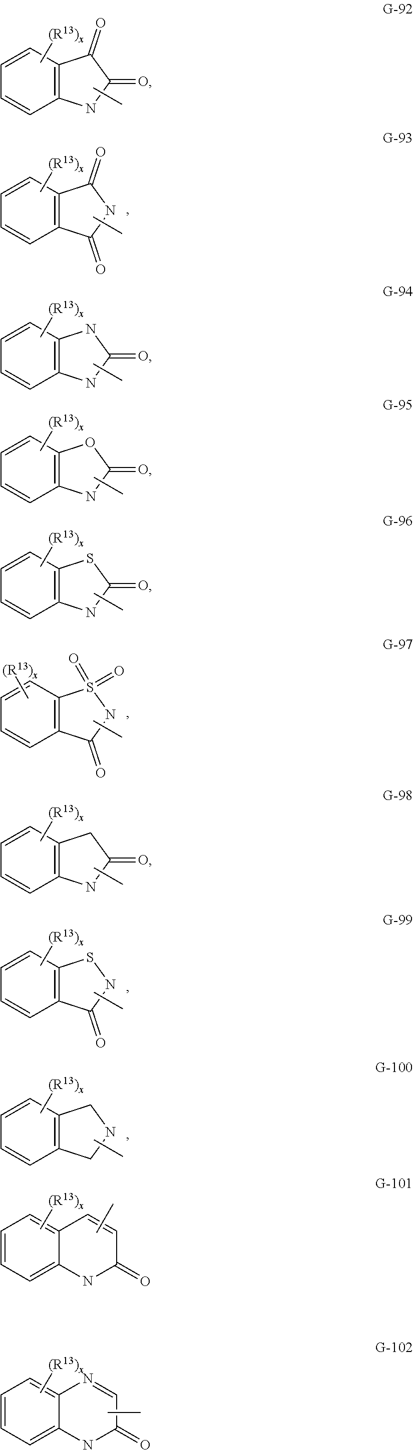

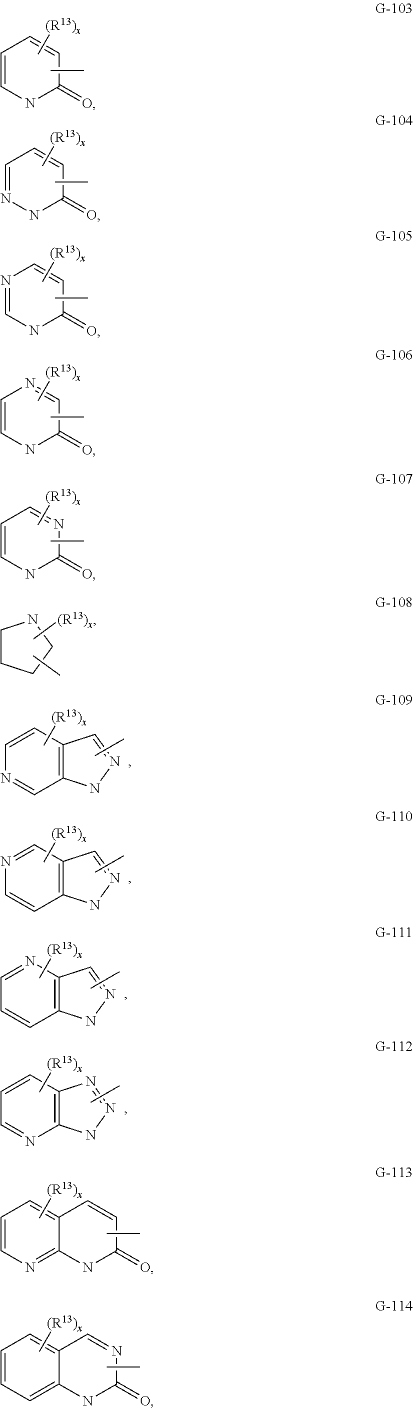

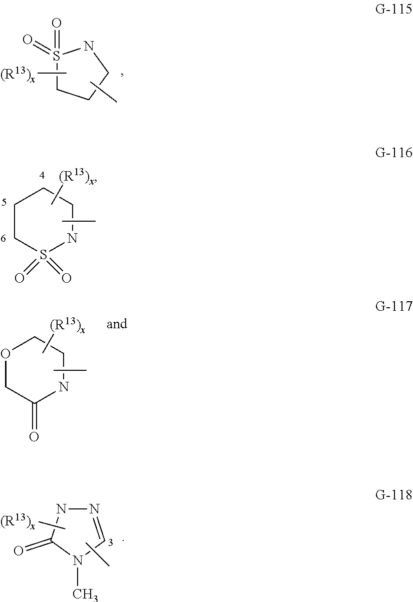

wherein the floating bond is connected to Z in Formula 1 through any available carbon or nitrogen atom of the depicted ring or ring system; and x is 0, 1, 2 or 3;
each R¹³ is independently C(═O)NR^{14a}R^{14b} or —U—V-Q; or C₁-C₆ alkyl, C₂-C₆ alkenyl, C₂-C₆ alkynyl, C₁-C₀ alkoxy, C₂-C₆ alkenyloxy, C₂-C₆ alkynyloxy, C₁-C₆ alkylsulfonyl, C₁-C₆ alkylsulfonyloxy, C₁-C₆ alkylsulfonylamino, C₂-C₆ alkylcarbonyl, C₂-C₆ alkoxycarbonyl, C₃-C₆ alkenyloxycarbonyl, C₃-C₆ alkynyloxycarbonyl, C₄-C₆ cycloalkoxycarbonyl or C₂-C₆ alkoxycarbonyloxy, each optionally substituted with up to 3 substituents independently selected from R¹⁹;
each R^{14a} is independently H, C₁-C₂ alkyl, C₂-C₄ alkenyl, C₂-C₄ alkynyl, C₂-C₄ alkylcarbonyl or C₂-C₄ alkoxycarbonyl;
each R^{14b} is independently H, C₁-C₄ alkyl, C₁-C₄ haloalkyl, C₂-C₄ alkenyl, C₂-C₄ haloalkenyl, C₂-C₄ alkynyl, C₃-C₅ cycloalkyl, C₄-C₀ cycloalkylalkyl, C₂-C₄ alkoxyalkyl, C₂-C₄ haloalkoxyalkyl, C₂-C₄ alkylaminoalkyl or C₃-C₅ dialkylaminoalkyl; or
R^{14a} and R^{14b} are taken together to form an azetidinyl, morpholinyl, pyrrolidinyl, piperidinyl, piperazinyl or thiomorpholinyl ring, each ring optionally substituted with up to 2 substituents independently selected from halogen or methyl;
each R¹⁹ is independently cyano, halogen, C₁-C₂ alkyl, C₁-C₂ haloalkyl, C₃-C₅ cycloalkyl, C₁-C₂ alkoxy, C₁-C₂ haloalkoxy, C₂-C₃ alkylcarbonyl, C₂-C₃ haloalkylcarbonyl or C₂-C₃ alkoxy carbonyl;
each U is independently a direct bond, C(═O)O or C(═O)N(R²⁵);
each V is independently a direct bond; or C₁-C₃ alkylene, each optionally substituted with up to 2 substituents independently selected from halogen, hydroxy, C₁-C₂ alkyl, C₁-C₂ alkoxy and C₁-C₂ haloalkoxy;
each Q is independently phenyl optionally substituted with up to 2 substituents independently selected from R²⁷; or pyridinyl, pyrazolyl, imidazolyl, triazolyl, thiazolyl or oxazolyl, each optionally substituted with up to 2 substituents independently selected from R²⁷;
each R²⁵ is independently H, cyano, hydroxy or C₁-C₂ alkyl;
each R²⁷ is independently halogen, cyano, C₁-C₂ alkyl, C₁-C₂ haloalkyl or C₁-C₂ alkoxy; and
Z is a direct bond, O, NH, C(═O), C(═O)NH, NHC(═O), NHC(═O)NH, OC(═O)NH, NHC(═O)O, S(═O)₂NH, NHS(═O)₂ or NHS(═O)₂NH.
   Embodiment AA. A compound of Embodiment A wherein
R¹ is CF₃;
X is O;
Y is O;
L is a direct bond or CH₂; and
Z is a direct bond.
   Embodiment AAA. A compound of Embodiment A wherein
R¹ is CF₃;
Z is a direct bond.
   Embodiment B. A compound of Embodiment A wherein
T is T-2 or T-3;
R¹ is CF₃;
X is O; Y is O;
R^{2a} and R^{2b} are each independently H or methyl; or
R^{2a} and R^{2b} are taken together with the atoms X and Y to which they are attached to form a 5-membered saturated ring containing ring members, in addition to the atoms X and Y, selected from carbon atoms, the ring optionally substituted with up to 1 substituent selected from halogen, methyl and halomethyl on a carbon atom ring member;
R^{2c} is methyl or ethyl;
R^{2d} is H;
A¹ is O;
A² is a direct bond, CH₂ or O;
R^{6a} and R^{6b} are each independently H, cyano hydroxy or methyl;
J is J-1 or J-6;
q is 0 or 1;
each R^{9a} and R^{9b} is independently H or methyl;
E¹ is C₁-C₃ alkoxy, C₂-C₃ alkylcarbonyl or C₂-C₃ alkoxycarbonyl, wherein each carbon atom is optionally substituted with up to 1 substituent selected from R^{10a} and up to 3 substituents independently selected from R^{10b};
R^{10a} is pyrazolyl, imidazolyl or triazolyl, each optionally substituted with up to 2 substituents independently selected from R^{11a} on carbon atom ring members;
each R^{10b} is independently halogen, C₁-C₂ alkyl, C₁-C₂ haloalkyl, C₁-C₂ alkoxy or C₂-C₄ alkoxycarbonyl;
G is G-1, G-3, G-12 or G-22;
x is 1 or 2;
each R¹³ is independently C(═O)NR^{14a}R^{14b} or —U—V-Q; or C₂-C₅ alkoxycarbonyl, C₃-C₅ alkenyloxycarbonyl, C₃-C₅ alkynyloxycarbonyl or C₄-C₅ cycloalkoxycarbonyl, each optionally substituted with up to 3 substituents independently selected from R¹⁹;
each R^{14a} is independently H or C₁-C₂ alkyl;
each R^{14b} is independently H, C₁-C₃ alkyl, C₁-C₃ haloalkyl, cyclopropylmethyl or C₂-C₄ alkoxy alkyl;
each R¹⁹ is independently cyano, halogen, cyclopropyl, cyclobutyl, methoxy, halomethoxy or methoxy carbonyl;
each U is independently a direct bond or C(═O)O;
each V is independently a direct bond or CH₂;
each Q is independently phenyl or pyridinyl, each optionally substituted with up to 2 substituents independently selected from R²⁷;
each R²⁷ is independently halogen, methyl or methoxy; and
Z is a direct bond, O, NH, C(═O), C(═O)NH or NHC(═O).
   Embodiment BB. A compound of Embodiment B wherein
L is a direct bond or CH₂;
G is G-1 or G-12; and
Z is a direct bond.
   Embodiment BBB. A compound of Embodiment B wherein
Z is a direct bond.
   Embodiment C. A compound of Embodiment B wherein
R^{2a} and R^{2b} are each H; or
R^{2a} and R^{2b} are taken together with the atoms X and Y to which they are attached to form a 5-membered saturated ring containing ring members, in addition to the atoms X and Y, selected from carbon atoms;
A² is a direct bond;
R^{6a} and R^{6b} are each H;
R⁸ is F or Cl;
L is a direct bond, CH₂ or CH₂CH₂;
E¹ is C₁-C₂ alkoxy or C₂-C₃ alkoxycarbonyl, wherein each carbon atom is optionally substituted with up to 1 substituent selected from R^{10a};
R^{10a} is pyrazolyl or imidazolyl, each optionally substituted with up to 2 substituents independently selected from R^{11a} on carbon atom ring members;
each R^{11a} is independently methoxycarbonyl or ethoxycarbonyl;
G is G-1 and the 2-position of G-1 is connected to Z and the 4-position is connected to R¹³; or G is G-12 and the 1-position of G-12 is connected to Z and the 4-position is connected to R¹³; or G is G-12 and the 1-position of G-12 is connected to Z and the 3-position is connected to R¹³;
x is 1;
R¹³ is C(═O)NR^{14a}R^{14b} or —U—V-Q; or C₂-C₅ alkoxycarbonyl, C₃-C₅ alkynyloxycarbonyl or C₄-C₆ cycloalkoxycarbonyl, each optionally substituted with up to 1 substituent selected from R¹⁹;
R^{14a} is H;
R^{14b} is H, methyl or cyclopropylmethyl;
R¹⁹ is cyano, halogen, cyclopropyl or methoxy;
U is C(═O)O;
V is CH₂;
Q is phenyl optionally substituted with up to 2 substituents independently selected from R²⁷; and
Z is a direct bond, O, NH or C(═O).
   Embodiment CC. A compound of Embodiment C wherein
L is a direct bond or CH₂; and
Z is a direct bond.
   Embodiment D. A compound of Embodiment C wherein
R⁸ is F;
L is a direct bond or CH₂;
E¹ is methoxy substituted with 1 substituent selected from R^{10a};
R^{10a} is pyrazolyl optionally substituted with up to 1 substituent selected from R^{11a} on a carbon atom ring member;
G is G-12 and the 1-position of G-12 is connected to Z and the 4-position is connected to R¹³; or G is G-12 and the 1-position of G-12 is connected to Z and the 3-position is connected to R¹³; and
R¹³ is C₂-C₅ alkoxycarbonyl optionally substituted with up to 1 substituent selected from R¹⁹;
R¹⁹ is cyano, Cl, F, cyclopropyl or methoxy; and
Z is a direct bond.
   Embodiment DD. A compound of Embodiment D wherein
L is a direct bond or CH₂, provided that when L is a direct bond, then E is E¹, and when L is CH₂, then E is E².
   Embodiment E. A compound of Embodiment D wherein
J is J-1;
q is 0;
L is CH₂;
E is E²;
G is G-12 and the 1-position of G-12 is connected to Z and the 4-position is connected to R¹³; and
R¹³ is methoxy carbonyl or ethoxy carbonyl.
   Embodiment F. A compound of any one of Embodiments A through E wherein
T is T-2; and
R¹³ is ethoxy carbonyl.
   Embodiment G. A compound of any one of Embodiments A through E wherein
T is T-3; and
R¹³ is ethoxy carbonyl.

Specific embodiments include compounds of Formula 1 selected from the group consisting of:
ethyl 1-[[4-(3,3,3-trifluoro-2,2-dihydroxypropoxy)phenyl]methyl]-1H-pyrazole-4-carboxylate (Compound 1);
ethyl 1-[[4-[[2-(trifluoromethyl)-1,3-dioxolan-2-yl]methoxy]phenyl]methyl]-1H-pyrazole-4-carboxylate (Compound 32);
ethyl 1-[[4-[[(1Z)-2-ethoxy-3,3,3-trifluoro-1-propen-1-yl]oxy]phenyl]methyl]-1H-pyrazole-4-carboxylate (Compound 64);
ethyl 1-[[4-[[(1Z)-2-ethoxy-3,3,3-trifluoro-1-propen-1-yl]oxy]phenyl]methyl]-1H-pyrazole-3-carboxylate (Compound 231);
ethyl 1-[[4-[[(1Z)-2-ethoxy-3,3,3-trifluoro-1-propen-1-yl]oxy]-3-fluorophenyl]methyl]-1H-pyrazole-4-carboxylate (Compound 262);
ethyl 1-[[3-[[(1Z)-2-ethoxy-3,3,3-trifluoro-1-propen-1-yl]oxy]phenyl]methyl]-1H-pyrazole-4-carboxylate (Compound 265);
ethyl 1-[[4-[[(1Z)-2-ethoxy-3,3,3-trifluoro-1-propen-1-yl]oxy]phenoxy]methyl]-1H-pyrazole-4-carboxylate (Compound 364);
N-(cyclopropylmethyl)-2-[[4-[[2-(trifluoromethyl)-1,3-dioxolan-2-yl]methoxy]phenyl]-methyl]thiazole-4-carboxamide (Compound 71);
2-methylpropyl 1-[[4-[[2-(trifluoromethyl)-1,3-dioxolan-2-yl]methoxy]phenyl]methyl]-1H-pyrazole-4-carboxylate (Compound 126);
cyclopropylmethyl 1-[[4-[[2-(trifluoromethyl)-1,3-dioxolan-2-yl]methoxy]phenyl]methyl]-1H-pyrazole-4-carboxylate (Compound 127);
ethyl 1-[2-[4-[[(1Z)-2-ethoxy-3,3,3-trifluoro-1-propen-1-yl]oxy]phenyl]ethyl]-1H-pyrazole-4-carboxylate (Compound 132);
2-methoxyethyl 1-[[4-[[2-(trifluoromethyl)-1,3-dioxolan-2-yl]methoxy]phenyl]methyl]-1H-pyrazole-4-carboxylate (Compound 162);
2-butyn-1-yl 1-[[4-[[2-(trifluoromethyl)-1,3-dioxolan-2-yl]methoxy]phenyl]methyl]-1H-pyrazole-4-carboxylate (Compound 163);
3-cyanopropyl 1-[[4-[[2-(trifluoromethyl)-1,3-dioxolan-2-yl]methoxy]phenyl]methyl]-1H-pyrazole-4-carboxylate (Compound 171);
phenylmethyl 1-[[4-[[(1Z)-2-ethoxy-3,3,3-trifluoro-1-propen-1-yl]oxy]phenyl]methyl]-1H-pyrazole-4-carboxylate (Compound 186);
butyl 1-[[4-[[(1Z)-2-ethoxy-3,3,3-trifluoro-1-propen-1-yl]oxy]phenyl]methyl]-1H-pyrazole-4-carboxylate (Compound 218);
3-chloropropyl 1-[[4-[[(1Z)-2-ethoxy-3,3,3-trifluoro-1-propen-1-yl]oxy]phenyl]methyl]-1H-pyrazole-4-carboxylate (Compound 221);
methyl 4-(3,3,3-trifluoro-2,2-dihydroxypropoxy)phenylcarboxylate (Compound 229);
ethyl 1-[[3-fluoro-4-[[2-(trifluoromethyl)-1,3-dioxolan-2-yl]methoxy]phenyl]methyl]-1H-pyrazole-4-carboxylate (Compound 263);
ethyl 1-[[4-[[(1Z)-2-ethoxy-3,3,3-trifluoro-1-propen-1-yl]oxy]phenylmethoxy]methyl]-1H-pyrazole-4-carboxylate (Compound 297);
methyl 1-[[3-[[(1Z)-2-ethoxy-3,3,3-trifluoro-1-propen-1-yl]oxy]phenyl]methyl]-1H-pyrazole-4-carboxylate (Compound 330); and
propyl 1-[[3-[[(1Z)-2-ethoxy-3,3,3-trifluoro-1-propen-1-yl]oxy]phenyl]methyl]-1H-pyrazole-4-carboxylate (Compound 331).

Embodiments of the present invention also include:
Embodiment B1. A compound of Formula 10 wherein R³⁰ is C₁-C₂ alkyl, C₁-C₄ haloalkyl, phenyl, 4-methylphenyl, 4-bromophenyl or 4-nitrophenyl.
Embodiment B2. A compound of Embodiment B1 wherein R³⁰ is CH₃, CF₃, CH₂CF₃, (CF₂)₃CF₃, phenyl or 4-methylphenyl.
Embodiment B3. A compound of Embodiment B2 wherein R³⁰ is CH₃, CF₃, CH₂CF₃, phenyl or 4-methylphenyl.
Embodiment B4. A compound of Embodiment B3 wherein R³⁰ is CH₃, CF₃ or 4-methylphenyl.
Embodiment B5. A compound of Embodiment B4 wherein R³⁰ is CF₃.

As noted in the Summary of the Invention, this invention also relates to a compound of Formula 10, or an N-oxide or salt thereof. Also noted is that the embodiments of this invention, including Embodiments 1-236 above, relate also to compounds of Formula 10. Accordingly, combinations of Embodiments 1-236 are further illustrated by: Embodiment C1. A compound of Formula 10, or an N-oxide or salt thereof, wherein
R¹ is CF₃, CCl₃ or CFCl₂;
X is O;
Y is O;
R^{2a} and R^{2b} are each independently H or methyl; or
R^{2a} and R^{2b} are taken together with the atoms X and Y to which they are attached to form a 5-membered saturated ring containing ring members, in addition to the atoms X and Y, selected from carbon atoms, wherein up to 1 carbon atom ring member is selected from C(═O), the ring optionally substituted with up to 2 substituents independently selected from halogen, cyano, methyl, halomethyl, methoxy and halomethoxy on carbon atom ring members;
R^{6a} and R^{6b} are each independently H, cyano, Br, Cl, F or methyl; and
R³⁰ is C₁-C₄ alkyl, C₁-C₄ haloalkyl, phenyl, 4-methylphenyl 4-bromophenyl or 4-nitrophenyl.
   Embodiment C2. A compound of Embodiment C1 wherein
R¹ is CF₃;
R^{2a} and R^{2b} are each H; or
R^{2a} and R^{2b} are taken together with the atoms X and Y to which they are attached to form a 5-membered saturated ring containing ring members, in addition to the atoms X and Y, selected from carbon atoms;
R^{6a} and R^{6b} are each independently H or methyl; and
R³⁰ is CH₃, CF₃, CH₂CF₃, (CF₂)₃CF₃, phenyl or 4-methylphenyl.
   Embodiment C3. A compound of Embodiment C2 wherein
R^{6a} and R^{6b} are H; and
R³⁰ is CH₃, CF₃ or 4-methylphenyl.

Embodiment C4. A compound of Embodiment C3 wherein
R³⁰ is CF₃.

Embodiment C5. A compound of Embodiment C4 wherein
R^{2a} and R^{2b} are taken together with the atoms X and Y to which they are attached to form a 5-membered saturated ring containing ring members, in addition to the atoms X and Y, selected from carbon atoms; and
R³⁰ is CF₃.

In addition to the embodiments described above, this invention also provides a fungicidal composition comprising a compound of Formula 1 (including all stereoisomers, N-oxides, and salts thereof), and at least one other fungicide. Of note as embodiments of such compositions are compositions comprising a compound corresponding to any of the compound embodiments described above.

This invention also provides a fungicidal composition comprising a compound of Formula 1 (including all stereoisomers, N-oxides, and salts thereof) (i.e. in a fungicidally effective amount), and at least one additional component selected from the group consisting of surfactants, solid diluents and liquid diluents. Of note as embodiments of such compositions are compositions comprising a compound corresponding to any of the compound embodiments described above.

This invention provides a method for controlling plant diseases caused by fungal plant pathogens comprising applying to the plant or portion thereof, or to a plant seed, a fungicidally effective amount of a compound of Formula 1 (including all stereoisomers, N-oxides, and salts thereof). Of note as embodiments of such methods are methods comprising applying a fungicidally effective amount of a compound corresponding to any of the compound embodiments describe above. Of particular note are embodiments where the compounds are applied as compositions of this invention.

One or more of the following methods and variations as described in Schemes 1-17 can be used to prepare the compounds of Formula 1. The definitions of E, L, A, A¹, A², J, T, X, Y, R¹, R^{2a}, R^{2b}, R^{2c}, R^{2d}, R^{6a}, R^{6b} and R²⁹ in compounds of Formulae 1-14 below are as defined above in the Summary of the Invention unless otherwise noted. Compounds of Formulae 1a-1a¹, 1b-1b⁶ and 1c-1c¹ are various subsets of Formula 1, and all substituents for Formulae 1a-1a¹, 1b-1b⁶ and 1c-1c¹ are as defined above for Formula 1 unless otherwise noted. As the synthetic literature includes many halomethyl ketone and hydrate-forming methods, which can readily be adapted to prepare compounds of the present invention, the following methods in Schemes 1-17 are simply representative examples of a wide variety of procedures useful for the preparation of the compounds of Formula 1. For reviews of ketone and hydrate-forming methods, see, for example, Tetrahedron 1991, 47, 3207-3258 and Chem. Communications 2013, 49(95), 11133-11148, and references cited therein. Also see the methods outlined in U.S. Pat. No. 6,350,892.

As shown in Scheme 1, Compounds of Formula 1a (i.e. Formula 1 wherein T is T-1 and W is O) wherein R¹ is CF₃ can be prepared by trifluoroacetylation of organometallic compounds of Formula 2. Typically, the ethyl ester of trifluoroacetic acid (i.e. ethyl trifluoroacetate) is used as the source of the trifluoroacetyl group in this method, but trifluoroacetonitrile and various trifluoroacetate salts can also be used. Depending on the reaction conditions, double-addition on the trifluoroacetyl compound can occur. Conducting the reaction at −65° C., or more preferably at −78° C., can reduce the occurrence of double addition adducts to trace amounts, particularly when using organometallic species of Formula 2 wherein M is Li or MgBr. Many other organometallic species yield similar results. For reaction conditions useful in the method of Scheme 1, as well as other well-established routes for the synthesize trifluoromethyl ketones see, for example, Journal of Organic Chemistry 1987, 52(22), 5026-5030; Chemical Communications 2013, 49(95), 11133-11148; and Journal of Fluorine Chemistry 1981, 18, 117-129. Conditions described in these references can easily be modified to prepare compounds of Formula 1a wherein R¹ is other than CF₃ (e.g., dihalo- or trichloro-moieties).

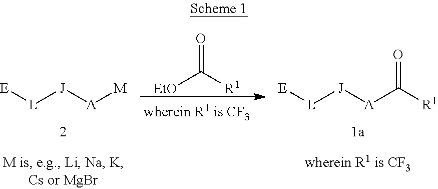

Compounds of Formula 1a (i.e. Formula 1 wherein T is T-1 and W is O) wherein R¹ is CF₃ can also be prepared via alkylation of ethyl 4,4,4-trifluoroacetoacetate (ETFAA) with compounds of Formula 3 wherein La is a leaving group such as halogen (e.g., Cl, Br) or sulfonate (e.g., mesylate). In this method ETFAA is first treated with a base such as sodium hydride in a polar aprotic solvent such tetrahydrofuran (THF), THF/hexamethylphosphoramide (HMPA) or acetone. The ETFAA anion then displaces the leaving group in compounds of Formula 3 to give an intermediate ester which undergoes hydrolysis and decarboxylation in the presence of lithium chloride (LiCl) and N,N-dimethylformamide (DMF) to give the ketone compound of Formula 1a. For reaction conditions see Journal Chemical Society, Chemical Communications 1989, (2), 83-84; Chemical Communications 2013, 49(95), 11133-11148; and Journal of Fluorine Chemistry 1989, 44, 377-394.

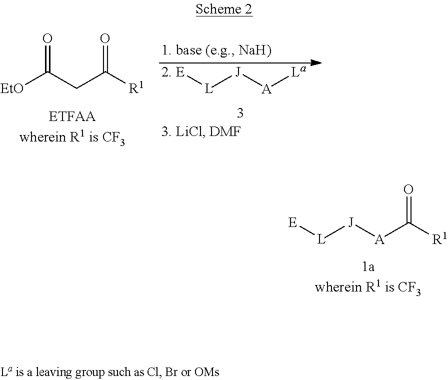

As shown in Scheme 3, compounds of Formula 1a (i.e. Formula 1 wherein T is T-1 and W is O) wherein R¹ is CF₃ can also be prepared by trifluoromethylation of an ester of Formula 5 with trifluoromethyltrimethylsilane (TMS-CF₃). The reaction is run in the present of a fluoride initiator such as tetrabutylammonium fluoride, and in an anhydrous solvent such as toluene or dichloromethane at about −78° C. (for reaction conditions see, for example, Angew. Chem., Int. Ed. 1998, 37(6), 820-821). Cesium fluoride can also be used as an initiator in a solvent such as 1,2-dimethoxyethane (glyme) at room temperature (for reaction conditions see, for example, J. Org. Chem., 1999, 64, 2873). The reaction proceeds through a trimethylsilicate intermediate, which is hydrolyzed with aqueous acid to give the desired trifluoromethyl ketone compound of Formula 1a. Weinreb amides may also be used in place of the starting esters (see, for example, Chem. Commun. 2012, 48, 9610).

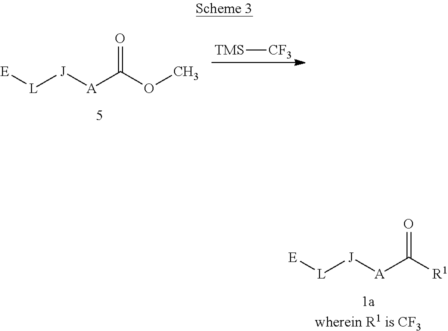

As shown in Scheme 4, compounds of Formula 1a¹ (i.e. Formula 1a wherein A is A¹-A²-CR^{6a}R^{6b}) wherein R¹ is CF₃ and at least one R^{6a} or R^{6b} is H can be prepared by reacting acid chlorides of Formula 6 with trifluoroacetic anhydride (TFAA) and pyridine in a solvent such as dichloromethane or toluene at a temperature between about 0 to 80° C. followed by aqueous hydrolysis (for reaction conditions see, for example, Tetrahedron 1995, 51, 2573-2584). Compounds of Formula 6 can be prepared from compounds of Formula 5 by ester hydrolysis to the corresponding carboxylic acid and treatment with oxalyl chloride, as known to one skilled in the art.

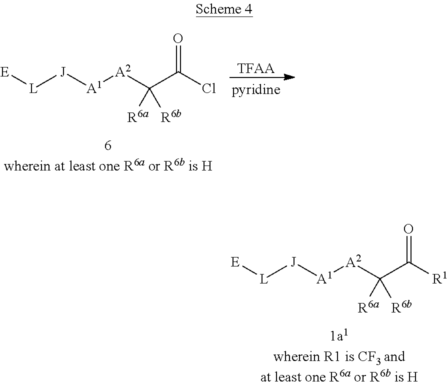

As shown in Scheme 5, compounds of Formula 1b (i.e. Formula 1 wherein T is T-2) wherein R^{2a}X and R^{2b}Y are OH can be prepared by oxidation of alcohols of Formula 4 to the corresponding dihydroxy. The oxidation reaction can be performed by a variety of means, such as by treatment of the alcohols of Formula 4 with manganese dioxide, Dess-Martin periodinane, pyridinium chlorochromate or pyridinium dichromate. For typical reaction conditions, see present Example 6, Step F and Example 8, Step F.

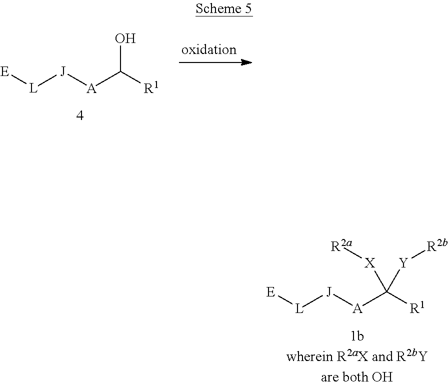

Scheme 6 illustrates a specific example of the general method of Scheme 5 for the preparation of a compound of Formula 1b¹ (i.e. Formula 1b wherein L is CH₂, J is phenyl (i.e. J-1), A is OCH₂ and R¹ is CF₃). In this method a compound of Formula 4a (i.e. Formula 4 wherein L is CH₂, J is phenyl (i.e. J-1), A is OCH₂ and R¹ is CF³) is reacted with an oxidizing reagent such as Dess-Martin periodinane in a solvent such as dichloromethane at a temperature between about 0 to 80° C. Present Example 1, Step C illustrates the method of Scheme 6.

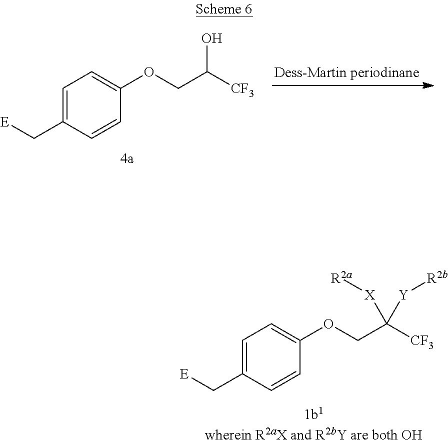

As shown in Scheme 7, compounds of Formula 4 can be prepared by reaction of compounds of Formula 2 with R¹CHO. For reactions conditions see, Tetrahedron Letters 2007, 48, 6372-6376.

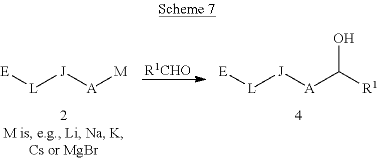

As shown in Scheme 8, compounds of Formula 4b (i.e. Formula 4 wherein A is OCR^{6a}R^{6b}) can be prepared by reacting a compound of Formula 7 with an epoxide of Formula 8. The reaction is typically carried out in a solvent such as acetonitrile with a catalytic amount of a base such as cesium or potassium carbonate at a temperature between about 20 to 80° C.; or in a solvent such as dichloromethane with a catalytic amount of a Lewis acid such as boron trifluoride etherate at a temperature between about 0 to 40° C. Present Example 8, Step E illustrates the method of Scheme 8. One skilled in the art will recognize that the method of Scheme 8 can also be performed when A is SCR^{6a}R^{6b} or N(R^{7a})CR^{6a}R^{6b}, thus providing other compounds of Formula 4b.

Compounds of Formulae 7 and 8 are available from commercial sources and can easily be prepared using commercial precursors and known methods. Present Example 1, Step A, Example 6, Step D and Example 8, Step D illustrate the preparation of a compound of Formula 7.

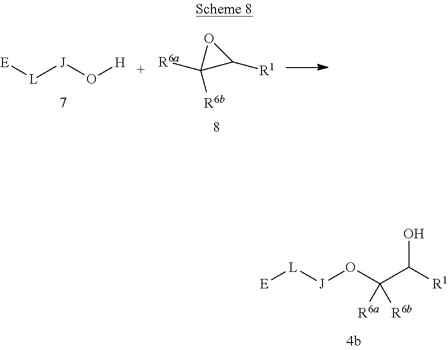

Scheme 9 illustrates a specific example of the general method of Scheme 8 for the preparation of a compound of Formula 4b¹ (i.e. Formula 4b wherein L is CH₂, J is phenyl (i.e. J-1), R^{6a} and R^{6b} are H and R¹ is CF₃) In this method a compound of Formula 7a (i.e. Formula 7 wherein L is CH₂ and J is phenyl (i.e. J-1)) is reacted with 2-(trifluoromethyl)oxirane (i.e. Formula 8a) in the presence of cesium carbonate in a solvent such as acetonitrile at a temperature between about 60 to 80° C. Present Example 1, Step B illustrates the method of Scheme 9.

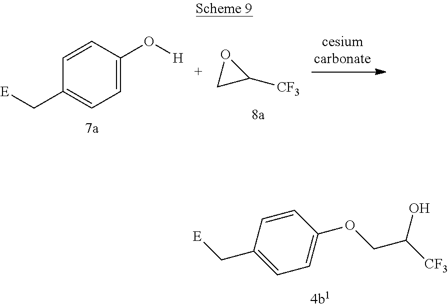

As illustrated in Scheme 10, ketones of Formula 1a (i.e. Formula 1 wherein T is T-1 and W is O) may exist in equilibrium with their corresponding ketone hydrates (i.e. dihydroxy) of Formula 1b (i.e. Formula 1 wherein T is T-2) wherein R^{2a}X and R^{2b}Y are OH. The predominance of Formula 1a or Formula 1b is dependent upon several factors, such as environment and structure. For example, in an aqueous environment ketones of Formula 1a can react with water to give ketone hydrates (also known as 1,1-geminal diols) of Formula 1b. Conversion back to the keto-form can usually be achieved by treatment with a dehydrating agent such as magnesium sulfate or molecular sieves. When the ketone moiety is in close proximity to an electron-withdrawing group, such as when R¹ is a trifluoromethyl group, the equilibrium typically favors the dihydrate form. In these cases, conversion back to the keto-form may require a strong dehydrating agent, such as phosphorus pentoxide (P₂O₅). For reaction conditions see, for example, Eur. J. Org. Chem. 2013, 3658-3661; and Chemical Communications 2013, 49(95), 11133-11148, and references cited therein.

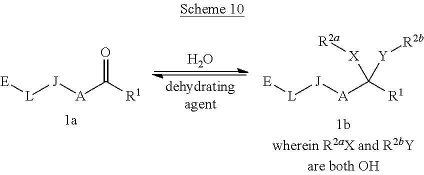

As shown in Scheme 11, ketones of Formula 1a may also exist in equilibrium with their hemiketals, hemithioketals and hemiaminals of Formula 1b² (i.e. Formula 1b wherein R^{2b}Y is OH and R^{2a} is other than H) along with their ketals, thioketals aminals of Formula 1b wherein R^{2a} and R^{2b} are other than H. Compounds of Formula 1b² can be prepared by reacting a compound of Formula 1a with a compound of formula R^{2a}X—H (e.g., alcohols for X being 0, thiols for X being S or amines for X being NR⁵a), usually in the presence of an catalysis, such as a Bronsted (i.e. protic) acid or Lewis acid (e.g. BF₃), (see, for example, Master Organic Chemistry (Online), On Acetals and Hemiacetals, May 28, 2010, www.masterorganic-chemistry.com/2010/05/28/on-acetals-and-hemiacetals). In a subsequent step, compounds of Formula 1b² can be treated with a compound of formula R^{2b}—H (e.g., alcohols for Y being 0, thiols for Y being S or amines for Y being NR^{5b}) under dehydrating conditions, or other means of water removal that will drive the equilibrium in the reaction to the right, to provide compounds of Formula 1b wherein R^{2a} and R^{2b} are other than H. Alternatively, ketones of Formula 1a can initially be treated with two equivalents (or an excess amount) of an alcohol, thiol or amine typically in the presence of a catalysis together with a dehydrating agent to provide compounds of Formula 1b directly (see, for example, the preparation of the dimethylketals using methanol and trimethyl orthoformate in U.S. Pat. No. 6,350,892).

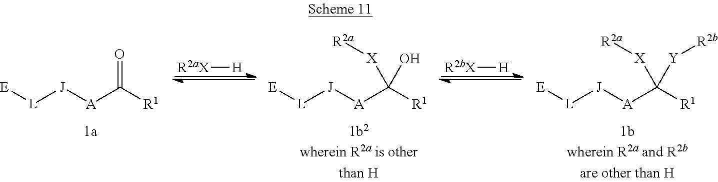

As illustrated in Scheme 12, cyclic ketals of Formula 1b³ (i.e. Formula 1b wherein X and Y are O, and R^{2a} and R^{2b} are taken together to form a 5- to 7-membered ring) can be prepared by treating the corresponding ketones of Formula 1a with haloalcohols (e.g., 2-chloroethanol or 2-bromopropanol) in the presence of a base such as potassium carbonate or potassium tert-butoxide and in as solvent such as acetonitrile or N,N-dimethylformamide (DMF). For reactions conditions see, Organic Letters 2006 8(17), 3745-3748.

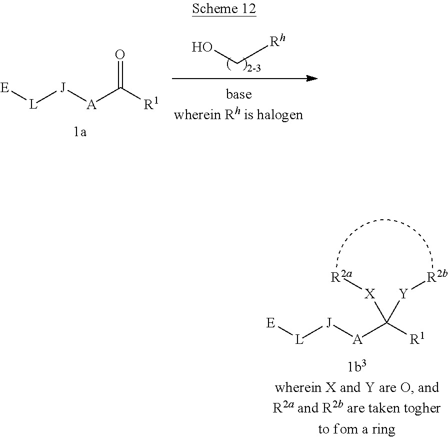

The method of Scheme 12 is also useful for preparing cyclic ketals stating from the corresponding ketone hydrate form. Scheme 13 illustrates a specific example where a ketone hydrate of Formula 1b⁴ (i.e. Formula 1b wherein L is CH₂, J is phenyl (i.e. J-1), A is OCH₂, R^{2a}X and R^{2b}Y are OH and R¹ is CF₃) is reacted with 2-chloroethanol in the presence of potassium carbonate in acetonitrile at a temperature between about 25 to 70° C. to provide a compound of Formula 1b⁵ (i.e. Formula 1b wherein L is CH₂, J is phenyl (i.e. J-1), A is OCH₂, X and Y are O, R^{2a} and R^{2b} are taken together to form a 5-membered ring and R¹ is CF₃). Present Example 2 illustrates the method of Scheme 13.

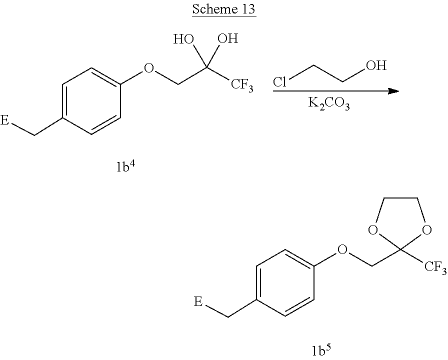

As shown in Scheme 14, Compounds of Formula 1b⁶ (i.e. compounds of Formula 1b wherein A is A¹-A²-CR^{6a}R^{6b}) wherein A¹ is N(R^{7a}), O or S and A² is a direct bond, or wherein A¹ is CR^{6c}R^{6d} and A² is N(R^{7b}), O or S can be prepared by reacting compounds of Formula 9 wherein A¹ is O, S or N(R^{7a}) and A² is a direct bond, or where A¹ is CR^{6c}R^{6d} and A² is O, S or N(R^{7b}) with compounds of Formula 10. The reaction is typically run in a solvent such N,N-dimethylformamide (DMF) or dimethyl sulfoxide with a base such as cesium or potassium carbonate or sodium hydride at a temperature between about 20 to 80° C. The method of Scheme 14 is illustrated in Example 4, Step D.

Of note as starting materials in the method of Scheme 14 are compounds of Formula 10 specifically disclosed in Table 3 below.

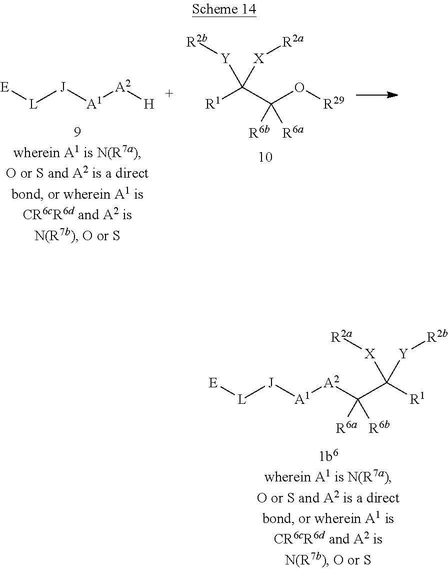

Compounds of Formula 10 can be prepared using commercial precursors and known methods. For example, as shown in Scheme 15, compounds of Formula 10a (i.e. Formula 10 wherein R^{6a} and R^{6b} are H, X and Y are O and R^{2a} and R^{2b} are taken together to form a 5-membered ring) can be prepared reacting compounds of Formula 11 with haloalcohols (e.g., 2-chloroethanol or 3-bromopropanol) under basic conditions (e.g., potassium tert-butoxide in a solvent such as N,N-dimethylformamide or tetrahydrofuran) to provide compounds of Formula 12. A variety of methods are disclosed in the chemical literature for the conversion of ketones to cyclic ketals and can be readily adapted to prepare compounds of Formula 12 (see, for example, G. Hilgetag and A. Martini, Ed., Preparative Organic Chemistry, pp 381-387: Wiley, New York, 1972, and references sited therein; also see present Example 4, Step A). The ester moiety of the resulting cyclic ketal of Formula 12 can be reduced to the corresponding alcohol of Formula 13 by standard methods known to one skilled in the art (Example 4, Step B illustrates a typical procedure). The hydroxy moiety in the compounds of Formula 13 can then be converted to a wide variety of R²⁹ groups to provide compounds of Formula 10a. For example, a mesylate or tosylate group can be installed by treating the alcohol with methanesulfonyl chloride (mesyl chloride) or 4-toluenesulfonyl chloride (tosyl chloride) in the presence of a base such as triethylamine at a temperature between about 0 to 40° C. and in a solvent such as dichloromethane. A triflate group can be installed by treating the alcohol with triflic anhydride (CF₃SO₂)₂O as illustrated in Example 4, Step C. Compounds of Formula 11 are known and can be prepared by methods known to one skilled in the art.

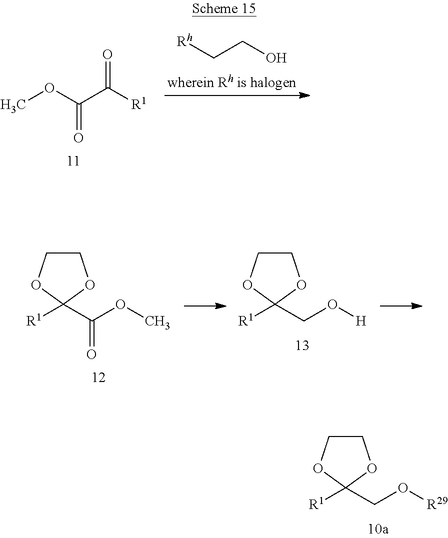

Compounds of Formula 1c (i.e. Formula 1 wherein T is T-3 and X is O) can be prepared by reacting a compound of Formula 1a (i.e. Formula 1 wherein T is T-1 and W is O) wherein at least one of R^{6a} and R^{6b} is H with a compound of Formula 14 in the presence of a base, as illustrated in Scheme 16. Suitable bases include cesium or potassium carbonate in a solvent such as N,N-dimethylformamide (DMF) or dimethyl sulfoxide at temperatures from about 20 to 80° C. In some cases, the method of Scheme 16 results in a mixture of O-alkylated product (typically as a mixture of (E)- and (Z)-isomers), along with C-alkylated product. Purification can be achieved using standard techniques such as column chromatography (see Magnetic Resonance in Chemistry 1991, 29, 675-678). Compounds of Formula 14 are commercially available and can be easily synthesized by general methods known to one skilled in the art.

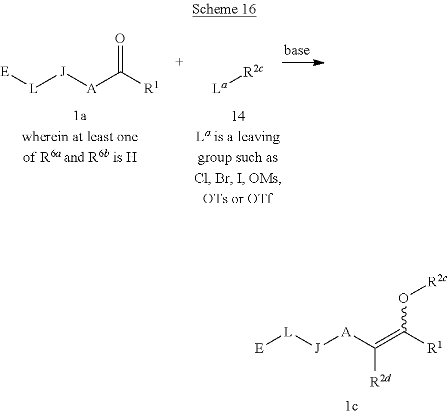

The method of Scheme 16 is also useful for preparing compounds of Formula 1c stating from the corresponding ketone hydrate. Scheme 17 illustrates a specific example where a ketone hydrate of Formula 1b⁴ (i.e. Formula 1b wherein L is CH₂, J is phenyl (i.e. J-1), A is OCH₂, R^{2a}X and R^{2b}Y are OH and R¹ is CF₃) is reacted with iodoethane in the presence of cesium carbonate in dimethyl sulfoxide at a temperature between about 25 to 75° C. to provide a compound of Formula 1c¹(i.e. Formula 1c wherein L is CH₂, J is phenyl (i.e. J-1), A is O, R^{2d} is H, XR^{2c} is OCH₂CH₃ and R¹ is CF₃). Present Example 5 illustrates the method of Scheme 17.

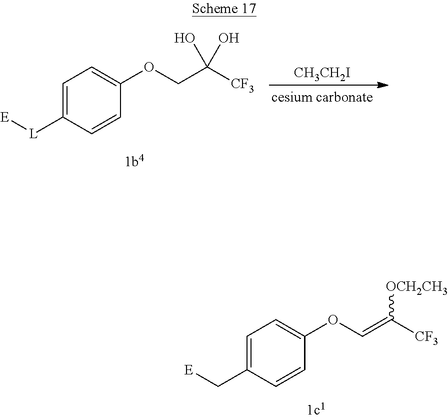

Compounds of Formula 1 wherein T is T-1 and W is S can be prepared from the corresponding compounds wherein W is O by treatment with phosphorus pentasulfide or 2,4-bis(4-methoxyphenyl)-1,3-dithia-2,4-diphosphetane-2,4-disulfide (Lawesson's reagent) in solvents such as toluene, xylene or tetrahydrofuran. One skilled in the art will also recognize that the compounds of Formula 1 wherein T is T-1 and W is NR³ can be prepared from the compounds of Formula 1 wherein T is T-1 and W is O or S by treatment with an amine of Formula R³NH₂ under dehydrating conditions.

The E-L-moieties present in the compounds of Formula 1 and the intermediate compounds of Formulae 2 through 7 and 9 are common organic functional groups whose methods of preparation have been documented in the literature. One skilled in the art will recognize that these well-known chemistry classes (esters, amides, sulfonamides, sulfones, ethers, carbamates, ureas, heterocycles) can be readily prepared by a variety of methods (see, for example, WO 2018/080859, WO 2018/118781, WO 2018/187553 and WO 2019/010192).

It is recognized that some reagents and reaction conditions described above for preparing compounds of Formula 1 may not be compatible with certain functionalities present in the intermediates. In these instances, the incorporation of protection/deprotection sequences or functional group interconversions into the synthesis will aid in obtaining the desired products. The use and choice of the protecting groups will be apparent to one skilled in chemical synthesis (see, for example, T. W. Greene and P. G. M. Wuts, Protective Groups in Organic Synthesis, 2^{nd} ed.; Wiley: New York, 1991). One skilled in the art will recognize that, in some cases, after the introduction of a given reagent as it is depicted in any individual scheme, it may be necessary to perform additional routine synthetic steps not described in detail to complete the synthesis of compounds of Formula 1. One skilled in the art will also recognize that it may be necessary to perform a combination of the steps illustrated in the above schemes in an order other than that implied by the particular sequence presented to prepare the compounds of Formula 1.

One skilled in the art will also recognize that compounds of Formula 1 and the intermediates described herein can be subjected to various electrophilic, nucleophilic, radical, organometallic, oxidation, and reduction reactions to add substituents or modify existing substituents.

Without further elaboration, it is believed that one skilled in the art using the preceding description can utilize the present invention to its fullest extent. The following Examples are, therefore, to be construed as merely illustrative, and not limiting of the disclosure in any way whatsoever. Steps in the following Examples illustrate a procedure for each step in an overall synthetic transformation, and the starting material for each step may not have necessarily been prepared by a particular preparative run whose procedure is described in other Examples or Steps. Percentages are by weight except for chromatographic solvent mixtures or where otherwise indicated. Parts and percentages for chromatographic solvent mixtures are by volume unless otherwise indicated. ¹H NMR spectra are reported in ppm downfield from tetramethylsilane; “s” means singlet, “br s” means broad singlet, “d” means doublet, “dd” means doublet of doublets, “t” means triplet, “q” means quartet and “m” means multiplet. ¹⁹F NMR spectra are reported in ppm using trichlorofluoromethane as the reference.

### Example 1

### Preparation of ethyl 1-[[4-(3,3,3-trifluoro-2,2-dihydroxypropoxy)phenyl]methyl]-1H-pyrazole-4-carboxylate (Compound 1)

### Step A: Preparation of ethyl 1-[(4-hydroxyphenyl)methyl]-1H-pyrazole-4-carboxylate

A mixture of ethyl 1H-pyrazole-4-carboxylate (1.40 g, 10 mmol), 4-(chloromethyl)phenyl acetate (2.0 g, 11 mmol) and potassium carbonate (1.6 g, 11 mmol) in N,N-dimethylformamide (10 mL) was stirred at room temperature for 16 h. Ethanol (10 mL) was added and the reaction mixture was heated at 65° C. for 16 h, cooled, and poured into ice water. The resulting precipitate was collected by filtration, washed with water and air dried. The resulting solid (2.0 g) was crystalized from acetonitrile to provide the title compound as a white solid melting at 113-115° C.

¹H NMR (CDCl₃): δ 1.32 (t, 3H), 3.10 (d, 1H), 4.10-4.40 (m, 5H), 5.24 (s, 2H), 6.91 (d, 2H), 7.22 (d, 2H), 7.83 (s, 1H), 7.93 (s, 1H).

### Step B: Preparation of ethyl 1-[[4-(3,3,3-trifluoro-2-hydroxypropoxy)phenyl]methyl]-1H-pyrazole-4-carboxylate

A mixture of ethyl 1-[(4-hydroxyphenyl)methyl]-1H-pyrazole-4-carboxylate (i.e. the product of Step A) (2.36 g, 9.6 mmol), 2-(trifluoromethyl)oxirane (1.3 g, 11.6 mmol) and cesium carbonate (50 mg, 0.15 mmol) in acetonitrile (20 mL) was heated at 65° C. After 3 days, the reaction mixture was cooled and concentrated under reduced pressure. The resulting material was purified by silica gel chromatography (eluting with a gradient of 0 to 50% ethyl acetate in hexanes) to provide the title compound as a white solid (2.46 g).

¹H NMR (CDCl₃): δ 1.33 (t, 3H), 4.29 (q, 2H), 5.21 (s, 2H), 5.95 (br s, 1H), 6.76 (d, 2H), 7.09 (d, 2H), 7.84 (s, 1H), 7.95 (s, 1H).

¹⁹F NMR (CDCl₃): δ −77.54.

### Step C: Preparation of ethyl 1-[[4-(3,3,3-trifluoro-2,2-dihydroxypropoxy)phenyl]methyl]-1H-pyrazole-4-carboxylate

A mixture of ethyl 1-[[4-(3,3,3-trifluoro-2-hydroxypropoxy)phenyl]methyl]-1H-pyrazole-4-carboxylate (i.e. the product of Step B) (1.23 g, 3.4 mmol) and Dess-Martin periodinane (2.2 g, 5.2 mmol) in dichloromethane (20 mL) was stirred at room temperature for 16 h, and then concentrated under reduced pressure. The resulting material was dissolved in ethyl acetate and washed with sodium bisulfite solution (2 M aqueous solution), followed by saturated aqueous sodium bicarbonate solution. The organic layer was dried, filtered and the filtrate was concentrated under reduced pressure. The resulting tan solid (1.77 g) was crystalized from acetonitrile to provide the title compound, a compound of the present invention, as solid needles melting at 120-123° C.

¹H NMR (CDCl₃): δ 1.32 (t, 3H), 3.80 (br s, 1.7H), 4.18 (s, 2H), 4.28 (q, 2H), 5.25 (s, 2H), 6.95 (d, 2H), 7.22 (d, 2H), 7.82 (s, 1H), 7.95 (s, 1H).

¹⁹F NMR (CDCl₃): δ −84.92.

### Example 2

### Preparation of ethyl 1-[[4-[[2-(trifluoromethyl)-1,3-dioxolan-2-yl]methoxy]phenyl]methyl]-1H-pyrazole-4-carboxylate (Compound 32)

A mixture of ethyl 1-[[4-(3,3,3-trifluoro-2,2-dihydroxypropoxy)phenyl]methyl]-1H-pyrazole-4-carboxylate (i.e. the product of Example 1) (1.07 g, 3.0 mmol), 2-chloroethanol (0.24 g, 3.0 mmol) and potassium carbonate (0.5 g, 3.6 mmol) in N,N-dimethylformamide (3.5 mL) was stirred at room temperature for 16 h, and then heated at 65° C. (briefly). After cooling to room temperature, the reaction mixture was concentrated under reduced pressure. The resulting material was diluted with diethyl ether and washed with saturated aqueous sodium chloride solution. The organic layer was dried, filtered and the filtrate was concentrated under reduced pressure to provide the title compound, a compound of the present invention, as a colorless oil (1.06 g).

¹H NMR (CDCl₃): δ 1.32 (t, 3H), 4.21 (s, 4H), 4.23 (s, 2H), 4.27 (q, 2H), 5.24 (s, 2H), 6.94 (d, 2H), 7.20 (d, 2H), 7.81 (s, 1H), 7.93 (s, 1H).

¹⁹F NMR (CDCl₃): δ− 81.39.

### Example 3

### Preparation of ethyl 1-[[4-[[4,4-dimethyl-2-(trifluoromethyl)-1,3-dioxolan-2-yl]methoxy]-phenyl]methyl]-1H-pyrazole-4-carboxylate (Compound 12)

The title compound was prepared by a procedure analogous to Example 2.

¹H NMR (CDCl₃): δ 1.32 (t, 3H), 1.13 (s, 3H), 1.45 (s, 3H), 3.95 (d, 1H), 4.00 (d, 1H), 4.18 (m, 2H), 4.27 (q, 2H), 5.24 (s, 2H), 6.94 (d, 2H), 7.20 (d, 2H), 7.81 (s, 1H), 7.93 (s, 1H).

¹⁹F NMR (CDCl₃): δ −81.01.

### Example 4

Alternative preparation of ethyl 1-[[4-[[2-(trifluoromethyl)-1,3-dioxolan-2-yl]methoxy]phenyl]-methyl]-1H-pyrazole-4-carboxylate (Compound 32)

### Step A: Preparation of methyl 2-(trifluoromethyl)-1,3-dioxolane-2-carboxylate

To a mixture of methyl 3,3,3-trifluoro-2-oxopropanoate (31.2 g, 200 mmol) in petroleum ether (100 mL) was added 2-bromoethanol (25.0 g, 200 mmol) over a period of 15 minutes. The reaction mixture was stirred at room temperature for 30 minutes, then cooled to 5° C. and potassium carbonate (28 g, 200 mmol) was added with vigorous stirring. Stirring was continued for an additional 4 h at 5° C., and then the reaction mixture was allowed to warm to room temperature, diluted with diethyl ether (100 mL) and filtered. The filtrate was concentrated under reduced pressure, and the resulting material was dissolved in diethyl ether (200 mL) and washed with saturated aqueous sodium chloride solution (3×). The organic layer was dried over magnesium sulfate, filtered and the filtrate was concentrated under reduced pressure to provide the title compound as a colorless oil (29 g).

¹H NMR (CDCl₃): δ 3.80 (s, 3H), 4.30 (m, 4H).

¹⁹F NMR (CDCl₃): δ −80.52.

### Step B: Preparation of 2-(trifluoromethyl)-1,3-dioxolane-2-methanol

To a mixture of methyl 2-(trifluoromethyl)-1,3-dioxolane-2-carboxylate (i.e. the product of Step A) (5 g, 25 mmol) in tetrahydrofuran (75 mL) was added sodium bis(2-methoxy-ethoxy)aluminum hydride (60% in toluene) (12.2 mL, 37.5 mmol). The reaction mixture was heated at 40° C. for 1.5 h, and then cooled to room temperature and a solution of ethyl acetate (3.30 g, 37.5 mmol) in tetrahydrofuran (15 mL) was added dropwise over a period of 15 minutes. The reaction mixture was stirred for 45 minutes and then concentrated under reduced pressure. The resulting material was diluted with diethyl ether (400 mL), washed with saturated aqueous sodium chloride solution (2×), dried over magnesium sulfate and filtered. The filtrate was concentrated under reduced pressure to provide the title compound as an oil (3.8 g).

¹H NMR (CDCl₃): δ 2.59 (t, 1H), 3.82 (d, 2H), 4.19 (m, 4H).

¹⁹F NMR (CDCl₃): δ −81.50.

### Step C: Preparation of [2-(trifluoromethyl)-1,3-dioxolan-2-yl]methyl 1,1,1-trifluoro-methanesulfonate

A mixture of 2-(trifluoromethyl)-1,3-dioxolane-2-methanol (i.e. the product of Step B) (1.67 g, 9.70 mmol) and triethylamine (1.5 mL, 10.8 mmol) in dichloromethane (50 mL) was cooled to −78° C., and then a solution of trifluoromethanesulfonic anhydride (1.81 mL, 10.8 mmol) in dichloromethane (50 mL) was added over a period of 30 minutes. The reaction mixture was stirred at −78° C. for 1.5 h, and then water (50 mL) was added dropwise while allowing the reaction to warm to room temperature. The resulting mixture was partitioned between dichloromethane-water, and the organic layer washed with water, dried over magnesium sulfate and filtered. The filtrated was concentrated under reduced pressure to provide the title compound as a colorless solid (3.0 g).

¹H NMR (CDCl₃): δ 4.24 (m, 4H), 4.60 (br s, 2H).

¹⁹F NMR (CDCl₃): δ −74.84, −81.50.

### Step D: Preparation of ethyl 1-[[4-[[2-(trifluoromethyl)-1,3-dioxolan-2-yl]methoxy]-phenyl]methyl]-1H-pyrazole-4-carboxylate

To a mixture of ethyl 1-[(4-hydroxyphenyl)methyl]-1H-pyrazole-4-carboxylate (i.e. the product of Example 1, Step A) (16.85 g, 68.0 mmol) and cesium carbonate (53.53 g, 164.5 mmol) in N,N-dimethylformamide (100 mL) was added [2-(trifluoromethyl)-1,3-dioxolan-2-yl]methyl 1,1,1-trifluoromethanesulfonate (i.e. the product of Step C) (24.9 g, 82.0 mmol). The reaction mixture was stirred for 24 h at room temperature, and then diluted with diethyl ether. The organic layer was washed with saturated aqueous sodium chloride solution, dried over magnesium sulfate, filtered and the filtrate was concentrated under reduced pressure. The resulting material was purified by silica gel chromatography (eluting with a gradient of 0 to 60% ethyl acetate in hexanes) to provide the title compound, a compound of the present invention, as a white solid (23 g) melting at 59-60° C.

¹H NMR (CDCl₃): δ 1.32 (t, 3H), 4.21 (s, 4H), 4.23 (s, 2H), 4.27 (q, 2H), 5.24 (s, 2H), 6.94 (d, 2H), 7.20 (d, 2H), 7.81 (s, 1H), 7.93 (s, 1H).

¹⁹F NMR (CDCl₃): δ −81.39.

### Example 5

### Preparation of ethyl 1-[[4-[[(1Z)-2-ethoxy-3,3,3-trifluoro-1-propen-1-yl]oxy]phenyl]methyl]-1H-pyrazole-4-carboxylate (Compound 64)

A mixture of ethyl 1-[[4-(3,3,3-trifluoro-2,2-dihydroxypropoxy)phenyl]methyl]-1H-pyrazole-4-carboxylate (i.e. the product of Example 1) (1.0 g, 2.67 mmol), iodoethane (2.5 g, 16 mmol) and cesium carbonate (1.75 g, 5.37 mmol) in dimethyl sulfoxide (10 mL) was heated at 40° C. for 45 minutes. The reaction mixture was diluted with diethyl ether, washed with water and saturated aqueous sodium chloride solution, dried and filtered. The filtrate was concentrated under reduced pressure to provide the title compound, a compound of the present invention, as a white solid (0.80 g). A portion of the solid was further purified by silica gel chromatography (eluting with a gradient of 0 to 50% ethyl acetate in hexanes) to provide a solid melting at 59-60° C. A nuclear Overhauser effect (NOE) was observed between the trifluoromethyl moiety and the vinyl proton indicating a cis-configuration.

¹H NMR (CDCl₃): δ 1.30-1.40 (m, 6H), 4.17 (q, 2H), 4.27 (q, 2H), 5.28 (s, 2H), 6.78 (q, 1H), 7.05 (m, 2H), 7.29 (m, 2H), 7.86 (s, 1H), 7.94 (s, 1H).

¹⁹F NMR (CDCl₃): δ −70.13.

### Example 6

### Preparation of ethyl 1-[[3-(3,3,3-trifluoro-2,2-dihydroxypropoxy)phenyl]methyl]-1H-pyrazole-4-carboxylate (Compound 266)

### Step A: Preparation of 3-(bromomethyl)phenol

A mixture of 1-(bromomethyl)-3-methoxybenzene (15.48 g, 76.99 mmol) in dichloromethane (150 mL) was cooled to −78° C., and then boron tribromide (1 M solution in dichloromethane) was added dropwise. The reaction mixture was allowed to warm to room temperature, stirred for 2 h, and then cooled to −20° C. and methanol (150 mL) was added dropwise. After warming to room temperature, the reaction mixture was concentrated under reduced pressure and the resulting material was diluted with dichloromethane and washed with saturated aqueous sodium bicarbonate solution. The organic layer was dried over magnesium sulfate, filtered and the filtrate was concentrated under reduced pressure. The resulting material was purified by silica gel chromatography (eluting with a gradient of 0 to 100% ethyl acetate in hexanes) to provide the title compound as a white solid (14.16 g).

¹H NMR (CDCl₃): δ 4.44 (s, 2H), 4.89 (s, 1H), 6.76 (dd, 1H), 6.87 (s, 1H), 6.95 (d, 1H), 7.19-7.23 (t, 1H).

### Step B: Preparation of 3-(bromomethyl)phenyl acetate

A solution of 3-(bromomethyl)phenol (i.e. the product of Step A) (14.16 g, 75.7 mmol) in dichloromethane (130 mL) was cooled to 0° C., and then acetic anhydride was added (12.96 g, 12 mL, 126.9 mmol), followed by concentrated sulfuric acid (5 drops). The reaction mixture was allowed to warm to room temperature, stirred for 1 h, and then saturated aqueous sodium bicarbonate solution (300 mL, 318 mmol) was added. The organic layer was separated, washed with water, dried over magnesium sulfate, filtered and the filtrate was concentrated under reduced pressure to provide the title compound as a solid (16.68 g).

¹H NMR (CDCl₃): δ 4.47 (s, 2H), 7.02-7.04 (m, 1H), 7.14 (s, 1H), 7.25 (m, 1H), 7.35 (t, 1H).

### Step C: Preparation of ethyl 1-[[3-(acetyloxy)phenyl]methyl]-1H-pyrazole-4-carboxylate

To a mixture of 3-(bromomethyl)phenyl acetate (i.e. the product of Step B) (16.68 g, 72.8 mmol) in acetonitrile (300 mL) was added ethyl 1H-pyrazole-4-carboxylate (10.61 g, 75.7 mmol) followed by potassium carbonate (19.35 g, 140 mmol). The reaction mixture was heated at 70° C. overnight, cooled to room temperature and filtered. The filtrate was concentrated under reduced pressure to provide the title compound as a yellow oil (20.5 g)

¹H NMR (CDCl₃): δ 2.30 (s, 3H), 4.47 (s, 2H), 7.02 (dd, 1H), 7.15 (s, 1H), 7.25 (m, 1H).

### Step D: Preparation of ethyl 1-[(3-hydroxyphenyl)methyl]-1H-pyrazole-4-carboxylate

To a mixture of ethyl 1-[[3-(acetyloxy)phenyl]methyl]-1H-pyrazole-4-carboxylate (i.e. the product of Step C) (20.5 g, 72.8 mmol) in ethanol was added potassium carbonate (10.1 g, 73 mmol). The reaction mixture was heated at reflux for 3 h, cooled to room temperature and filtered. The filtrate was concentrated under reduced pressure and the resulting material was purified by MPLC silica gel chromatography (eluting with a gradient of 0 to 100% ethyl acetate in hexanes) to provide the title compound as a white solid (10.02 g).

¹H NMR (CDCl₃): δ 1.33 (t, 3H) 4.29 (q, 2H), 5.20 (br s, 1H), 5.25 (s, 2H), 6.66 (m, 1H), 6.78-6.81 (m, 2H), 7.21-7.24 (m, 1H), 7.87 (s, 1H), 7.94 (s, 1H).

### Step E: Preparation of ethyl 1-[[3-(3,3,3-trifluoro-2-hydroxypropoxy)phenyl]methyl]-1H-pyrazole-4-carboxylate

To a mixture of ethyl 1-[(3-hydroxyphenyl)methyl]-1H-pyrazole-4-carboxylate (i.e. the product of Step D) (2.38 g 9.66 mmol) in acetonitrile (100 mL) was added 3-bromo-1,1,1-trifluoro-2-propanol (1.93 g, 1.04 mL, 10 mmol) followed by potassium carbonate (2.86 g, 20.7 mmol). The reaction mixture was heated at reflux for 48 h, cooled to room temperature, filtered and the filtrate was concentrated under reduced pressure. The resulting material was purified by MPLC silica gel chromatography, (eluting with a gradient of 0 to 100% ethyl acetate in hexanes) to provide the title compound as a solid (2.75 g).

¹H NMR (CDCl₃): δ 1.33 (q, 3H), 4.1-4.4 (m, 5H), 5.27 (s, 2H), 6.80 (m, 1H), 6.87-6.89 (m, 2H), 7.28-7.31 (m, 1H), 7.88 (s, 1H), 7.94 (s, 1H). ¹⁹F NMR (CDCl₃): δ −77.53.

### Step F: Preparation of ethyl 1-[[3-(3,3,3-trifluoro-2,2-dihydroxypropoxy)phenyl]methyl]-1H-pyrazole-4-carboxylate

To a mixture of ethyl 1-[[3-(3,3,3-trifluoro-2-hydroxypropoxy)phenyl]methyl]-1H-pyrazole-4-carboxylate (i.e. the product of Step E) (5.7 g, 14.9 mmol) in dichloromethane (300 mL) was added Dess-Martin periodinane (9.13 g, 20.3 mmol) in one portion. After 3 h, the reaction mixture was concentrated under reduced pressure, diluted with ethyl acetate and washed with sodium bisulfite solution (10% aqueous solution), saturated aqueous sodium bicarbonate solution and saturated aqueous sodium chloride solution. The organic layer was dried over magnesium sulfate, filtered and the filtrate was concentrated under reduced pressure. The resulting material was triturated with 1-chlorobutane to provide the title compound, a compound of the present invention, as a white solid (4.89 g).

¹H NMR (DMSO-d₆): δ 1.27 (t, 3H), 4.01 (s, 2H), 4.20 (m, 2H), 5.33 (s, 2H), 6.86-6.92 (m, 3H), 7.26-7.29 (m, 1H), 7.31 (s, 2H,), 7.87 (s, 1H), 8.48 (s, 1H).

¹⁹F NMR (DMSO-d₆): δ −81.82.

### Example 7

### Preparation of ethyl 1-[[3-[[(1Z)-2-ethoxy-3,3,3-trifluoro-1-propen-1-yl]oxy]phenyl]methyl]-1H-pyrazole-4-carboxylate (Compound 265)

To a mixture of ethyl 1-[[3-(3,3,3-trifluoro-2,2-dihydroxypropoxy)phenyl]methyl]-1H-pyrazole-4-carboxylate (i.e. the product of Example 6) (2.94 g, 7.85 mmol) in dimethyl sulfoxide (24 mL) was added iodoethane (2.39 g, 15.3 mmol). The reaction mixture was heated at 65° C., and then cesium carbonate (4.21 g, 12.92 mmol) was added. After 45 minutes, the reaction mixture was cooled to room temperature, and poured into diethyl ether/water (400 mL, 1:1 ratio).

The organic layer was separated and washed with water, saturated aqueous sodium chloride solution, dried over magnesium sulfate, filtered and the filtrate was concentrated under reduced pressure. The resulting material was purified by silica gel chromatography (eluting with a gradient of 0 to 100% ethyl acetate in hexanes) to provide the title compound, a compound of the present invention, as a white solid (2.59 g) melting at 41-43° C.

¹H NMR (CDCl₃): δ 1.32 (m, 6H), 4.16 (m, 2H,), 4.30 (m, 2H,), 5.31 (s, 2H,), 6.76 (s, 1H), 6.93 (m, 1H), 7.00-7.03 (m, 2H), 7.34-7.37 (m, 1H), 7.90 (s, 1H), 7.95 (s, 1H).

¹⁹F NMR (CDCl₃): δ −70.09.

### Example 8

### Preparation of ethyl 1-[[4-(3,3,3-trifluoro-2,2-dihydroxypropoxy)phenoxy]methyl]-1H-pyrazole-4-carboxylate (Compound 366)

### Step A: Preparation of ethyl 1-(hydroxymethyl)-1H-pyrazole-4-carboxylate

A mixture of ethyl 1H-pyrazole-4-carboxylate (6.0 g, 43 mmol), formaldehyde (37% aqueous solution, 12 mL) and ethanol (50 mL) was heated at reflux overnight. The reaction mixture was cooled to room temperature and concentrated under reduced pressure. The resulting material was triturated with 1-chlorobutane to provide the title compound as a white solid (6.2 g).

¹H NMR (DMSO-d₆): δ 1.27 (t, 3H) 4.22 (q, 2H), 5.41 (s, 2H), 7.89 (s, 1H), 8.36 (s, 1H).

### Step B: Preparation of ethyl 1-(chloromethyl)-1H-pyrazole-4-carboxylate

To a mixture of ethyl 1-(hydroxymethyl)-1H-pyrazole-4-carboxylate (i.e. the product of Step A) (6.2 g, 36 mmol) in dichloroethane (100 mL) was added N,N-dimethylformamide (2 drops), followed by thionyl chloride (5.3 mL, 73 mmol) dropwise. After 3 h, the reaction mixture, was concentrated under reduced pressure to provide the title compound as a yellow solid (6.2 g).

¹H NMR (CDCl₃): δ 1.35 (t, 3H), 4.31 (q, 2H), 5.85 (s, 2H), 7.99 (s, 1H), 8.11 (s, 1H).

### Step C: Preparation of ethyl 1-[(4-methoxyphenoxy)methyl]-1H-pyrazole-4-carboxylate

A mixture of ethyl 1-(chloromethyl)-1H-pyrazole-4-carboxylate (i.e. the product of Step B) (2.0 g, 11 mmol), 4-methoxyphenol (1.24 g, 10 mmol), potassium carbonate (2.8 g, 20 mmol) and N,N-dimethylformamide (25 mL) was stirred at room temperature. After 3 days, the reaction mixture was poured into ice water (150 mL) and extracted with diethyl ether (2×100 mL). The combined organic layers were washed with water (50 mL), saturated aqueous sodium chloride solution (25 mL), dried over magnesium sulfate, filtered and the filtrate was concentrated under reduced pressure. The resulting material was purified by silica gel chromatography (eluting with a gradient of 10 to 100% ethyl acetate in hexanes) to provide the title compound as a colorless oil (2.7 g).

¹H NMR (CDCl₃): δ 1.34 (t, 3H), 3.78 (s, 3H), 4.29 (q, 2H), 5.90 (s, 2H), 6.80-6.84 (m, 2H), 6.88-6.91 (m, 2H), 7.96 (s, 1H), 8.05 (s, 1H).

### Step D: Preparation of ethyl 1-[(4-hydroxyphenoxy)methyl]-1H-pyrazole-4-carboxylate

To a mixture of ethyl 1-[(4-methoxyphenoxy)methyl]-1H-pyrazole-4-carboxylate (i.e. the product of Step C) (1.7 g, 6.2 mmol) in dichloromethane (3 mL) was added boron tribromide solution (1 M in dichloromethane, 12.4 mL, 12.4 mmol). After 4 h, saturated aqueous ammonium chloride solution (25 mL) was added to the reaction mixture and stirring was continued for another 15 minutes. The reaction mixture was diluted with dichloromethane (25 mL) and saturated aqueous ammonium chloride solution (25 mL). The organic layer was separated and washed with saturated aqueous sodium bicarbonate solution (25 mL) and saturated aqueous sodium chloride solution (25 mL), drying over magnesium sulfate and filtered. The filtrate was concentrated under reduced pressure to provide the title compound as a solid (1.65 g).

¹H NMR (DMSO-d₆): δ 1.26 (t, 3H), 4.21 (q, 2H), 5.76 (s, 1H), 5.96 (s, 2H), 6.62-6.71 (m, 2H), 6.82-6.88 (m, 2H), 7.93 (d, 1H), 8.48 (d, 1H).

### Step E: Preparation of ethyl 1-[[4-(3,3,3-trifluoro-2-hydroxypropoxy)phenoxy]methyl]-1H-pyrazole-4-carboxylate

To a mixture of ethyl 1-[(4-hydroxyphenoxy)methyl]-1H-pyrazole-4-carboxylate (i.e. the product of Step D) (6.2 mmol) in acetonitrile (20 mL) was added 2-(trifluoromethyl)oxirane (0.62 mL, 7.6 mmol) and cesium carbonate (approximately 10 mg). The reaction mixture was heated at 75° C. overnight, and then cooled to room temperature and concentrated under reduced pressure. The resulting material was purified by silica gel chromatography (eluting with a gradient of 10 to 100% ethyl acetate in hexanes) to provide the title compound as a white solid (0.95 g).

¹H NMR (DMSO-d₆): δ 1.26 (t, 3H), 3.96-4.08 (m, 1H), 4.12 (dd, 1H), 4.22 (q, 2H), 4.33-4.36 (m, 1H), 6.04 (s, 2H), 6.62 (d, 1H), 6.88-6.97 (m, 2H), 7.00-7.03 (m, 2H), 7.95 (s, 1H), 8.54 (s, 1H).

### Step F: Preparation of ethyl 1-[[4-(3,3,3-trifluoro-2,2-dihydroxypropoxy)phenoxy]-methyl]-1H-pyrazole-4-carboxylate

To a mixture of ethyl 1-[[4-(3,3,3-trifluoro-2-hydroxypropoxy)phenoxy]methyl]-1H-pyrazole-4-carboxylate (i.e. the product of Step E) (0.95 g, 2.5 mmol) in dichloromethane (25 mL) was added Dess-Martin periodinane (1.5 g, 3.5 mmol) in one portion. The reaction mixture was stirred for 2.5 h, and then saturated aqueous sodium thiosulfate solution (30 mL) was added and the mixture was concentrated under reduced pressure. The resulting mixture was extracted with ethyl acetate (150 mL) and the combined organic layers were washed with saturated aqueous sodium thiosulfate solution (50 mL), saturated aqueous sodium bicarbonate solution (50 mL) and saturated aqueous sodium chloride solution (25 mL), drying over magnesium sulfate and filtered. The filtered was concentrated under reduced pressure and the resulting material was triturated with dichloromethane to provide the title compound, a compound of the present invention, as a solid (0.65 g).

¹H NMR (DMSO-d₆): δ 1.26 (t, 3H), 3.98 (s, 2H), 4.21 (q, 2H), 6.03 (s, 2H), 6.86-6.94 (m, 2H), 6.95-7.06 (m, 2H), 7.27 (s, 2H), 7.94 (s, 1H), 8.53 (s, 1H).

### Example 9

### Preparation of ethyl 1-[[4-[[(1Z)-2-ethoxy-3,3,3-trifluoro-1-propen-1-yl]oxy]phenoxy]methyl]-1H-pyrazole-4-carboxylate (Compound 364)

A mixture of iodoethane (2.7 mL, 34 mmol), potassium carbonate (0.84 g, 6.1 mmol) and dimethyl sulfoxide (7 mL) was stirred at room temperature for 20 minutes, and then a solution of ethyl 1-[[4-(3,3,3-trifluoro-2,2-dihydroxypropoxy)phenoxy]methyl]-1H-pyrazole-4-carboxylate (i.e. the product of Example 8) (0.64 g, 1.6 mmol) in dimethyl sulfoxide (7 mL) was added portionwise over 20 minutes. After stirring at room temperature for 1.5 hours, the reaction mixture was poured into ice water (150 mL) and extracted with ethyl acetate (125 mL). The organic layer was washed with water (2×50 mL) and saturated aqueous sodium chloride solution (50 mL), drying over magnesium sulfate and filtered. The filtrate was concentrated under reduced pressure and the resulting material was purified by silica gel chromatography (eluting with a gradient of 10 to 100% ethyl acetate in hexanes) to provide the title compound, a compound of the present invention, as a colorless oil (0.46 g).

¹H NMR (DMSO-d₆): δ 1.23-1.27 (m, 6H), 4.11 (q, 2H), 4.22 (q, 2H), 6.10 (s, 2H), 7.07-7.24 (m, 4H) 7.95 (s, 1H) 8.58 (s, 1H).

By the procedures described herein, together with methods known in the art, the following compounds of Tables 1, 1A-48A, 2, 1B-48B and 3 can be prepared. The following abbreviations are used in the Tables: t means tertiary, s means secondary, n means normal, i means iso, c means cyclo, Me means methyl, Et means ethyl, Pr means propyl, i-Pr means isopropyl, c-Pr means cyclopropyl, Bu means butyl, i-Bu means isobutyl, t-Bu means tert-butyl, and Ph means phenyl.

In the above formula, E is equal to E², E² is equal G-Z—, and G is optionally substituted with R¹³. The definitions of G are as defined Exhibit A in the above Embodiments. In the column G, the number in parentheses refers to the attachment point of the G-ring to Z. The (R¹³)ₓ column refers to the substituent(s) attached to the G-ring as shown in Exhibit A above. A dash “-” in the (R¹³)ₓ column means that no R¹³ substituent is present and the remaining valences on the G-ring are occupied by hydrogen atoms.

TABLE 1

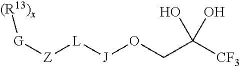

| G | (R¹³)ₓ |
|---|---|
| G-1 (4) | — |
| G-1 (4) | 2-Me |
| G-1 (4) | 2-Et |
| G-1 (4) | 2-n-Pr |
| G-1 (4) | 2-i-Pr |
| G-1 (4) | 2-c-Pr |
| G-1 (4) | 2-n-Bu |
| G-1 (4) | 2-i-Bu |
| G-1 (4) | 2-t-Bu |
| G-1 (4) | 2-F |
| G-1 (4) | 2-Cl |
| G-1 (4) | 2-Br |
| G-1 (4) | 2-CF₃ |
| G-1 (4) | 2-HO |
| G-1 (4) | 2-N≡C |
| G-1 (4) | 2-N≡CCH₂ |
| G-1 (4) | 2-(MeO) |
| G-1 (4) | 2-(MeOCH₂) |
| G-1 (4) | 2-(EtOCH₂) |
| G-1 (4) | 2-(CH(═O)) |
| G-1 (4) | 2-(HOC(═O)) |
| G-1 (4) | 2-(MeOC(═O)) |
| G-1 (4) | 2-(EtOC(═O)) |
| G-1 (4) | 2-(i-PrOC(═O)) |
| G-1 (4) | 2-(n-PrOC(═O)) |
| G-1 (4) | 2-(BuOC(═O)) |
| G-1 (4) | 2-(i-BuOC(═O)) |
| G-1 (4) | 2-(t-BuOC(═O)) |
| G-1 (4) | 2-(CF₃CH₂OC(═O) |
| G-1 (4) | 2-(CH₂═CHOC(═O)) |
| G-1 (4) | 2-(CH₂═CHCH₂OC(═O)) |
| G-1 (4) | 2-(CH₂═CBrCH₂OC(═O)) |
| G-1 (4) | 2-(CH₂═CHCF₂OC(═O)) |
| G-1 (4) | 2-(Me₂C═CHCH₂OC(═O)) |
| G-1 (4) | 2-(CH₂═C(Me)CH₂OC(═O)) |
| G-1 (4) | 2-(CH≡CCH₂OC(═O)) |
| G-1 (4) | 2-(N≡CCH₂OC(═O)) |
| G-1 (4) | 2-(MeNHC(═O)) |
| G-1 (4) | 2-(Me₂NC(═O)) |
| G-1 (4) | 2-(MeNHC(═O)) |
| G-1 (4) | 2-(EtNHC(═O)) |
| G-1 (4) | 2-(PrNHC(═O)) |
| G-1 (4) | 2-(i-PrNHC(═O)) |
| G-1 (4) | 2-(BuNHC(═O)) |
| G-1 (4) | 2-(t-BuNHC(═O)) |
| G-1 (4) | 2-(i-BuNHC(═O)) |
| G-1 (4) | 2-(CF₃CH₂NHC(═O)) |
| G-1 (4) | 2-(c-PrCH₂NHC(═O)) |
| G-1 (4) | 2-(MeOCH₂NHC(═O)) |
| G-1 (4) | 2-(MeOCH₂CH₂NHC(═O)) |
| G-1 (4) | 2-(CH₂═CHCH₂NHC(═O)) |
| G-1 (4) | 2-(N≡CCH₂NHC(═O)) |
| G-1 (4) | 2-(OH—N═CH) |
| G-1 (4) | 2-(Me₂NN═CH) |
| G-1 (4) | 2-(MeOC(═O)NHN═CH) |
| G-1 (4) | 2-(OHC(═O)CH₂ON═CH) |
| G-1 (2) | — |
| G-1 (2) | 4-Me |
| G-1 (2) | 4-Et |
| G-1 (2) | 4-n-Pr |
| G-1 (2) | 4-i-Pr |
| G-1 (2) | 4-c-Pr |
| G-1 (2) | 4-n-Bu |
| G-1 (2) | 4-i-Bu |
| G-1 (2) | 4-t-Bu |
| G-1 (2) | 4-F |
| G-1 (2) | 4-Cl |
| G-1 (2) | 4-Br |
| G-1 (2) | 4-CF₃ |
| G-1 (2) | 4-HO |
| G-1 (2) | 4-N≡C |
| G-1 (2) | 4-N≡CCH₂ |
| G-1 (2) | 4-(MeO) |
| G-1 (2) | 4-(MeOCH₂) |
| G-1 (2) | 4-(EtOCH₂) |
| G-1 (2) | 4-(CH(═O)) |
| G-1 (2) | 4-(HOC(═O)) |
| G-1 (2) | 4-(MeOC(═O)) |
| G-1 (2) | 4-(EtOC(═O)) |
| G-1 (2) | 4-(i-PrOC(═O)) |
| G-1 (2) | 4-(n-PrOC(═O)) |
| G-1 (2) | 4-(BuOC(═O)) |
| G-1 (2) | 4-(i-BuOC(═O)) |
| G-1 (2) | 4-(t-BuOC(═O)) |
| G-1 (2) | 4-(CF₃CH₂OC(═O) |
| G-1 (2) | 4-(CH₂═CHOC(═O)) |
| G-1 (2) | 4-(CH₂═CHCH₂OC(═O)) |
| G-1 (2) | 4-(CH₂═CBrCH₂OC(═O)) |
| G-1 (2) | 4-(CH₂═CHCF₂OC(═O)) |
| G-1 (2) | 4-(Me₂C═CHCH₂OC(═O)) |
| G-1 (2) | 4-(CH₂═C(Me)CH₂OC(═O)) |
| G-1 (2) | 4-(CH≡CCH₂OC(═O)) |
| G-1 (2) | 4-(N≡CCH₂OC(═O)) |
| G-1 (2) | 4-(MeNHC(═O)) |
| G-1 (2) | 4-(Me₂NC(═O)) |
| G-1 (2) | 4-(MeNHC(═O)) |
| G-1 (2) | 4-(EtNHC(═O)) |
| G-1 (2) | 4-(PrNHC(═O)) |
| G-1 (2) | 4-(i-PrNHC(═O)) |
| G-1 (2) | 4-(BuNHC(═O)) |
| G-1 (2) | 4-(t-BuNHC(═O)) |
| G-1 (2) | 4-(i-BuNHC(═O)) |
| G-1 (2) | 4-(CF₃CH₂NHC(═O)) |
| G-1 (2) | 4-(c-PrCH₂NHC(═O)) |
| G-1 (2) | 4-(MeOCH₂NHC(═O)) |
| G-1 (2) | 4-(MeOCH₂CH₂NHC(═O)) |
| G-1 (2) | 4-(CH₂═CHCH₂NHC(═O)) |
| G-1 (2) | 4-(N≡CCH₂NHC(═O)) |
| G-1 (2) | 4-(OH—N═CH) |
| G-1 (2) | 4-(Me₂NN═CH) |
| G-1 (2) | 4-(MeOC(═O)NHN═CH) |
| G-1 (2) | 4-(OHC(═O)CH₂ON═CH) |
| G-3 (1) | — |
| G-3 (1) | 4-Me |
| G-3 (1) | 4-Et |
| G-3 (1) | 4-n-Pr |
| G-3 (1) | 4-i-Pr |
| G-3 (1) | 4-c-Pr |
| G-3 (1) | 4-n-Bu |
| G-3 (1) | 4-i-Bu |
| G-3 (1) | 4-t-Bu |
| G-3 (1) | 4-F |
| G-3 (1) | 4-Cl |
| G-3 (1) | 4-Br |
| G-3 (1) | 4-CF₃ |
| G-3 (1) | 4-HO |
| G-3 (1) | 4-N≡C |
| G-3 (1) | 4-N≡CCH₂ |
| G-3 (1) | 4-(MeO) |
| G-3 (1) | 4-(MeOCH₂) |
| G-3 (1) | 4-(EtOCH₂) |
| G-3 (1) | 4-(CH(═O)) |
| G-3 (1) | 4-(HOC(═O)) |
| G-3 (1) | 4-(MeOC(═O)) |
| G-3 (1) | 4-(EtOC(═O)) |
| G-3 (1) | 4-(i-PrOC(═O)) |
| G-3 (1) | 4-(n-PrOC(═O)) |
| G-3 (1) | 4-(BuOC(═O)) |
| G-3 (1) | 4-(i-BuOC(═O)) |
| G-3 (1) | 4-(t-BuOC(═O)) |
| G-3 (1) | 4-(CF₃CH₂OC(═O) |
| G-3 (1) | 4-(CH₂═CHOC(═O)) |
| G-3 (1) | 4-(CH₂═CHCH₂OC(═O)) |
| G-3 (1) | 4-(CH₂═CBrCH₂OC(═O)) |
| G-3 (1) | 4-(CH₂═CHCF₂OC(═O)) |
| G-3 (1) | 4-(Me₂C═CHCH₂OC(═O)) |
| G-3 (1) | 4-(CH₂═C(Me)CH₂OC(═O)) |
| G-3 (1) | 4-(CH≡CCH₂OC(═O)) |
| G-3 (1) | 4-(N≡CCH₂OC(═O)) |
| G-3 (1) | 4-(MeNHC(═O)) |
| G-3 (1) | 4-(Me₂NC(═O)) |
| G-3 (1) | 4-(MeNHC(═O)) |
| G-3 (1) | 4-(EtNHC(═O)) |
| G-3 (1) | 4-(PrNHC(═O)) |
| G-3 (1) | 4-(i-PrNHC(═O)) |
| G-3 (1) | 4-(BuNHC(═O)) |
| G-3 (1) | 4-(t-BuNHC(═O)) |
| G-3 (1) | 4-(i-BuNHC(═O)) |
| G-3 (1) | 4-(CF₃CH₂NHC(═O)) |
| G-3 (1) | 4-(c-PrCH₂NHC(═O)) |
| G-3 (1) | 4-(MeOCH₂NHC(═O)) |
| G-3 (1) | 4-(MeOCH₂CH₂NHC(═O)) |
| G-3 (1) | 4-(CH₂═CHCH₂NHC(═O)) |
| G-3 (1) | 4-(N≡CCH₂NHC(═O)) |
| G-3 (1) | 4-(OH—N═CH) |
| G-3 (1) | 4-(Me₂NN═CH) |
| G-3 (1) | 4-(MeOC(═O)NHN═CH) |
| G-3 (1) | 4-(OHC(═O)CH₂ON═CH) |
| G-9 (1) | — |
| G-9 (1) | 3-Me |
| G-9 (1) | 3-Et |
| G-9 (1) | 3-n-Pr |
| G-9 (1) | 3-i-Pr |
| G-9 (1) | 3-c-Pr |
| G-9 (1) | 3-n-Bu |
| G-9 (1) | 3-i-Bu |
| G-9 (1) | 3-t-Bu |
| G-9 (1) | 3-F |
| G-9 (1) | 3-Cl |
| G-9 (1) | 3-Br |
| G-9 (1) | 3-CF₃ |
| G-9 (1) | 3-HO |
| G-9 (1) | 3-N≡C |
| G-9 (1) | 3-N≡CCH₂ |
| G-9 (1) | 3-(MeO) |
| G-9 (1) | 3-(MeOCH₂) |
| G-9 (1) | 3-(EtOCH₂) |
| G-9 (1) | 3-(CH(═O)) |
| G-9 (1) | 3-(HOC(═O)) |
| G-9 (1) | 3-(MeOC(═O)) |
| G-9 (1) | 3-(EtOC(═O)) |
| G-9 (1) | 3-(i-PrOC(═O)) |
| G-9 (1) | 3-(n-PrOC(═O)) |
| G-9 (1) | 3-(BuOC(═O)) |
| G-9 (1) | 3-(i-BuOC(═O)) |
| G-9 (1) | 3-(t-BuOC(═O)) |
| G-9 (1) | 3-(CF₃CH₂OC(═O) |
| G-9 (1) | 3-(CH₂═CHOC(═O)) |
| G-9 (1) | 3-(CH₂═CHCH₂OC(═O)) |
| G-9 (1) | 3-(CH₂═CBrCH₂OC(═O)) |
| G-9 (1) | 3-(CH₂═CHCF₂OC(═O)) |
| G-9 (1) | 3-(Me₂C═CHCH₂OC(═O)) |
| G-9 (1) | 3-(CH₂═C(Me)CH₂OC(═O)) |
| G-9 (1) | 3-(CH≡CCH₂OC(═O)) |
| G-9 (1) | 3-(N≡CCH₂OC(═O)) |
| G-9 (1) | 3-(MeNHC(═O)) |
| G-9 (1) | 3-(Me₂NC(═O)) |
| G-9 (1) | 3-(MeNHC(═O)) |
| G-9 (1) | 3-(EtNHC(═O)) |
| G-9 (1) | 3-(PrNHC(═O)) |
| G-9 (1) | 3-(i-PrNHC(═O)) |
| G-9 (1) | 3-(BuNHC(═O)) |
| G-9 (1) | 3-(t-BuNHC(═O)) |
| G-9 (1) | 3-(i-BuNHC(═O)) |
| G-9 (1) | 3-(CF₃CH₂NHC(═O)) |
| G-9 (1) | 3-(c-PrCH₂NHC(═O)) |
| G-9 (1) | 3-(MeOCH₂NHC(═O)) |
| G-9 (1) | 3-(MeOCH₂CH₂NHC(═O)) |
| G-9 (1) | 3-(CH₂═CHCH₂NHC(═O)) |
| G-9 (1) | 3-(N≡CCH₂NHC(═O)) |
| G-9 (1) | 3-(OH—N═CH) |
| G-9 (1) | 3-(Me₂NN═CH) |
| G-9 (1) | 3-(MeOC(═O)NHN═CH) |
| G-9 (1) | 3-(OHC(═O)CH₂ON═CH) |
| G-12 (1) | — |
| G-12 (1) | 4-Me |
| G-12 (1) | 4-Et |
| G-12 (1) | 4-n-Pr |
| G-12 (1) | 4-i-Pr |
| G-12 (1) | 4-c-Pr |
| G-12 (1) | 4-n-Bu |
| G-12 (1) | 4-i-Bu |
| G-12 (1) | 4-t-Bu |
| G-12 (1) | 4-F |
| G-12 (1) | 4-Cl |
| G-12 (1) | 4-Br |
| G-12 (1) | 4-CF₃ |
| G-12 (1) | 4-HO |
| G-12 (1) | 4-N≡C |
| G-12 (1) | 4-N≡CCH₂ |
| G-12 (1) | 4-(MeO) |
| G-12 (1) | 4-(MeOCH₂) |
| G-12 (1) | 4-(EtOCH₂) |
| G-12 (1) | 4-(CH(═O)) |
| G-12 (1) | 4-(HOC(═O)) |
| G-12 (1) | 4-(MeOC(═O)) |
| G-12 (1) | 4-(EtOC(═O)) |
| G-12 (1) | 4-(i-PrOC(═O)) |
| G-12 (1) | 4-(n-PrOC(═O)) |
| G-12 (1) | 4-(BuOC(═O)) |
| G-12 (1) | 4-(i-BuOC(═O)) |
| G-12 (1) | 4-(t-BuOC(═O)) |
| G-12 (1) | 4-(CF₃CH₂OC(═O) |
| G-12 (1) | 4-(CH₂═CHOC(═O)) |
| G-12 (1) | 4-(CH₂═CHCH₂OC(═O)) |
| G-12 (1) | 4-(CH₂═CBrCH₂OC(═O)) |
| G-12 (1) | 4-(CH₂═CHCF₂OC(═O)) |
| G-12 (1) | 4-(Me₂C═CHCH₂OC(═O)) |
| G-12 (1) | 4-(CH₂═C(Me)CH₂OC(═O)) |
| G-12 (1) | 4-(CH≡CCH₂OC(═O)) |
| G-12 (1) | 4-(N≡CCH₂OC(═O)) |
| G-12 (1) | 4-(MeNHC(═O)) |
| G-12 (1) | 4-(Me₂NC(═O)) |
| G-12 (1) | 4-(MeNHC(═O)) |
| G-12 (1) | 4-(EtNHC(═O)) |
| G-12 (1) | 4-(PrNHC(═O)) |
| G-12 (1) | 4-(i-PrNHC(═O)) |
| G-12 (1) | 4-(BuNHC(═O)) |
| G-12 (1) | 4-(t-BuNHC(═O)) |
| G-12 (1) | 4-(i-BuNHC(═O)) |
| G-12 (1) | 4-(CF₃CH₂NHC(═O)) |
| G-12 (1) | 4-(c-PrCH₂NHC(═O)) |
| G-12 (1) | 4-(MeOCH₂NHC(═O)) |
| G-12 (1) | 4-(MeOCH₂CH₂NHC(═O)) |
| G-12 (1) | 4-(CH₂═CHCH₂NHC(═O)) |
| G-12 (1) | 4-(N≡CCH₂NHC(═O)) |
| G-12 (1) | 4-(OH—N═CH) |
| G-12 (1) | 4-(Me₂NN═CH) |
| G-12 (1) | 4-(MeOC(═O)NHN═CH) |
| G-12 (1) | 4-(OHC(═O)CH₂ON═CH) |
| G-12 (1) | 5-Me, 3-(EtOC(═O)) |
| G-12 (1) | 3-Me |
| G-12 (1) | 3-Et |
| G-12 (1) | 3-n-Pr |
| G-12 (1) | 3-i-Pr |
| G-12 (1) | 3-c-Pr |
| G-12 (1) | 3-n-Bu |
| G-12 (1) | 3-i-Bu |
| G-12 (1) | 3-t-Bu |
| G-12 (1) | 3-F |
| G-12 (1) | 3-Cl |
| G-12 (1) | 3-Br |
| G-12 (1) | 3-CF₃ |
| G-12 (1) | 3-HO |
| G-12 (1) | 3-N≡C |
| G-12 (1) | 3-N≡CCH₂ |
| G-12 (1) | 3-(MeO) |
| G-12 (1) | 3-(MeOCH₂) |
| G-12 (1) | 3-(EtOCH₂) |
| G-12 (1) | 3-(CH(═O)) |
| G-12 (1) | 3-(HOC(═O)) |
| G-12 (1) | 3-(MeOC(═O)) |
| G-12 (1) | 3-(EtOC(═O)) |
| G-12 (1) | 3-(i-PrOC(═O)) |
| G-12 (1) | 3-(n-PrOC(═O)) |
| G-12 (1) | 3-(BuOC(═O)) |
| G-12 (1) | 3-(i-BuOC(═O)) |
| G-12 (1) | 3-(t-BuOC(═O)) |
| G-12 (1) | 3-(CF₃CH₂OC(═O) |
| G-12 (1) | 3-(CH₂═CHOC(═O)) |
| G-12 (1) | 3-(CH₂═CHCH₂OC(═O)) |
| G-12 (1) | 3-(CH₂═CBrCH₂OC(═O)) |
| G-12 (1) | 3-(CH₂═CHCF₂OC(═O)) |
| G-12 (1) | 3-(Me₂C═CHCH₂OC(═O)) |
| G-12 (1) | 3-(CH₂═C(Me)CH₂OC(═O)) |
| G-12 (1) | 3-(CH≡CCH₂OC(═O)) |
| G-12 (1) | 3-(N≡CCH₂OC(═O)) |
| G-12 (1) | 3-(MeNHC(═O)) |
| G-12 (1) | 3-(Me₂NC(═O)) |
| G-12 (1) | 3-(MeNHC(═O)) |
| G-12 (1) | 3-(EtNHC(═O)) |
| G-12 (1) | 3-(PrNHC(═O)) |
| G-12 (1) | 3-(i-PrNHC(═O)) |
| G-12 (1) | 3-(BuNHC(═O)) |
| G-12 (1) | 3-(t-BuNHC(═O)) |
| G-12 (1) | 3-(i-BuNHC(═O)) |
| G-12 (1) | 3-(CF₃CH₂NHC(═O)) |
| G-12 (1) | 3-(c-PrCH₂NHC(═O)) |
| G-12 (1) | 3-(MeOCH₂NHC(═O)) |
| G-12 (1) | 3-(MeOCH₂CH₂NHC(═O)) |
| G-12 (1) | 3-(CH₂═CHCH₂NHC(═O)) |
| G-12 (1) | 3-(N≡CCH₂NHC(═O)) |
| G-12 (1) | 3-(OH—N═CH) |
| G-12 (1) | 3-(Me₂NN═CH) |
| G-12 (1) | 3-(MeOC(═O)NHN═CH) |
| G-12 (1) | 3-(OHC(═O)CH₂ON═CH) |
| G-13 (1) | — |
| G-13 (1) | 5-Me |
| G-13 (1) | 5-Et |
| G-13 (1) | 5-n-Pr |
| G-13 (1) | 5-i-Pr |
| G-13 (1) | 5-c-Pr |
| G-13 (1) | 5-n-Bu |
| G-13 (1) | 5-i-Bu |
| G-13 (1) | 5-t-Bu |
| G-13 (1) | 5-F |
| G-13 (1) | 5-Cl |
| G-13 (1) | 5-Br |
| G-13 (1) | 5-CF₃ |
| G-13 (1) | 5-HO |
| G-13 (1) | 5-N≡C |
| G-13 (1) | 5-N≡CCH₂ |
| G-13 (1) | 5-(MeO) |
| G-13 (1) | 5-(MeOCH₂) |
| G-13 (1) | 5-(EtOCH₂) |
| G-13 (1) | 5-(CH(═O)) |
| G-13 (1) | 5-(HOC(═O)) |
| G-13 (1) | 5-(MeOC(═O)) |
| G-13 (1) | 5-(EtOC(═O)) |
| G-13 (1) | 5-(i-PrOC(═O)) |
| G-13 (1) | 5-(n-PrOC(═O)) |
| G-13 (1) | 5-(BuOC(═O)) |
| G-13 (1) | 5-(i-BuOC(═O)) |
| G-13 (1) | 5-(t-BuOC(═O)) |
| G-13 (1) | 5-(CF₃CH₂OC(═O) |
| G-13 (1) | 5-(CH₂═CHOC(═O)) |
| G-13 (1) | 5-(CH₂═CHCH₂OC(═O)) |
| G-13 (1) | 5-(CH₂═CBrCH₂OC(═O)) |
| G-13 (1) | 5-(CH₂═CHCF₂OC(═O)) |
| G-13 (1) | 5-(Me₂C═CHCH₂OC(═O)) |
| G-13 (1) | 5-(CH₂═C(Me)CH₂OC(═O)) |
| G-13 (1) | 5-(CH≡CCH₂OC(═O)) |
| G-13 (1) | 5-(N≡CCH₂OC(═O)) |
| G-13 (1) | 5-(MeNHC(═O)) |
| G-13 (1) | 5-(Me₂NC(═O)) |
| G-13 (1) | 5-(MeNHC(═O)) |
| G-13 (1) | 5-(EtNHC(═O)) |
| G-13 (1) | 5-(PrNHC(═O)) |
| G-13 (1) | 5-(i-PrNHC(═O)) |
| G-13 (1) | 5-(BuNHC(═O)) |
| G-13 (1) | 5-(t-BuNHC(═O)) |
| G-13 (1) | 5-(i-BuNHC(═O)) |
| G-13 (1) | 5-(CF₃CH₂NHC(═O)) |
| G-13 (1) | 5-(c-PrCH₂NHC(═O)) |
| G-13 (1) | 5-(MeOCH₂NHC(═O)) |
| G-13 (1) | 5-(MeOCH₂CH₂NHC(═O)) |
| G-13 (1) | 5-(CH₂═CHCH₂NHC(═O)) |
| G-13 (1) | 5-(N≡CCH₂NHC(═O)) |
| G-13 (1) | 5-(OH—N═CH) |
| G-13 (1) | 5-(Me₂NN═CH) |
| G-13 (1) | 5-(MeOC(═O)NHN═CH) |
| G-13 (1) | 5-(OHC(═O)CH₂ON═CH) |
| G-17 (1) | — |
| G-17 (1) | 4-Me |
| G-17 (1) | 4-Et |
| G-17 (1) | 4-n-Pr |
| G-17 (1) | 4-i-Pr |
| G-17 (1) | 4-c-Pr |
| G-17 (1) | 4-n-Bu |
| G-17 (1) | 4-i-Bu |
| G-17 (1) | 4-t-Bu |
| G-17 (1) | 4-F |
| G-17 (1) | 4-Cl |
| G-17 (1) | 4-Br |
| G-17 (1) | 4-CF₃ |
| G-17 (1) | 4-HO |
| G-17 (1) | 4-N≡C |
| G-17 (1) | 4-N≡CCH₂ |
| G-17 (1) | 4-(MeO) |
| G-17 (1) | 4-(MeOCH₂) |
| G-17 (1) | 4-(EtOCH₂) |
| G-17 (1) | 4-(CH(═O)) |
| G-17 (1) | 4-(HOC(═O)) |
| G-17 (1) | 4-(MeOC(═O)) |
| G-17 (1) | 4-(EtOC(═O)) |
| G-17 (1) | 4-(i-PrOC(═O)) |
| G-17 (1) | 4-(n-PrOC(═O)) |
| G-17 (1) | 4-(BuOC(═O)) |
| G-17 (1) | 4-(i-BuOC(═O)) |
| G-17 (1) | 4-(t-BuOC(═O)) |
| G-17 (1) | 4-(CF₃CH₂OC(═O) |
| G-17 (1) | 4-(CH₂═CHOC(═O)) |
| G-17 (1) | 4-(CH₂═CHCH₂OC(═O)) |
| G-17 (1) | 4-(CH₂═CBrCH₂OC(═O)) |
| G-17 (1) | 4-(CH₂═CHCF₂OC(═O)) |
| G-17 (1) | 4-(Me₂C═CHCH₂OC(═O)) |
| G-17 (1) | 4-(CH₂═C(Me)CH₂OC(═O)) |
| G-17 (1) | 4-(CH≡CCH₂OC(═O)) |
| G-17 (1) | 4-(N≡CCH₂OC(═O)) |
| G-17 (1) | 4-(MeNHC(═O)) |
| G-17 (1) | 4-(Me₂NC(═O)) |
| G-17 (1) | 4-(MeNHC(═O)) |
| G-17 (1) | 4-(EtNHC(═O)) |
| G-17 (1) | 4-(PrNHC(═O)) |
| G-17 (1) | 4-(i-PrNHC(═O)) |
| G-17 (1) | 4-(BuNHC(═O)) |
| G-17 (1) | 4-(t-BuNHC(═O)) |
| G-17 (1) | 4-(i-BuNHC(═O)) |
| G-17 (1) | 4-(CF₃CH₂NHC(═O)) |
| G-17 (1) | 4-(c-PrCH₂NHC(═O)) |
| G-17 (1) | 4-(MeOCH₂NHC(═O)) |
| G-17 (1) | 4-(MeOCH₂CH₂NHC(═O)) |
| G-17 (1) | 4-(CH₂═CHCH₂NHC(═O)) |
| G-17 (1) | 4-(N≡CCH₂NHC(═O)) |
| G-17 (1) | 4-(OH—N═CH) |
| G-17 (1) | 4-(Me₂NN═CH) |
| G-17 (1) | 4-(MeOC(═O)NHN═CH) |
| G-17 (1) | 4-(OHC(═O)CH₂ON═CH) |

J is J-1, L is CH₂ and Z is a direct bond.

The present disclosure also includes Tables 1A through 48A, each of which is constructed the same as Table 1 above, except that the row heading in Table 1 (i.e. “J is J-1, L is CH₂ and Z is a direct bond”) is replaced with the respective row headings shown below.

| Table | Row Heading |
|---|---|
| 1A | J is J-1, L is CH₂CH₂ and Z is a direct bond. |
| 2A | J is J-1, L is CH₂(Me) and Z is a direct bond. |
| 3A | J is J-1, L is (CH₂)₃ and Z is a direct bond. |
| 4A | J is J-1, L is CH₂ and Z is O. |
| 5A | J is J-2, L is CH₂ and Z is a direct bond. |
| 6A | J is J-2, L is CH₂CH₂ and Z is a direct bond. |
| 7A | J is J-2, L is CH₂(Me) and Z is a direct bond. |
| 8A | J is J-2, L is (CH₂)₃ and Z is a direct bond. |
| 9A | J is J-2, L is CH₂ and Z is O. |
| 10A | J is J-6, L is CH₂ and Z is a direct bond. |
| 11A | J is J-6, L is CH₂CH₂ and Z is a direct bond. |
| 12A | J is J-6, L is CH₂(Me) and Z is a direct bond. |
| 13A | J is J-6, L is (CH₂)₃ and Z is a direct bond. |
| 14A | J is J-6, L is CH₂ and Z is O. |
| 15A | J is J-7, L is CH₂ and Z is a direct bond. |
| 16A | J is J-7, L is CH₂CH₂ and Z is a direct bond. |
| 17A | J is J-7, L is CH₂(Me) and Z is a direct bond. |
| 18A | J is J-7, L is (CH₂)₃ and Z is a direct bond. |
| 19A | J is J-7, L is CH₂ and Z is O. |
| 20A | J is J-8, L is CH₂ and Z is a direct bond. |
| 21A | J is J-8, L is CH₂CH₂ and Z is a direct bond. |
| 22A | J is J-8, L is CH₂(Me) and Z is a direct bond. |
| 23A | J is J-8, L is (CH₂)₃ and Z is a direct bond. |
| 24A | J is J-8, L is CH₂ and Z is O. |
| 25A | J is J-10, L is CH₂ and Z is a direct bond. |
| 26A | J is J-10, L is CH₂CH₂ and Z is a direct bond. |
| 27A | J is J-10, L is CH₂(Me) and Z is a direct bond. |
| 28A | J is J-10, L is (CH₂)₃ and Z is a direct bond. |
| 29A | J is J-10, L is CH₂ and Z is O. |
| 30A | J is J-14, L is CH₂ and Z is a direct bond. |
| 31A | J is J-14, L is CH₂CH₂ and Z is a direct bond. |
| 32A | J is J-14, L is CH₂(Me) and Z is a direct bond. |
| 33A | J is J-14, L is (CH₂)₃ and Z is a direct bond. |
| 34A | J is J-14, L is CH₂ and Z is O. |
| 35A | J is J-3, L is CH₂ and Z is a direct bond. |
| 36A | J is J-3, L is CH₂CH₂ and Z is a direct bond. |
| 37A | J is J-3, L is CH₂(Me) and Z is a direct bond. |
| 38A | J is J-3, L is (CH₂)₃ and Z is a direct bond. |
| 39A | J is J-3, L is CH₂ and Z is O. |
| 40A | J is J-4, L is CH₂ and Z is a direct bond. |
| 41A | J is J-4, L is CH₂CH₂ and Z is a direct bond. |
| 42A | J is J-4, L is CH₂(Me) and Z is a direct bond. |
| 43A | J is J-4, L is (CH₂)₃ and Z is a direct bond. |
| 44A | J is J-4, L is CH₂ and Z is O. |
| 45A | J is J-5, L is CH₂ and Z is a direct bond. |
| 46A | J is J-5, L is CH₂CH₂ and Z is a direct bond. |
| 47A | J is J-5, L is CH₂(Me) and Z is a direct bond. |
| 48A | J is J-5, L is (CH₂)₃ and Z is a direct bond. |

In the above formula, E is equal to E², E² is equal G-Z—, and G is optionally substituted with R¹³. The definitions of G are as defined Exhibit A in the above Embodiments. In the column G, the number in parentheses refers to the attachment point of the G-ring to Z. The (R¹³)ₓ column refers to the substituent(s) attached to the G-ring as shown in Exhibit A above. A dash “-” in the (R¹³)ₓ column means that no R¹³ substituent is present and the remaining-valences on the G-ring are occupied by hydrogen atoms.

TABLE 2

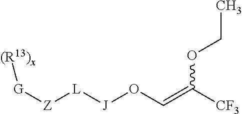

| G | (R¹³)ₓ |
|---|---|
| G-1 (4) | — |
| G-1 (4) | 2-Me |
| G-1 (4) | 2-Et |
| G-1 (4) | 2-n-Pr |
| G-1 (4) | 2-i-Pr |
| G-1 (4) | 2-c-Pr |
| G-1 (4) | 2-n-Bu |
| G-1 (4) | 2-i-Bu |
| G-1 (4) | 2-t-Bu |
| G-1 (4) | 2-F |
| G-1 (4) | 2-Cl |
| G-1 (4) | 2-Br |
| G-1 (4) | 2-CF₃ |
| G-1 (4) | 2-HO |
| G-1 (4) | 2-N≡C |
| G-1 (4) | 2-N≡CCH₂ |
| G-1 (4) | 2-(MeO) |
| G-1 (4) | 2-(MeOCH₂) |
| G-1 (4) | 2-(EtOCH₂) |
| G-1 (4) | 2-(CH(═O)) |
| G-1 (4) | 2-(HOC(═O)) |
| G-1 (4) | 2-(MeOC(═O)) |
| G-1 (4) | 2-(EtOC(═O)) |
| G-1 (4) | 2-(i-PrOC(═O)) |
| G-1 (4) | 2-(n-PrOC(═O)) |
| G-1 (4) | 2-(BuOC(═O)) |
| G-1 (4) | 2-(i-BuOC(═O)) |
| G-1 (4) | 2-(t-BuOC(═O)) |
| G-1 (4) | 2-(CF₃CH₂OC(═O) |
| G-1 (4) | 2-(CH₂═CHOC(═O)) |
| G-1 (4) | 2-(CH₂═CHCH₂OC(═O)) |
| G-1 (4) | 2-(CH₂═CBrCH₂OC(═O)) |
| G-1 (4) | 2-(CH₂═CHCF₂OC(═O)) |
| G-1 (4) | 2-(Me₂C═CHCH₂OC(═O)) |
| G-1 (4) | 2-(CH₂═C(Me)CH₂OC(═O)) |
| G-1 (4) | 2-(CH≡CCH₂OC(═O)) |
| G-1 (4) | 2-(N≡CCH₂OC(═O)) |
| G-1 (4) | 2-(MeNHC(═O)) |
| G-1 (4) | 2-(Me₂NC(═O)) |
| G-1 (4) | 2-(MeNHC(═O)) |
| G-1 (4) | 2-(EtNHC(═O)) |
| G-1 (4) | 2-(PrNHC(═O)) |
| G-1 (4) | 2-(i-PrNHC(═O)) |
| G-1 (4) | 2-(BuNHC(═O)) |
| G-1 (4) | 2-(t-BuNHC(═O)) |
| G-1 (4) | 2-(i-BuNHC(═O)) |
| G-1 (4) | 2-(CF₃CH₂NHC(═O)) |
| G-1 (4) | 2-(c-PrCH₂NHC(═O)) |
| G-1 (4) | 2-(MeOCH₂NHC(═O)) |
| G-1 (4) | 2-(MeOCH₂CH₂NHC(═O)) |
| G-1 (4) | 2-(CH₂═CHCH₂NHC(═O)) |
| G-1 (4) | 2-(N≡CCH₂NHC(═O)) |
| G-1 (4) | 2-(OH—N═CH) |
| G-1 (4) | 2-(Me₂NN═CH) |
| G-1 (4) | 2-(MeOC(═O)NHN═CH) |
| G-1 (4) | 2-(OHC(═O)CH₂ON═CH) |
| G-1 (2) | — |
| G-1 (2) | 4-Me |
| G-1 (2) | 4-Et |
| G-1 (2) | 4-n-Pr |
| G-1 (2) | 4-i-Pr |
| G-1 (2) | 4-c-Pr |
| G-1 (2) | 4-n-Bu |
| G-1 (2) | 4-i-Bu |
| G-1 (2) | 4-t-Bu |
| G-1 (2) | 4-F |
| G-1 (2) | 4-Cl |
| G-1 (2) | 4-Br |
| G-1 (2) | 4-CF₃ |
| G-1 (2) | 4-HO |
| G-1 (2) | 4-N≡C |
| G-1 (2) | 4-N≡CCH₂ |
| G-1 (2) | 4-(MeO) |
| G-1 (2) | 4-(MeOCH₂) |
| G-1 (2) | 4-(EtOCH₂) |
| G-1 (2) | 4-(CH(═O)) |
| G-1 (2) | 4-(HOC(═O)) |
| G-1 (2) | 4-(MeOC(═O)) |
| G-1 (2) | 4-(EtOC(═O)) |
| G-1 (2) | 4-(i-PrOC(═O)) |
| G-1 (2) | 4-(n-PrOC(═O)) |
| G-1 (2) | 4-(BuOC(═O)) |
| G-1 (2) | 4-(i-BuOC(═O)) |
| G-1 (2) | 4-(t-BuOC(═O)) |
| G-1 (2) | 4-(CF₃CH₂OC(═O) |
| G-1 (2) | 4-(CH₂═CHOC(═O)) |
| G-1 (2) | 4-(CH₂═CHCH₂OC(═O)) |
| G-1 (2) | 4-(CH₂═CBrCH₂OC(═O)) |
| G-1 (2) | 4-(CH₂═CHCF₂OC(═O)) |
| G-1 (2) | 4-(Me₂C═CHCH₂OC(═O)) |
| G-1 (2) | 4-(CH₂═C(Me)CH₂OC(═O)) |
| G-1 (2) | 4-(CH≡CCH₂OC(═O)) |
| G-1 (2) | 4-(N≡CCH₂OC(═O)) |
| G-1 (2) | 4-(MeNHC(═O)) |
| G-1 (2) | 4-(Me₂NC(═O)) |
| G-1 (2) | 4-(MeNHC(═O)) |
| G-1 (2) | 4-(EtNHC(═O)) |
| G-1 (2) | 4-(PrNHC(═O)) |
| G-1 (2) | 4-(i-PrNHC(═O)) |
| G-1 (2) | 4-(BuNHC(═O)) |
| G-1 (2) | 4-(t-BuNHC(═O)) |
| G-1 (2) | 4-(i-BuNHC(═O)) |
| G-1 (2) | 4-(CF₃CH₂NHC(═O)) |
| G-1 (2) | 4-(c-PrCH₂NHC(═O)) |
| G-1 (2) | 4-(MeOCH₂NHC(═O)) |
| G-1 (2) | 4-(MeOCH₂CH₂NHC(═O)) |
| G-1 (2) | 4-(CH₂═CHCH₂NHC(═O)) |
| G-1 (2) | 4-(N≡CCH₂NHC(═O)) |
| G-1 (2) | 4-(OH—N═CH) |
| G-1 (2) | 4-(Me₂NN═CH) |
| G-1 (2) | 4-(MeOC(═O)NHN═CH) |
| G-1 (2) | 4-(OHC(═O)CH₂ON═CH) |
| G-3 (1) | — |
| G-3 (1) | 4-Me |
| G-3 (1) | 4-Et |
| G-3 (1) | 4-n-Pr |
| G-3 (1) | 4-i-Pr |
| G-3 (1) | 4-c-Pr |
| G-3 (1) | 4-n-Bu |
| G-3 (1) | 4-i-Bu |
| G-3 (1) | 4-t-Bu |
| G-3 (1) | 4-F |
| G-3 (1) | 4-Cl |
| G-3 (1) | 4-Br |
| G-3 (1) | 4-CF₃ |
| G-3 (1) | 4-HO |
| G-3 (1) | 4-N≡C |
| G-3 (1) | 4-N≡CCH₂ |
| G-3 (1) | 4-(MeO) |
| G-3 (1) | 4-(MeOCH₂) |
| G-3 (1) | 4-(EtOCH₂) |
| G-3 (1) | 4-(CH(═O)) |
| G-3 (1) | 4-(HOC(═O)) |
| G-3 (1) | 4-(MeOC(═O)) |
| G-3 (1) | 4-(EtOC(═O)) |
| G-3 (1) | 4-(i-PrOC(═O)) |
| G-3 (1) | 4-(n-PrOC(═O)) |
| G-3 (1) | 4-(BuOC(═O)) |
| G-3 (1) | 4-(i-BuOC(═O)) |
| G-3 (1) | 4-(t-BuOC(═O)) |
| G-3 (1) | 4-(CF₃CH₂OC(═O) |
| G-3 (1) | 4-(CH₂═CHOC(═O)) |
| G-3 (1) | 4-(CH₂═CHCH₂OC(═O)) |
| G-3 (1) | 4-(CH₂═CBrCH₂OC(═O)) |
| G-3 (1) | 4-(CH₂═CHCF₂OC(═O)) |
| G-3 (1) | 4-(Me₂C═CHCH₂OC(═O)) |
| G-3 (1) | 4-(CH₂═C(Me)CH₂OC(═O)) |
| G-3 (1) | 4-(CH≡CCH₂OC(═O)) |
| G-3 (1) | 4-(N≡CCH₂OC(═O)) |
| G-3 (1) | 4-(MeNHC(═O)) |
| G-3 (1) | 4-(Me₂NC(═O)) |
| G-3 (1) | 4-(MeNHC(═O)) |
| G-3 (1) | 4-(EtNHC(═O)) |
| G-3 (1) | 4-(PrNHC(═O)) |
| G-3 (1) | 4-(i-PrNHC(═O)) |
| G-3 (1) | 4-(BuNHC(═O)) |
| G-3 (1) | 4-(t-BuNHC(═O)) |
| G-3 (1) | 4-(i-BuNHC(═O)) |
| G-3 (1) | 4-(CF₃CH₂NHC(═O)) |
| G-3 (1) | 4-(c-PrCH₂NHC(═O)) |
| G-3 (1) | 4-(MeOCH₂NHC(═O)) |
| G-3 (1) | 4-(MeOCH₂CH₂NHC(═O)) |
| G-3 (1) | 4-(CH₂═CHCH₂NHC(═O)) |
| G-3 (1) | 4-(N≡CCH₂NHC(═O)) |
| G-3 (1) | 4-(OH—N═CH) |
| G-3 (1) | 4-(Me₂NN═CH) |
| G-3 (1) | 4-(MeOC(═O)NHN═CH) |
| G-3 (1) | 4-(OHC(═O)CH₂ON═CH) |
| G-9 (1) | — |
| G-9 (1) | 3-Me |
| G-9 (1) | 3-Et |
| G-9 (1) | 3-n-Pr |
| G-9 (1) | 3-i-Pr |
| G-9 (1) | 3-c-Pr |
| G-9 (1) | 3-n-Bu |
| G-9 (1) | 3-i-Bu |
| G-9 (1) | 3-t-Bu |
| G-9 (1) | 3-F |
| G-9 (1) | 3-Cl |
| G-9 (1) | 3-Br |
| G-9 (1) | 3-CF₃ |
| G-9 (1) | 3-HO |
| G-9 (1) | 3-N≡C |
| G-9 (1) | 3-N≡CCH₂ |
| G-9 (1) | 3-(MeO) |
| G-9 (1) | 3-(MeOCH₂) |
| G-9 (1) | 3-(EtOCH₂) |
| G-9 (1) | 3-(CH(═O)) |
| G-9 (1) | 3-(HOC(═O)) |
| G-9 (1) | 3-(MeOC(═O)) |
| G-9 (1) | 3-(EtOC(═O)) |
| G-9 (1) | 3-(i-PrOC(═O)) |
| G-9 (1) | 3-(n-PrOC(═O)) |
| G-9 (1) | 3-(BuOC(═O)) |
| G-9 (1) | 3-(i-BuOC(═O)) |
| G-9 (1) | 3-(t-BuOC(═O)) |
| G-9 (1) | 3-(CF₃CH₂OC(═O) |
| G-9 (1) | 3-(CH₂═CHOC(═O)) |
| G-9 (1) | 3-(CH₂═CHCH₂OC(═O)) |
| G-9 (1) | 3-(CH₂═CBrCH₂OC(═O)) |
| G-9 (1) | 3-(CH₂═CHCF₂OC(═O)) |
| G-9 (1) | 3-(Me₂C═CHCH₂OC(═O)) |
| G-9 (1) | 3-(CH₂═C(Me)CH₂OC(═O)) |
| G-9 (1) | 3-(CH≡CCH₂OC(═O)) |
| G-9 (1) | 3-(N≡CCH₂OC(═O)) |
| G-9 (1) | 3-(MeNHC(═O)) |
| G-9 (1) | 3-(Me₂NC(═O)) |
| G-9 (1) | 3-(MeNHC(═O)) |
| G-9 (1) | 3-(EtNHC(═O)) |
| G-9 (1) | 3-(PrNHC(═O)) |
| G-9 (1) | 3-(i-PrNHC(═O)) |
| G-9 (1) | 3-(BuNHC(═O)) |
| G-9 (1) | 3-(t-BuNHC(═O)) |
| G-9 (1) | 3-(i-BuNHC(═O)) |
| G-9 (1) | 3-(CF₃CH₂NHC(═O)) |
| G-9 (1) | 3-(c-PrCH₂NHC(═O)) |
| G-9 (1) | 3-(MeOCH₂NHC(═O)) |
| G-9 (1) | 3-(MeOCH₂CH₂NHC(═O)) |
| G-9 (1) | 3-(CH₂═CHCH₂NHC(═O)) |
| G-9 (1) | 3-(N≡CCH₂NHC(═O)) |
| G-9 (1) | 3-(OH—N═CH) |
| G-9 (1) | 3-(Me₂NN═CH) |
| G-9 (1) | 3-(MeOC(═O)NHN═CH) |
| G-9 (1) | 3-(OHC(═O)CH₂ON═CH) |
| G-12 (1) | — |
| G-12 (1) | 4-Me |
| G-12 (1) | 4-Et |
| G-12 (1) | 4-n-Pr |
| G-12 (1) | 4-i-Pr |
| G-12 (1) | 4-c-Pr |
| G-12 (1) | 4-n-Bu |
| G-12 (1) | 4-i-Bu |
| G-12 (1) | 4-t-Bu |
| G-12 (1) | 4-F |
| G-12 (1) | 4-Cl |
| G-12 (1) | 4-Br |
| G-12 (1) | 4-CF₃ |
| G-12 (1) | 4-HO |
| G-12 (1) | 4-N≡C |
| G-12 (1) | 4-N≡CCH₂ |
| G-12 (1) | 4-(MeO) |
| G-12 (1) | 4-(MeOCH₂) |
| G-12 (1) | 4-(EtOCH₂) |
| G-12 (1) | 4-(CH(═O)) |
| G-12 (1) | 4-(HOC(═O)) |
| G-12 (1) | 4-(MeOC(═O)) |
| G-12 (1) | 4-(EtOC(═O)) |
| G-12 (1) | 4-(i-PrOC(═O)) |
| G-12 (1) | 4-(n-PrOC(═O)) |
| G-12 (1) | 4-(BuOC(═O)) |
| G-12 (1) | 4-(i-BuOC(═O)) |
| G-12 (1) | 4-(t-BuOC(═O)) |
| G-12 (1) | 4-(CF₃CH₂OC(═O) |
| G-12 (1) | 4-(CH₂═CHOC(═O)) |
| G-12 (1) | 4-(CH₂═CHCH₂OC(═O)) |
| G-12 (1) | 4-(CH₂═CBrCH₂OC(═O)) |
| G-12 (1) | 4-(CH₂═CHCF₂OC(═O)) |
| G-12 (1) | 4-(Me₂C═CHCH₂OC(═O)) |
| G-12 (1) | 4-(CH₂═C(Me)CH₂OC(═O)) |
| G-12 (1) | 4-(CH≡CCH₂OC(═O)) |
| G-12 (1) | 4-(N≡CCH₂OC(═O)) |
| G-12 (1) | 4-(MeNHC(═O)) |
| G-12 (1) | 4-(Me₂NC(═O)) |
| G-12 (1) | 4-(MeNHC(═O)) |
| G-12 (1) | 4-(EtNHC(═O)) |
| G-12 (1) | 4-(PrNHC(═O)) |
| G-12 (1) | 4-(i-PrNHC(═O)) |
| G-12 (1) | 4-(BuNHC(═O)) |
| G-12 (1) | 4-(t-BuNHC(═O)) |
| G-12 (1) | 4-(i-BuNHC(═O)) |
| G-12 (1) | 4-(CF₃CH₂NHC(═O)) |
| G-12 (1) | 4-(c-PrCH₂NHC(═O)) |
| G-12 (1) | 4-(MeOCH₂NHC(═O)) |
| G-12 (1) | 4-(MeOCH₂CH₂NHC(═O)) |
| G-12 (1) | 4-(CH₂═CHCH₂NHC(═O)) |
| G-12 (1) | 4-(N≡CCH₂NHC(═O)) |
| G-12 (1) | 4-(OH—N═CH) |
| G-12 (1) | 4-(Me₂NN═CH) |
| G-12 (1) | 4-(MeOC(═O)NHN═CH) |
| G-12 (1) | 4-(OHC(═O)CH₂ON═CH) |
| G-12 (1) | 5-Me, 3-(EtOC(═O)) |
| G-12 (1) | 3-Me |
| G-12 (1) | 3-Et |
| G-12 (1) | 3-n-Pr |
| G-12 (1) | 3-i-Pr |
| G-12 (1) | 3-c-Pr |
| G-12 (1) | 3-n-Bu |
| G-12 (1) | 3-i-Bu |
| G-12 (1) | 3-t-Bu |
| G-12 (1) | 3-F |
| G-12 (1) | 3-Cl |
| G-12 (1) | 3-Br |
| G-12 (1) | 3-CF₃ |
| G-12 (1) | 3-HO |
| G-12 (1) | 3-N≡C |
| G-12 (1) | 3-N≡CCH₂ |
| G-12 (1) | 3-(MeO) |
| G-12 (1) | 3-(MeOCH₂) |
| G-12 (1) | 3-(EtOCH₂) |
| G-12 (1) | 3-(CH(═O)) |
| G-12 (1) | 3-(HOC(═O)) |
| G-12 (1) | 3-(MeOC(═O)) |
| G-12 (1) | 3-(EtOC(═O)) |
| G-12 (1) | 3-(i-PrOC(═O)) |
| G-12 (1) | 3-(n-PrOC(═O)) |
| G-12 (1) | 3-(BuOC(═O)) |
| G-12 (1) | 3-(i-BuOC(═O)) |
| G-12 (1) | 3-(t-BuOC(═O)) |
| G-12 (1) | 3-(CF₃CH₂OC(═O) |
| G-12 (1) | 3-(CH₂═CHOC(═O)) |
| G-12 (1) | 3-(CH₂═CHCH₂OC(═O)) |
| G-12 (1) | 3-(CH₂═CBrCH₂OC(═O)) |
| G-12 (1) | 3-(CH₂═CHCF₂OC(═O)) |
| G-12 (1) | 3-(Me₂C═CHCH₂OC(═O)) |
| G-12 (1) | 3-(CH₂═C(Me)CH₂OC(═O)) |
| G-12 (1) | 3-(CH≡CCH₂OC(═O)) |
| G-12 (1) | 3-(N≡CCH₂OC(═O)) |
| G-12 (1) | 3-(MeNHC(═O)) |
| G-12 (1) | 3-(Me₂NC(═O)) |
| G-12 (1) | 3-(MeNHC(═O)) |
| G-12 (1) | 3-(EtNHC(═O)) |
| G-12 (1) | 3-(PrNHC(═O)) |
| G-12 (1) | 3-(i-PrNHC(═O)) |
| G-12 (1) | 3-(BuNHC(═O)) |
| G-12 (1) | 3-(t-BuNHC(═O)) |
| G-12 (1) | 3-(i-BuNHC(═O)) |
| G-12 (1) | 3-(CF₃CH₂NHC(═O)) |
| G-12 (1) | 3-(c-PrCH₂NHC(═O)) |
| G-12 (1) | 3-(MeOCH₂NHC(═O)) |
| G-12 (1) | 3-(MeOCH₂CH₂NHC(═O)) |
| G-12 (1) | 3-(CH₂═CHCH₂NHC(═O)) |
| G-12 (1) | 3-(N≡CCH₂NHC(═O)) |
| G-12 (1) | 3-(OH—N═CH) |
| G-12 (1) | 3-(Me₂NN═CH) |
| G-12 (1) | 3-(MeOC(═O)NHN═CH) |
| G-12 (1) | 3-(OHC(═O)CH₂ON═CH) |
| G-13 (1) | — |
| G-13 (1) | 5-Me |
| G-13 (1) | 5-Et |
| G-13 (1) | 5-n-Pr |
| G-13 (1) | 5-i-Pr |
| G-13 (1) | 5-c-Pr |
| G-13 (1) | 5-n-Bu |
| G-13 (1) | 5-i-Bu |
| G-13 (1) | 5-t-Bu |
| G-13 (1) | 5-F |
| G-13 (1) | 5-Cl |
| G-13 (1) | 5-Br |
| G-13 (1) | 5-CF₃ |
| G-13 (1) | 5-HO |
| G-13 (1) | 5-N≡C |
| G-13 (1) | 5-N≡CCH₂ |
| G-13 (1) | 5-(MeO) |
| G-13 (1) | 5-(MeOCH₂) |
| G-13 (1) | 5-(EtOCH₂) |
| G-13 (1) | 5-(CH(═O)) |
| G-13 (1) | 5-(HOC(═O)) |
| G-13 (1) | 5-(MeOC(═O)) |
| G-13 (1) | 5-(EtOC(═O)) |
| G-13 (1) | 5-(i-PrOC(═O)) |
| G-13 (1) | 5-(n-PrOC(═O)) |
| G-13 (1) | 5-(BuOC(═O)) |
| G-13 (1) | 5-(i-BuOC(═O)) |
| G-13 (1) | 5-(t-BuOC(═O)) |
| G-13 (1) | 5-(CF₃CH₂OC(═O) |
| G-13 (1) | 5-(CH₂═CHOC(═O)) |
| G-13 (1) | 5-(CH₂═CHCH₂OC(═O)) |
| G-13 (1) | 5-(CH₂═CBrCH₂OC(═O)) |
| G-13 (1) | 5-(CH₂═CHCF₂OC(═O)) |
| G-13 (1) | 5-(Me₂C═CHCH₂OC(═O)) |
| G-13 (1) | 5-(CH₂═C(Me)CH₂OC(═O)) |
| G-13 (1) | 5-(CH≡CCH₂OC(═O)) |
| G-13 (1) | 5-(N≡CCH₂OC(═O)) |
| G-13 (1) | 5-(MeNHC(═O)) |
| G-13 (1) | 5-(Me₂NC(═O)) |
| G-13 (1) | 5-(MeNHC(═O)) |
| G-13 (1) | 5-(EtNHC(═O)) |
| G-13 (1) | 5-(PrNHC(═O)) |
| G-13 (1) | 5-(i-PrNHC(═O)) |
| G-13 (1) | 5-(BuNHC(═O)) |
| G-13 (1) | 5-(t-BuNHC(═O)) |
| G-13 (1) | 5-(i-BuNHC(═O)) |
| G-13 (1) | 5-(CF₃CH₂NHC(═O)) |
| G-13 (1) | 5-(c-PrCH₂NHC(═O)) |
| G-13 (1) | 5-(MeOCH₂NHC(═O)) |
| G-13 (1) | 5-(MeOCH₂CH₂NHC(═O)) |
| G-13 (1) | 5-(CH₂═CHCH₂NHC(═O)) |
| G-13 (1) | 5-(N≡CCH₂NHC(═O)) |
| G-13 (1) | 5-(OH—N═CH) |
| G-13 (1) | 5-(Me₂NN═CH) |
| G-13 (1) | 5-(MeOC(═O)NHN═CH) |
| G-13 (1) | 5-(OHC(═O)CH₂ON═CH) |
| G-17 (1) | — |
| G-17 (1) | 4-Me |
| G-17 (1) | 4-Et |
| G-17 (1) | 4-n-Pr |
| G-17 (1) | 4-i-Pr |
| G-17 (1) | 4-c-Pr |
| G-17 (1) | 4-n-Bu |
| G-17 (1) | 4-i-Bu |
| G-17 (1) | 4-t-Bu |
| G-17 (1) | 4-F |
| G-17 (1) | 4-Cl |
| G-17 (1) | 4-Br |
| G-17 (1) | 4-CF₃ |
| G-17 (1) | 4-HO |
| G-17 (1) | 4-N≡C |
| G-17 (1) | 4-N≡CCH₂ |
| G-17 (1) | 4-(MeO) |
| G-17 (1) | 4-(MeOCH₂) |
| G-17 (1) | 4-(EtOCH₂) |
| G-17 (1) | 4-(CH(═O)) |
| G-17 (1) | 4-(HOC(═O)) |
| G-17 (1) | 4-(MeOC(═O)) |
| G-17 (1) | 4-(EtOC(═O)) |
| G-17 (1) | 4-(i-PrOC(═O)) |
| G-17 (1) | 4-(n-PrOC(═O)) |
| G-17 (1) | 4-(BuOC(═O)) |
| G-17 (1) | 4-(i-BuOC(═O)) |
| G-17 (1) | 4-(t-BuOC(═O)) |
| G-17 (1) | 4-(CF₃CH₂OC(═O) |
| G-17 (1) | 4-(CH₂═CHOC(═O)) |
| G-17 (1) | 4-(CH₂═CHCH₂OC(═O)) |
| G-17 (1) | 4-(CH₂═CBrCH₂OC(═O)) |
| G-17 (1) | 4-(CH₂═CHCF₂OC(═O)) |
| G-17 (1) | 4-(Me₂C═CHCH₂OC(═O)) |
| G-17 (1) | 4-(CH₂═C(Me)CH₂OC(═O)) |
| G-17 (1) | 4-(CH≡CCH₂OC(═O)) |
| G-17 (1) | 4-(N≡CCH₂OC(═O)) |
| G-17 (1) | 4-(MeNHC(═O)) |
| G-17 (1) | 4-(Me₂NC(═O)) |
| G-17 (1) | 4-(MeNHC(═O)) |
| G-17 (1) | 4-(EtNHC(═O)) |
| G-17 (1) | 4-(PrNHC(═O)) |
| G-17 (1) | 4-(i-PrNHC(═O)) |
| G-17 (1) | 4-(BuNHC(═O)) |
| G-17 (1) | 4-(t-BuNHC(═O)) |
| G-17 (1) | 4-(i-BuNHC(═O)) |
| G-17 (1) | 4-(CF₃CH₂NHC(═O)) |
| G-17 (1) | 4-(c-PrCH₂NHC(═O)) |
| G-17 (1) | 4-(MeOCH₂NHC(═O)) |
| G-17 (1) | 4-(MeOCH₂CH₂NHC(═O)) |
| G-17 (1) | 4-(CH₂═CHCH₂NHC(═O)) |
| G-17 (1) | 4-(N≡CCH₂NHC(═O)) |
| G-17 (1) | 4-(OH—N═CH) |
| G-17 (1) | 4-(Me₂NN═CH) |
| G-17 (1) | 4-(MeOC(═O)NHN═CH) |
| G-17 (1) | 4-(OHC(═O)CH₂ON═CH) |

J is J-1, L is CH₂ and Z is a direct bond.

The present disclosure also includes Tables 1B through 48B, each of which is constructed the same as Table 2 above, except that the row heading in Table 2 (i.e. “J is J-1, L is CH₂, and Z is a direct bond”) is replaced with the respective row headings shown below.

| Table | Row Heading |
|---|---|
| 1B | J is J-1, L is CH₂CH₂ and Z is a direct bond. |
| 2B | J is J-1, L is CH₂(Me) and Z is a direct bond. |
| 3B | J is J-1, L is (CH₂)₃ and Z is a direct bond. |
| 4B | J is J-1, L is CH₂ and Z is O. |
| 5B | J is J-2, L is CH₂ and Z is a direct bond. |
| 6B | J is J-2, L is CH₂CH₂ and Z is a direct bond. |
| 7B | J is J-2, L is CH₂(Me) and Z is a direct bond. |
| 8B | J is J-2, L is (CH₂)₃ and Z is a direct bond. |
| 9B | J is J-2, L is CH₂ and Z is O. |
| 10B | J is J-6, L is CH₂ and Z is a direct bond. |
| 11B | J is J-6, L is CH₂CH₂ and Z is a direct bond. |
| 12B | J is J-6, L is CH₂(Me) and Z is a direct bond. |
| 13B | J is J-6, L is (CH₂)₃ and Z is a direct bond. |
| 14B | J is J-6, L is CH₂ and Z is O. |
| 15B | J is J-7, L is CH₂ and Z is a direct bond. |
| 16B | J is J-7, L is CH₂CH₂ and Z is a direct bond. |
| 17B | J is J-7, L is CH₂(Me) and Z is a direct bond. |
| 18B | J is J-7, L is (CH₂)₃ and Z is a direct bond. |
| 19B | J is J-7, L is CH₂ and Z is O. |
| 20B | J is J-8, L is CH₂ and Z is a direct bond. |
| 21B | J is J-8, L is CH₂CH₂ and Z is a direct bond. |
| 22B | J is J-8, L is CH₂(Me) and Z is a direct bond. |
| 23B | J is J-8, L is (CH₂)₃ and Z is a direct bond. |
| 24B | J is J-8, L is CH₂ and Z is O. |
| 25B | J is J-10, L is CH₂ and Z is a direct bond. |
| 26B | J is J-10, L is CH₂CH₂ and Z is a direct bond. |
| 27B | J is J-10, L is CH₂(Me) and Z is a direct bond. |
| 28B | J is J-10, L is (CH₂)₃ and Z is a direct bond. |
| 29B | J is J-10, L is CH₂ and Z is O. |
| 30B | J is J-14, L is CH₂ and Z is a direct bond. |
| 31B | J is J-14, L is CH₂CH₂ and Z is a direct bond. |
| 32B | J is J-14, L is CH₂(Me) and Z is a direct bond. |
| 33B | J is J-14, L is (CH₂)₃ and Z is a direct bond. |
| 34B | J is J-14, L is CH₂ and Z is O. |
| 35B | J is J-3, L is CH₂ and Z is a direct bond. |
| 36B | J is J-3, L is CH₂CH₂ and Z is a direct bond. |
| 37B | J is J-3, L is CH₂(Me) and Z is a direct bond. |
| 38B | J is J-3, L is (CH₂)₃ and Z is a direct bond. |
| 39B | J is J-3, L is CH₂ and Z is O. |
| 40B | J is J-4, L is CH₂ and Z is a direct bond. |
| 41B | J is J-4, L is CH₂CH₂ and Z is a direct bond. |
| 42B | J is J-4, L is CH₂(Me) and Z is a direct bond. |
| 43B | J is J-4, L is (CH₂)₃ and Z is a direct bond. |
| 44B | J is J-4, L is CH₂ and Z is O. |
| 45B | J is J-5, L is CH₂ and Z is a direct bond. |
| 46B | J is J-5, L is CH₂CH₂ and Z is a direct bond. |
| 47B | J is J-5, L is CH₂(Me) and Z is a direct bond. |
| 48B | J is J-5, L is (CH₂)₃ and Z is a direct bond. |

Table 3 discloses specific compounds Formula 10 which are useful as process intermediates for preparing compounds of Formula 1, as described in Scheme 14 above.

TABLE 3

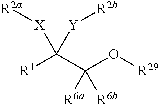

| R¹ | R^{6a} | R^{6b} | X | Y | R^{2a} | R^{2b} | R³⁰ |
|---|---|---|---|---|---|---|---|
| CF₃ | H | H | O | O | H | H | CF₃ |
| CF₃ | H | H | O | O | H | H | CH₃ |
| CF₃ | H | H | O | O | H | H | CH₂CF₃ |
| CF₃ | H | H | O | O | H | H | (CF₂)₃CF₃ |
| CF₃ | H | H | O | O | H | H | Ph |
| CF₃ | H | H | O | O | H | H | 4-Me—Ph |
| CF₃ | H | H | O | O | H | H | 4-Br—Ph |
| CF₃ | H | H | O | O | H | H | 4-NO₂—Ph |
| CF₃ | H | H | O | NH | H | H | CF₃ |
| CF₃ | H | H | O | NH | H | H | CH₃ |
| CF₃ | H | H | O | NH | H | H | CH₂CF₃ |
| CF₃ | H | H | O | NH | H | H | (CF₂)₃CF₃ |
| CF₃ | H | H | O | NH | H | H | Ph |
| CF₃ | H | H | O | NH | H | H | 4-Me—Ph |
| CF₃ | H | H | O | NH | H | H | 4-Br—Ph |
| CF₃ | H | H | O | NH | H | H | 4-NO₂—Ph |
| CF₃ | H | H | O | NH | H | Me | CF₃ |
| CF₃ | H | H | O | NH | H | Me | CH₃ |
| CF₃ | H | H | O | NH | H | Me | CH₂CF₃ |
| CF₃ | H | H | O | NH | H | Me | (CF₂)₃CF₃ |
| CF₃ | H | H | O | NH | H | Me | Ph |
| CF₃ | H | H | O | NH | H | Me | 4-Me—Ph |
| CF₃ | H | H | O | NH | H | Me | 4-Br—Ph |
| CF₃ | H | H | O | NH | H | Me | 4-NO₂—Ph |
| CF₃ | H | Me | O | O | H | H | CF₃ |
| CF₃ | H | Me | O | O | H | H | CH₃ |
| CF₃ | H | Me | O | O | H | H | CH₂CF₃ |
| CF₃ | H | Me | O | O | H | H | (CF₂)₃CF₃ |
| CF₃ | H | Me | O | O | H | H | Ph |
| CF₃ | H | Me | O | O | H | H | 4-Me—Ph |
| CF₃ | H | Me | O | O | H | H | 4-Br—Ph |
| CHF₂ | H | Me | O | O | H | H | 4-NO₂—Ph |
| CHF₂ | H | H | O | O | H | H | CF₃ |
| CHF₂ | H | H | O | O | H | H | CH₃ |
| CHF₂ | H | H | O | O | H | H | CH₂CF₃ |
| CHF₂ | H | H | O | O | H | H | (CF₂)₃CF₃ |
| CHF₂ | H | H | O | O | H | H | Ph |
| CHF₂ | H | H | O | O | H | H | 4-Me—Ph |
| CHF₂ | H | H | O | O | H | H | 4-Br—Ph |
| CHF₂ | H | H | O | O | H | H | 4-NO₂—Ph |
| CCl₃ | H | H | O | O | H | H | CF₃ |
| CCl₃ | H | H | O | O | H | H | CH₃ |
| CCl₃ | H | H | O | O | H | H | CH₂CF₃ |
| CCl₃ | H | H | O | O | H | H | (CF₂)₃CF₃ |
| CCl₃ | H | H | O | O | H | H | Ph |
| CCl₃ | H | H | O | O | H | H | 4-Me—Ph |
| CCl₃ | H | H | O | O | H | H | 4-Br—Ph |
| CCl₃ | H | H | O | O | H | H | 4-NO₂—Ph |
| CF₃ | Me | Me | O | O | H | H | CF₃ |
| CF₃ | Me | Me | O | O | H | H | CH₃ |
| CF₃ | Me | Me | O | O | H | H | CH₂CF₃ |
| CF₃ | Me | Me | O | O | H | H | (CF₂)₃CF₃ |
| CF₃ | Me | Me | O | O | H | H | Ph |
| CF₃ | Me | Me | O | O | H | H | 4-Me—Ph |
| CF₃ | Me | Me | O | O | H | H | 4-Br—Ph |
| CF₃ | Me | Me | O | O | H | H | 4-NO₂—Ph |

| | | | | | | |
|---|---|---|---|---|---|---|
| CF₃ | H | H | O | O | —CH₂CH₂— | CF₃ |
| CF₃ | H | H | O | O | —CH₂CH₂— | CH₃ |
| CF₃ | H | H | O | O | —CH₂CH₂— | CH₂CF₃ |
| CF₃ | H | H | O | O | —CH₂CH₂— | (CF₂)₃CF₃ |
| CF₃ | H | H | O | O | —CH₂CH₂— | Ph |
| CF₃ | H | H | O | O | —CH₂CH₂— | 4-Me—Ph |
| CF₃ | H | H | O | O | —CH₂CH₂— | 4-Br—Ph |
| CF₃ | H | H | O | O | —CH₂CH₂— | 4-NO₂—Ph |
| CF₃ | H | H | O | O | —CH₂CH(═O)— | CF₃ |
| CF₃ | H | H | O | O | —CH₂CH(═O)— | CH₃ |
| CF₃ | H | H | O | O | —CH₂CH(═O)— | CH₂CF₃ |
| CF₃ | H | H | O | O | —CH₂CH(═O)— | (CF₂)₃CF₃ |
| CF₃ | H | H | O | O | —CH₂CH(═O)— | Ph |
| CF₃ | H | H | O | O | —CH₂CH(═O)— | 4-Me—Ph |
| CF₃ | H | H | O | O | —CH₂CH(═O)— | 4-Br—Ph |
| CF₃ | H | H | O | O | —CH₂CH₂— | 4-NO₂—Ph |
| CF₃ | H | H | O | O | —CH₂CH(Me)— | CF₃ |
| CF₃ | H | H | O | O | —CH₂CH(Me)— | CH₃ |
| CF₃ | H | H | O | O | —CH₂CH(Me)— | CH₂CF₃ |
| CF₃ | H | H | O | O | —CH₂CH(Me)— | (CF₂)₃CF₃ |
| CF₃ | H | H | O | O | —CH₂CH(Me)— | Ph |
| CF₃ | H | H | O | O | —CH₂CH(Me)— | 4-Me—Ph |
| CF₃ | H | H | O | O | —CH₂CH(Me)— | 4-Br—Ph |
| CF₃ | H | H | O | O | —CH₂CH(Me)— | 4-NO₂—Ph |

R²⁹ is S(═O)₂R³⁰.

### Formulation/Utility

A compound of Formula 1 of this invention (including N-oxides and salts thereof) will generally be used as a fungicidal active ingredient in a composition, i.e. formulation, with at least one additional component selected from the group consisting of surfactants, solid diluents and liquid diluents, which serve as a carrier. The formulation or composition ingredients are selected to be consistent with the physical properties of the active ingredient, mode of application and environmental factors such as soil type, moisture and temperature.

Useful formulations include both liquid and solid compositions. Liquid compositions include solutions (including emulsifiable concentrates), suspensions, emulsions (including microemulsions, oil-in-water emulsions, flowable concentrates and/or suspoemulsions) and the like, which optionally can be thickened into gels. The general types of aqueous liquid compositions are soluble concentrate, suspension concentrate, capsule suspension, concentrated emulsion, microemulsion, oil-in-water emulsion, flowable concentrate and suspoemulsion. The general types of nonaqueous liquid compositions are emulsifiable concentrate, microemulsifiable concentrate, dispersible concentrate and oil dispersion.

The general types of solid compositions are dusts, powders, granules, pellets, prills, pastilles, tablets, filled films (including seed coatings) and the like, which can be water-dispersible (“wettable”) or water-soluble. Films and coatings formed from film-forming solutions or flowable suspensions are particularly useful for seed treatment. Active ingredient can be (micro)encapsulated and further formed into a suspension or solid formulation; alternatively the entire formulation of active ingredient can be encapsulated (or “overcoated”). Encapsulation can control or delay release of the active ingredient. An emulsifiable granule combines the advantages of both an emulsifiable concentrate formulation and a dry granular formulation. High-strength compositions are primarily used as intermediates for further formulation.

Sprayable formulations are typically extended in a suitable medium before spraying. Such liquid and solid formulations are formulated to be readily diluted in the spray medium, usually water, but occasionally another suitable medium like an aromatic or paraffinic hydrocarbon or vegetable oil. Spray volumes can range from about one to several thousand liters per hectare, but more typically are in the range from about ten to several hundred liters per hectare. Sprayable formulations can be tank mixed with water or another suitable medium for foliar treatment by aerial or ground application, or for application to the growing medium of the plant. Liquid and dry formulations can be metered directly into drip irrigation systems or metered into the furrow during planting. Liquid and solid formulations can be applied onto seeds of crops and other desirable vegetation as seed treatments before planting to protect developing roots and other subterranean plant parts and/or foliage through systemic uptake.

The formulations will typically contain effective amounts of active ingredient, diluent and surfactant within the following approximate ranges which add up to 100 percent by weight.

| | Weight Percent | | |
|---|---|---|---|
| | Active | | |
| | Ingredient | Diluent | Surfactant |
| Water-Dispersible and Water- | 0.001-90 | 0-99.999 | 0-15 |
| soluble Granules, Tablets and | | | |
| Powders | | | |
| Oil Dispersions, Suspensions, | 1-50 | 40-99 | 0-50 |
| Emulsions, Solutions | | | |
| (including Emulsifiable | | | |
| Concentrates) | | | |
| Dusts | 1-25 | 70-99 | 0-5 |
| Granules and Pellets | 0.001-95 | 5-99.999 | 0-15 |
| High Strength Compositions | 90-99 | 0-10 | 0-2 |

Solid diluents include, for example, clays such as bentonite, montmorillonite, attapulgite and kaolin, gypsum, cellulose, titanium dioxide, zinc oxide, starch, dextrin, sugars (e.g., lactose, sucrose), silica, talc, mica, diatomaceous earth, urea, calcium carbonate, sodium carbonate and bicarbonate, and sodium sulfate. Typical solid diluents are described in Watkins et al., Handbook of Insecticide Dust Diluents and Carriers, 2nd Ed., Dorland Books, Caldwell, New Jersey.

Liquid diluents include, for example, water, N,N-dimethylalkanamides (e.g., N,N-dimethylformamide), limonene, dimethyl sulfoxide, N-alkylpyrrolidones (e.g., N-methylpyrrolidinone), alkyl phosphates (e.g., triethyl phosphate), ethylene glycol, triethylene glycol, propylene glycol, dipropylene glycol, polypropylene glycol, propylene carbonate, butylene carbonate, paraffins (e.g., white mineral oils, normal paraffins, isoparaffins), alkylbenzenes, alkylnaphthalenes, glycerine, glycerol triacetate, sorbitol, aromatic hydrocarbons, dearomatized aliphatics, alkylbenzenes, alkylnaphthalenes, ketones such as cyclohexanone, 2-heptanone, isophorone and 4-hydroxy-4-methyl-2-pentanone, acetates such as isoamyl acetate, hexyl acetate, heptyl acetate, octyl acetate, nonyl acetate, tridecyl acetate and isobornyl acetate, other esters such as alkylated lactate esters, dibasic esters, alkyl and aryl benzoates and γ-butyrolactone, and alcohols, which can be linear, branched, saturated or unsaturated, such as methanol, ethanol, n-propanol, isopropyl alcohol, n-butanol, isobutyl alcohol, n-hexanol, 2-ethylhexanol, n-octanol, decanol, isodecyl alcohol, isooctadecanol, cetyl alcohol, lauryl alcohol, tridecyl alcohol, oleyl alcohol, cyclohexanol, tetrahydrofurfuryl alcohol, diacetone alcohol, cresol and benzyl alcohol. Liquid diluents also include glycerol esters of saturated and unsaturated fatty acids (typically C₆-C₂₂), such as plant seed and fruit oils (e.g., oils of olive, castor, linseed, sesame, corn (maize), peanut, sunflower, grapeseed, safflower, cottonseed, soybean, rapeseed, coconut and palm kernel), animal-sourced fats (e.g., beef tallow, pork tallow, lard, cod liver oil, fish oil), and mixtures thereof. Liquid diluents also include alkylated fatty acids (e.g., methylated, ethylated, butylated) wherein the fatty acids may be obtained by hydrolysis of glycerol esters from plant and animal sources, and can be purified by distillation. Typical liquid diluents are described in Marsden, Solvents Guide, 2nd Ed., Interscience, New York, 1950.

The solid and liquid compositions of the present invention often include one or more surfactants. When added to a liquid, surfactants (also known as “surface-active agents”) generally modify, most often reduce, the surface tension of the liquid. Depending on the nature of the hydrophilic and lipophilic groups in a surfactant molecule, surfactants can be useful as wetting agents, dispersants, emulsifiers or defoaming agents.

Surfactants can be classified as nonionic, anionic or cationic. Nonionic surfactants useful for the present compositions include, but are not limited to: alcohol alkoxylates such as alcohol alkoxylates based on natural and synthetic alcohols (which may be branched or linear) and prepared from the alcohols and ethylene oxide, propylene oxide, butylene oxide or mixtures thereof; amine ethoxylates, alkanolamides and ethoxylated alkanolamides; alkoxylated triglycerides such as ethoxylated soybean, castor and rapeseed oils; alkylphenol alkoxylates such as octylphenol ethoxylates, nonylphenol ethoxylates, dinonyl phenol ethoxylates and dodecyl phenol ethoxylates (prepared from the phenols and ethylene oxide, propylene oxide, butylene oxide or mixtures thereof); block polymers prepared from ethylene oxide or propylene oxide and reverse block polymers where the terminal blocks are prepared from propylene oxide; ethoxylated fatty acids; ethoxylated fatty esters and oils; ethoxylated methyl esters; ethoxylated tristyrylphenol (including those prepared from ethylene oxide, propylene oxide, butylene oxide or mixtures thereof); fatty acid esters, glycerol esters, lanolin-based derivatives, polyethoxylate esters such as polyethoxylated sorbitan fatty acid esters, polyethoxylated sorbitol fatty acid esters and polyethoxylated glycerol fatty acid esters; other sorbitan derivatives such as sorbitan esters; polymeric surfactants such as random copolymers, block copolymers, alkyd peg (polyethylene glycol) resins, graft or comb polymers and star polymers; polyethylene glycols (pegs); polyethylene glycol fatty acid esters; silicone-based surfactants; and sugar-derivatives such as sucrose esters, alkyl polyglycosides and alkyl polysaccharides.

Useful anionic surfactants include, but are not limited to: alkylaryl sulfonic acids and their salts; carboxylated alcohol or alkylphenol ethoxylates; diphenyl sulfonate derivatives; lignin and lignin derivatives such as lignosulfonates; maleic or succinic acids or their anhydrides; olefin sulfonates; phosphate esters such as phosphate esters of alcohol alkoxylates, phosphate esters of alkylphenol alkoxylates and phosphate esters of styryl phenol ethoxylates; protein-based surfactants; sarcosine derivatives; styryl phenol ether sulfate; sulfates and sulfonates of oils and fatty acids; sulfates and sulfonates of ethoxylated alkylphenols; sulfates of alcohols; sulfates of ethoxylated alcohols; sulfonates of amines and amides such as N,N-alkyltaurates; sulfonates of benzene, cumene, toluene, xylene, and dodecyl and tridecylbenzenes; sulfonates of condensed naphthalenes; sulfonates of naphthalene and alkyl naphthalene; sulfonates of fractionated petroleum; sulfosuccinamates; and sulfosuccinates and their derivatives such as dialkyl sulfosuccinate salts.

Useful cationic surfactants include, but are not limited to: amides and ethoxylated amides; amines such as N-alkyl propanediamines, tripropylenetriamines and dipropylenetetramines, and ethoxylated amines, ethoxylated diamines and propoxylated amines (prepared from the amines and ethylene oxide, propylene oxide, butylene oxide or mixtures thereof); amine salts such as amine acetates and diamine salts; quaternary ammonium salts such as quaternary salts, ethoxylated quaternary salts and diquaternary salts; and amine oxides such as alkyldimethylamine oxides and bis-(2-hydroxyethyl)-alkylamine oxides.

Also useful for the present compositions are mixtures of nonionic and anionic surfactants or mixtures of nonionic and cationic surfactants. Nonionic, anionic and cationic surfactants and their recommended uses are disclosed in a variety of published references including McCutcheon's Emulsifiers and Detergents, annual American and International Editions published by McCutcheon's Division, The Manufacturing Confectioner Publishing Co.; Sisely and Wood, Encyclopedia of Surface Active Agents, Chemical Publ. Co., Inc., New York, 1964; and A. S. Davidson and B. Milwidsky, Synthetic Detergents, Seventh Edition, John Wiley and Sons, New York, 1987.

Compositions of this invention may also contain formulation auxiliaries and additives, known to those skilled in the art as formulation aids (some of which may be considered to also function as solid diluents, liquid diluents or surfactants). Such formulation auxiliaries and additives may control: pH (buffers), foaming during processing (antifoams such polyorganosiloxanes), sedimentation of active ingredients (suspending agents), viscosity (thixotropic thickeners), in-container microbial growth (antimicrobials), product freezing (antifreezes), color (dyes/pigment dispersions), wash-off (film formers or stickers), evaporation (evaporation retardants), and other formulation attributes. Film formers include, for example, polyvinyl acetates, polyvinyl acetate copolymers, polyvinylpyrrolidone-vinyl acetate copolymer, polyvinyl alcohols, polyvinyl alcohol copolymers and waxes. Examples of formulation auxiliaries and additives include those listed in McCutcheon's Volume 2: Functional Materials, annual International and North American editions published by McCutcheon's Division, The Manufacturing Confectioner Publishing Co.; and PCT Publication WO 03/024222.

The compound of Formula 1 and any other active ingredients are typically incorporated into the present compositions by dissolving the active ingredient in a solvent or by grinding in a liquid or dry diluent. Solutions, including emulsifiable concentrates, can be prepared by simply mixing the ingredients. If the solvent of a liquid composition intended for use as an emulsifiable concentrate is water-immiscible, an emulsifier is typically added to emulsify the active-containing solvent upon dilution with water. Active ingredient slurries, with particle diameters of up to 2,000 μm can be wet milled using media mills to obtain particles with average diameters below 3 μm. Aqueous slurries can be made into finished suspension concentrates (see, for example, U.S. Pat. No. 3,060,084) or further processed by spray drying to form water-dispersible granules. Dry formulations usually require dry milling processes, which produce average particle diameters in the 2 to 10 μm range. Dusts and powders can be prepared by blending and usually grinding (such as with a hammer mill or fluid-energy mill). Granules and pellets can be prepared by spraying the active material upon preformed granular carriers or by agglomeration techniques. See Browning, “Agglomeration”, Chemical Engineering, Dec. 4, 1967, pp 147-48, Perry's Chemical Engineer's Handbook, 4th Ed., McGraw-Hill, New York, 1963, pp 8-57 and following, and WO 91/13546. Pellets can be prepared as described in U.S. Pat. No. 4,172,714. Water-dispersible and water-soluble granules can be prepared as taught in U.S. Pat. Nos. 4,144,050, 3,920,442 and DE 3,246,493. Tablets can be prepared as taught in U.S. Pat. Nos. 5,180,587, 5,232,701 and 5,208,030. Films can be prepared as taught in GB 2,095,558 and U.S. Pat. No. 3,299,566.

One embodiment of the present invention relates to a method for controlling fungal pathogens, comprising diluting the fungicidal composition of the present invention (a compound of Formula 1 formulated with surfactants, solid diluents and liquid diluents or a formulated mixture of a compound of Formula 1 and at least one other fungicide) with water, and optionally adding an adjuvant to form a diluted composition, and contacting the fungal pathogen or its environment with an effective amount of said diluted composition.

Although a spray composition formed by diluting with water a sufficient concentration of the present fungicidal composition can provide sufficient efficacy for controlling fungal pathogens, separately formulated adjuvant products can also be added to spray tank mixtures. These additional adjuvants are commonly known as “spray adjuvants” or “tank-mix adjuvants”, and include any substance mixed in a spray tank to improve the performance of a pesticide or alter the physical properties of the spray mixture. Adjuvants can be anionic or nonionic surfactants, emulsifying agents, petroleum-based crop oils, crop-derived seed oils, acidifiers, buffers, thickeners or defoaming agents. Adjuvants are used to enhancing efficacy (e.g., biological availability, adhesion, penetration, uniformity of coverage and durability of protection), or minimizing or eliminating spray application problems associated with incompatibility, foaming, drift, evaporation, volatilization and degradation. To obtain optimal performance, adjuvants are selected with regard to the properties of the active ingredient, formulation and target (e.g., crops, insect pests).

The amount of adjuvants added to spray mixtures is generally in the range of about 2.5% to 0.1% by volume. The application rates of adjuvants added to spray mixtures are typically between about 1 to 5 L per hectare. Representative examples of spray adjuvants include: Adigor® (Syngenta) 47% methylated rapeseed oil in liquid hydrocarbons, Silwet® (Helena Chemical Company) polyalkyleneoxide modified heptamethyltrisiloxane and Assist® (BASF) 17% surfactant blend in 83% paraffin based mineral oil.

One method of seed treatment is by spraying or dusting the seed with a compound of the invention (i.e. as a formulated composition) before sowing the seeds. Compositions formulated for seed treatment generally comprise a film former or adhesive agent. Therefore typically a seed coating composition of the present invention comprises a biologically effective amount of a compound of Formula 1 and a film former or adhesive agent. Seeds can be coated by spraying a flowable suspension concentrate directly into a tumbling bed of seeds and then drying the seeds. Alternatively, other formulation types such as wetted powders, solutions, suspoemulsions, emulsifiable concentrates and emulsions in water can be sprayed on the seed. This process is particularly useful for applying film coatings on seeds. Various coating machines and processes are available to one skilled in the art. Suitable processes include those listed in P. Kosters et al., Seed Treatment: Progress and Prospects, 1994 BCPC Mongraph No. 57, and references listed therein.

For further information regarding the art of formulation, see T. S. Woods, “The Formulator's Toolbox—Product Forms for Modern Agriculture” in Pesticide Chemistry and Bioscience, The Food-Environment Challenge, T. Brooks and T. R. Roberts, Eds., Proceedings of the 9th International Congress on Pesticide Chemistry, The Royal Society of Chemistry, Cambridge, 1999, pp. 120-133. Also see U.S. Pat. No. 3,235,361, Col. 6, line 16 through Col. 7, line 19 and Examples 10-41; U.S. Pat. No. 3,309,192, Col. 5, line 43 through Col. 7, line 62 and Examples 8, 12, 15, 39, 41, 52, 53, 58, 132, 138-140, 162-164, 166, 167 and 169-182; U.S. Pat. No. 2,891,855, Col. 3, line 66 through Col. 5, line 17 and Examples 1-4; Klingman, Weed Control as a Science, John Wiley and Sons, Inc., New York, 1961, pp 81-96; Hance et al., Weed Control Handbook, 8th Ed., Blackwell Scientific Publications, Oxford, 1989; and Developments in formulation technology, PJB Publications, Richmond, U K, 2000.

In the following Examples, all percentages are by weight and all formulations are prepared in conventional ways. Active ingredient refers to the compounds in Index Tables A-L disclosed herein. Without further elaboration, it is believed that one skilled in the art using the preceding description can utilize the present invention to its fullest extent. The following Examples are, therefore, to be constructed as merely illustrative, and not limiting of the disclosure in any way whatsoever.

### Example A

High Strength Concentrate

| | |
|---|---|
| Compound 262 | 98.5% |
| silica aerogel | 0.5% |
| synthetic amorphous fine silica | 1.0% |

### Example B

Wettable Powder

| | |
|---|---|
| Compound 231 | 65.0% |
| dodecylphenol polyethylene glycol ether | 2.0% |
| sodium ligninsulfonate | 4.0% |
| sodium silicoaluminate | 6.0% |
| montmorillonite (calcined) | 23.0% |

### Example C

Granule

| | |
|---|---|
| Compound 64 | 10.0% |
| attapulgite granules (low volatile matter, 0.71/0.30 mm; | 90.0% |
| U.S.S. No. 25-50 sieves) | |

### Example D

Extruded Pellet

| | |
|---|---|
| Compound 32 | 25.0% |
| anhydrous sodium sulfate | 10.0% |
| crude calcium ligninsulfonate | 5.0% |
| sodium alkylnaphthalenesulfonate | 1.0% |
| calcium/magnesium bentonite | 59.0% |

### Example E

Emulsifiable Concentrate

| | |
|---|---|
| Compound 64 | 10.0% |
| polyoxyethylene sorbitol hexoleate | 20.0% |
| C₆-C₁₀ fatty acid methyl ester | 70.0% |

### Example F

Microemulsion

| | |
|---|---|
| Compound 231 | 5.0% |
| polyvinylpyrrolidone-vinyl acetate copolymer | 30.0% |
| alkylpolyglycoside | 30.0% |
| glyceryl monooleate | 15.0% |
| water | 20.0% |

### Example G

Seed Treatment

| | |
|---|---|
| Compound 262 | 20.00% |
| polyvinylpyrrolidone-vinyl acetate copolymer | 5.00% |
| montan acid wax | 5.00% |
| calcium ligninsulfonate | 1.00% |
| polyoxyethylene/polyoxypropylene block copolymers | 1.00% |
| stearyl alcohol (POE 20) | 2.00% |
| polyorganosilane | 0.20% |
| colorant red dye | 0.05% |
| water | 65.75% |

### Example H

Fertilizer Stick

| | |
|---|---|
| Compound 32 | 2.50% |
| pyrrolidone-styrene copolymer | 4.80% |
| tristyrylphenyl 16-ethoxylate | 2.30% |
| talc | 0.80% |
| corn starch | 5.00% |
| slow-release fertilizer | 36.00% |
| kaolin | 38.00% |
| water | 10.60% |

### Example I

Suspension Concentrate

| | |
|---|---|
| Compound 64 | 35% |
| butyl polyoxyethylene/polypropylene block copolymer | 4.0% |
| stearic acid/polyethylene glycol copolymer | 1.0% |
| styrene acrylic polymer | 1.0% |
| xanthan gum | 0.1% |
| propylene glycol | 5.0% |
| silicone based defoamer | 0.1% |
| 1,2-benzisothiazolin-3-one | 0.1% |
| water | 53.7% |

### Example J

Emulsion in Water

| | |
|---|---|
| Compound 33 | 10.0% |
| butyl polyoxyethylene/polypropylene block copolymer | 4.0% |
| stearic acid/polyethylene glycol copolymer | 1.0% |
| styrene acrylic polymer | 1.0% |
| xanthan gum | 0.1% |
| propylene glycol | 5.0% |
| silicone based defoamer | 0.1% |
| 1,2-benzisothiazolin-3-one | 0.1% |
| aromatic petroleum based hydrocarbon | 20.0 |
| water | 58.7% |

### Example K

Oil Dispersion

| | |
|---|---|
| Compound 3 | 25% |
| polyoxyethylene sorbitol hexaoleate | 15% |
| organically modified bentonite clay | 2.5% |
| fatty acid methyl ester | 57.5% |

### Example L

Suspoemulsion

| | |
|---|---|
| Compound 9 | 10.0% |
| imidacloprid | 5.0% |
| butyl polyoxyethylene/polypropylene block copolymer | 4.0% |
| stearic acid/polyethylene glycol copolymer | 1.0% |
| styrene acrylic polymer | 1.0% |
| xanthan gum | 0.1% |
| propylene glycol | 5.0% |
| silicone based defoamer | 0.1% |
| 1,2-benzisothiazolin-3-one | 0.1% |
| aromatic petroleum based hydrocarbon | 20.0% |
| water | 53.7% |

Water-soluble and water-dispersible formulations are typically diluted with water to form aqueous compositions before application. Aqueous compositions for direct applications to the plant or portion thereof (e.g., spray tank compositions) typically contain at least about 1 ppm or more (e.g., from 1 ppm to 100 ppm) of the compound(s) of this invention.

Seed is normally treated at a rate of from about 0.001 g (more typically about 0.1 g) to about 10 g per kilogram of seed (i.e. from about 0.0001 to 1% by weight of the seed before treatment). A flowable suspension formulated for seed treatment typically comprises from about 0.5 to about 70% of the active ingredient, from about 0.5 to about 30% of a film-forming adhesive, from about 0.5 to about 20% of a dispersing agent, from 0 to about 5% of a thickener, from 0 to about 5% of a pigment and/or dye, from 0 to about 2% of an antifoaming agent, from 0 to about 1% of a preservative, and from 0 to about 75% of a volatile liquid diluent.

The compounds of this invention are useful as plant disease control agents. The present invention therefore further comprises a method for controlling plant diseases caused by fungal plant pathogens comprising applying to the plant or portion thereof to be protected, or to the plant seed to be protected, an effective amount of a compound of the invention or a fungicidal composition containing said compound. The compounds and/or compositions of this invention provide control of diseases caused by a broad spectrum of fungal plant pathogens in the Ascomycota, Basidiomycota, Zygomycota phyla, and the fungal-like Oomycata class. They are effective in controlling a broad spectrum of plant diseases, particularly foliar pathogens of ornamental, turf, vegetable, field, cereal, and fruit crops. These pathogens include but are not limited to those listed in Table 1-1. For Ascomycetes and Basidiomycetes, names for both the sexual/teleomorph/perfect stage as well as names for the asexual/anamorph/imperfect stage (in parentheses) are listed where known. Synonymous names for pathogens are indicated by an equal sign. For example, the sexual/teleomorph/perfect stage name Phaeosphaeria nodorum is followed by the corresponding asexual/anamorph/imperfect stage name Stagnospora nodorum and the synonymous older name Septoria nodorum.

TABLE 1-1
Ascomycetes in the order Pleosporales including Alternaria solani, A. alternata and A. brassicae,
Guignardia bidwellii, Venturia inaequalis, Pyrenophora tritici-repentis (Dreschlera tritici-repentis =
Helminthosporium tritici-repentis) and Pyrenophora teres (Dreschlera teres = Helminthosporium
teres), Corynespora cassiicola, Phaeosphaeria nodorum (Stagonospora nodorum = Septoria
nodorum), Cochliobolus carbonum and C. heterostrophus, Leptosphaeria biglobosa and L.
maculans;
Ascomycetes in the order Mycosphaerellales including Mycosphaerella graminicola (Zymoseptoria
tritici = Septoria tritici), M. berkeleyi (Cercosporidium personatum), M. arachidis (Cercospora
arachidicola), Passalora sojina (Cercospora sojina), Cercospora zeae-maydis and C. beticola;
Ascomycetes in the order Erysiphales (the powdery mildews) such as Blumeria graminis f. sp. tritici
and Blumeria graminis f. sp. hordei, Erysiphe polygoni, E. necator (=Uncinula necator),
Podosphaera fuliginea (=Sphaerotheca fuliginea), and Podosphaera leucotricha (=Sphaerotheca
fuliginea);
Ascomycetes in the order Helotiales such as Botryotinia fuckeliana (Botrytis cinerea), Oculimacula
yallundae (=Tapesia yallundae; anamorph Helgardia herpotrichoides = Pseudocercosporella
herpetrichoides), Monilinia fructicola, Sclerotinia sclerotiorum, Sclerotinia minor, and Sclerotinia
homoeocarpa;
Ascomycetes in the order Hypocreales such as Giberella zeae (Fusarium graminearum), G.
monoliformis (Fusarium moniliforme), Fusarium solani and Verticillium dahliae;
Ascomycetes in the order Eurotiales such as Aspergillus flavus and A. parasiticus;
Ascomycetes in the order Diaporthales such as Cryptosphorella viticola (=Phomopsis viticola),
Phomopsis longicolla, and Diaporthe phaseolorum;
Other Ascomycete pathogens including Magnaporthe grisea, Gaeumannomyces graminis,
Rhynchosporium secalis, and anthracnose pathogens such as Glomerella acutata (Colletotrichum
acutatum), G. graminicola (C. graminicola) and G. lagenaria (C. orbiculare);
Basidiomycetes in the order Urediniales (the rusts) including Puccinia recondita, P. striiformis,
Puccinia hordei, P. graminis and P. arachidis), Hemileia vastatrix and Phakopsora pachyrhizi;
Basidiomycetes in the order Ceratobasidiales such as Thanatophorum cucumeris (Rhizoclonia solani)
and Ceratobasidium oryzae-sativae (Rhizoclonia oryzae);
Basidiomycetes in the order Polyporales such as Athelia rolfsii (Sclerotium rolfsii);
Basidiomycetes in the order Ustilaginales such as Ustilago maydis;
Zygomycetes in the order Mucorales such as Rhizopus stolonifer;
Oomycetes in the order Pythiales, including Phytophthora infestans, P. megasperma, P. parasitica,
P. sojae, P. cinnamomi and P. capsici, and Pythium pathogens such as Pythium aphanidermatum, P.
graminicola, P. irregulare, P. ultimum and P. dissoticum;
Oomycetes in the order Peronosporales such as Plasmopara viticola, P. halstedii, Peronospora
hyoscyami (=Peronospora tabacina), P. manshurica, Hyaloperonospora parasitica (=Peronospora
parasitica), Pseudoperonospora cubensis and Bremia lactucae;
and other genera and species closely related to all of the above pathogens.

In addition to their fungicidal activity, the compositions or combinations also have activity against bacteria such as Erwinia amylovora, Xanthomonas campestris, Pseudomonas syringae, and other related species. By controlling harmful microorganisms, the compounds of the invention are useful for improving (i.e. increasing) the ratio of beneficial to harmful microorganisms in contact with crop plants or their propagules (e.g., seeds, corms, bulbs, tubers, cuttings) or in the agronomic environment of the crop plants or their propagules.

Compounds of the invention are useful in treating all plants, plant parts and seeds. Plant and seed varieties and cultivars can be obtained by conventional propagation and breeding methods or by genetic engineering methods. Genetically modified plants or seeds (transgenic plants or seeds) are those in which a heterologous gene (transgene) has been stably integrated into the plant's or seed's genome. A transgene that is defined by its particular location in the plant genome is called a transformation or transgenic event.

Genetically modified plant cultivars which can be treated according to the invention include those that are resistant against one or more biotic stresses (pests such as nematodes, insects, mites, fungi, etc.) or abiotic stresses (drought, cold temperature, soil salinity, etc.), or that contain other desirable characteristics. Plants can be genetically modified to exhibit traits of, for example, herbicide tolerance, insect-resistance, modified oil profiles or drought tolerance.

Treatment of genetically modified plants and seeds with compounds of the invention may result in super-additive or enhanced effects. For example, reduction in application rates, broadening of the activity spectrum, increased tolerance to biotic/abiotic stresses or enhanced storage stability may be greater than expected from just simple additive effects of the application of compounds of the invention on genetically modified plants and seeds.

Compounds of this invention are useful in seed treatments for protecting seeds from plant diseases. In the context of the present disclosure and claims, treating a seed means contacting the seed with a biologically effective amount of a compound of this invention, which is typically formulated as a composition of the invention. This seed treatment protects the seed from soil-borne disease pathogens and generally can also protect roots and other plant parts in contact with the soil of the seedling developing from the germinating seed. The seed treatment may also provide protection of foliage by translocation of the compound of this invention or a second active ingredient within the developing plant. Seed treatments can be applied to all types of seeds, including those from which plants genetically transformed to express specialized traits will germinate. Representative examples include those expressing proteins toxic to invertebrate pests, such as Bacillus thuringiensis toxin or those expressing herbicide resistance such as glyphosate acetyltransferase, which provides resistance to glyphosate. Seed treatments with compounds of this invention can also increase vigor of plants growing from the seed.

Compounds of this invention and their compositions, both alone and in combination with other fungicides, nematicides and insecticides, are particularly useful in seed treatment for crops including, but not limited to, maize or corn, soybeans, cotton, cereal (e.g., wheat, oats, barley, rye and rice), potatoes, vegetables and oilseed rape.

Furthermore, the compounds of this invention are useful in treating postharvest diseases of fruits and vegetables caused by fungi and bacteria. These infections can occur before, during and after harvest. For example, infections can occur before harvest and then remain dormant until some point during ripening (e.g., host begins tissue changes in such a way that infection can progress); also infections can arise from surface wounds created by mechanical or insect injury. In this respect, the compounds of this invention can reduce losses (i.e. losses resulting from quantity and quality) due to postharvest diseases which may occur at any time from harvest to consumption. Treatment of postharvest diseases with compounds of the invention can increase the period of time during which perishable edible plant parts (e.g., fruits, seeds, foliage, stems, bulbs, tubers) can be stored refrigerated or un-refrigerated after harvest, and remain edible and free from noticeable or harmful degradation or contamination by fungi or other microorganisms. Treatment of edible plant parts before or after harvest with compounds of the invention can also decrease the formation of toxic metabolites of fungi or other microorganisms, for example, mycotoxins such as aflatoxins.

Plant disease control is ordinarily accomplished by applying an effective amount of a compound of this invention either pre- or post-infection, to the portion of the plant to be protected such as the roots, stems, foliage, fruits, seeds, tubers or bulbs, or to the media (soil or sand) in which the plants to be protected are growing. The compounds can also be applied to seeds to protect the seeds and seedlings developing from the seeds. The compounds can also be applied through irrigation water to treat plants. Control of postharvest pathogens which infect the produce before harvest is typically accomplished by field application of a compound of this invention, and in cases where infection occurs after harvest the compounds can be applied to the harvested crop as dips, sprays, fumigants, treated wraps and box liners.

The compounds can also be applied using an unmanned aerial vehicle (UAV) for the dispension of the compositions disclosed herein over a planted area. In some embodiments the planted area is a crop-containing area. In some embodiments, the crop is selected from a monocot or dicot. In some embodiments, the crop is selected form rice, corn, barley, sobean, wheat, vegetable, tobacco, tea tree, fruit tree and sugar cane. In some embodiments, the compositions disclosed herein are formulated for spraying at an ultra-low volume. Products applied by drones may use water or oil as the spray carrier. Typical spray volume (including product) used for drone applications globally. 5.0 liters/ha-100 liters/ha (approximately 0.5-10 gpa). This includes the range of ultra low spray volume (ULV) to low spray volume (LV). Although not common there may be situations where even lower spray volumes could be used as low as 1.0 liter/ha (0.1 gpa).

Rates of application for these compounds (i.e. a fungicidally effective amount) can be influenced by factors such as the plant diseases to be controlled, the plant species to be protected, ambient moisture and temperature and should be determined under actual use conditions. One skilled in the art can easily determine through simple experimentation the fungicidally effective amount necessary for the desired level of plant disease control. Foliage can normally be protected when treated at a rate of from less than about 1 g/ha to about 5,000 g/ha of active ingredient. Seed and seedlings can normally be protected when seed is treated at a rate of from about 0.001 g (more typically about 0.1 g) to about 10 g per kilogram of seed.

Compounds of this invention can also be mixed with one or more other biologically active compounds or agents including fungicides, insecticides, nematocides, bactericides, acaricides, herbicides, herbicide safeners, growth regulators such as insect molting inhibitors and rooting stimulants, chemosterilants, semiochemicals, repellents, attractants, pheromones, feeding stimulants, plant nutrients, other biologically active compounds or entomopathogenic bacteria, virus or fungi to form a multi-component pesticide giving an even broader spectrum of agricultural protection. Thus the present invention also pertains to a composition comprising a compound of Formula 1 (in a fungicidally effective amount) and at least one additional biologically active compound or agent (in a biologically effective amount) and can further comprise at least one of a surfactant, a solid diluent or a liquid diluent. The other biologically active compounds or agents can be formulated in compositions comprising at least one of a surfactant, solid or liquid diluent. For mixtures of the present invention, one or more other biologically active compounds or agents can be formulated together with a compound of Formula 1, to form a premix, or one or more other biologically active compounds or agents can be formulated separately from the compound of Formula 1, and the formulations combined together before application (e.g., in a spray tank) or, alternatively, applied in succession.

As mentioned in the Summary of the Invention, one aspect of the present invention is a fungicidal composition comprising (i.e. a mixture or combination of) a compound of Formula 1, an N-oxide, or a salt thereof (i.e. component a), and at least one other fungicide (i.e. component b). Of note is such a combination where the other fungicidal active ingredient has different site of action from the compound of Formula 1. In certain instances, a combination with at least one other fungicidal active ingredient having a similar spectrum of control but a different site of action will be particularly advantageous for resistance management. Thus, a composition of the present invention can further comprise a fungicidally effective amount of at least one additional fungicidal active ingredient having a similar spectrum of control but a different site of action.

Of note is a composition which in addition to the Formula 1 compound of component (a), includes as component (b) at least one fungicidal compound selected from the group consisting of the FRAC-defined mode of action (MOA) classes (A) nucleic acid synthesis, (B) mitosis and cell division, (C) respiration, (D) amino acid and protein synthesis, (E) signal transduction, (F) lipid synthesis and membrane integrity, (G) sterol biosynthesis in membranes, (H) cell wall biosynthesis in membranes, (I) melanin synthesis in cell wall, (P) host plant defense induction, multi-site contact activity and unknown mode of action.

FRAC-recognized or proposed target sites of action along with their FRAC target site codes belonging to the above MOA classes are (A1) RNA polymerase I, (A2) adenosine deaminase, (A3) DNA/RNA synthesis (proposed), (A4) DNA topoisomerase, (B1-B3) β-tubulin assembly in mitosis, (B4) cell division (proposed), (B5) delocalization of spectrin-like proteins, (C1) complex I NADH odxido-reductase, (C2) complex II: succinate dehydrogenase, (C3) complex III: cytochrome bcl (ubiquinol oxidase) at Qo site, (C4) complex III: cytochrome bcl (ubiquinone reductase) at Qi site, (C5) uncouplers of oxidative phosphorylation, (C6) inhibitors of oxidative phosphorylation, ATP synthase, (C7) ATP production (proposed), (C8) complex III: cytochrome bcl (ubiquinone reductase) at Qx (unknown) site, (D1) methionine biosynthesis (proposed), (D2-D5) protein synthesis, (E1) signal transduction (mechanism unknown), (E2-E3) MAP/histidine kinase in osmotic signal transduction, (F2) phospholipid biosynthesis, methyl transferase, (F3) lipid peroxidation (proposed), (F4) cell membrane permeability, fatty acids (proposed), (F6) microbial disrupters of pathogen cell membranes, (F7) cell membrane disruption (proposed), (G1) C14-demethylase in sterol biosynthesis, (G2) A14-reductase and Δ8→Δ7-isomerase in sterol biosynthesis, (G3) 3-keto reductase, C4-demethylation, (G4) squalene epoxidase in sterol biosynthesis, (H3) trehalase and inositol biosynthesis, (H4) chitin synthase, (H5) cellulose synthase, (I1) reductase in melanin biosynthesis and (I2) dehydratase in melanin biosynthesis.

Of particular note is a composition which in addition to the Formula 1 compound of component (a), includes as component (b) at least one fungicidal compound selected from the group consisting of the classes (b1) methyl benzimidazole carbamate (MBC) fungicides; (b2) dicarboximide fungicides; (b3) demethylation inhibitor (DMI) fungicides; (b4) phenylamide fungicides; (b5) amine/morpholine fungicides; (b6) phospholipid biosynthesis inhibitor fungicides; (b7) succinate dehydrogenase inhibitor fungicides; (b8) hydroxy(2-amino-)pyrimidine fungicides; (b9) anilinopyrimidine fungicides; (b10)N-phenyl carbamate fungicides; (b11) quinone outside inhibitor (QoI) fungicides; (b12) phenylpyrrole fungicides; (b13) azanaphthalene fungicides; (b14) lipid peroxidation inhibitor fungicides; (b15) melanin biosynthesis inhibitor-reductase (MI-R) fungicides; (b6) melanin biosynthesis inhibitor-dehydratase (MI-D) fungicides; (b17) sterol biosynthesis inhibitor (SBI): Class III fungicides; (b18) squalene-epoxidase inhibitor fungicides; (b19) polyoxin fungicides; (b20) phenylurea fungicides; (b21) quinone inside inhibitor (QiI) fungicides; (b22) benzamide and thiazole carboxamide fungicides; (b23) enopyranuronic acid antibiotic fungicides; (b24) hexopyranosyl antibiotic fungicides; (b25) glucopyranosyl antibiotic: protein synthesis fungicides; (b26) glucopyranosyl antibiotic: trehalase and inositol biosynthesis fungicides; (b27) cyanoacetamideoxime fungicides; (b28) carbamate fungicides; (b29) oxidative phosphorylation uncoupling fungicides; (b30) organo tin fungicides; (b31) carboxylic acid fungicides; (b32) heteroaromatic fungicides; (b33) phosphonate fungicides; (b34) phthalamic acid fungicides; (b35) benzotriazine fungicides; (b36) benzene-sulfonamide fungicides; (b37) pyridazinone fungicides; (b38) thiophene-carboxamide fungicides; (b39) complex I NADH oxidoreductase inhibitor fungicides; (b40) carboxylic acid amide (CAA) fungicides; (b41) tetracycline antibiotic fungicides; (b42) thiocarbamate fungicides; (b43) benzamide fungicides; (b44) microbial fungicides; (b45) QxI fungicides; (b46) plant extract fungicides; (b47) host plant defense induction fungicides; (b48) multi-site contact activity fungicides; (b49) fungicides other than fungicides of classes (b1) through (b48); and salts of compounds of classes (b1) through (b48).

Further descriptions of these classes of fungicidal compounds are provided below.

(b1) “Methyl benzimidazole carbamate (MBC) fungicides” (FRAC code 1) inhibit mitosis by binding to β-tubulin during microtubule assembly. Inhibition of microtubule assembly can disrupt cell division, transport within the cell and cell structure. Methyl benzimidazole carbamate fungicides include benzimidazole and thiophanate fungicides. The benzimidazoles include benomyl, carbendazim, fuberidazole and thiabendazole. The thiophanates include thiophanate and thiophanate-methyl.

(b2) “Dicarboximide fungicides” (FRAC code 2) inhibit a MAP/histidine kinase in osmotic signal transduction. Examples include chlozolinate, iprodione, procymidone and vinclozolin.

(b3) “Demethylation inhibitor (DI) fungicides” (FRAC code 3) (Sterol Biosynthesis Inhibitors (SBI): Class I) inhibit C14-demethylase, which plays a role in sterol production. Sterols, such as ergosterol, are needed for membrane structure and function, making them essential for the development of functional cell walls. Therefore, exposure to these fungicides results in abnormal growth and eventually death of sensitive fungi. DMI fungicides are divided between several chemical classes: azoles (including triazoles and imidazoles), pyrimidines, piperazines, pyridines and triazolinthiones. The triazoles include azaconazole, bitertanol, bromuconazole, cyproconazole, difenoconazole, diniconazole (including diniconazole-M), epoxiconazole, etaconazole, fenbuconazole, fluquinconazole, flusilazole, flutriafol, hexaconazole, imibenconazole, ipconazole, mefentrifluconazole, metconazole, myclobutanil, penconazole, propiconazole, quinconazole, simeconazole, tebuconazole, tetraconazole, triadimefon, triadimenol, triticonazole, uniconazole, uniconazole-P, α-(1-chlorocyclopropyl)-α-[2-(2,2-dichlorocyclopropyl)ethyl]-1H-1,2,4-triazole-1-ethanol, rel-1-[[(2R,3S)-3-(2-chlorophenyl)-2-(2,4-difluorophenyl)-2-oxiranyl]methyl]-1H-1,2,4-triazole, rel-2-[[(2R,3S)-3-(2-chlorophenyl)-2-(2,4-difluorophenyl)-2-oxiranyl]methyl]-1,2-dihydro-3H-1,2,4-triazole-3-thione, and rel-1-[[(2R,3S)-3-(2-chlorophenyl)-2-(2,4-difluorophenyl)-2-oxiranyl]methyl]-5-(2-propen-1-ylthio)-1H-1,2,4-triazole. The imidazoles include econazole, imazalil, oxpoconazole, prochloraz, pefurazoate and triflumizole. The pyrimidines include fenarimol, nuarimol and triarimol. The piperazines include triforine. The pyridines include buthiobate, pyrifenox, pyrisoxazole (3-[(3R)-5-(4-chlorophenyl)-2,3-dimethyl3-isoxazolidinyl]pyridine, mixture of 3R,5R- and 3R,5S-isomers) and (αS)-[3-(4-chloro-2-fluorophenyl)5-(2,4-difluorophenyl)-4-isoxazolyl]-3-pyridinemethanol. The triazolinthiones include prothioconazole and 2-[2-(1-chlorocyclopropyl)-4-(2,2-dichlorocyclopropyl)2-hydroxybutyl]-1,2-dihydro-3H-1,2,4-triazole-3-thione. Biochemical investigations have shown that all of the above mentioned fungicides are DMI fungicides as described by K. H. Kuck et al. in Modern Selective Fungicides—Properties, Applications and Mechanisms of Action, H. Lyr (Ed.), Gustav Fischer Verlag: New York, 1995, 205-258.

(b4) “Phenylamide fungicides” (FRAC code 4) are specific inhibitors of RNA polymerase in Oomycete fungi. Sensitive fungi exposed to these fungicides show a reduced capacity to incorporate uridine into rRNA. Growth and development in sensitive fungi is prevented by exposure to this class of fungicide. Phenylamide fungicides include acylalanine, oxazolidinone and butyrolactone fungicides. The acylalanines include benalaxyl, benalaxyl-M (also known as kiralaxyl), furalaxyl, metalaxyl and metalaxyl-M (also known as mefenoxam). The oxazolidinones include oxadixyl. The butyrolactones include ofurace.

(b5) “Amine/morpholine fungicides” (FRAC code 5) (SBI: Class II) inhibit two target sites within the sterol biosynthetic pathway, Δ⁸→Δ⁷ isomerase and Δ¹⁴ reductase. Sterols, such as ergosterol, are needed for membrane structure and function, making them essential for the development of functional cell walls. Therefore, exposure to these fungicides results in abnormal growth and eventually death of sensitive fungi. Amine/morpholine fungicides (also known as non-DI sterol biosynthesis inhibitors) include morpholine, piperidine and spiroketal-amine fungicides. The morpholines include aldimorph, dodemorph, fenpropimorph, tridemorph and trimorphamide. The piperidines include fenpropidin and piperalin. The spiroketal-amines include spiroxamine.

(b6) “Phospholipid biosynthesis inhibitor fungicides” (FRAC code 6) inhibit growth of fungi by affecting phospholipid biosynthesis. Phospholipid biosynthesis fungicides include phophorothiolate and dithiolane fungicides. The phosphorothiolates include edifenphos, iprobenfos and pyrazophos. The dithiolanes include isoprothiolane.

(b7) “Succinate dehydrogenase inhibitor (SDHI) fungicides” (FRAC code 7) inhibit Complex II fungal respiration by disrupting a key enzyme in the Krebs Cycle (TCA cycle) named succinate dehydrogenase. Inhibiting respiration prevents the fungus from making ATP, and thus inhibits growth and reproduction. SDHI fungicides include phenylbenzamide, furan carboxamide, oxathiin carboxamide, thiazole carboxamide, pyrazole-4-carboxamide, pyridine carboxamide, phenyl oxoethyl thiophene amides and pyridinylethyl benzamides. The benzamides include benodanil, flutolanil and mepronil. The furan carboxamides include fenfuram. The oxathiin carboxamides include carboxin and oxycarboxin. The thiazole carboxamides include thifluzamide. The pyrazole-4-carboxamides include benzovindiflupyr (N-[9-(dichloro-methylene)-1,2,3,4-tetrahydro-1,4-methanonaphthalen-5-yl]-3-(difluoromethyl)-1-methyl-1H-pyrazole-4-carboxamide), bixafen, fluindapyr, fluxapyroxad (3-(difluoromethyl)-1-methyl-N-(3′,4′,5′-trifluoro[1,1′-biphenyl]-2-yl)-1H-pyrazole-4-carboxamide), furametpyr, isoflucypram, isopyrazam (3-(difluoromethyl)-1-methyl-N-[1,2,3,4-tetrahydro-9-(1-methylethyl)-1,4-methano-naphthalen-5-yl]-1H-pyrazole-4-carboxamide), penflufen (N-[2-(1,3-dimethylbutyl)phenyl]-5-fluoro-1,3-dimethyl-1H-pyrazole-4-carboxamide), penthiopyrad, pydiflumetofen, sedaxane (N-[2-[1,1′-bicyclopropyl]-2-ylphenyl]-3-(difluoromethyl)-1-methyl-1H-pyrazole-4-carboxamide), N-[2-(1S,2R)-[1,1′-bicyclopropyl]-2-ylphenyl]-3-(difluoromethyl)-1-methyl-1H-pyrazole-4-carboxamide, 3-(difluoromethyl)-N-(2,3-dihydro-1,1,3-trimethyl-1H-inden-4-yl)-1-methyl-1H-pyrazole-4-carboxamide, N-[2-(2,4-dichlorophenyl)2-methoxy-1-methylethyl]-3-(difluoro-methyl)-1-methyl-1H-pyrazole-4-carboxamide and N-cyclopropyl-3-(difluoromethyl)-5-fluoro-1-methyl-N-[[2-(1-methylethyl)phenyl]methyl]-1H-pyrazole-4-carboxamide. The pyridine carboxamides include boscalid. The phenyl oxoethyl thiophene amides include isofetamid (N-[1,1-dimethyl-2-[2-methyl-4-(1-methylethoxy)phenyl]-2-oxoethyl]-3-methyl-2-thiophenecarboxamide). The pyridinylethyl benzamides include fluopyram.

(b8) “Hydroxy-(2-amino-)pyrimidine fungicides” (FRAC code 8) inhibit nucleic acid synthesis by interfering with adenosine deaminase. Examples include bupirimate, dimethirimol and ethirimol.

(b9) “Anilinopyrimidine fungicides” (FRAC code 9) are proposed to inhibit biosynthesis of the amino acid methionine and to disrupt the secretion of hydrolytic enzymes that lyse plant cells during infection. Examples include cyprodinil, mepanipyrim and pyrimethanil.

(b10) “N-Phenyl carbamate fungicides” (FRAC code 10) inhibit mitosis by binding to 3-tubulin and disrupting microtubule assembly. Inhibition of microtubule assembly can disrupt cell division, transport within the cell and cell structure. Examples include diethofencarb.

(b11) “Quinone outside inhibitor (QoI) fungicides” (FRAC code 11) inhibit Complex III mitochondrial respiration in fungi by affecting ubiquinol oxidase. Oxidation of ubiquinol is blocked at the “quinone outside” (Qo) site of the cytochrome bc₁ complex, which is located in the inner mitochondrial membrane of fungi. Inhibiting mitochondrial respiration prevents normal fungal growth and development. Quinone outside inhibitor fungicides include methoxyacrylate, methoxycarbamate, oximinoacetate, oximinoacetamide and dihydrodioxazine fungicides (collectively also known as strobilurin fungicides), and oxazolidinedione, imidazolinone and benzylcarbamate fungicides. The methoxyacrylates include azoxystrobin, coumoxystrobin (methyl (αE)-2-[[(3-butyl-4-methyl-2-oxo-2H-1-benzopyran-7-yl)oxy]methyl]α-(methoxy-methylene)benzeneacetate), enoxastrobin (methyl (αE)-2-[[[(E)-[(2E)-3-(4-chlorophenyl)-1-methyl-2-propen-1-ylidene]amino]oxy]methyl]-α-(methoxymethylene)benzeneaceate) (also known as enestroburin), flufenoxystrobin (methyl (αE)-2-[[2-chloro-4-(trifluoromethyl)-phenoxy]methyl]-α-(methoxymethylene)benzeneacetate), picoxystrobin, and pyraoxystrobin (methyl (αE)-2-[[[3-(4-chlorophenyl)-1-methyl-1H-pyrazol-5-yl]oxy]methyl]-α-(methoxy-methylene)benzeneacetate). The methoxycarbamates include pyraclostrobin, pyrametostrobin (methyl N-[2-[[(1,4-dimethyl-3-phenyl-1H-pyrazol-5-yl)oxy]methyl]phenyl]-N-methoxycarbamate) and triclopyricarb (methyl N-methoxy-N-[2-[[(3,5,6-trichloro-2-pyridinyl)oxy]-methyl]phenyl]carbamate). The oximinoacetates include kresoxim-methyl and trifloxystrobin. The oximinoacetamides include dimoxystrobin, fenaminstrobin ((αE)-2-[[[(E)-[(2E)-3-(2,6-dichlorophenyl)-1-methyl-2-propen-1-ylidene]amino]oxy]methyl]-α-(methoxyimino)-N-methyl-benzeneacetamide), metominostrobin, orysastrobin and α-[methoxyimino]-N-methyl-2-[[[1-[3-(trifluoromethyl)phenyl]ethoxy]imino]methyl]benzeneacetamide. The dihydrodioxazines include fluoxastrobin. The oxazolidinediones include famoxadone. The imidazolinones include fenamidone. The benzylcarbamates include pyribencarb. Class (b11) also includes mandestrobin (2-[(2,5-dimethylphenoxy)methyl]-α-methoxy-N-benzeneacetamide).

(b12) “Phenylpyrrole fungicides” (FRAC code 12) inhibit a MAP/histidine kinase associated with osmotic signal transduction in fungi. Fenpiclonil and fludioxonil are examples of this fungicide class.

(b13) “Azanaphthalene fungicides” (FRAC code 13) are proposed to inhibit signal transduction by a mechanism which is as yet unknown. They have been shown to interfere with germination and/or appressorium formation in fungi that cause powdery mildew diseases. Azanaphthalene fungicides include aryloxyquinolines and quinazolinones. The aryloxyquinolines include quinoxyfen. The quinazolinones include proquinazid.

(b14) “Lipid peroxidation inhibitor fungicides” (FRAC code 14) are proposed to inhibit lipid peroxidation which affects membrane synthesis in fungi. Members of this class, such as etridiazole, may also affect other biological processes such as respiration and melanin biosynthesis. Lipid peroxidation fungicides include aromatic hydrocarbon and 1,2,4-thiadiazole fungicides. The aromatic hydrocarboncarbon fungicides include biphenyl, chloroneb, dicloran, quintozene, tecnazene and tolclofos-methyl. The 1,2,4-thiadiazoles include etridiazole.

(b15) “Melanin biosynthesis inhibitors-reductase (MBI-R) fungicides” (FRAC code 16.1) inhibit the naphthal reduction step in melanin biosynthesis. Melanin is required for host plant infection by some fungi. Melanin biosynthesis inhibitors-reductase fungicides include isobenzofuranone, pyrroloquinolinone and triazolobenzothiazole fungicides. The isobenzofuranones include fthalide. The pyrroloquinolinones include pyroquilon. The triazolobenzothiazoles include tricyclazole.

(b16) “Melanin biosynthesis inhibitors-dehydratase (MBI-D) fungicides” (FRAC code 16.2) inhibit scytalone dehydratase in melanin biosynthesis. Melanin in required for host plant infection by some fungi. Melanin biosynthesis inhibitors-dehydratase fungicides include cyclopropanecarboxamide, carboxamide and propionamide fungicides. The cyclopropanecarboxamides include carpropamid. The carboxamides include diclocymet. The propionamides include fenoxanil.

(b17) “Sterol Biosynthesis Inhibitor (SBI): Class III fungicides (FRAC code 17) inhibit 3-ketoreductase during C4-demethylation in sterol production. SBI: Class III inhibitors include hydroxyanilide fungicides and amino-pyrazolinone fungicides. Hydroxyanilides include fenhexamid. Amino-pyrazolinones include fenpyrazamine (S-2-propen-1-yl 5-amino-2,3-di-hydro-2-(1-methylethyl)-4-(2-methylphenyl)-3-oxo-1H-pyrazole-1-carbothioate).

(b18) “Squalene-epoxidase inhibitor fungicides” (FRAC code 18) (SBI: Class IV) inhibit squalene-epoxidase in the sterol biosynthesis pathway. Sterols such as ergosterol are needed for membrane structure and function, making them essential for the development of functional cell walls. Therefore exposure to these fungicides results in abnormal growth and eventually death of sensitive fungi. Squalene-epoxidase inhibitor fungicides include thiocarbamate and allylamine fungicides. The thiocarbamates include pyributicarb. The allylamines include naftifine and terbinafine.

(b19) “Polyoxin fungicides” (FRAC code 19) inhibit chitin synthase. Examples include polyoxin.

(b20) “Phenylurea fungicides” (FRAC code 20) are proposed to affect cell division. Examples include pencycuron.

(b21) “Quinone inside inhibitor (QiI) fungicides” (FRAC code 21) inhibit Complex III mitochondrial respiration in fungi by affecting ubiquinone reductase. Reduction of ubiquinone is blocked at the “quinone inside” (Qj) site of the cytochrome bc₁ complex, which is located in the inner mitochondrial membrane of fungi. Inhibiting mitochondrial respiration prevents normal fungal growth and development. Quinone inside inhibitor fungicides include cyanoimidazole and sulfamoyltriazole fungicides. The cyanoimidazoles include cyazofamid. The sulfamoyltriazoles include amisulbrom.

(b22) “Benzamide and thiazole carboxamide fungicides” (FRAC code 22) inhibit mitosis by binding to β-tubulin and disrupting microtubule assembly. Inhibition of microtubule assembly can disrupt cell division, transport within the cell and cell structure. The benzamides include zoxamide. The thiazole carboxamides include ethaboxam.

(b23) “Enopyranuronic acid antibiotic fungicides” (FRAC code 23) inhibit growth of fungi by affecting protein biosynthesis. Examples include blasticidin-S.

(b24) “Hexopyranosyl antibiotic fungicides” (FRAC code 24) inhibit growth of fungi by affecting protein biosynthesis. Examples include kasugamycin.

(b25) “Glucopyranosyl antibiotic: protein synthesis fungicides” (FRAC code 25) inhibit growth of fungi by affecting protein biosynthesis. Examples include streptomycin.

(b26) “Glucopyranosyl antibiotic: trehalase and inositol biosynthesis fungicides” (FRAC code 26) inhibit trehalase and inositol biosynthesis. Examples include validamycin.

(b27) “Cyanoacetamideoxime fungicides (FRAC code 27) include cymoxanil.

(b28) “Carbamate fungicides” (FRAC code 28) are considered multi-site inhibitors of fungal growth. They are proposed to interfere with the synthesis of fatty acids in cell membranes, which then disrupts cell membrane permeability. Propamacarb, iodocarb, and prothiocarb are examples of this fungicide class.

(b29) “Oxidative phosphorylation uncoupling fungicides” (FRAC code 29) inhibit fungal respiration by uncoupling oxidative phosphorylation. Inhibiting respiration prevents normal fungal growth and development. This class includes 2,6-dinitroanilines such as fluazinam, and dinitrophenyl crotonates such as dinocap, meptyldinocap and binapacryl.

(b30) “Organo tin fungicides” (FRAC code 30) inhibit adenosine triphosphate (ATP) synthase in oxidative phosphorylation pathway. Examples include fentin acetate, fentin chloride and fentin hydroxide.

(b31) “Carboxylic acid fungicides” (FRAC code 31) inhibit growth of fungi by affecting deoxyribonucleic acid (DNA) topoisomerase type II (gyrase). Examples include oxolinic acid.

(b32) “Heteroaromatic fungicides” (Fungicide Resistance Action Committee (FRAC) code 32) are proposed to affect DNA/ribonucleic acid (RNA) synthesis. Heteroaromatic fungicides include isoxazoles and isothiazolones. The isoxazoles include hymexazole and the isothiazolones include octhilinone.

(b33) “Phosphonate fungicides” (FRAC code 33) include phosphorous acid and its various salts, including fosetyl-aluminum.

(b34) “Phthalamic acid fungicides” (FRAC code 34) include teclofthalam.

(b35) “Benzotriazine fungicides” (FRAC code 35) include triazoxide.

(b36) “Benzene-sulfonamide fungicides” (FRAC code 36) include flusulfamide.

(b37) “Pyridazinone fungicides” (FRAC code 37) include diclomezine.

(b38) “Thiophene-carboxamide fungicides” (FRAC code 38) are proposed to affect ATP production. Examples include silthiofam.

(b39) “Complex I NADH oxidoreductase inhibitor fungicides” (FRAC code 39) inhibit electron transport in mitochondria and include pyrimidinamines such as diflumetorim, and pyrazole-5-carboxamides such as tolfenpyrad.

(b40) “Carboxylic acid amide (CAA) fungicides” (FRAC code 40) inhibit cellulose synthase which prevents growth and leads to death of the target fungus. Carboxylic acid amide fungicides include cinnamic acid amide, valinamide and other carbamate, and mandelic acid amide fungicides. The cinnamic acid amides include dimethomorph, flumorph and pyrimorph (3-(2-chloro-4-pyridinyl)-3-[4-(1,1-dimethylethyl)phenyl]-1-(4-morpholinyl)-2-propene-1-one). The valinamide and other carbamates include benthiavalicarb, benthiavalicarb-isopropyl, iprovalicarb, tolprocarb (2,2,2-trifluoroethyl N-[(1S)-2-methyl-1-[[(4-methylbenzoyl)amino]methyl]propyl]-carbamate) and valifenalate (methyl N-[(1-methylethoxy)carbonyl]-L-valyl-3-(4-chlorophenyl)-β-alaninate) (also known as valiphenal). The mandelic acid amides include mandipropamid, N-[2-[4-[[3-(4-chlorophenyl)-2-propyn-1-yl]oxy]-3-methoxyphenyl]ethyl]-3-methyl-2-[(methylsulfonyl)amino]butanamide and N-[2-[4-[[3-(4-chlorophenyl)-2-propyn-1-yl]oxy]-3-methoxyphenyl]ethyl]-3-methyl-2-[(ethylsulfonyl)amino]butanamide.

(b41) “Tetracycline antibiotic fungicides” (FRAC code 41) inhibit growth of fungi by affecting protein synthesis. Examples include oxytetracycline.

(b42) “Thiocarbamate fungicides” (FRAC code 42) include methasulfocarb.

(b43) “Benzamide fungicides” (FRAC code 43) inhibit growth of fungi by delocalization of spectrin-like proteins. Examples include pyridinylmethyl benzamide fungicides such as fluopicolide (now FRAC code 7, pyridinylethyl benzamides).

(b44) “Microbial fungicides” (FRAC code 44) disrupt fungal pathogen cell membranes. Microbial fungicides include Bacillus species such as Bacillus amyloliquefaciens strains QST 713, FZB24, MBB1600, D747 and the fungicidal lipopeptides which they produce.

(b45) “QxI fungicides” (FRAC code 45) inhibit Complex III mitochondrial respiration in fungi by affecting ubiquinone reductase at an unknown (Qx) site of the cytochrome bc₁ complex. Inhibiting mitochondrial respiration prevents normal fungal growth and development. QₓI fungicides include triazolopyrimidylamines such as ametoctradin (5-ethyl-6-octyl[1,2,4]triazolo[1,5-a]pyrimidin-7-amine).

(b46) “Plant extract fungicides” are proposed to act by cell membrane disruption. Plant extract fungicides include terpene hydrocarbons and terpene alcohols such as the extract from Melaleuca alternifolia (tea tree).

(b47) “Host plant defense induction fungicides” (FRAC code P) induce host plant defense mechanisms. Host plant defense induction fungicides include benzothiadiazoles, benzisothiazole and thiadiazole-carboxamide fungicides. The benzothiadiazoles include acibenzolar-S-methyl. The benzisothiazoles include probenazole. The thiadiazole-carboxamides include tiadinil and isotianil.

(b48) “Multi-site contact fungicides” inhibit fungal growth through multiple sites of action and have contact/preventive activity. This class of fungicides includes: (b48.1) “copper fungicides” (FRAC code M1)”, (b48.2) “sulfur fungicides” (FRAC code M2), (b48.3) “dithiocarbamate fungicides” (FRAC code M3), (b48.4) “phthalimide fungicides” (FRAC code M4), (b48.5) “chloronitrile fungicides” (FRAC code M5), (b48.6) “sulfamide fungicides” (FRAC code M6), (b48.7) multi-site contact “guanidine fungicides” (FRAC code M7), (b48.8) “triazine fungicides” (FRAC code M8), (b48.9) “quinone fungicides” (FRAC code M9), (b48.10) “quinoxaline fungicides” (FRAC code M10) and (b48.11) “maleimide fungicides” (FRAC code M11). “Copper fungicides” are inorganic compounds containing copper, typically in the copper(II) oxidation state; examples include copper oxychloride, copper sulfate and copper hydroxide, including compositions such as Bordeaux mixture (tribasic copper sulfate). “Sulfur fungicides” are inorganic chemicals containing rings or chains of sulfur atoms; examples include elemental sulfur. “Dithiocarbamate fungicides” contain a dithiocarbamate molecular moiety; examples include mancozeb, metiram, propineb, ferbam, maneb, thiram, zineb and ziram. “Phthalimide fungicides” contain a phthalimide molecular moiety; examples include folpet, captan and captafol. “Chloronitrile fungicides” contain an aromatic ring substituted with chloro and cyano; examples include chlorothalonil. “Sulfamide fungicides” include dichlofluanid and tolyfluanid. Multi-site contact “guanidine fungicides” include, guazatine, iminoctadine albesilate and iminoctadine triacetate. “Triazine fungicides” include anilazine. “Quinone fungicides” include dithianon. “Quinoxaline fungicides” include quinomethionate (also known as chinomethionate). “Maleimide fungicides” include fluoroimide.

(b49) “Fungicides other than fungicides of classes (b1) through (b48)” include certain fungicides whose mode of action may be unknown. These include: (b49.1), “phenyl-acetamide fungicides” (FRAC code U6), (b49.2) “aryl-phenyl-ketone fungicides” (FRAC code U8), (b49.3) “guanidine fungicides” (FRAC code U12), (b49.4) “thiazolidine fungicides” (FRAC code U13), (b49.5) “pyrimidinone-hydrazone fungicides” (FRAC code U14) and (b49.6) compounds that bind to oxysterol-binding protein as described in PCT Patent Publication WO 2013/009971. The phenyl-acetamides include cyflufenamid and N-[[(cyclopropylmethoxy)amino][6-(difluoromethoxy)-2,3-difluorophenyl]-methylene]benzeneacetamide. The aryl-phenyl ketones include benzophenones such as metrafenone, and benzoylpyridines such as pyriofenone (5-chloro-2-methoxy-4-methyl-3-pyridinyl)(2,3,4-trimethoxy-6-methylphenyl)methanone). The quanidines include dodine. The thiazolidines include flutianil ((2Z)-2-[[2-fluoro-5-(trifluoromethyl)phenyl]thio]-2-[3-(2-methoxyphenyl)-2-thiazolidinylidene]acetonitrile). The pyrimidinonehydrazones include ferimzone. The (b49.6) class includes oxathiapiprolin (1-[4-[4-[5-(2,6-difluorophenyl)-4,5-dihydro-3-isoxazolyl]-2-thiazolyl]-1-piperidinyl]-2-[5-methyl-3-(trifluoromethyl)-1H-pyrazol-1-yl]ethanone) and its R-enantiomer which is 1-[4-[4-[5R-(2,6-difluorophenyl)-4,5-dihydro-3-isoxazolyl]-2-thiazolyl]-1-piperidinyl]-2-[5-methyl-3-(trifluoro-methyl)-1H-pyrazol-1-yl]ethanone (Registry Number 1003319-79-6). The (b49) class also includes bethoxazin, flometoquin (2-ethyl-3,7-dimethyl-6-[4-(trifluoromethoxy)phenoxy]-4-quinolinyl methyl carbonate), fluoroimide, neo-asozin (ferric methanearsonate), picarbutrazox (1,1-dimethylethyl N-[6-[[[[((Z)1-methyl-1H-tetrazol-5-yl)phenylmethylene]amino]oxy]-methyl]-2-pyridinyl]carbamate), pyrrolnitrin, quinomethionate, tebufloquin (6-(1,1-dimethylethyl)-8-fluoro-2,3-dimethyl-4-quinolinyl acetate), tolnifanide (N-(4-chloro-2-nitro-phenyl)-N-ethyl-4-methylbenzenesulfonamide), 2-butoxy-6-iodo-3-propyl-4H-1-benzopyran-4-one, 3-butyn-1-yl, N-[6-[[[[(1-methyl-1H-tetrazol-5-yl)phenylmethylene]amino]oxy]methyl]-2-pyridinyl]carbamate, (N-(4-chloro-2-nitrophenyl)-N-ethyl-4-methylbenzenesulfonamide), N-[4-[4-chloro-3-(trifluoromethyl)phenoxy]-2,5-dimethylphenyl]-N-ethyl-N-methylmethanimidamide, N-[[(cyclopropylmethoxy)amino][6-(difluoromethoxy)-2,3-difluorophenyl]methylene]-benzeneacetamide, 2,6-dimethyl-1H,5H-[1,4]dithiino[2,3-c:5,6-c′]dipyrrole-1,3,5,7(2H,6H)-tetrone, 5-fluoro-2-[(4-methylphenyl)methoxy]-4-pyrimidinamine, 5-fluoro-2-[(4-fluoro-phenyl)methoxy]-4-pyrimidinamine and 4-fluorophenyl N-[1-[[[1-(4-cyanophenyl)ethyl]-sulfonyl]methyl]propyl]carbamate, pentyl N-[6-[[[[(1-methyl-1H-tetrazol-5-yl)phenylmethylene]amino]oxy]methyl]-2-pyridinyl]carbamate, pentyl N-[4-[[[[(1-methyl-1H-tetrazol-5-yl)phenylmethylene]amino]oxy]methyl]-2-thiazolyl]carbamate and pentyl N-[6-[[[[(Z)-(1-methyl-1H-tetrazol-5-yl)phenylmethylene]amino]oxy]methyl]-2-pyridinyl]carbamate. The (b46) class further includes mitosis- and cell division-inhibiting fungicides besides those of the particular classes described above (e.g., (b1), (b10) and (b22)).

Additional “Fungicides other than fungicides of classes (1) through (46)” whose mode of action may be unknown, or may not yet be classified include a fungicidal compound selected from components (b49.7) through (b49.13), as shown below.

Component (b49.7) relates to a compound of Formula b49.7

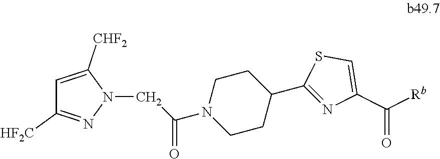

wherein R^{b1} is

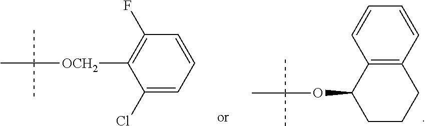

Examples of a compound of Formula b49.7 include (b49.7a) (2-chloro-6-fluorophenyl)methyl 2-[1-[2-[3,5-bis(difluoromethyl)-1H-pyrazol-1-yl]acetyl]-4-piperidinyl]-4-thiazolecarboxylate (Registry Number 1299409-40-7) and (b49.7b) (1R)-1,2,3,4-tetrahydro-1-naphthalenyl 2-[1-[2-[3,5-bis(difluoromethyl)-1H-pyrazol-1-yl]acetyl]-4-piperidinyl]-4-thiazolecarboxylate (Registry Number 1299409-42-9). Methods for preparing compounds of Formula b46.2 are described in PCT Patent Publications WO 2009/132785 and WO 2011/051243.

Component (b49.8) relates to a compound of Formula b49.8

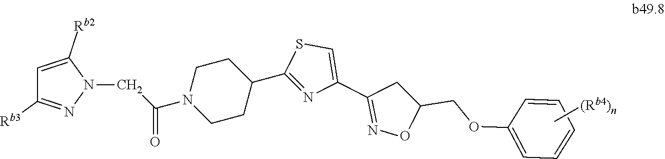

wherein R^{b2} is CH₃, CF₃ or CHF₂; R^{b3} is CH₃, CF₃ or CHF₂; R^{b4} is halogen or cyano; and n is 0, 1, 2 or 3.

Examples of a compound of Formula b49.8 include (b49.8a) 1-[4-[4-[5-[(2,6-difluorophenoxy)-methyl]-4,5-dihydro-3-isoxazolyl]-2-thiazolyl]-1-piperdinyl]-2-[5-methyl-3-(trifluoromethyl)-1H-pyrazol-1-yl]ethanone. Methods for preparing compounds of Formula b49.8 are described in PCT Patent Application PCT/US 11/64324.

Component (b4799) relates to a compound of Formula b49.9

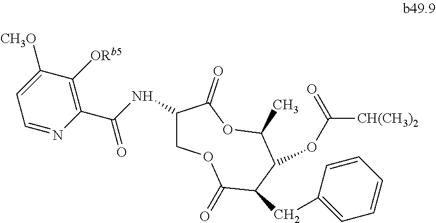

wherein R^{b5} is —CH₂OC(O)CH(CH₃)₂, —C(O)CH₃, —CH₂OC(O)CH₃, —C(O)OCH₂CH(CH₃)₂ or 2

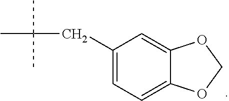

Examples of a compound of Formula b49.9 include (b49.9a) [[4-methoxy-2-[[[(3S,7R,8R,9S)-9-methyl-8-(2-methyl-1-oxopropoxy)-2,6-dioxo-7-(phenylmethyl)-1,5-dioxonan-3-yl]amino]-carbonyl]-3-pyridinyl]oxy]methyl 2-methylpropanoate (Registry Number 517875-34-2; common name fenpicoxamid), (b49.9b) (3S,6S,7R,8R)-3-[[[3-(acetyloxy)-4-methoxy-2-pyridinyl]-carbonyl]amino]-6-methyl-4,9-dioxo-8-(phenylmethyl)-1,5-dioxonan-7-yl 2-methylpropanoate (Registry Number 234112-93-7), (b49.9c) (3S,6S,7R,8R)-3[[[3[(acetyloxy)methoxy]-4-methoxy-2-pyridinyl]carbonyl]amino]-6-methyl-4,9-dioxo-8-(phenylmethyl)-1,5-dioxonan-7-yl 2-methylpropanoate (Registry Number 517875-31-9), (b49.9d) (3S,6S,7R,8R)-3-[[[4-methoxy-3-[[(2-methylpropoxy)carbonyl]oxy]-2-pyridinyl]carbonyl]amino]6-methyl-4,9-dioxo-8-(phenylmethyl)-1,5-dioxonan-7-yl 2-methylpropanoate (Registry Number 328256-72-0), and (b49.9e)N-[[3-(1,3-benzodioxol-5-ylmethoxy)-4-methoxy-2-pyridinyl]carbonyl]-O-[2,5-dideoxy-3-O-(2-methyl-1-oxopropyl)-2-(phenylmethyl)L-arabinonoyl]-L-serine, (1→4′)-lactone (Registry Number 1285706-70-8). Methods for preparing compounds of Formula b49.9 are described in PCT Patent Publications WO 99/40081, WO 2001/014339, WO 2003/035617 and WO 2011044213.

Component (b49.10) relates to a compound of Formula b49.10

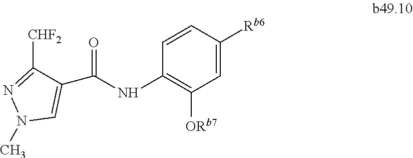

wherein R^{b6} is H or F, and R^{b7} is —CF₂CHFCF₃ or —CF₂CF₂H. Examples of a compound of Formula b49.10 are (b49.10a) 3-(difluoromethyl)-N-[4-fluoro-2-(1,1,2,3,3,3-hexafluoro-propoxy)phenyl]-1-methyl-1H-pyrazole-4-carboxamide (Registry Number 1172611-40-3) and (b49.10b) 3-(difluoromethyl)-1-methyl-N-[2-(1,1,2,2-tetrafluoroethoxy)phenyl]-1H-pyrazole-4-carboxamide (Registry Number 923953-98-4). Compounds of Formula 49.10 can be prepared by methods described in PCT Patent Publication WO 2007/017450.

Component b49.11 relates a compound of Formula b49.11

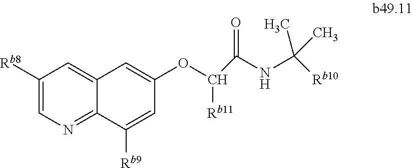

wherein
R^{b8} is halogen, C₁-C₄ alkoxy or C₂-C₄ alkynyl;
R^{b9} is H, halogen or C₁-C₄ alkyl;
R^{b10} is C₁-C₁₂ alkyl, C₁-C₁₂ haloalkyl, C₁-C₁₂ alkoxy, C₂-C₁₂ alkoxyalkyl, C₂-C₁₂ alkenyl, C₂-C₁₂ alkynyl, C₄-C₁₂ alkoxyalkenyl, C₄-C₁₂ alkoxyalkynyl, C₁-C₁₂ alkylthio or C₂-C₁₂ alkylthioalkyl;
R^{b11} is methyl or —Y^{b13}—R^{b12};
R^{b12} is C₁-C₂ alkyl; and
Y^{b13} is CH₂, O or S.
   Examples of compounds of Formula b49.11 include (b49.11a) 2-[(3-bromo-6-quinolinyl)oxy]-N-(1,1-dimethyl-2-butyn-1-yl)-2-(methylthio)acetamide, (b49.11b) 2[(3-ethynyl-6-quinolinyl)oxy]-N-[1-(hydroxymethyl)-1-methyl-2-propyn-1-yl]-2-(methylthio)acetamide, (b49.11c)N-(1,1-dimethyl-2-butyn-1-yl)-2-[(3-ethynyl-6-quinolinyl)oxy]-2-(methylthio)acetamide, (b49.11d) 2-[(3-bromo-8-methyl-6-quinolinyl)oxy]-N-(1,1-dimethyl-2-propyn-1-yl)-2-(methylthio)acetamide and (b49.11e) 2-[(3-bromo-6-quinolinyl)oxy]-N-(1,1-dimethylethyl)-butanamide. Compounds of Formula b49.11, their use as fungicides and methods of preparation are generally known; see, for example, PCT Patent Publications WO 2004/047538, WO 2004/108663, WO 2006/058699, WO 2006/058700, WO 2008/110355, WO 2009/030469, WO 2009/049716 and WO 2009/087098.

Component 49.12 relates to N-[4-[[3-[(4-chlorophenyl)methyl]-1,2,4-thiadiazol-5-yl]oxy]-2,5-dimethylphenyl]-N-ethyl-N-methylmethanimidamide, which is believed to inhibit C24-methyl transferase involved in the biosynthesis of sterols.

Component 49.13 relates to (1S)-2,2-bis(4-fluorophenyl)-1-methylethyl N-[[3-(acetyloxy)-4-methoxy-2-pyridinyl]carbonyl]-L-alaninate (Registry Number 1961312-55-9, common name florylpicoxamid), which is believed to be a Quinone inside inhibitor (QiI) fungicide (FRAC code 21) inhibiting the Complex III mitochondrial respiration in fungi.

Therefore of note is a mixture (i.e. composition) comprising a compound of Formula 1 and at least one fungicidal compound selected from the group consisting of the aforedescribed classes (1) through (49). Also of note is a composition comprising said mixture (in fungicidally effective amount) and further comprising at least one additional component selected from the group consisting of surfactants, solid diluents and liquid diluents. Of particular note is a mixture (i.e. composition) comprising a compound of Formula 1 and at least one fungicidal compound selected from the group of specific compounds listed above in connection with classes (1) through (49). Also of particular note is a composition comprising said mixture (in fungicidally effective amount) and further comprising at least one additional surfactant selected from the group consisting of surfactants, solid diluents and liquid diluents.

Examples of component (b) fungicides include acibenzolar-S-methyl, aldimorph, ametoctradin, amisulbrom, anilazine, azaconazole, azoxystrobin, benalaxyl (including benalaxyl-M), benodanil, benomyl, benthiavalicarb (including benthiavalicarb-isopropyl), benzovindiflupyr, bethoxazin, binapacryl, biphenyl, bitertanol, bixafen, blasticidin-S, boscalid, bromuconazole, bupirimate, buthiobate, captafol, captan, carbendazim, carboxin, carpropamid, chloroneb, chlorothalonil, chlozolinate, clotrimazole, copper hydroxide, copper oxychloride, copper sulfate, coumoxystrobin, cyazofamid, cyflufenamid, cymoxanil, cyproconazole, cyprodinil, dichlofluanid, diclocymet, diclomezine, dicloran, diethofencarb, difenoconazole, diflumetorim, dimethirimol, dimethomorph, dimoxystrobin, diniconazole (including diniconazole-M), dinocap, dithianon, dithiolanes, dodemorph, dodine, econazole, edifenphos, enoxastrobin (also known as enestroburin), epoxiconazole, etaconazole, ethaboxam, ethirimol, etridiazole, famoxadone, fenamidone, fenarimol, fenaminstrobin, fenbuconazole, fenfuram, fenhexamid, fenoxanil, fenpiclonil, fenpropidin, fenpropimorph, fenpyrazamine, fentin acetate, fentin chloride, fentin hydroxide, ferbam, ferimzone, flometoquin, florylpicoxamid, fluazinam, fludioxonil, flufenoxystrobin, fluindapyr, flumorph, fluopicolide, fluopyram, flouroimide, fluoxastrobin, fluquinconazole, flusilazole, flusulfamide, flutianil, flutolanil, flutriafol, fluxapyroxad, folpet, fthalide, fuberidazole, furalaxyl, furametpyr, guazatine, hexaconazole, hymexazole, imazalil, imibenconazole, iminoctadine albesilate, iminoctadine triacetate, iodocarb, ipconazole, iprobenfos, iprodione, iprovalicarb, isoconazole, isofetamid, isoprothiolane, isoflucypram, isopyrazam, isotianil, kasugamycin, kresoxim-methyl, mancozeb, mandepropamid, mandestrobin, maneb, mepanipyrim, mepronil, meptyldinocap, metalaxyl (including metalaxyl-M/mefenoxam), mefentrifluconazole, metconazole, methasulfocarb, metiram, metominostrobin, metrafenone, miconazole, myclobutanil, naftifine, neo-asozin, nuarimol, octhilinone, ofurace, orysastrobin, oxadixyl, oxathiapiprolin, oxolinic acid, oxpoconazole, oxycarboxin, oxytetracycline, pefurazoate, penconazole, pencycuron, penflufen, penthiopyrad, phosphorous acid (including salts thereof, e.g., fosetyl-aluminum), picarbutrazox, picoxystrobin, piperalin, polyoxin, probenazole, prochloraz, procymidone, propamacarb, propiconazole, propineb, proquinazid, prothiocarb, prothioconazole, pyraclostrobin, pyrametostrobin, pyraoxystrobin, pyrazophos, pyribencarb, pyributicarb, pyrifenox, pyrimethanil, pyriofenone, pyrisoxazole, pyroquilon, pyrrolnitrin, quinconazole, quinomethionate, quinoxyfen, quintozene, sedaxane, silthiofam, simeconazole, spiroxamine, streptomycin, sulfur, tebuconazole, tebufloquin, teclofthalam, tecnazene, terbinafine, tetraconazole, thiabendazole, thifluzamide, thiophanate, thiophanate-methyl, thiram, tiadinil, tolclofos-methyl, tolnifanide, tolprocarb, tolyfluanid, triadimefon, triadimenol, triarimol, triticonazole, triazoxide, tribasic copper sulfate, tricyclazole, triclopyricarb, tridemorph, trifloxystrobin, triflumizole, triforine, trimorphamide, uniconazole, uniconazole-P, validamycin, valifenalate (also known as valiphenal), vinclozolin, zineb, ziram, zoxamide, (3S,6S,7R,8R)-3-[[[3-[(acetyloxy)methoxy]-4-methoxy-2-pyridinyl]carbonyl]amino]-6-methyl-4,9-dioxo-8-(phenylmethyl)-1,5-dioxonan-7-yl 2-methylpropanoate, (3S,6S,7R,8R)-3-[[[3-(acetyloxy)-4-methoxy-2-pyridinyl]carbonyl]amino]-6-methyl-4,9-dioxo-8-(phenylmethyl)-1,5-dioxonan-7-yl 2-methylpropanoate, N-[[3-(1,3-benzodioxol-5-ylmethoxy)-4-methoxy-2-pyridinyl]carbonyl]-O-[2,5-dideoxy-3-O-(2-methyl-1-oxopropyl)-2-(phenylmethyl)-L-arabinonoyl]-L-serine, (1->4′)-lactone, N-[2-(1S,2R)-[1,1′-bicyclopropyl]-2-ylphenyl]-3-(difluoromethyl)-1-methyl-1H-pyrazole-4-carboxamide, 2-[(3-bromo-6-quinolinyl)oxy]-N-(1,1-dimethyl-2-butyn-1-yl)-2-(methylthio)acetamide, 2-[(3-bromo-6-quinolinyl)oxy]-N-(1,1-dimethylethyl)butanamide, 2-[(3-bromo-8-methyl-6-quinolinyl)oxy]-N-(1,1-dimethyl-2-propyn-1-yl)-2-(methylthio)acetamide, 2-butoxy-6-iodo-3-propyl-4H-1-benzopyran-4-one, 3-butyn-1-yl N-[6-[[[[(1-methyl-1H-tetrazol-5-yl)phenylmethylene]amino]oxy]methyl]-2-pyridinyl]-carbamate, α-(1-chlorocyclopropyl)-α-[2-(2,2-dichlorocyclopropyl)ethyl]-1H-1,2,4-triazole-1-ethanol, 2-[2-(1-chlorocyclopropyl)-4-(2,2-dichlorocyclopropyl)-2-hydroxybutyl]-1,2-dihydro-3H-1,2,4-triazole-3-thione, (αS)-[3-(4-chloro-2-fluorophenyl)-5-(2,4-difluorophenyl)-4-isoxazolyl]-3-pyridinemethanol, rel-1-[[(2R,3S)-3-(2-chlorophenyl)-2-(2,4-difluorophenyl)-2-oxiranyl]methyl]-1H-1,2,4-triazole, rel-2-[[(2R,3S)-3-(2-chlorophenyl)-2-(2,4-difluorophenyl)-2-oxiranyl]methyl]-1,2-dihydro-3H-1,2,4-triazole-3-thione, rel-1-[[(2R,3S)-3-(2-chlorophenyl)-2-(2,4-difluorophenyl)-2-oxiranyl]methyl]-5-(2-propen-1-ylthio)-1H-1,2,4-triazole, 3-[5-(4-chlorophenyl)-2,3-dimethyl-3-isoxazolidinyl]pyridine, (2-chloro-6-fluorophenyl)methyl 2-[1-[2-[3,5-bis(difluoromethyl)-1H-pyrazol-1-yl]acetyl]-4-piperidinyl]-4-thiazolecarboxylate, N-[4-[[3-[(4-chlorophenyl)methyl]-1,2,4-thiadiazol-5-yl]oxy]-2,5-dimethylphenyl]-N-ethyl-N-methylmethanimidamide, N-[2-[4-[[3-(4-chlorophenyl)-2-propyn-1-yl]oxy]-3-methoxyphenyl]ethyl]-3-methyl-2-[(methylsulfonyl)amino]butanamide, N-[2-[4-[[3-(4-chlorophenyl)-2-propyn-1-yl]oxy]-3-methoxyphenyl]ethyl]-3-methyl-2-[(ethylsulfonyl)amino]butanamide, N′-[4-[4-chloro-3-(trifluoromethyl)phenoxy]-2,5-dimethylphenyl]-N-ethyl-N-methylmethanimidamide, N-cyclopropyl-3-(difluoromethyl)-5-fluoro-1-methyl-N-[[2-(1-methylethyl)phenyl]methyl]-1H-pyrazole-4-carboxamide, N-[[(cyclopropylmethoxy)amino][6-(difluoromethoxy)-2,3-difluorophenyl]methylene]benzeneacetamide, N-[2-(2,4-dichlorophenyl)-2-methoxy-1-methylethyl]-3-(difluoromethyl)-1-methyl-1H-pyrazole-4-carboxamide, N-(3′,4′-difluoro[1,1′-biphenyl]-2-yl)-3-(trifluoromethyl)-2-pyrazinecarboxamide, 3-(difluoromethyl)-N-(2,3-dihydro-1,1,3-trimethyl-1H-inden-4-yl)-1-methyl-1H-pyrazole-4-carboxamide, 3-(difluoromethyl)-N-[4-fluoro-2-(1,1,2,3,3,3-hexafluoropropoxy)phenyl]-1-methyl-1H-pyrazole-4-carboxamide, 5,8-difluoro-N-[2-[3-methoxy-4-[[4-(trifluoromethyl)-2-pyridinyl]oxy]phenyl]ethyl]-4-quinazolinamine, 3-(difluoromethyl)-1-methyl-N-[2-(1,1,2,2-tetrafluoroethoxy)phenyl]-1H-pyrazole-4-carboxamide, 1-[4-[4-[5R-[(2,6-difluorophenoxy)methyl]-4,5-dihydro-3-isoxazolyl]-2-thiazolyl]-1-piperdinyl]-2-[5-methyl-3-(trifluoromethyl)-1H-pyrazol-1-yl]ethanone, N-(1,1-dimethyl-2-butyn-1-yl)-2-[(3-ethynyl-6-quinolinyl)oxy]-2-(methylthio)acetamide, 2,6-dimethyl-1H,5H-[1,4]dithiino[2,3-c:5,6-c′]dipyrrole-1,3,5,7(2H,6H)-tetrone, 2-[(3-ethynyl-6-quinolinyl)oxy]-N-[1-(hydroxymethyl)-1-methyl-2-propyn-1-yl]-2-(methylthio)acetamide, 4-fluorophenyl N-[1-[[[1-(4-cyanophenyl)ethyl]sulfonyl]methyl]propyl]carbamate, 5-fluoro-2-[(4-fluorophenyl)-methoxy]-4-pyrimidinamine, 5-fluoro-2-[(4-methylphenyl)methoxy]-4-pyrimidinamine, (3S,6S,7R,8R)-3-[[[4-methoxy-3-[[(2-methylpropoxy)carbonyl]oxy]-2-pyridinyl]carbonyl]-amino]-6-methyl-4,9-dioxo-8-(phenylmethyl)-1,5-dioxonan-7-yl-2-methylpropanoate, α-(methoxyimino)-N-methyl-2-[[[1-[3-(trifluoromethyl)phenyl]ethoxy]imino]methyl]benzeneacetamide, [[4-methoxy-2-[[[(3S,7R,8R,9S)-9-methyl-8-(2-methyl-1-oxopropoxy)-2,6-dioxo-7-(phenylmethyl)-1,5-dioxonan-3-yl]amino]carbonyl]-3-pyridinyl]oxy]methyl 2-methylpropanoate, pentyl N-[6-[[[[(1-methyl-1H-tetrazol-5-yl)phenylmethylene]amino]oxy]methyl]-2-pyridinyl]carbamate, pentyl N-[4-[[[[(1-methyl-1H-tetrazol-5-yl)phenylmethylene]amino]-oxy]methyl]-2-thiazolyl]carbamate, and pentyl N-[6-[[[[(Z)-(1-methyl-1H-tetrazol-5-yl)phenylmethylene]amino]oxy]methyl]-2-pyridinyl]carbamate and (1R)-1,2,3,4-tetrahydro-1-naphthalenyl 2-[1-[2-[3,5-bis(difluoromethyl)-1H-pyrazol-1-yl]acetyl]-4-piperidinyl]-4-thiazolecarboxylate. Therefore of note is a fungicidal composition comprising as component (a) a compound of Formula 1 (or an N-oxide or salt thereof) and as component (b) at least one fungicide selected from the preceding list.

Of particular note are combinations of compounds of Formula 1 (or an N-oxide or salt thereof) (i.e. Component (a) in compositions) with azoxystrobin, benzovindiflupyr, bixafen, captan, carpropamid, chlorothalonil, copper hydroxide, copper oxychloride, copper sulfate, cymoxanil, cyproconazole, cyprodinil, diethofencarb, difenoconazole, dimethomorph, epoxiconazole, ethaboxam, fenarimol, fenhexamid, fluazinam, fludioxonil, fluindapyr, fluopyram, flusilazole, flutianil, flutriafol, fluxapyroxad, folpet, iprodione, isofetamid, isoflucypram, isopyrazam, kresoxim-methyl, mancozeb, mandestrobin, meptyldinocap, metalaxyl (including metalaxyl-M/mefenoxam), mefentrifluconazole, metconazole, metrafenone, myclobutanil, oxathiapiprolin, penflufen, penthiopyrad, phosphorous acid (including salts thereof, e.g., fosetyl-aluminum), picoxystrobin, propiconazole, proquinazid, prothioconazole, pyraclostrobin, pyrimethanil, sedaxane spiroxamine, sulfur, tebuconazole, thiophanate-methyl, trifloxystrobin, zoxamide, α-(1-chlorocyclopropyl)-α-[2-(2,2-dichlorocyclopropyl)ethyl]-1H-1,2,4-triazole-1-ethanol, 2-[2-(1-chlorocyclopropyl)-4-(2,2-dichlorocyclopropyl)-2-hydroxybutyl]-1,2-dihydro-3H-1,2,4-triazole-3-thione, N-[2-(2,4-dichlorophenyl)-2-methoxy-1-methylethyl]-3-(difluoromethyl)-1-methyl-1H-pyrazole-4-carboxamide, 3-(difluoromethyl)-N-(2,3-dihydro-1,1,3-trimethyl-1H-inden-4-yl)-1-methyl-1H-pyrazole-4-carboxamide, 1-[4-[4-[5R-(2,6-difluorophenyl)-4,5-dihydro-3-isoxazolyl]-2-thiazolyl]-1-piperidinyl]-2-[5-methyl-3-(trifluoromethyl)-1H-pyrazol-1-yl]ethanone, 1,1-dimethylethyl N-[6-[[[[(1-methyl-1H-tetrazol-5-yl)phenylmethylene]amino]oxy]methyl]-2-pyridinyl]carbamate, 2,6-dimethyl-1H,5H-[1,4]dithiino[2,3-c:5,6-c′]dipyrrole-1,3,5,7(2H,6H)-tetrone, 5-fluoro-2-[(4-fluorophenyl)methoxy]-4-pyrimidinamine, 5-fluoro-2-[(4-methylphenyl)methoxy]-4-pyrimidinamine, (αS)-[3-(4-chloro-2-fluorophenyl)-5-(2,4-difluorophenyl)-4-isoxazolyl]-3-pyridinemethanol, rel-1-[[(2R,3S)-3-(2-chlorophenyl)-2-(2,4-difluorophenyl)-2-oxiranyl]methyl]-1H-1,2,4-triazole, rel-2-[[(2R,3S)-3-(2-chlorophenyl)-2-(2,4-difluorophenyl)-2-oxiranyl]methyl]-1,2-dihydro-3H-1,2,4-triazole-3-thione, and rel-1-[[(2R,3S)-3-(2-chlorophenyl)-2-(2,4-difluorophenyl)-2-oxiranyl]methyl]-5-(2-propen-1-ylthio)-1H-1,2,4-triazole (i.e. as Component (b) in compostions).

Examples of other biologically active compounds or agents with which compounds of this invention can be formulated are: invertebrate pest control compounds or agents such as abamectin, acephate, acetamiprid, acrinathrin, afidopyropen ([(3S,4R,4aR,6S,6aS,12R,12aS,12bS)-3-[(cyclopropylcarbonyl)oxy]-1,3,4,4a,5,6,6a,12,12a,12b-decahydro-6,12-dihydroxy-4,6a,12b-trimethyl-11-oxo-9-(3-pyridinyl)-2H,11H-naphtho[2,1-b]pyrano[3,4-e]pyran-4-yl]methyl cyclopropanecarboxylate), amidoflumet (S-1955), avermectin, azadirachtin, azinphos-methyl, bifenthrin, bifenazate, buprofezin, carbofuran, cartap, chlorantraniliprole, chlorfenapyr, chlorfluazuron, chlorpyrifos, chlorpyrifos-methyl, chromafenozide, clothianidin, cyantraniliprole (3-bromo-1-(3-chloro-2-pyridinyl)-N-[4-cyano-2-methyl-6-[(methylamino)carbonyl]phenyl]-1H-pyrazole-5-carboxamide), cyclaniliprole (3-bromo-N-[2-bromo-4-chloro-6-[[(1-cyclopropylethyl)amino]carbonyl]phenyl]-1-(3-chloro-2-pyridinyl)-1H-pyrazole-5-carboxamide), cycloxaprid ((5S,8R)-1-[(6-chloro-3-pyridinyl)methyl]-2,3,5,6,7,8-hexahydro-9-nitro-5,8-epoxy-1H-imidazo[1,2-a]azepine), cyflumetofen, cyfluthrin, beta-cyfluthrin, cyhalothrin, lambda-cyhalothrin, cypermethrin, cyromazine, deltamethrin, diafenthiuron, diazinon, dieldrin, diflubenzuron, dimefluthrin, dimethoate, dinotefuran, diofenolan, emamectin, endosulfan, esfenvalerate, ethiprole, fenothiocarb, fenoxycarb, fenpropathrin, fenvalerate, fipronil, flonicamid, flubendiamide, flucythrinate, flufenoxystrobin (methyl (αE)-2-[[2-chloro-4-(trifluoromethyl)phenoxy]methyl]-α-(methoxymethylene)benzeneacetate), fluensulfone (5-chloro-2-[(3,4,4-trifluoro-3-buten-1-yl)sulfonyl]thiazole), flupiprole (1-[2,6-dichloro-4-(trifluoromethyl)phenyl]-5-[(2-methyl-2-propen-1-yl)amino]-4-[(trifluoro-methyl)sulfinyl]-1H-pyrazole-3-carbonitrile), flupyradifurone (4-[[(6-chloro-3-pyridinyl)-methyl](2,2-difluoroethyl)amino]-2(5H)-furanone), tau-fluvalinate, flufenerim (UR-50701), flufenoxuron, fonophos, halofenozide, heptafluthrin ([2,3,5,6-tetrafluoro-4-(methoxymethyl)-phenyl]methyl 2,2-dimethyl-3-[(1Z)-3,3,3-trifluoro-1-propen-1-yl]cyclopropanecarboxylate), hexaflumuron, hydramethylnon, imidacloprid, indoxacarb, isofenphos, lufenuron, malathion, meperfluthrin ([2,3,5,6-tetrafluoro-4-(methoxymethyl)phenyl]methyl (1R,3S)-3-(2,2-dichloro-ethenyl)-2,2-dimethylcyclopropanecarboxylate), metaflumizone, metaldehyde, methamidophos, methidathion, methomyl, methoprene, methoxychlor, methoxyfenozide, metofluthrin, milbemycin oxime, momfluorothrin ([2,3,5,6-tetrafluoro-4-(methoxymethyl)phenyl]methyl-3-(2-cyano-1-propen-1-yl)-2,2-dimethylcyclopropanecarboxylate), monocrotophos, nicotine, nitenpyram, nithiazine, novaluron, noviflumuron (XDE-007), oxamyl, pyflubumide (1,3,5-trimethyl-N-(2-methyl-1-oxopropyl)-N-[3-(2-methylpropyl)-4-[2,2,2-trifluoro-1-methoxy-1-(trifluoromethyl)ethyl]phenyl]-1H-pyrazole-4-carboxamide), parathion, parathion-methyl, permethrin, phorate, phosalone, phosmet, phosphamidon, pirimicarb, profenofos, profluthrin, pymetrozine, pyrafluprole, pyrethrin, pyridalyl, pyrifluquinazon, pyriminostrobin (methyl (aE)-2-[[[2-[(2,4-dichlorophenyl)amino]-6-(trifluoromethyl)-4-pyrimidinyl]oxy]methyl]-α-(methoxy-methylene)benzeneacetate), pyriprole, pyriproxyfen, rotenone, ryanodine, spinetoram, spinosad, spirodiclofen, spiromesifen (BSN 2060), spirotetramat, sulfoxaflor, sulprofos, tebufenozide, teflubenzuron, tefluthrin, terbufos, tetrachlorvinphos, tetramethylfluthrin, thiacloprid, thiamethoxam, thiodicarb, thiosultap-sodium, tolfenpyrad, tralomethrin, triazamate, trichlorfon and triflumuron; and biological agents including entomopathogenic bacteria, such as Bacillus thuringiensis subsp. aizawai, Bacillus thuringiensis subsp. kurstaki, and the encapsulated delta-endotoxins of Bacillus thuringiensis (e.g., Cellcap, MPV, MPVII); entomopathogenic fungi, such as green muscardine fungus; and entomopathogenic virus including baculovirus, nucleopolyhedro virus (NPV) such as HzNPV, AfNPV; and granulosis virus (GV) such as CpGV.

Compounds of this invention and compositions thereof can be applied to plants genetically transformed to express proteins toxic to invertebrate pests (such as Bacillus thuringiensis delta-endotoxins). The effect of the exogenously applied fungicidal compounds of this invention may provide an enhanced effect with the expressed toxin proteins.

General references for agricultural protectants (i.e. insecticides, fungicides, nematocides, acaricides, herbicides and biological agents) include The Pesticide Manual, 13th Edition, C. D. S. Tomlin, Ed., British Crop Protection Council, Farnham, Surrey, U. K., 2003 and The BioPesticide Manual, 2nd Edition, L. G. Copping, Ed., British Crop Protection Council, Farnham, Surrey, U. K., 2001.

For embodiments where one or more of these various mixing partners are used, the weight ratio of these various mixing partners (in total) to the compound of Formula 1 is typically between about 1:3000 and about 3000:1. Of note are weight ratios between about 1:300 and about 300:1 (for example ratios between about 1:30 and about 30:1). One skilled in the art can easily determine through simple experimentation the biologically effective amounts of active ingredients necessary for the desired spectrum of biological activity. It will be evident that including these additional components may expand the spectrum of diseases controlled beyond the spectrum controlled by the compound of Formula 1 alone.

In certain instances, combinations of a compound of this invention with other biologically active (particularly fungicidal) compounds or agents (i.e. active ingredients) can result in a greater-than-additive (i.e. enhanced) effect. Reducing the quantity of active ingredients released in the environment while ensuring effective pest control is always desirable. When an enhanced effect of fungicidal active ingredients occurs at application rates giving agronomically satisfactory levels of fungal control, such combinations can be advantageous for reducing crop production cost and decreasing environmental load.

Also in certain instances, combinations of a compound of the invention with other biologically active compounds or agents can result in a less-than-additive (i.e. safening) effect on organisms beneficial to the agronomic environment. For example, a compound of the invention may safen a herbicide on crop plants or protect a beneficial insect species (e.g., insect predators, pollinators such as bees) from an insecticide.

Fungicides of note for formulation with compounds of Formula 1 to provide mixtures useful in seed treatment include but are not limited to amisulbrom, azoxystrobin, boscalid, carbendazim, carboxin, cymoxanil, cyproconazole, difenoconazole, dimethomorph, florylpicoxamid, fluazinam, fludioxonil, flufenoxystrobin, fluquinconazole, fluopicolide, fluoxastrobin, flutriafol, fluxapyroxad, ipconazole, iprodione, metalaxyl, mefenoxam, mefentrifluconazole, metconazole, myclobutanil, paclobutrazole, penflufen, picoxystrobin, prothioconazole, pyraclostrobin, sedaxane, silthiofam, tebuconazole, thiabendazole, thiophanate-methyl, thiram, trifloxystrobin and triticonazole.

Invertebrate pest control compounds or agents with which compounds of Formula 1 can be formulated to provide mixtures useful in seed treatment include but are not limited to abamectin, acetamiprid, acrinathrin, afidopyropen, amitraz, avermectin, azadirachtin, bensultap, bifenthrin, buprofezin, cadusafos, carbaryl, carbofuran, cartap, chlorantraniliprole, chlorfenapyr, chlorpyrifos, clothianidin, cyantraniliprole, cyclaniliprole, cyfluthrin, beta-cyfluthrin, cyhalothrin, gamma-cyhalothrin, lambda-cyhalothrin, cypermethrin, alpha-cypermethrin, zeta-cypermethrin, cyromazine, deltamethrin, dieldrin, dinotefuran, diofenolan, emamectin, endosulfan, esfenvalerate, ethiprole, etofenprox, etoxazole, fenothiocarb, fenoxycarb, fenvalerate, fipronil, flonicamid, flubendiamide, fluensulfone, flufenoxuron, flufiprole, flupyradifurone, fluvalinate, formetanate, fosthiazate, heptafluthrin, hexaflumuron, hydramethylnon, imidacloprid, indoxacarb, lufenuron, meperfluthrin, metaflumizone, methiocarb, methomyl, methoprene, methoxyfenozide, momfluorothrin, nitenpyram, nithiazine, novaluron, oxamyl, pyflubumide, pymetrozine, pyrethrin, pyridaben, pyriminostrobin, pyridalyl, pyriproxyfen, ryanodine, spinetoram, spinosad, spirodiclofen, spiromesifen, spirotetramat, sulfoxaflor, tebufenozide, tetramethrin, tetramethylfluthrin, thiacloprid, thiamethoxam, thiodicarb, thiosultap-sodium, tralomethrin, triazamate, triflumuron, Bacillus thuringiensis delta-endotoxins, strains of Bacillus thuringiensis and strains of Nucleo polyhydrosis viruses.

Compositions comprising compounds of Formula 1 useful for seed treatment can further comprise bacteria and fungi that have the ability to provide protection from the harmful effects of plant pathogenic fungi or bacteria and/or soil born animals such as nematodes. Bacteria exhibiting nematicidal properties may include but are not limited to Bacillus firmus, Bacillus cereus, Bacillius subtiliis and Pasteuria penetrans. A suitable Bacillus firmus strain is strain CNCM I-1582 (GB-126) which is commercially available as BioNem™. A suitable Bacillus cereus strain is strain NCMM I-1592. Both Bacillus strains are disclosed in U.S. Pat. No. 6,406,690. Other suitable bacteria exhibiting nematicidal activity are B. amyloliquefaciens IN937a and B. subtilis strain GB03. Bacteria exhibiting fungicidal properties may include but are not limited to B. pumilus strain GB34. Fungal species exhibiting nematicidal properties may include but are not limited to Myrothecium verrucaria, Paecilomyces lilacinus and Purpureocillium lilacinum.

Seed treatments can also include one or more nematicidal agents of natural origin such as the elicitor protein called harpin which is isolated from certain bacterial plant pathogens such as Erwinia amylovora. An example is the Harpin-N-Tek seed treatment technology available as N-Hibit™ Gold CST.

Seed treatments can also include one or more species of legume-root nodulating bacteria such as the microsymbiotic nitrogen-fixing bacteria Bradyrhizobium japonicum. These inocculants can optionally include one or more lipo-chitooligosaccharides (LCOs), which are nodulation (Nod) factors produced by rhizobia bacteria during the initiation of nodule formation on the roots of legumes. For example, the Optimize® brand seed treatment technology incorporates LCO Promoter Technology™ in combination with an inocculant.

Seed treatments can also include one or more isoflavones which can increase the level of root colonization by mycorrhizal fungi. Mycorrhizal fungi improve plant growth by enhancing the root uptake of nutrients such as water, sulfates, nitrates, phosphates and metals. Examples of isoflavones include, but are not limited to, genistein, biochanin A, formononetin, daidzein, glycitein, hesperetin, naringenin and pratensein. Formononetin is available as an active ingredient in mycorrhizal inocculant products such as PHC Colonize® AG.

Seed treatments can also include one or more plant activators that induce systemic acquired resistance in plants following contact by a pathogen. An example of a plant activator which induces such protective mechanisms is acibenzolar-S-methyl.

The following TESTS demonstrate the control efficacy of compounds of this invention on specific pathogens. The pathogen control protection afforded by the compounds is not limited, however, to these species. See Index Tables A through L below for compound descriptions. The following abbreviations are used in the Index Tables: Me means methyl, Et means ethyl, n-Pr means n-propyl, i-Pr means iso-propyl, c-Pr means cyclopropyl, n-Bu means n-butyl, i-Bu means iso-butyl, c-Bu means cyclobutyl, c-hexyl means cyclohexyl, Ph means phenyl, MeO means methoxy and EtO means ethoxy. The abbreviation “Cmpd. No.” stands for “Compound Number”, and the abbreviation “Ex.” stands for “Example” and is followed by a number indicating in which example the compound is prepared. The abbreviation “m.p.” stands for melting point. The numerical value reported in the column “MS” is the molecular weight of the highest isotopic abundance positively charged parent ion (M+1) formed by addition of H⁺ (molecular weight of 1) to the molecule having the highest isotopic abundance, or the highest isotopic abundance negatively charged ion (M-1) formed by loss of H⁺ (molecular weight of 1). The presence of molecular ions containing one or more higher atomic weight isotopes of lower abundance (e.g., ³⁷Cl, ⁸¹Br) is not reported. The reported MS peaks were observed by mass spectrometry using electrospray ionization (ESI) or atmospheric pressure chemical ionization (APCI).

INDEX TABLE A

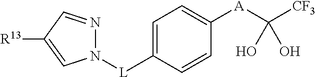

| Cmpd. No. | R¹³ | L | A | NMR | MS |
|---|---|---|---|---|---|
| 1 | EtOC(═O) | CH₂ | OCH₂ | * | |
| (Ex. 1) | | | | | |
| 5 | EtOC(═O) | CH₂ | OCH(Me) | * | |
| 35 | EtOC(═O) | CH(Me) | OCH₂ | * | |
| 36 | c-PrCH₂NHC(═O) | CH₂ | OCH₂ | * | 400 (M + 1) |
| 37 | MeOC(═O) | CH₂ | OCH₂ | * | 361 (M + 1) |
| 38 | CH₂═CHCF₂OC(═O) | CH₂ | OCH₂ | | 423 (M + 1) |
| 39 | i-PrOC(═O) | CH₂ | OCH₂ | * | 389 (M + 1) |
| 40 | CH₂═C(Me)CH₂OC(═O) | CH₂ | OCH₂ | * | 401 (M + 1) |
| 42 | n-PrOC(═O) | CH₂ | OCH₂ | * | 389 (M + 1) |
| 43 | CH₂═CHCH₂OC(═O) | CH₂ | OCH₂ | * | 387 (M + 1) |
| 44 | HC≡CCH₂OC(═O) | CH₂ | OCH₂ | * | 385 (M + 1) |
| 45 | CF₃CH₂OC(═O) | CH₂ | OCH₂ | * | 429 (M + 1) |
| 46 | CF₃CF₂CH₂OC(═O) | CH₂ | OCH₂ | * | 479 (M + 1) |
| 47 | PhCH₂OC(═O) | CH₂ | OCH₂ | * | 437 (M + 1) |
| 48 | CH₃(CH₂)₅OC(═O) | CH₂ | OCH₂ | * | 431 (M + 1) |
| 49 | 3,4-di-Cl—PhCH₂OC(═O) | CH₂ | OCH₂ | * | 505 (M + 1) |
| 50 | 3,4-di-F—PhCH₂OC(═O) | CH₂ | OCH₂ | * | 473 (M + 1) |
| 51 | EtOC(═O) | CH₂ | CH(OH)CH₂ | * | |
| 66 | EtOC(═O) | CH₂ | CH₂CH₂ | * | 355 (M + 1) |
| 131 | EtOC(═O) | CH₂CH₂ | OCH₂ | * | |
| 206 | N≡C | CH₂ | OCH₂ | * | |
| 227 | EtOC(═O) | CH₂ | OCF₂ | * | |
| 286 | EtOC(═O) | (CH₂)₃ | OCH₂ | * | 403 (M + 1) |
| 322 | Br | CH₂ | OCH₂ | * | 383 (M + 1) |
| 329 | HOC(═O) | CH₂ | OCH₂ | * | 327 (M − 1) |

*See Index Table M for ¹⁹F NMR data.

INDEX TABLE A1

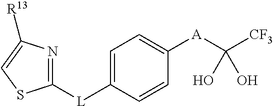

| Cmpd. | | | | | m.p. |
|---|---|---|---|---|---|
| No. | R¹³ | L | A | MS | (° C.) |
| 68 | CF₃CH₂NHC(═O) | CH₂ | OCH₂ | 445 (M + 1) | 116-120 |
| 69 | c-PrCH₂NHC(═O) | CH₂ | OCH₂ | 417 (M + 1) | |
| 135 | 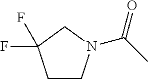 | CH₂ | OCH₂ | 453 (M + 1) | |
| 136 | EtOC(═O) | CH₂ | OCH₂ | 392 (M + 1) | |
| 215 | 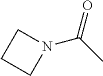 | CH₂ | OCH₂ | 403 (M + 1) | 140-144 |
| 223 | MeOCH₂CH₂NHC(═O) | CH₂ | OCH₂ | 421 (M + 1) | |

INDEX TABLE B

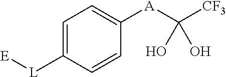

| | | | | | | m.p. |
|---|---|---|---|---|---|---|
| Cmpd. No. | E | L | A | NMR | MS | (° C.) |
| 2 | CF₃C(OH)₂CH₂O | — | OCH₂ | * | | 105-109 |
| 29 | CH₃CH₂S(═O)₂ | CH₂ | OCH₂ | * | | |
| 30 | 1-indolyl | CH₂ | CH₂CH₂ | | 332 (M + 1) | |
| 31 | MeS(═O)₂ | — | OCH₂ | * | | 102-106 |
| 33 | MeOC(═O)NHN═CH | — | OCH₂ | * | | 140-145 |
| 34 | Me₂NS(═O)₂ | — | OCH₂ | * | | 142-146 |
| 41 | 3-(Me₂NC(═O))-4,5-dihydro-5-isoxazolyl | — | OCH₂ | * | | |
| 80 | CH₃C(═O) | — | OCH₂ | * | | |
| 97 | EtOC(═O)CH═CHCH₂O | — | OCH₂ | * | | |
| 142 | N≡C | — | OCH₂ | * | | |
| 177 | HC(═O) | — | OCH₂ | * | | |
| 229 | MeOC(═O) | — | OCH₂ | | 279 (M − 1) | |
| 233 | 3-(EtOC(═O))-1H-pyrazol-1-yl | CH₂ | OCH₂ | * | | |
| 234 | 5-(EtOC(═O))-1H-pyrazol-1-yl | CH₂ | OCH₂ | * | | |
| 247 | 5-(c-PrCH₂NHC(═O))-2-oxazolyl | CH₂ | OCH₂ | | 401 (M + 1) | |
| 256 | 5-(c-PrCH₂NHC(═O))-2-thiazolyl | CH₂ | OCH₂ | | 417 (M + 1) | 126-130 |
| 274 | 5-(CF₃CH₂NHC(═O))-2-oxazolyl | CH₂ | OCH₂ | | 429 (M + 1) | 75-79 |
| 277 | 5-(CF₃CH₂NHC(═O))-2-thiazolyl | CH₂ | OCH₂ | | 445 (M + 1) | 110-114 |
| 280 | NO₂ | — | OCH₂ | * | | |
| 295 | 5-(F₂CHCH₂NHC(═O))-2-thiazolyl | CH₂ | OCH₂ | | 427 (M + 1) | |
| 366 | 4-(EtOC(═O))-1H-pyrazol-1-yl-CH₂O | — | OCH₂ | ** | | |
| (Ex. 8) | | | | | | |

*See Index Table M for ¹⁹F NMR data.
**See Index Table N for ¹H NMR data.
A dash “—” in the L column means that L is a direct bond.

INDEX TABLE C

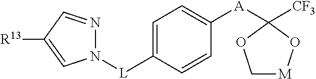

| Cmpd. No. | R¹³ | L | A | M | NMR | MS |
|---|---|---|---|---|---|---|
| 3 | n-PrOC(═O) | CH₂ | OCH₂ | CH₂ | * | 415 (M + 1) |
| 4 | EtOC(═O) | CH₂ | OCH₂ | C(═O) | * | 415 (M + 1) |
| 6 | EtOC(═O) | CH₂ | OCH(Me) | CH₂ | * | |
| 7 | EtOC(═O) | CH₂ | OCH₂ | CH(Me) | * | |
| [Note 1] | | | | | | |
| 8 | EtOC(═O) | CH₂ | OCH₂ | CH(Me) | * | |
| [Note 2] | | | | | | |
| 12 | EtOC(═O) | CH₂ | OCH₂ | C(Me)₂ | * | |
| (Ex. 3) | | | | | | |
| 13 | MeOC(═O) | CH₂ | OCH₂ | CH₂ | * | 387 (M + 1) |
| 14 | i-PrOC(═O) | CH₂ | OCH₂ | CH₂ | * | 415 (M + 1) |
| 15 | HC≡CCH₂OC(═O) | CH₂ | OCH₂ | CH₂ | * | 411 (M + 1) |
| 16 | CH₃(CH₂)₅OC(═O) | CH₂ | OCH₂ | CH₂ | * | 457 (M + 1) |
| 17 | 3,4-di-Cl—PhCH₂OC(═O) | CH₂ | OCH₂ | CH₂ | * | 531 (M + 1) |
| 18 | 3,4-di-F—PhCH₂OC(═O) | CH₂ | OCH₂ | CH₂ | * | 499 (M + 1) |
| 26 | EtOC(═O) | CH(CH₃) | OCH₂ | CH₂ | * | 413 (M − 1) |
| 32 | EtOC(═O) | CH₂ | OCH₂ | CH₂ | * | |
| (Ex. 2) | | | | | | |
| and | | | | | | |
| (Ex. 4) | | | | | | |
| 67 | EtOC(═O) | CH₂ | CH₂CH₂ | CH₂ | * | 397 (M − 1) |
| 93 | HOC(═O) | CH₂ | OCH₂ | CH₂ | * | |
| 115 | EtOC(═O) | CH₂ | SCH₂ | CH₂ | | 417 (M + 1) |
| 125 | n-BuOC(═O) | CH₂ | OCH₂ | CH₂ | | 429 (M + 1) |
| 126 | i-BuOC(═O) | CH₂ | OCH₂ | CH₂ | * | 429 (M + 1) |
| 127 | c-PrCH₂OC(═O) | CH₂ | OCH₂ | CH₂ | * | 427 (M + 1) |
| 133 | EtOC(═O) | CH₂CH₂ | OCH₂ | CH₂ | * | |
| 134 | CF₃CH₂OC(═O) | CH₂ | OCH₂ | CH₂ | * | 453 (M − 1) |
| 141 | EtOC(═O) | CH₂ | CH₂OCH₂ | CH₂ | * | |
| 161 | Cl(CH₂)₃OC(═O) | CH₂ | OCH₂ | CH₂ | * | 449 (M + 1) |
| 162 | MeOCH₂CH₂OC(═O) | CH₂ | OCH₂ | CH₂ | * | 431 (M + 1) |
| 163 | CH₃C≡CCH₂OC(═O) | CH₂ | OCH₂ | CH₂ | * | 425 (M + 1) |
| 164 | N≡CCH₂NHC(═O) | CH₂ | OCH₂ | CH₂ | * | 411 (M + 1) |
| 169 | CH₃C(═O)CH₂OC(═O) | CH₂ | OCH₂ | CH₂ | * | 429 (M + 1) |
| 170 | PhC(═O)CH₂OC(═O) | CH₂ | OCH₂ | CH₂ | * | 491 (M + 1) |
| 171 | N≡C(CH₂)₃OC(═O) | CH₂ | OCH₂ | CH₂ | * | 440 (M + 1) |
| 172 | N≡CCH₂OC(═O) | CH₂ | OCH₂ | CH₂ | * | |
| 188 | CH₂═CHCH₂OC(═O) | CH₂ | OCH₂ | CH₂ | | 413 (M + 1) |
| 197 | CF₃CH₂NHC(═O) | CH₂ | OCH₂ | CH₂ | * | 454 (M + 1) |
| 198 | Me₂NC(═O) | CH₂ | OCH₂ | CH₂ | * | 400 (M + 1) |
| 199 | 2-pyridyl-CH₂OC(═O) | CH₂ | OCH₂ | CH₂ | * | 464 (M + 1) |
| 200 | 3-pyridyl-CH₂OC(═O) | CH₂ | OCH₂ | CH₂ | * | 464 (M + 1) |
| 201 | 4-pyridyl-CH₂OC(═O) | CH₂ | OCH₂ | CH₂ | * | 464 (M + 1) |
| 202 | c-hexyl-C(═O)CH₂OC(═O) | CH₂ | OCH₂ | CH₂ | * | 497 (M + 1) |
| 203 | MeOC(═O)CH₂OC(═O) | CH₂ | OCH₂ | CH₂ | * | 445 (M + 1) |
| 204 | 1,3-dioxolan-2-yl-CH₂OC(═O) | CH₂ | OCH₂ | CH₂ | * | 459 (M + 1) |
| 209 | N≡C | CH₂ | OCH₂ | CH₂ | * | |
| 228 | CH₂═C(CH₃)CH₂OC(═O) | CH₂ | OCH₂ | CH₂ | * | 427 (M + 1) |
| 240 | EtOCH₂ | CH₂ | OCH₂ | CH₂ | * | 387 (M + 1) |
| 249 | c-BuCH₂OC(═O) | CH₂ | OCH₂ | CH₂ | * | 441 (M + 1) |
| 272 | c-hexyl-CH₂OC(═O) | CH₂ | OCH₂ | CH₂ | * | 469 (M + 1) |
| 284 | c-pentyl-CH₂OC(═O) | CH₂ | OCH₂ | CH₂ | * | 455 (M + 1) |
| 285 | CH₃CH═CHCH₂OC(═O) | CH₂ | OCH₂ | CH₂ | * | 427 (M + 1) |
| [Note 4] | | | | | | |
| 296 | EtOC(═O) | (CH₂)₃ | OCH₂ | CH₂ | * | 429 (M + 1) |
| 318 | c-BuOC(═O) | CH₂ | OCH₂ | CH₂ | * | 427 (M + 1) |
| 319 | EtOC(═O) | CH₂ | OCF₂ | CH₂ | * | 437 (M + 1) |

*See Index Table M for ¹⁹F NMR data.
Note 1:
87:13 mixture of diastereomers.
Note 2:
33:67 mixture of diastereomers.
Note 4:
60:40 mixture of cis-trans isomers.

INDEX TABLE D

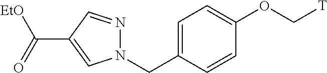

| Cmpd. | | | |
|---|---|---|---|
| No. | T | NMR | MS |
| 9 | 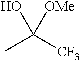 | * | |
| 10 | 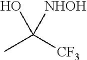 | * | |
| 20 | 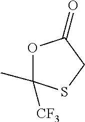 | * | |
| 21 | 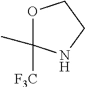 | * | 400 (M + 1) |
| 52 | 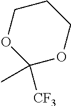 | * | |
| 187 | 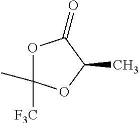 | | 429 (M + 1) |

*See Index Table M for ¹⁹F NMR data.

INDEX TABLE E

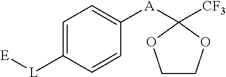

| Cmpd. No. | E | L | A | NMR | MS |
|---|---|---|---|---|---|
| 22 | 3-(Me₂NC(═O))-4,5-dihydro-5-isoxazolyl | — | OCH₂ | * | |
| 23 | CH₃CH₂S(═O)₂ | CH₂ | OCH₂ | * | |
| 24 | CH₃S(═O)₂ | — | OCH₂ | * | |
| 25 | (Me)₂NS(═O)₂ | — | OCH₂ | * | |
| 27 | MeOC(═O)NHN═CH | — | OCH₂ | * | |
| 28 | 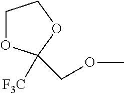 | — | OCH₂ | * | |
| 76 | i-BuS(═O)₂NH | CH₂ | OCH₂ | | 396 (M − 1) |
| 77 | c-hexyl-NHC(═S)NH | CH₂ | OCH₂ | | 419 (M + 1) |
| 78 | EtOC(═O)NH | CH₂ | OCH₂ | | 350 (M + 1) |
| 98 | N≡C | — | OCH₂ | * | 274 (M + 1) |
| 112 | i-BuOC(═O)NH | CH₂ | OCH₂ | * | |
| 160 | 5-(EtOC(═O)-1-indazolyl | CH₂ | OCH₂ | * | |
| 166 | NO₂ | — | OCH₂ | * | |
| 173 | 5-(CF₃)-1,2,4-oxadiazol-3-yl | — | OCH₂ | | 385 (M + 1) |
| 174 | NH₂ | CH₂ | OCH₂ | * | |
| 175 | i-PrC(═O)NH | CH₂ | OCH₂ | | 348 (M + 1) |
| 176 | c-PrC(═O)NH | CH₂ | OCH₂ | * | |
| 178 | OH | CH₂ | OCH₂ | | 301 (M + 23) |
| 179 | 3-CF₃—PhC(═O)NH | CH₂ | OCH₂ | * | |
| 189 | MeOC(═O) | — | OCH₂ | * | 307 (M + 1) |
| 190 | Ph | — | OCH₂ | * | |
| 191 | PhO | — | OCH₂ | * | |
| 192 | Ph | CH₂ | OCH₂ | * | |
| 207 | CF₃S(═O)₂NH | CH₂ | OCH₂ | | 408 (M − 1) |
| 212 | CF₃CH₂C(═O)NH | CH₂ | OCH₂ | | 388 (M + 1) |
| 214 | MeOCH(CH₃)C(═O)NH | CH₂ | OCH₂ | * | |
| 232 | 3-(EtOC(═O))-1H-pyrazol-1-yl | CH₂ | OCH₂ | * | |
| 235 | i-PrC(═O)N(OMe) | CH₂ | OCH₂ | * | |
| 236 | c-PrC(═O)N(OMe) | CH₂ | OCH₂ | * | |
| 237 | n-PrC(═O)N(OMe) | CH₂ | OCH₂ | * | |
| 238 | t-BuOC(═O)N(OMe) | CH₂ | OCH₂ | | 430 (M + 23) |
| 239 | N≡C | CH₂ | OCH₂ | * | |
| 281 | 2-(MeS)-4-pyrimidinyl | CH₂ | OCH₂ | | 387 (M + 1) |
| 282 | 2-(MeS(═O)₂)-4-pyrimidinyl | CH₂ | OCH₂ | | 419 (M + 1) |
| 283 | 2-(F₂CHCH₂O)-4-pyrimidinyl | CH₂ | OCH₂ | | 421 (M + 1) |
| 289 | 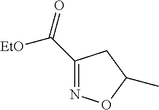 | CH₂ | OCH₂ | * | |
| 290 | 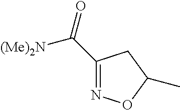 | CH₂ | OCH₂ | * | |
| 314 | 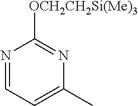 | CH₂ | OCH₂ | | 457 (M + 1) |

*See Index Table M for ¹⁹F NMR data.
A dash “—” in the L column means that L is a direct bond.

INDEX TABLE E1

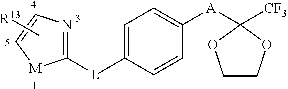

| Cmpd. | | | | | | m.p. |
|---|---|---|---|---|---|---|
| No. | R¹³ | M | L | A | MS | (° C.) |
| 70 | 4-(CF₃CH₂NHC(═O)) | S | CH₂ | OCH₂ | 471 | |
| | | | | | (M + 1) | |
| 71 | 4-(c-PrCH₂NHC(═O)) | S | CH₂ | OCH₂ | 443 | 88-92 |
| | | | | | (M + 1) | |
| 137 | 4-(EtOC(═O)) | S | CH₂ | OCH₂ | 418 | 74-78 |
| | | | | | (M + 1) | |
| 248 | 5-(c-PrCH₂NHC(═O)) | O | CH₂ | OCH₂ | 427 | |
| | | | | | (M + 1) | |
| 257 | 5-(CF₃CH₂NHC(═O)) | O | CH₂ | OCH₂ | 455 | 99-103 |
| | | | | | (M + 1) | |
| 259 | 5-(c-PrCH₂NHC(═O)) | S | CH₂ | OCH₂ | 443 | 117-121 |
| | | | | | (M + 1) | |
| 275 | 5-(CF₃CH₂NHC(═O)) | S | CH₂ | OCH₂ | 471 | |
| | | | | | (M + 1) | |

*See Index Table M for ¹⁹F NMR data.

INDEX TABLE F

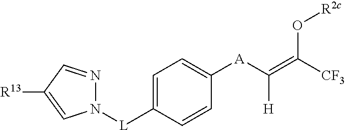

| Cmpd. | | | | | | MS | |
|---|---|---|---|---|---|---|---|
| No. | R¹³ | L | A | R^{2c} | NMR | (M + 1) | m.p. (° C.) |
| 53 | EtOC(═O) | CH₂ | O | CH₃ | * | | |
| 54 | EtOC(═O) | CH₂ | O | CH₂CH₂OMe | * | | |
| 55 | EtOC(═O) | CH₂ | O | CH₂CH═CH₂ | * | | |
| 56 | EtOC(═O) | CH₂ | O | CH₂≡CCH | * | | |
| 57 | EtOC(═O) | CH₂ | O | CH(CH₃)₂ | * | | |
| 64 | EtOC(═O) | CH₂ | O | CH₂CH₃ | * | | 58-59 |
| (Ex. 5) | | | | | | | |
| 65 | EtOC(═O) | CH₂ | O | CH₂CH₂CH₃ | * | | |
| 89 | t-BuOC(═O) | CH₂ | O | CH₂CH₃ | * | | |
| 106 | CF₃CH₂NHC(═O) | CH₂ | O | CH₂CH₃ | | | 109-113 |
| 107 | N≡CCH₂NHC(═O) | CH₂ | O | CH₂CH₃ | | | 114-118 |
| 116 | HOC(═O) | CH₂ | O | CH₂CH₃ | * | | 127-131 |
| 117 | c-PrCH₂NHC(═O) | CH₂ | O | CH₂CH₃ | | | 106-110 |
| 118 | i-PrNHC(═O) | CH₂ | O | CH₂CH₃ | | | 114-118 |
| 132 | EtOC(═O) | (CH₂)₂ | O | CH₂CH₃ | * | | |
| 138 | MeOC(═O) | CH₂ | O | CH₂CH₃ | | | 105-109 |
| 139 | i-PrOC(═O) | CH₂ | O | CH₂CH₃ | | 399 | |
| 140 | EtOC(═O) | CH₂ | CH₂ | CH₂CH₃ | * | | |
| 145 | EtOC(═O) | CH₂ | O | CH₂CH₂OH | * | | |
| 151 | — | CH₂ | O | CH₂CH₃ | * | | |
| 167 | EtOC(═O) | CH₂ | CH₂O | CH₂CH₃ | * | | |
| 180 | Cl | CH₂ | O | CH₂CH₃ | * | | |
| 182 | CF₃ | CH₂ | O | CH₂CH₃ | * | | |
| 183 | Br | CH₂ | O | CH₂CH₃ | * | | |
| 185 | CH≡CCH₂OC(═O) | CH₂ | O | CH₂CH₃ | | | 69-73 |
| 186 | PhCH₂OC(═O) | CH₂ | O | CH₂CH₃ | | | 63-67 |
| 205 | N≡C | CH₂ | O | CH₂CH₃ | * | | |
| 216 | MeC(═O)CH₂OC(═O) | CH₂ | O | CH₂CH₃ | | | 64-68 |
| 217 | n-PrOC(═O) | CH₂ | O | CH₂CH₃ | | 399 | |
| 218 | n-BuOC(═O) | CH₂ | O | CH₂CH₃ | | 413 | |
| 219 | i-BuOC(═O) | CH₂ | O | CH₂CH₃ | | 413 | |
| 220 | c-PrCH₂OC(═O) | CH₂ | O | CH₂CH₃ | | 411 | |
| 221 | Cl(CH₂)₃OC(═O) | CH₂ | O | CH₂CH₃ | | 433 | |
| 222 | MeOCH₂CH₂OC(═O) | CH₂ | O | CH₂CH₃ | | | 79-83 |
| 242 | CH₂═C(Me)CH₂OC(═O) | CH₂ | O | CH₂CH₃ | | 411 | |
| 243 | CH₃≡CCH₂OC(═O) | CH₂ | O | CH₂CH₃ | | | 105-109 |
| 244 | PhC(═O)CH₂OC(═O) | CH₂ | O | CH₂CH₃ | | | 114-118 |
| 245 | N≡CCH₂CH₂OC(═O) | CH₂ | O | CH₂CH₃ | * | | 81-85 |
| 246 | CH₂═CHCH₂OC(═O) | CH₂ | O | CH₂CH₃ | | 397 | |
| 267 | EtOC(═O) | CH(Me) | O | CH₂CH₃ | * | | |
| 294 | 4-(EtOC(═O)) | (CH₂)₃ | O | CH₂CH₃ | * | 413 | |
| 298 | EtOC(═O) | CH₂ | O | CH₂CF₃ | * | | |
| 316 | EtOC(═O) | — | O | CH₂CH₃ | | 371 | |
| 328 | HOC(═O) | (CH₂)₂ | O | CH₂CH₃ | * | | |
| 354 | MeOC(═O) | (CH₂)₂ | O | CH₂CH₃ | * | 385 | |

*See Index Table M for ¹⁹F NMR data.
A dash “—” in the R¹³ column means no R¹³ substituent is present and the remaining carbon valence is occupied by a hydrogen atom. A dash “—” in the L column means that L is a direct bond. Unless otherwise indicated, the configuration of substituents about that double bond in the above structure are as shown.

INDEX TABLE G

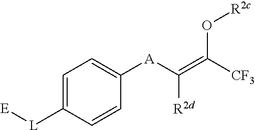

| Cmpd. No. | E | L | A | R^{2d} | R^{2c} | NMR | MS |
|---|---|---|---|---|---|---|---|
| 58 | 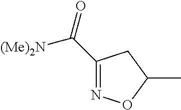 | — | O | H | CH₂CH₃ | * | |
| 60 | CH₃S(═O)₂ | — | O | H | CH₂CH₃ | * | |
| 61 | (Me)₂NS(═O)₂ | — | O | H | CH₂CH₃ | * | |
| 62 | CH₃CH₂S(═O)₂ | CH₂ | O | H | CH₂CH₃ | * | |
| 72 | HOC(═O) | — | O | H | CH₂CH₃ | * | 275 (M − 1) |
| 73 | i-PrOC(═O) | — | O | H | CH₂CH₃ | * | 318 (M + 1) |
| 74 | CH₂═CHCH₂OC(═O) | — | O | H | CH₂CH₃ | * | 317 (M + 1) |
| 75 | CH≡CCH₂OC(═O) | — | O | H | CH₂CH₃ | * | 313 (M − 1) |
| 79 | CH₃C(═O) | — | O | H | CH₂CH₃ | * | |
| 83 | 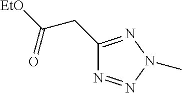 | CH₂ | O | H | CH₂CH₃ | * | 401 (M + 1) |
| 84 | 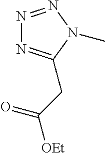 | CH₂ | O | H | CH₂CH₃ | * | 401 (M + 1) |
| 85 | 5-(CH₃OC(═O))-1H-imidazol-1-yl | CH₂ | O | H | CH₂CH₃ | * | 371 (M + 1) |
| 86 | 4-(CH₃OC(═O))-1H-imidazol-1-yl | CH₂ | O | H | CH₂CH₃ | * | 371 (M + 1) |
| 87 | 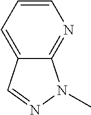 | CH₂ | O | H | CH₂CH₃ | * | 364 (M + 1) |
| 88 | 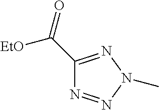 | CH₂ | O | H | CH₂CH₃ | * | 364 (M + 1) |
| 90 | 3-(EtOC(═O)-5-(MeO))-1H-pyrazol-1-yl | CH₂ | O | H | CH₂CH₃ | * | 415 (M + 1) |
| 91 | 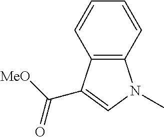 | CH₂ | O | H | CH₂CH₃ | * | 420 (M + 1) |
| 92 | 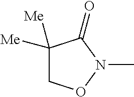 | CH₂ | O | H | CH₂CH₃ | * | 360 (M + 1) |
| 94 | n-PrOC(═O) | — | O | H | CH₂CH₃ | * | |
| 95 | EtOC(═O) | — | O | H | CH₂CH₃ | * | 305 (M + 1) |
| 96 | EtOC(═O)CH═CHCH₂O | — | O | H | CH₂CH₃ | * | |
| 99 | NH₂C(═O) | — | O | H | CH₂CH₃ | * | 274 (M + 1) |
| 100 | c-PrNHC(═O) | — | O | H | CH₂CH₃ | * | 316 (M + 1) |
| 101 | i-PrNHC(═O) | — | O | H | CH₂CH₃ | * | 318 (M + 1) |
| 102 | CH≡CCH₂NHC(═O) | — | O | H | CH₂CH₃ | * | 314 (M + 1) |
| 103 | CH₂═CHCH₂NHC(═O) | — | O | H | CH₂CH₃ | * | 316 (M + 1) |
| 104 | n-PrNHC(═O) | — | O | H | CH₂CH₃ | * | 318 (M + 1) |
| 105 | MeNHC(═O) | — | O | H | CH₂CH₃ | * | 290 (M + 1) |
| 109 | t-BuOC(═O)NH | CH₂ | O | H | CH₂CH₃ | | 261 (M + 1) |
| 110 | NH₂ | CH₂ | O | H | CH₂CH₃ | | 262 (M + 1) |
| 111 | EtOC(═O)NH | CH₂ | O | H | CH₂CH₃ | * | |
| 113 | CF₃CH₂C(═O)NH | CH₂ | O | H | CH₂CH₃ | * | |
| 114 | MeOCH(CH₃)C(═O)NH | CH₂ | O | H | CH₂CH₃ | * | |
| 119 | 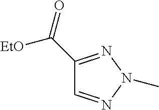 | CH₂ | O | H | CH₂CH₃ | * | 386 (M + 1) |
| 120 | 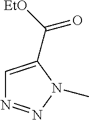 | CH₂ | O | H | CH₂CH₃ | * | 386 (M + 1) |
| 121 | 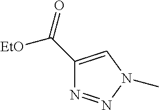 | CH₂ | O | H | CH₂CH₃ | * | 386 (M + 1) |
| 143 | 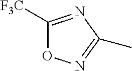 | — | O | H | CH₂CH₃ | * | |
| 144 | N≡C | — | O | H | CH₂CH₃ | * | |
| 148 | HC(═O) | — | O | H | CH₂CH₃ | | |
| 149 | EtS(═O)₂NH | CH₂ | O | H | CH₂CH₃ | * | |
| 150 | EtC(═O)NH | CH₂ | O | H | CH₂CH₃ | * | |
| 152 | 3,5-di-Me-1H-pyrazol-1-yl | CH₂ | O | H | CH₂CH₃ | * | 341 (M + 1) |
| 153 | 4-(EtOC(═O))-1H-imidazol-1-yl | CH₂ | O | H | CH₂CH₃ | * | 385 (M + 1) |
| 154 | N≡C | CH₂ | O | H | CH₂CH₃ | * | |
| 155 | N≡CS | CH₂ | O | H | CH₂CH₃ | * | |
| 156 | 1H-imidazol-1-yl | CH₂ | O | H | CH₂CH₃ | * | 313 (M + 1) |
| 184 | 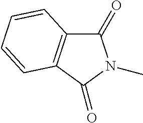 | CH₂ | O | H | CH₂CH₃ | * | |
| 193 | NH₂C(═O)O | CH₂ | O | H | CH₂CH₃ | * | |
| 195 | OH | CH₂ | O | H | CH₂CH₃ | * | |
| 196 | EtNHC(═O)O | CH₂ | O | H | CH₂CH₃ | * | |
| 210 | i-PrC(═O)NH | CH₂ | O | H | CH₂CH₃ | * | 332 (M + 1) |
| 211 | c-PrC(═O)NH | CH₂ | O | H | CH₂CH₃ | | 331 (M + 1) |
| 224 | 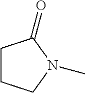 | CH₂ | O | H | CH₂CH₃ | * | 330 (M + 1) |
| 225 | 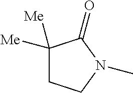 | CH₂ | O | H | CH₂CH₃ | * | 358 (M + 1) |
| 226 | 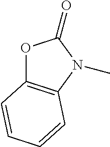 | CH₂ | O | H | CH₂CH₃ | * | 380 (M + 1) |
| 230 | MeOC(═O) | — | O | H | CH₂CH₃ | * | 291 (M + 1) |
| 268 | 4-(EtOC(═O))-1-piperidinyl | CH₂ | O | H | CH₂CH₃ | * | 402 (M + 1) |
| 269 | 3-(EtOC(═O))-1-piperidinyl | CH₂ | O | H | CH₂CH₃ | * | 402 (M + 1) |
| 270 | 4-(EtOC(═O))-pyridin-1-yl | CH₂ | O | H | CH₂CH₃ | * | |
| [Note 7] | | | | | | | |
| 271 | 3-(EtOC(═O))-pyridin-1-yl | CH₂ | O | H | CH₂CH₃ | * | |
| [Note 7] | | | | | | | |
| 278 | 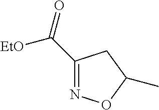 | — | O | H | CH₂CH₃ | * | |
| 279 | NO₂ | — | O | H | CH₂CH₃ | * | |
| 287 | 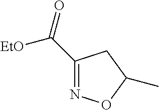 | CH₂ | O | H | CH₂CH₃ | * | |
| 288 | 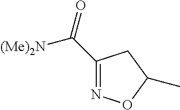 | CH₂ | O | H | CH₂CH₃ | * | |
| 297 | 4-(EtOC(═O))-1H-pyrazol-1-yl-CH₂O | CH₂ | O | H | CH₂CH₃ | * | 412 (M − 1) |
| 306 | 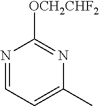 | CH₂ | O | H | CH₂CH₃ | | 405 (M + 1) |
| 307 | 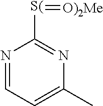 | CH₂ | O | H | CH₂CH₃ | | 403 (M + 1) |
| 308 | 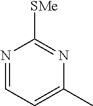 | CH₂ | O | H | CH₂CH₃ | | 371 (M + 1) |
| 309 | 4-(MeOC(═O))-1-piperidinyl | CH₂ | O | H | CH₂CH₃ | * | 388 (M + 1) |
| 310 | 3-(MeOC(═O))-1-pyrrolidinyl | CH₂ | O | H | CH₂CH₃ | * | 374 (M + 1) |
| 311 | 4-(N≡C)-1-piperidinyl | CH₂ | O | H | CH₂CH₃ | * | 355 (M + 1) |
| 312 | 4-(MeO)-1-piperidinyl | CH₂ | O | H | CH₂CH₃ | * | 360 (M + 1) |
| 313 | 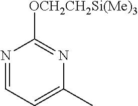 | CH₂ | O | H | CH₂CH₃ | | 441 (M + 1) |
| 364 | 4-(EtOC(═O))-1H-pyrazol-1-yl-CH₂O | — | O | H | CH₂CH₃ | | 401 (M + 1) |
| (Ex. 9) | | | | | | | |

*See Index Table M for ¹⁹F NMR data.
Note 7:
HBr salt.
A dash “—” in the L column means that L is a direct bond. Unless otherwise indicated, the configuration of substituents about that double bond in the above structure are as shown.

INDEX TABLE H

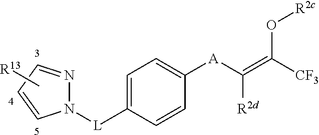

| Cmpd. No. | R¹³ | L | A | R^{2d} | R^{2c} | NMR | MS |
|---|---|---|---|---|---|---|---|
| 158 | 3-CH₃ | CH₂ | O | H | CH₂CH₃ | * | 371 (M + 1) |
| 181 | 3-CF₃ | CH₂ | O | H | CH₂CH₃ | * | 379 (M − 1) |
| 231 | 3-(EtOC(═O)) | CH₂ | O | H | CH₂CH₃ | * | |
| 241 | 5-(EtOC(═O)) | CH₂ | O | H | CH₂CH₃ | * | |
| 250 | 5-(MeOC(═O)) | CH₂ | O | H | CH₂CH₃ | * | 371 (M + 1) |
| 251 | 3-(MeOC(═O)) | CH₂ | O | H | CH₂CH₃ | * | 371 (M + 1) |
| 252 | 5-(t-BuOC(═O)) | CH₂ | O | H | CH₂CH₃ | * | 411 (M − 1) |
| 253 | 3-(t-BuOC(═O)) | CH₂ | O | H | CH₂CH₃ | * | 411 (M − 1) |
| 291 | 3-(CH₂═CHCH₂OC(═O)) | CH₂ | O | H | CH₂CH₃ | * | 397 (M + 1) |
| 292 | 3-(n-PrOC(═O)) | CH₂ | O | H | CH₂CH₃ | * | 399 (M + 1) |
| 293 | 3-(CH₃C≡CCH₂OC(═O)) | CH₂ | O | H | CH₂CH₃ | * | 409 (M + 1) |
| 299 | 3-(HOC(═O)) | CH₂ | O | H | CH₂CH₃ | * | |
| 325 | 3-(HC≡CCH₂OC(═O)) | CH₂ | O | H | CH₂CH₃ | * | 395 (M + 1) |
| 326 | 3-(CH₃CH₂C≡CCH₂OC(═O)) | CH₂ | O | H | CH₂CH₃ | * | 423 (M + 1) |
| 327 | 3-(i-PrOC(═O)) | CH₂ | O | H | CH₂CH₃ | * | 399 (M + 1) |
| 356 | 3-(n-BuOC(═O)) | CH₂ | O | H | CH₂CH₃ | * | 413 (M + 1) |
| 357 | 3-(i-BuOC(═O)) | CH₂ | O | H | CH₂CH₃ | * | 413 (M + 1) |
| 358 | 3-(CH₂═C(Me)CH₂OC(═O)) | CH₂ | O | H | CH₂CH₃ | * | 411 (M + 1) |
| 359 | 3-(CH₃C(═O)CH₂OC(═O)) | CH₂ | O | H | CH₂CH₃ | * | 413 (M + 1) |
| 360 | 3-(MeOCH₂CH₂OC(═O)) | CH₂ | O | H | CH₂CH₃ | * | 415 (M + 1) |
| 361 | 3-(c-BuCH₂OC(═O)) | CH₂ | O | H | CH₂CH₃ | * | 425 (M + 1) |
| 362 | 3-(ClCH₂CH₂CH₂OC(═O)) | CH₂ | O | H | CH₂CH₃ | * | 433 (M + 1) |

*See Index Table M for ¹⁹F NMR data.
Unless otherwise indicated, the configuration of substituents about that double bond in the above structure are as shown.

INDEX TABLE I

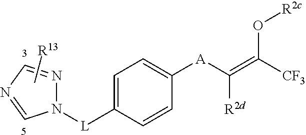

| Cmpd. | | | | | | | |
|---|---|---|---|---|---|---|---|
| No. | R¹³ | L | A | R^{2d} | R^{2c} | NMR | MS |
| 81 | 5-CN | CH₂ | O | H | CH₂CH₃ | * | 337 (M − 1) |
| 82 | 3-CN | CH₂ | O | H | CH₂CH₃ | * | 337 (M − 1) |
| 122 | 5-(MeOC(═O)) | CH₂ | O | H | CH₂CH₃ | * | 370 (M − 1) |
| 123 | 3-(MeOC(═O)) | CH₂ | O | H | CH₂CH₃ | * | 370 (M − 1) |
| 157 | — | CH₂ | O | H | CH₂CH₃ | * | 312 (M − 1) |
| 260 | 5-MeS | CH₂ | O | H | CH₂CH₃ | * | 358 (M − 1) |
| 261 | 3-MeS | CH₂ | O | H | CH₂CH₃ | * | 360 (M + 1) |

*See Index Table M for ¹⁹F NMR data.
A dash “—” in the R¹³ column means no R¹³ substituent is present and the remaining carbon valences are occupied by hydrogen atoms. Unless otherwise indicated, the configuration of substituents about that double bond in the above structure are as shown.

INDEX TABLE J

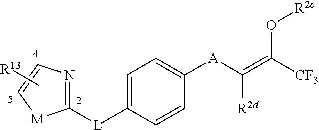

| Cmpd. | | | | | | | | |
|---|---|---|---|---|---|---|---|---|
| No. | R¹³ | M | L | A | R^{2d} | R^{2c} | NMR | MS |
| 129 | 4-(CF₃CH₂NHC(═O)) | S | CH₂ | O | H | CH₂CH₃ | * | |
| 130 | 4-(c-PrCH₂NHC(═O)) | S | CH₂ | O | H | CH₂CH₃ | * | |
| 254 | 5-(c-PrCH₂NHC(═O)) | S | CH₂ | O | H | CH₂CH₃ | | 427 (M + 1) |
| 255 | 5-(CHF₂CH₂NHC(═O)) | O | CH₂ | O | H | CH₂CH₃ | | 421 (M + 1) |
| 258 | 5-(c-PrCH₂NHC(═O)) | O | CH₂ | O | H | CH₂CH₃ | | 411 (M + 1) |
| 276 | 5-(CHF₂CH₂NHC(═O)) | S | CH₂ | O | H | CH₂CH₃ | | 437 (M + 1) |

*See Index Table M for ¹⁹F NMR data.
Unless otherwise indicated, the configuration of substituents about that double bond in the above structure are as shown.

INDEX TABLE K

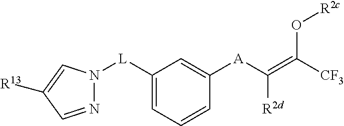

| Cmpd. No. | R¹³ | L | R^{2d} | R^{2c} | NMR | MS |
|---|---|---|---|---|---|---|
| 265 (Ex. 7) | EtOC(═O) | CH₂ | H | CH₂CH₃ | * | 385 (M + 1) |
| 300 | CH₃C≡CCH₂OC(═O) | CH₂ | H | CH₂CH₃ | * | 409 (M + 1) |
| 304 | EtOC(═O) | CH(Me) | H | CH₂CH₃ | | 399 (M + 1) |
| 317 | EtOC(═O) | — | H | CH₂CH₃ | | 371 (M + 1) |
| 323 | HOC(═O) | CH₂ | H | CH₂CH₃ | * | 357 (M + 1) |
| 324 | EtOC(═O) | CH₂ | H | n-Pr | | 399 (M + 1) |
| 330 | MeOC(═O) | CH₂ | H | CH₂CH₃ | * | 371 (M + 1) |
| 331 | n-PrOC(═O) | CH₂ | H | CH₂CH₃ | * | 399 (M + 1) |
| 332 | i-PrOC(═O) | CH₂ | H | CH₂CH₃ | * | 399 (M + 1) |
| 333 | CH₂═CHCH₂OC(═O) | CH₂ | H | CH₂CH₃ | * | 397 (M + 1) |
| 334 | i-BuOC(═O) | CH₂ | H | CH₂CH₃ | * | 413 (M + 1) |
| 335 | CH₂═C(Me)CH₂OC(═O) | CH₂ | H | CH₂CH₃ | * | 411 (M + 1) |
| 336 | CH≡CCH₂OC(═O) | CH₂ | H | CH₂CH₃ | * | 395 (M + 1) |
| 337 | CH₃C(═O)CH₂OC(═O) | CH₂ | H | CH₂CH₃ | * | 413 (M + 1) |
| 338 | Cl(CH₂)₃OC(═O) | CH₂ | H | CH₂CH₃ | * | 433 (M + 1) |
| 339 | n-BuOC(═O) | CH₂ | H | CH₂CH₃ | * | 413 (M + 1) |
| 340 | CH₃O(CH₂)₂OC(═O) | CH₂ | H | CH₂CH₃ | * | 415 (M + 1) |
| 341 | c-PrCH₂OC(═O) | CH₂ | H | CH₂CH₃ | * | 411 (M + 1) |
| 342 | c-BuCH₂OC(═O) | CH₂ | H | CH₂CH₃ | * | 425 (M + 1) |
| 343 | EtNHC(═O) | CH₂ | H | CH₂CH₃ | * | 382 (M + 1) |
| 345 | EtOC(═O) | CH₂CH₂ | H | CH₂CH₃ | * | 399 (M + 1) |
| 347 | CHF₂CH₂OC(═O) | CH₂ | H | CH₂CH₃ | * | 421 (M + 1) |
| 348 | CF₃CH₂CH₂OC(═O) | CH₂ | H | CH₂CH₃ | * | 453 (M + 1) |
| 349 | CF₂═CFCH₂CH₂OC(═O) | CH₂ | H | CH₂CH₃ | * | 465 (M + 1) |
| 350 | (Me)₂CH(CH₂)₂OC(═O) | CH₂ | H | CH₂CH₃ | * | |
| 351 | CH₃O(CH₂)₃OC(═O) | CH₂ | H | CH₂CH₃ | * | 429 (M + 1) |
| 352 | CF₃O(CH₂)₂OC(═O) | CH₂ | H | CH₂CH₃ | * | 469 (M + 1) |
| 355 | CF₃(CH₂)₃OC(═O) | CH₂ | H | CH₂CH₃ | * | 467 (M + 1) |
| 365 | EtOC(═O) | CH₂ | H | CH₃ | * | |

*See Index Table M for ¹⁹F NMR data.
A dash “—” in the L column means that L is a direct bond. Unless otherwise indicated, the configuration of substituents about that double bond in the above structure are as shown.

INDEX TABLE L

| | | | MS | |
|---|---|---|---|---|
| Cmpd. No. | Structure | NMR | (M + 1) | m.p. (° C.) |
| 63 [Note 6] | 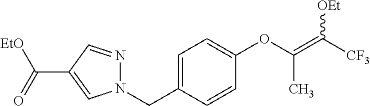 [SMILES: CCOC(=O)c1cnn(Cc2ccc(OC(C)=C(OCC)C(F)(F)F)cc2)c1] | * | | |
| 124 | 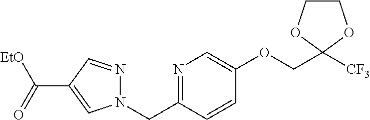 [SMILES: CCOC(=O)c1cnn(Cc2ccc(OCC3(C(F)(F)F)OCCO3)cn2)c1] | | 402 | |
| 146 | 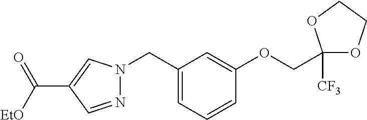 [SMILES: CCOC(=O)c1cnn(Cc2cccc(OCC3(C(F)(F)F)OCCO3)c2)c1] | * | 401 | |
| 147 | 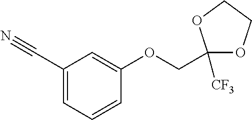 [SMILES: N#Cc1cccc(OCC2(C(F)(F)F)OCCO2)c1] | * | 274 | |
| 165 | 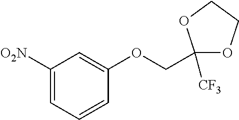 [SMILES: O=[N+]([O-])c1cccc(OCC2(C(F)(F)F)OCCO2)c1] | * | | |
| 168 | 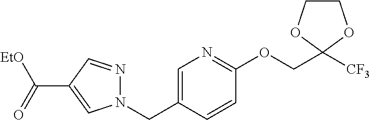 [SMILES: CCOC(=O)c1cnn(Cc2ccc(OCC3(C(F)(F)F)OCCO3)nc2)c1] | | 402 | |
| 262 | 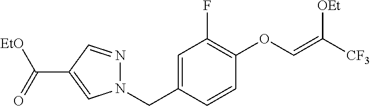 [SMILES: CCOC(=O)c1cnn(Cc2ccc(O/C=C(\OCC)C(F)(F)F)c(F)c2)c1] | * | | |
| 263 | 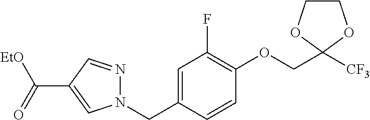 [SMILES: CCOC(=O)c1cnn(Cc2ccc(OCC3(C(F)(F)F)OCCO3)c(F)c2)c1] | * | | |
| 264 | 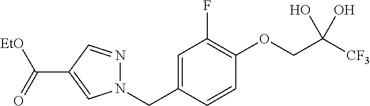 [SMILES: CCOC(=O)c1cnn(Cc2ccc(OCC(O)(O)C(F)(F)F)c(F)c2)c1] | * | | |
| 266 (Ex. 6) | 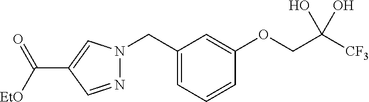 [SMILES: CCOC(=O)c1cnn(Cc2cccc(OCC(O)(O)C(F)(F)F)c2)c1] | * | | |
| 273 | 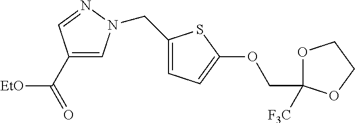 [SMILES: CCOC(=O)c1cnn(Cc2ccc(OCC3(C(F)(F)F)OCCO3)s2)c1] | ** | | |
| 301 | 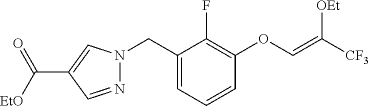 [SMILES: CCOC(=O)c1cnn(Cc2cccc(O/C=C(\OCC)C(F)(F)F)c2F)c1] | | 403 | |
| 302 | 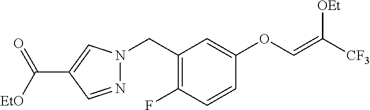 [SMILES: CCOC(=O)c1cnn(Cc2cc(O/C=C(\OCC)C(F)(F)F)ccc2F)c1] | | 403 | |
| 303 | 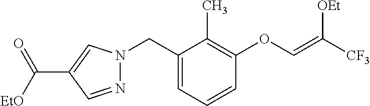 [SMILES: CCOC(=O)c1cnn(Cc2cccc(O/C=C(\OCC)C(F)(F)F)c2C)c1] | | 399 | |
| 305 | 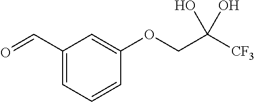 [SMILES: O=Cc1cccc(OCC(O)(O)C(F)(F)F)c1] | * | | |
| 320 | 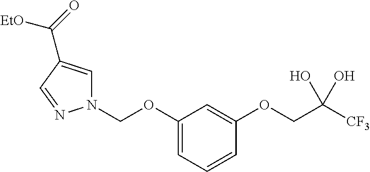 [SMILES: CCOC(=O)c1cnn(COc2cccc(OCC(O)(O)C(F)(F)F)c2)c1] | | 391 | |
| 321 | 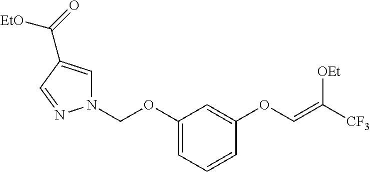 [SMILES: CCOC(=O)c1cnn(COc2cccc(O/C=C(\OCC)C(F)(F)F)c2)c1] | * | | |
| 344 | 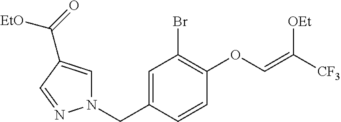 [SMILES: CCOC(=O)c1cnn(Cc2ccc(O/C=C(\OCC)C(F)(F)F)c(Br)c2)c1] | * | 463 | 88-89 |
| 346 | 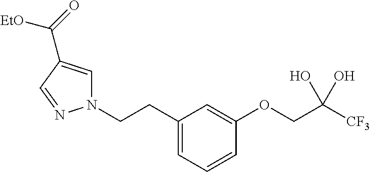 [SMILES: CCOC(=O)c1cnn(CCc2cccc(OCC(O)(O)C(F)(F)F)c2)c1] | * | 389 | |
| 353 | 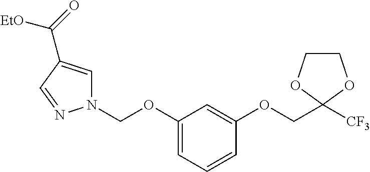 [SMILES: CCOC(=O)c1cnn(COc2cccc(OCC3(C(F)(F)F)OCCO3)c2)c1] | * | 417 | |

Note 6:
3:2 mixture of geometric isomers.
*See Index Table M for ¹⁹F NMR data.
**See Index Table N for ¹H NMR data.

INDEX TABLE M

| Cmpd. | |
|---|---|
| No. | ¹⁹F NMR Data^{a} |
| 1 | δ −84.92 (s). |
| 2 | δ (DMSO-d₆) −81.80 (s). |
| 3 | δ −81.39 (s). |
| 4 | δ (DMSO-d₆) −81.50 (s). |
| 5 | δ −82.61 (s), −75.48 (s). |
| 6 | δ −79.03 (s). |
| 7 | δ −81.50 (s), −81.71 (s). |
| 8 | δ −81.50 (s), −81.71 (s). |
| 9 | δ −81.27 (s). |
| 10 | δ −79.64 (s). |
| 12 | δ −81.01 (s). |
| 13 | δ −81.38 (s). |
| 14 | δ −81.39 (s). |
| 15 | δ −81.38 (s). |
| 16 | δ −81.39 (s). |
| 17 | δ −81.38 (s). |
| 18 | δ −81.38 (s). |
| 20 | δ −78.49 (s). |
| 21 | δ −80.99 (s). |
| 22 | δ −81.39 (s). |
| 23 | δ −81.37 (s). |
| 24 | δ −81.37 (s). |
| 25 | δ −81.37 (s). |
| 26 | δ −81.39 (s). |
| 27 | δ −81.41 (s). |
| 28 | δ −81.38 (s). |
| 29 | δ −84.92 (s). |
| 31 | δ (DMSO-d₆) −81.91 (s). |
| 32 | δ −81.39 (s). |
| 33 | δ (DMSO-d₆) −81.83 (s). |
| 34 | δ (DMSO-d₆) −81.89 (s). |
| 35 | δ −84.94 (s). |
| 36 | δ (DMSO-d₆) −81.37 (s). |
| 37 | δ (DMSO-d₆) −81.82 (s). |
| 39 | δ −84.93 (s). |
| 40 | δ (acetone-d₆) −83.12 (s). |
| 41 | δ −84.81 (s). |
| 42 | δ (DMSO-d₆) −81.82 (s). |
| 43 | δ (DMSO-d₆) −81.82 (s). |
| 44 | δ −84.21 (s). |
| 45 | δ (DMSO-d₆) −81.82 (s), −72.33 (t). |
| 46 | δ (DMSO-d₆) −81.83 (s), −82.96 (s), −122.29 (t). |
| 47 | δ (DMSO-d₆) −81.82 (s). |
| 48 | δ (DMSO-d₆) −81.82 (s). |
| 49 | δ (DMSO-d₆) −81.82 (s). |
| 50 | δ (DMSO-d₆) −81.82 (s), −138.53 (m), −139.66 (m). |
| 51 | δ −87.93 (s). |
| 52 | δ −76.89 (s). |
| 53 | δ −70.09 (s). |
| 54 | δ −69.99 (s). |
| 55 | δ −69.95 (s). |
| 56 | δ −69.73 (s). |
| 57 | δ −69.75 (s). |
| 58 | δ −70.10 (s). |
| 60 | δ −70.30 (s). |
| 61 | δ −70.26 (s). |
| 62 | δ −70.15 (s). |
| 63 | δ −63.30 (s), −63.67 (s). |
| 64 | δ −70.13 (s). |
| 65 | δ −70.03 (s). |
| 66 | δ −85.74 (s). |
| 67 | δ −82.36 (s). |
| 72 | δ −70.24 (s). |
| 73 | δ −70.20 (s). |
| 74 | δ −70.22 (s). |
| 75 | δ −70.22 (s). |
| 79 | δ −70.22 (s). |
| 80 | δ −84.87 (s). |
| 81 | δ −70.17 (s). |
| 82 | δ −70.14 (s). |
| 83 | δ −70.17 (s). |
| 84 | δ −70.16 (s). |
| 85 | δ −70.14 (s). |
| 86 | δ −70.12 (s). |
| 87 | δ −70.11 (s). |
| 88 | δ −70.19 (s). |
| 89 | δ −70.13 (s). |
| 90 | δ −70.16 (s). |
| 91 | δ −70.12 (s). |
| 92 | δ −70.13 (s). |
| 93 | δ −81.42 (s). |
| 94 | δ −70.21 (s). |
| 95 | δ −70.21 (s). |
| 96 | δ −69.98 (s). |
| 97 | δ −84.95 (s). |
| 98 | δ −81.40 (s). |
| 99 | δ −70.19 (s). |
| 100 | δ −70.16 (s). |
| 101 | δ −70.15 (s). |
| 102 | δ −70.18 (s). |
| 103 | δ −70.16 (s). |
| 104 | δ −70.16 (s). |
| 105 | δ −70.16 (s). |
| 111 | δ −70.08 (s). |
| 112 | δ −81.43 (s). |
| 113 | δ −63.01 (s), −70.10 (s). |
| 114 | δ −70.09 (s). |
| 116 | δ −70.14 (s). |
| 119 | δ −70.16 (s). |
| 120 | δ −70.15 (s). |
| 121 | δ −70.16 (s). |
| 122 | δ −70.14 (s). |
| 123 | δ −70.15 (s). |
| 126 | δ −81.38 (s). |
| 127 | δ −81.38 (s). |
| 129 | δ −70.06 (s), −72.25 (s). |
| 130 | δ 70.06 (s). |
| 131 | δ −84.93 (s). |
| 132 | δ −70.05 (s). |
| 133 | δ −81.40 (s). |
| 134 | δ −81.40 (s), −73.70 (s). |
| 140 | δ −69.16 (s). |
| 164 | δ −81.33 (s). |
| 165 | δ −81.34 (s). |
| 166 | δ −81.38 (s). |
| 167 | δ −69.60 (s). |
| 141 | δ −81.28 (s). |
| 142 | δ −84.83 (s). |
| 143 | δ −60.46 (s), −70.25 (s). |
| 144 | δ −70.30 (s). |
| 145 | δ −69.79 (s). |
| 146 | δ −81.37 (s). |
| 147 | δ −81.36 (s). |
| 149 | δ −70.07 (s). |
| 150 | δ −70.07 (s). |
| 151 | δ −70.09 (s). |
| 152 | δ −70.09 (s). |
| 153 | δ −70.12 (s). |
| 154 | δ −70.10 (s). |
| 155 | δ −70.05 (s). |
| 156 | δ −70.05 (s). |
| 157 | δ −70.19 (s). |
| 158 | δ −70.10 (s). |
| 160 | δ −81.44 (s). |
| 161 | δ −81.38 (s). |
| 162 | δ (DMSO-d₆) −80.02 (s) |
| 163 | δ −81.38 (s). |
| 169 | δ −81.38 (s). |
| 170 | δ −81.38 (s). |
| 171 | δ −81.37 (s). |
| 172 | δ −81.37 (s). |
| 174 | δ −80.00 (s). |
| 176 | δ −81.40 (s). |
| 177 | δ −84.85 (s). |
| 179 | δ −62.79 (s), −81.40 (s). |
| 180 | δ −70.10 (s). |
| 181 | δ −61.85 (s). |
| 182 | δ −56.40 (s). |
| 183 | δ −70.09 (s). |
| 184 | δ −70.11 (s). |
| 189 | δ −81.42 (s). |
| 190 | δ −81.38 (s). |
| 191 | δ −81.42 (s). |
| 192 | δ −81.39 (s). |
| 193 | δ −70.01 (s). |
| 195 | δ −70.04 (s). |
| 196 | δ −70.08 (s).. |
| 197 | δ −72.38 (s), −81.38 (s). |
| 198 | δ −81.38 (s). |
| 199 | δ −81.38 (s). |
| 200 | δ −81.38 (s). |
| 201 | δ −81.38 (s). |
| 202 | δ −81.39 (s). |
| 203 | δ −81.39 (s). |
| 204 | δ −81.38 (s). |
| 205 | δ −70.14 (s). |
| 206 | δ −84.95 (s). |
| 209 | δ −81.36 (s). |
| 210 | δ −70.08 (s). |
| 214 | δ −81.43 (s). |
| 224 | δ −70.09 (s). |
| 225 | δ −70.08 (s). |
| 226 | δ −70.10 (s). |
| 227 | δ −81.57 (s), −84.95 (s). |
| 228 | δ −81.39 (s). |
| 230 | δ −70.21 (s). |
| 231 | δ −70.12 (s). |
| 232 | δ −81.39 (s). |
| 233 | δ −84.87 (s). |
| 234 | δ −84.95 (s). |
| 235 | δ −81.45 (s). |
| 236 | δ −81.46 (s). |
| 237 | δ −81.44 (s). |
| 239 | δ −81.42 (s). |
| 240 | δ −81.39 (s) |
| 241 | δ −70.11 (s). |
| 245 | δ −68.61 (s). |
| 249 | δ −81.39 (s). |
| 250 | δ −70.11 (s). |
| 251 | δ −70.13 (s). |
| 252 | δ −70.09 (s). |
| 253 | δ −70.11 (s). |
| 260 | δ −70.12 (s). |
| 261 | δ −70.12 (s). |
| 262 | δ −70.33 (s). |
| 263 | δ −81.60 (s), −132.13 (s). |
| 264 | δ −84.92 (s). |
| 265 | δ −70.09 (s). |
| 266 | δ −84.88 (s). |
| 267 | δ −70.13 (s). |
| 268 | δ −70.03 (s). |
| 269 | δ −70.03 (s). |
| 270 | δ −70.12 (s). |
| 271 | δ −70.12 (s). |
| 272 | δ −69.99 (s). |
| 278 | δ −70.11 (s). |
| 279 | δ −70.31 (s). |
| 280 | δ −84.80 (s). |
| 284 | δ −81.38 (s). |
| 285 | δ −81.39 (s). |
| 286 | δ −84.94 (s). |
| 287 | δ −70.05 (s). |
| 288 | δ −70.04 (s). |
| 289 | δ −81.39 (s). |
| 290 | δ −81.37 (s). |
| 291 | δ −70.12 (s). |
| 292 | δ −70.13 (s). |
| 293 | δ −70.13 (s). |
| 294 | δ −70.01 (s). |
| 296 | δ −81.38 (s). |
| 297 | δ −70.01 (s). |
| 298 | δ −70.05 (s), −74.96 (s). |
| 299 | δ −70.12 (s). |
| 300 | δ −70.10 (s). |
| 305 | δ −84.85 (s). |
| 309 | δ −70.02 (s). |
| 310 | δ −70.04 (s). |
| 311 | δ −70.01 (s). |
| 312 | δ −70.03 (s). |
| 318 | δ −81.38 (s). |
| 319 | δ −79.29 (s), 82.67 (s). |
| 321 | δ −68.60 (s). |
| 322 | δ −81.70 (s). |
| 323 | δ −70.10 (s). |
| 325 | δ −70.11 (s). |
| 326 | δ −70.13 (s). |
| 327 | δ −70.13 (s). |
| 328 | δ −68.54 (s). |
| 329 | δ −77.08 (s). |
| 330 | δ −70.09 (s). |
| 331 | δ −70.10 (s). |
| 332 | δ −70.08 (s). |
| 333 | δ −70.09 (s). |
| 334 | δ −70.09 (s). |
| 335 | δ −70.09 (s). |
| 336 | δ −70.08 (s). |
| 337 | δ −70.08 (s). |
| 338 | δ −70.06 (s). |
| 339 | δ −70.10 (s). |
| 340 | δ −70.09 (s). |
| 341 | δ −70.09 (s). |
| 342 | δ −70.11 (s). |
| 343 | δ −70.11 (s). |
| 344 | δ −70.50 (s). |
| 345 | δ −68.59 (s). |
| 346 | δ −81.82 (s). |
| 347 | δ −70.09 (s), −125.54 (s). |
| 348 | δ −64.93 (s), −70.09 (s). |
| 349 | δ −70.13 (s), −103.21 (s), −123.98 (s), −175.15 (s). |
| 350 | δ −70.09 (s). |
| 351 | δ −70.09 (s). |
| 352 | δ −60.95 (s), −70.12 (s). |
| 353 | δ −81.41 (s). |
| 354 | δ −70.03 (s). |
| 355 | δ −66.44 (s), −70.09 (s). |
| 356 | δ −70.14 (s). |
| 357 | δ −70.13 (s). |
| 358 | δ −70.12 (s). |
| 359 | δ −70.11 (s). |
| 360 | δ −70.12 (s). |
| 361 | δ −70.13 (s). |
| 362 | δ −70.11 (s). |
| 365 | δ −70.05 (s). |

^{a19}F NMR spectra are reported in ppm relative to CF₃CCl₃, in CDCl₃ solution unless indicated otherwise. Couplings are designated by (s)-singlet, (t)-triplet and (m)-multiplet.

INDEX TABLE N

| Compound | |
|---|---|
| No. | ¹H NMR Data^{a} |
| 273 | (CDCl₃): δ 1.36-1.32 (t, 3H), 4.21 (s, 6H), 4.32-4.25 (q, 2H), 5.32 (s, 2H), 6.20 (s, 1H), |
| | 6.70 (s, 1H), 7.89 (s, 1H), 7.95 (s, 1H). |
| 366 | (DMSO-d₆): δ 1.26 (t, 3H), 3.98 (s, 2H), 4.21 (q, 2H), 6.03 (s, 2H), 6.86-6.94 (m, 2H), |
| | 6.95-7.06 (m, 2H), 7.27 (s, 2H), 7.94 (s, 1H), 8.53 (s, 1H). |

^{a1}H NMR data are reported in ppm downfield from tetramethylsilane. Couplings are designated by (s)-singlet, (t)-triplet, (q)-quartet.

### BIOLOGICAL EXAMPLES OF THE INVENTION

General protocol for preparing test suspensions for Tests A-C: the test compounds were first dissolved in acetone in an amount equal to 3% of the final volume and then suspended at the desired concentration (in ppm) in acetone and purified water (50/50 mix by volume) containing 250 ppm of the surfactant PEG400 (polyhydric alcohol esters). The resulting test suspensions were then used in Tests A-C.

### Test A

The test solution was sprayed to the point of run-off on soybean seedlings. The following day the seedlings were inoculated with a spore suspension of Phakopsora pachyrhizi (the causal agent of Asian soybean rust) and incubated in a saturated atmosphere at 22° C. for 24 h, and then moved to a growth chamber at 22° C. for 8 days, after which time visual disease ratings were made.

### Test B

The test solution was sprayed to the point of run-off on wheat seedlings. The following day the seedlings were inoculated with a spore suspension of Puccinia recondita f. sp. tritici (the causal agent of wheat leaf rust) and incubated in a saturated atmosphere at 20° C. for 24 h, and then moved to a growth chamber at 20° C. for 6 days, after which time disease ratings were made.

### Test C

The test solution was sprayed to the point of run-off on grape seedlings. The following day the seedlings were inoculated with a spore suspension of Uncinula necator (the causal agent of grape powdery mildew) and incubated in a growth chamber at 20° C. for 12 days, after which time disease ratings were made.

Results for Tests A-C are given in Table A. In the Table, a rating of 100 indicates 100% disease control and a rating of 0 indicates no disease control (relative to the controls). An asterisk “*” or a double asterisk “**” next to the rating value indicates a 50 ppm or 10 ppm test suspension was used, respectively. A dash (-) indicates the compound was not tested.

TABLE A

| Cmpd. No. | Rate in ppm | Test A | Test B | Test C |
|---|---|---|---|---|
| 1 | 250 | 100 | 91 | — |
| 2 | 10 | 77 | 32 | — |
| 3 | 10 | 100 | 0 | — |
| 4 | 10 | 100 | 89 | 100* |
| 5 | 10 | 0 | 0 | — |
| 6 | 10 | 0 | 0 | — |
| 7 | 10 | 94 | 0 | — |
| 8 | 10 | 96 | 9 | — |
| 9 | 10 | 98 | 86 | — |
| 10 | 10 | 100 | 86 | — |
| 12 | 10 | 13 | 0 | — |
| 13 | 10 | 99 | 0 | — |
| 14 | 10 | 100 | 0 | — |
| 15 | 10 | 100 | 0 | — |
| 16 | 10 | 99 | 0 | — |
| 17 | 10 | 100 | 0 | — |
| 18 | 10 | 100 | 0 | — |
| 20 | 10 | 98 | 86 | — |
| 21 | 10 | 84 | 23 | — |
| 22 | 10 | 38 | 0 | — |
| 23 | 10 | 0 | 19 | — |
| 24 | 10 | 81 | 91 | — |
| 25 | 10 | 0 | 0 | — |
| 26 | 10 | 99 | 0 | — |
| 27 | 10 | 0 | 0 | — |
| 28 | 10 | 0 | 0 | — |
| 29 | 10 | 84 | 28 | — |
| 30 | 10 | 0 | 0 | — |
| 31 | 10 | 44 | 93 | — |
| 32 | 50 | 100 | 68 | 100 |
| 33 | 10 | 96 | 86 | — |
| 34 | 10 | 0 | 80 | — |
| 35 | 10 | 99 | 32 | — |
| 36 | 10 | 70 | 0 | — |
| 37 | 10 | 99 | 86 | — |
| 38 | 10 | 97 | 89 | — |
| 39 | 10 | 91 | 79 | — |
| 40 | 10 | 91 | 68 | — |
| 41 | 10 | 92 | 23 | — |
| 42 | 10 | 99 | 85 | — |
| 43 | 10 | 96 | 60 | — |
| 44 | 10 | 97 | 85 | — |
| 45 | 10 | 87 | 85 | — |
| 46 | 10 | 64 | 85 | — |
| 47 | 50 | 100 | 95 | — |
| 48 | 50 | 100 | 91 | — |
| 49 | 10 | 0 | 86 | — |
| 50 | 10 | 54 | 91 | — |
| 51 | 10 | 0 | 0 | — |
| 52 | 10 | 99 | 0 | — |
| 53 | 50 | 100 | 67 | — |
| 54 | 10 | 0 | 0 | — |
| 55 | 10 | 0 | 0 | — |
| 56 | 10 | 38 | 0 | — |
| 57 | 250 | 100 | 0 | 100* |
| 58 | 10 | 0 | 0 | — |
| 60 | 10 | 0 | 0 | — |
| 61 | 10 | 0 | 0 | — |
| 62 | 10 | 0 | 0 | — |
| 63 | 10 | 0 | 0 | — |
| 64 | 250 | 100 | 99 | 100** |
| 65 | 10 | 100 | 0 | — |
| 66 | 50 | 92 | 89 | — |
| 67 | 50 | 65 | 0 | — |
| 68 | 10 | 100 | 95 | — |
| 69 | 10 | 98 | 96 | — |
| 70 | 250 | 100 | 100 | — |
| 71 | 10 | 100 | 96 | — |
| 72 | 50 | 13 | 0 | — |
| 73 | 10 | 0 | 0 | — |
| 74 | 10 | 0 | 0 | — |
| 75 | 10 | 0 | 0 | — |
| 76 | 10 | 0 | 0 | — |
| 77 | 10 | 0 | 0 | — |
| 78 | 10 | 94 | 0 | — |
| 79 | 50 | 0 | 0 | — |
| 80 | 50 | 99 | 100 | — |
| 81 | 10 | 0 | 0 | — |
| 82 | 10 | 97 | 0 | — |
| 83 | 10 | 82 | 0 | — |
| 84 | 10 | 0 | 0 | — |
| 85 | 10 | 89 | 0 | — |
| 86 | 10 | 99 | 0 | — |
| 87 | 10 | 0 | 0 | — |
| 88 | 10 | 65 | 0 | — |
| 89 | 10 | 0 | 0 | — |
| 90 | 10 | 100 | 0 | — |
| 91 | 10 | 0 | 0 | — |
| 92 | 10 | 0 | 0 | — |
| 93 | 10 | 96 | 0 | — |
| 94 | 10 | 0 | 0 | — |
| 95 | 10 | 0 | 0 | — |
| 96 | 10 | 13 | 0 | — |
| 97 | 50 | 94 | 0 | — |
| 98 | 250 | 100 | 100 | — |
| 99 | 10 | 0 | 80 | — |
| 100 | 10 | 0 | 0 | — |
| 101 | 10 | 0 | 0 | — |
| 102 | 10 | 0 | 0 | — |
| 103 | 10 | 0 | 0 | — |
| 104 | 10 | 0 | 80 | — |
| 105 | 10 | 0 | 68 | — |
| 106 | 10 | 0 | 0 | — |
| 107 | 10 | 0 | 0 | — |
| 109 | 10 | 0 | 0 | — |
| 110 | 50 | 79 | 0 | — |
| 111 | 10 | 0 | 0 | — |
| 112 | 10 | 0 | 0 | — |
| 113 | 10 | 0 | 0 | — |
| 114 | 50 | 44 | 0 | — |
| 115 | 250 | 0 | 0 | — |
| 116 | 10 | 100 | 0 | — |
| 117 | 10 | 23 | 0 | — |
| 118 | 10 | 0 | 0 | — |
| 119 | 10 | 97 | 0 | — |
| 120 | 10 | 0 | 0 | — |
| 121 | 10 | 100 | 0 | — |
| 122 | 10 | 0 | 0 | — |
| 123 | 10 | 98 | 0 | — |
| 124 | 50 | 100 | 0 | 100 |
| 125 | 10 | 100 | 0 | — |
| 126 | 10 | 100 | 0 | 100* |
| 127 | 10 | 100 | 0 | 100* |
| 129 | 10 | 100 | 0 | — |
| 130 | 10 | 100 | 0 | — |
| 131 | 50 | 99 | 86 | — |
| 132 | 10 | 100 | 0 | 98* |
| 133 | 10 | 79 | 0 | — |
| 134 | 10 | 89 | 32 | — |
| 135 | 10 | 86 | 61 | — |
| 136 | 10 | 98 | 83 | — |
| 137 | 10 | 79 | 0 | — |
| 138 | 10 | 100 | 0 | 100* |
| 139 | 10 | 100 | 0 | — |
| 140 | 10 | 98 | 23 | — |
| 141 | 10 | 0 | 0 | — |
| 142 | 10 | 92 | 100 | — |
| 143 | 10 | 0 | 0 | — |
| 144 | 10 | 78 | 0 | — |
| 145 | 10 | 82 | 0 | — |
| 146 | 50 | 60 | 0 | — |
| 147 | 50 | 77 | 0 | — |
| 148 | 10 | 0 | 0 | — |
| 149 | 10 | 0 | 0 | — |
| 150 | 10 | 33 | 0 | — |
| 151 | 10 | 0 | 0 | — |
| 152 | 10 | 0 | 0 | — |
| 153 | 10 | 100 | 0 | — |
| 154 | 10 | 0 | 0 | — |
| 155 | 10 | 0 | 0 | — |
| 156 | 10 | 0 | 0 | — |
| 157 | 10 | 0 | 9 | — |
| 158 | 10 | 0 | 0 | — |
| 160 | 10 | 13 | 0 | — |
| 161 | 10 | 100 | 0 | — |
| 162 | 10 | 100 | 0 | 100* |
| 163 | 10 | 100 | 0 | 100* |
| 164 | 10 | 31 | 0 | — |
| 165 | 10 | 0 | 0 | — |
| 166 | 10 | 100 | 95 | — |
| 167 | 10 | 38 | 0 | — |
| 168 | 10 | 0 | 0 | — |
| 169 | 10 | 100 | 0 | — |
| 170 | 10 | 100 | 0 | 100* |
| 171 | 10 | 100 | 0 | 99* |
| 172 | 10 | 100 | 0 | — |
| 173 | 10 | 0 | 0 | — |
| 174 | 10 | 0 | 0 | — |
| 175 | 10 | 0 | 0 | — |
| 176 | 10 | 0 | 0 | — |
| 177 | 50 | 100 | 89 | — |
| 178 | 10 | 0 | 0 | — |
| 179 | 10 | 0 | 0 | — |
| 180 | 10 | 0 | 0 | — |
| 181 | 10 | 0 | 0 | — |
| 182 | 10 | 0 | 0 | — |
| 183 | 10 | 0 | 0 | — |
| 184 | 10 | 0 | 0 | — |
| 185 | 10 | 100 | 0 | 100* |
| 186 | 10 | 100 | 0 | 100* |
| 187 | 10 | 93 | 74 | — |
| 188 | 10 | 100 | 0 | — |
| 189 | 10 | 0 | 0 | — |
| 190 | 10 | 0 | 0 | — |
| 191 | 10 | 0 | 0 | — |
| 192 | 10 | 0 | 0 | — |
| 193 | 10 | 0 | 86 | — |
| 195 | 10 | 0 | 0 | — |
| 196 | 10 | 0 | 74 | — |
| 197 | 10 | 0 | 0 | — |
| 198 | 10 | 0 | 0 | — |
| 199 | 10 | 100 | 0 | — |
| 200 | 10 | 100 | 0 | — |
| 201 | 10 | 100 | 0 | — |
| 202 | 10 | 100 | 0 | — |
| 203 | 10 | 50 | 0 | — |
| 204 | 10 | 100 | 0 | — |
| 205 | 10 | 0 | 0 | — |
| 206 | 50 | 100 | 98 | — |
| 207 | 10 | 0 | 0 | — |
| 209 | 10 | 89 | 0 | — |
| 210 | 10 | 0 | 0 | — |
| 211 | 10 | 0 | 0 | — |
| 212 | 10 | 44 | 0 | — |
| 214 | 10 | 0 | 0 | — |
| 215 | 10 | 80 | 55 | — |
| 216 | 10 | 100 | 0 | 100* |
| 217 | 10 | 100 | 0 | 100* |
| 218 | 10 | 100 | 0 | 100* |
| 219 | 10 | 100 | 0 | 100* |
| 220 | 10 | 100 | 0 | 100* |
| 221 | 10 | 100 | 0 | 100* |
| 222 | 10 | 100 | 0 | — |
| 223 | 10 | 75 | 0 | — |
| 224 | 10 | 0 | 0 | — |
| 225 | 10 | 0 | 0 | — |
| 226 | 10 | 0 | 0 | — |
| 227 | 10 | 99 | 55 | — |
| 228 | 10 | 100 | 0 | — |
| 229 | 50 | 99 | 100 | — |
| 230 | 10 | 0 | 0 | — |
| 231 | 10 | 100 | 0 | — |
| 232 | 10 | 98 | 0 | — |
| 233 | 50 | 100 | 96 | — |
| 234 | 50 | 100 | 92 | — |
| 235 | 10 | 97 | 0 | — |
| 236 | 10 | 75 | 0 | — |
| 237 | 10 | 97 | 0 | — |
| 238 | 10 | 73 | 0 | — |
| 239 | 10 | 0 | 0 | — |
| 240 | 10 | 99 | 0 | — |
| 241 | 10 | 0 | 0 | — |
| 242 | 10 | 100 | 0 | — |
| 243 | 10 | 100 | 0 | — |
| 244 | 10 | 100 | 0 | — |
| 245 | 10 | 100 | 0 | 98* |
| 246 | 10 | 100 | 0 | — |
| 247 | 10 | 13 | 0 | — |
| 248 | 10 | 71 | 0 | — |
| 249 | 10 | 100 | 0 | — |
| 250 | 10 | 0 | 0 | — |
| 251 | 10 | 100 | 9 | — |
| 252 | 10 | 0 | 0 | — |
| 253 | 10 | 0 | 0 | — |
| 254 | 10 | 73 | 0 | — |
| 255 | 10 | 89 | 0 | — |
| 256 | 10 | 73 | 0 | — |
| 257 | 10 | 54 | 0 | — |
| 258 | 10 | 96 | 0 | — |
| 259 | 10 | 25 | 0 | — |
| 260 | 10 | 0 | 0 | — |
| 261 | 10 | 0 | 0 | — |
| 262 | 10 | 100 | 0 | 99* |
| 263 | 10 | 100 | 0 | — |
| 264 | 10 | 99 | 80 | — |
| 265 | 50 | 100 | 0 | 100 |
| 266 | 50 | 86 | 74 | — |
| 267 | 10 | 94 | 0 | — |
| 268 | 10 | 99 | 0 | — |
| 269 | 10 | 100 | 0 | — |
| 270 | 10 | 25 | 0 | — |
| 271 | 10 | 0 | 0 | — |
| 272 | 10 | 100 | 0 | — |
| 273 | 50 | 100 | 0 | — |
| 274 | 10 | 48 | 0 | — |
| 275 | 10 | 0 | 0 | — |
| 276 | 10 | 13 | 0 | 100* |
| 277 | 10 | 50 | 45 | — |
| 278 | 50 | 94 | 0 | — |
| 279 | 50 | 0 | 0 | — |
| 280 | 50 | 100 | 100 | — |
| 281 | 50 | 95 | 98 | — |
| 282 | 50 | 0 | 0 | — |
| 283 | 50 | 100 | 99 | — |
| 284 | 10 | 100 | 0 | — |
| 285 | 10 | 100 | 0 | — |
| 286 | 10 | 87 | 55 | — |
| 287 | 50 | 77 | 0 | — |
| 288 | 50 | 96 | 0 | — |
| 289 | 10 | 0 | 0 | — |
| 290 | — | — | — | — |
| 291 | 10 | 100 | 0 | — |
| 292 | 10 | 100 | 0 | — |
| 293 | 10 | 100 | 0 | — |
| 294 | 10 | 100 | 0 | — |
| 295 | 50 | 99 | 80 | — |
| 296 | 10 | 0 | 0 | — |
| 297 | 10 | 100 | 0 | — |
| 298 | 10 | 0 | 0 | — |
| 299 | 10 | 100 | 0 | — |
| 300 | 10 | 100 | 0 | — |
| 301 | 50 | 100 | 0 | — |
| 302 | 50 | 99 | 0 | — |
| 303 | 50 | 0 | 0 | — |
| 304 | 50 | 96 | 0 | — |
| 305 | 10 | 70 | 0 | — |
| 306 | — | — | — | — |
| 307 | 50 | 0 | 0 | — |
| 308 | 50 | 25 | 0 | — |
| 309 | 10 | 100 | 0 | — |
| 310 | 10 | 100 | 0 | — |
| 311 | 10 | 0 | 0 | — |
| 312 | 10 | 0 | 0 | — |
| 313 | 50 | 0 | 0 | — |
| 314 | 50 | 0 | 0 | — |
| 316 | 50 | 100 | 9 | — |
| 317 | — | — | — | — |
| 318 | 10 | 100 | 0 | — |
| 319 | 50 | 100 | 41 | — |
| 320 | — | — | — | — |
| 321 | — | — | — | — |
| 322 | — | — | — | — |
| 323 | 50 | 100 | 0 | — |
| 324 | 10 | 0 | 0 | — |
| 325 | 10 | 100 | 0 | — |
| 326 | 10 | 100 | 0 | — |
| 327 | 10 | 100 | 0 | — |
| 328 | — | — | — | — |
| 329 | 50 | 100 | 41 | — |
| 330 | 10 | 100 | 0 | — |
| 331 | 10 | 100 | 0 | — |
| 332 | 10 | 100 | 0 | — |
| 333 | 10 | 100 | 0 | — |
| 334 | 10 | 100 | 0 | — |
| 335 | 10 | 100 | 0 | — |
| 336 | 10 | 100 | 0 | — |
| 337 | 10 | 100 | 0 | — |
| 338 | 10 | 100 | 0 | — |
| 339 | 10 | 100 | 0 | — |
| 340 | 10 | 100 | 0 | — |
| 341 | 10 | 100 | 0 | — |
| 342 | 10 | 100 | 0 | — |
| 343 | 10 | 0 | 0 | — |
| 344 | — | — | — | — |
| 345 | — | — | — | — |
| 346 | — | — | — | — |
| 347 | — | — | — | — |
| 348 | — | — | — | — |
| 349 | — | — | — | — |
| 350 | — | — | — | — |
| 351 | — | — | — | — |
| 352 | — | — | — | — |
| 353 | — | — | — | — |
| 354 | — | — | — | — |
| 355 | — | — | — | — |
| 356 | 10 | 100 | 0 | — |
| 357 | 10 | 100 | 9 | — |
| 358 | 10 | 100 | 0 | — |
| 359 | 10 | 100 | 0 | — |
| 360 | 10 | 100 | 0 | — |
| 361 | 10 | 100 | 0 | — |
| 362 | 10 | 100 | 0 | — |
| 364 | 50 | 100 | 0 | — |
| 365 | 10 | 100 | 0 | — |
| 366 | | | | |

What is claimed is:

1. A compound selected from Formula 1, tautomers, A-oxides, and salts thereof, 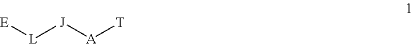
wherein
T is selected from the group consisting of: 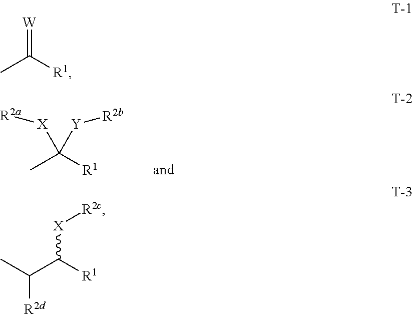
wherein the bond extending to the left is attached to A;
R¹ is CF₃, CHF₂, CCl₃, CHCl₂, CF₂Cl, CFCl₂ or CHFCl;
W is O, S or NR³;
R³ is H, cyano, nitro, C(═O)OH, benzyl, C₃-C₄ alkyl, C₂-C₄ alkylcarbonyl, C₂-C₄ haloalkylcarbonyl, OR^{3a} or NR^{3b}R^{3c};
R^{3a} is H, benzyl, C₃-C₄ alkyl, C₂-C₄ alkylcarbonyl or C₂-C₄ haloalkylcarbonyl;
R^{3b} is H, C₃-C₄ alkyl, C₂-C₄ alkylcarbonyl or C₂-C₄ haloalkylcarbonyl;
R^{3c} is H or C₃-C₄ alkyl; or
R^{3b} and R^{3c} are taken together to form a 4- to 6-membered fully saturated heterocyclic ring, each ring containing ring members, in addition to the connecting nitrogen atom, selected from carbon atoms and up to 2 heteroatoms independently selected from up to 2 O, up to 2 S and up to 2 N atoms, each ring optionally substituted with up to 2 methyl groups;
X is O, S or NR^{5a};
Y is O, S or NR^{5b};
R^{5a} and R^{5b} are each independently H, hydroxy or C₁-C₄ alkyl;
R^{2a} and R^{2b} are each independently H, C₁-C₄ alkyl, C₂-C₄ alkenyl, (CR^{4a}R^{4b})ₚ—OH, (CR^{4a}R^{4b})ₚ—SH, (CR^{4a}R^{4b})ₚ—Cl or (CR^{4a}R^{4b})ₚ—Br; or
R^{2a} and R^{2b} are taken together with the atoms X and Y to which they are attached to form a 5- to 7-membered saturated ring containing ring members, in addition to the atoms X and Y, selected from carbon atoms, wherein up to 2 carbon atom ring members are independently selected from C(═O) and C(═S), the ring optionally substituted with up to 2 substituents independently selected from halogen, cyano, C₁-C₂ alkyl, C₁-C₂ haloalkyl, alkoxy and C₁-C₂ haloalkoxy on carbon atom ring members;
R^{2c} is C₁-C₄ alkyl, C₁-C₄ haloalkyl, C₂-C₄ alkenyl, C₂-C₄ haloalkenyl, C₂-C₄ alkynyl or C₂-C₄ haloalkynyl, each optionally substituted with up 2 substituents independently selected from cyano, hydroxy, SC≡N and C₁-C₂ alkoxy;
R^{2d} is H, cyano, halogen or C₁-C₄ alkyl;
each R^{4a} and R^{4b} is independently H or C₁-C₄ alkyl;
p is 2 or 3;
when T is T-1 or T-2, then A is A¹-A²-CR^{6a}R^{6b}, wherein A¹ is connected to J, and CR^{6a}R^{6b} is connected to T;
when T is T-3, then A is A¹-A², wherein A¹ is connected to J, and A² is connected to T;
A¹ is CR^{6c}R^{6d}, N(R^{7a}), O or S;
A² is a direct bond, CR^{6e}R^{6f}, N(R^{7b}), O or S;
R^{6a}, R^{6b}, R^{6c}, R^{6d}, R^{6e} and R^{6f} are each independently H, cyano, hydroxy, halogen or C₁-C₄ alkyl;
R^{7a} and R^{7b} are each independently H, C(═O)H, C₁-C₄ alkyl or C₂-C₄ alkyl carbonyl;
J is selected from the group consisting of: 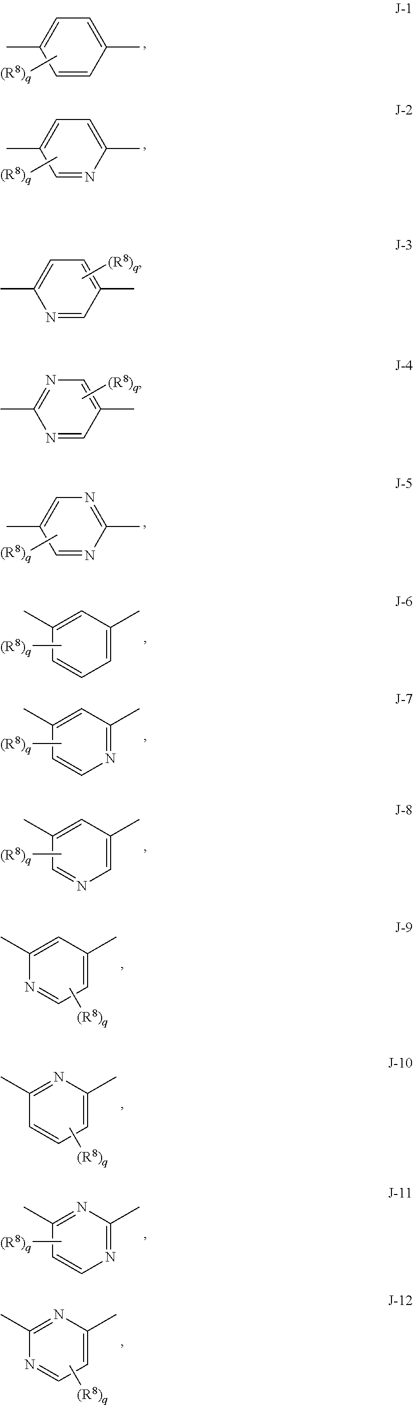 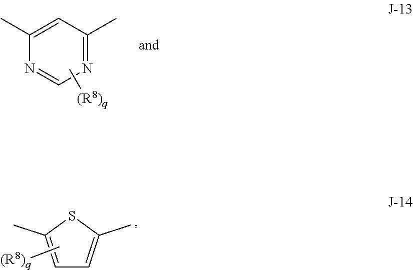
wherein the bond extending to the left is attached to L, and the bond extending to the right is attached to A;
each R⁸ is independently F, Cl, methyl or methoxy;
q is 0, 1 or 2;
L is (CR^{9a}R^{9b})ₙ;
each R^{9a} and R^{9b} is independently H, halogen, cyano, hydroxy, nitro, C₁-C₃ alkyl, C₁-C₃ haloalkyl, C₁-C₃ alkoxy or C₁-C₃ haloalkoxy;
n is 0, 1, 2 or 3;
E is E¹ or E²;
E¹ is amino, cyano, hydroxy, nitro, CH(═O), C(═O)OH, C(═O)NH₂, C(═S)NH₂, OC(═O)NH₂, OC(═S)NH₂, NHC(═O)NH₂, NHC(═S)NH₂, SCAN, —CH═NNHC(═O)OC₁-C₆ alkyl or —N(OCH₃)C(═O)C₁-C₆ alkyl; or
E¹ is C₁-C₆ alkoxy, C₂-C₆ alkenyloxy, C₂-C₆ alkynyloxy, C₁-C₆ alkylthio, C₂-C₆ alkenylthio, C₂-C₆ alkynylthio, C₁-C₆ alkylsulfinyl, C₂-C₆ alkenylsulfinyl, C₂-C₆ alkynylsulfinyl, C₁-C₆ alkylsulfonyl, C₂-C₆ alkenylsulfonyl, C₂-C₆ alkynylsulfonyl, C₁-C₆ alkylsulfonylamino, C₂-C₆ alkenylsulfonylamino, C₂-C₆ alkynylsulfonylamino, C₁-C₆ alkylaminosulfonyl, C₂-C₆ dialkylaminosulfonyl, C₂-C₆ alkenylaminosulfonyl, C₂-C₆ alkynylaminosulfonyl, C₁-C₆ alkylaminosulfonylamino, C₂-C₆ alkenylaminosulfonylamino, C₂-C₆ alkynylaminosulfonylamino, C₂-C₆ alkylcarbonyl, C₃-C₆ alkenylcarbonyl, C₃-C₆ alkynylcarbonyl, C₂-C₆ alkylaminocarbonyl, C₃-C₆ alkenylaminocarbonyl, C₃-C₆ alkynylaminocarbonyl, C₂-C₆ alkylcarbonylamino, C₃-C₆ alkenylcarbonylamino, C₃-C₆ alkynylcarbonylamino, C₂-C₆ alkylaminocarbonylamino, C₃-C₆ alkenylaminocarbonylamino, C₃-C₆ alkynylaminocarbonylamino, C₂-C₆ alkylcarbonyloxy, C₃-C₆ alkenylcarbonyloxy, C₃-C₆ alkynylcarbonyloxy, C₂-C₆ alkoxycarbonyl, C₃-C₆ alkenyloxycarbonyl, C₃-C₆ alkynyloxycarbonyl, C₂-C₆ alkylaminocarbonyloxy, C₃-C₆ alkenylaminocarbonyloxy, C₃-C₆ alkynylaminocarbonyloxy, C₂-C₆ alkoxycarbonylamino, C₃-C₆ alkenyloxycarbonylamino, C₃-C₆ alkynyloxycarbonylamino, C₂-C₆ alkylamino(thiocarbonyl)oxy, C₃-C₆ alkenylamino(thiocarbonyl)oxy, C₃-C₆ alkynylamino(thiocarbonyl)oxy, C₂-C₆ alkoxy(thiocarbonyl)amino, C₃-C₆ alkenyloxy(thiocarbonyl)amino, C₃-C₆ alkynyloxy(thiocarbonyl)amino, C₂-C₆ alkyl(thiocarbonyl), C₂-C₆ (alkylthio)carbonyl, C₃-C₆ alkenyl(thiocarbonyl), C₃-C₆ (alkenylthio)carbonyl, C₃-C₆ alkynyl(thiocarbonyl), C₃-C₆ (alkynylthio)carbonyl, C₂-C₆ alkylamino(thiocarbonyl), C₃-C₆ alkenylamino(thiocarbonyl), C₃-C₆ alkynylamino(thiocarbonyl), C₂-C₆ alkyl(thiocarbonyl)amino, C₂-C₆ (alkylthio)carbonylamino, C₃-C₆ alkenyl(thiocarbonyl)amino, C₃-C₆ (alkenylthio)carbonylamino, C₃-C₆ alkynyl(thiocarbonyl)amino, C₃-C₆ (alkynylthio)carbonylamino, C₂-C₆ alkylamino(thiocarbonyl)amino, C₃-C₆ alkenylamino(thiocarbonyl)amino or C₃-C₆ alkynylamino(thiocarbonyl)amino, wherein each carbon atom is optionally substituted with up to 1 substituent selected from R^{10a} and up to 3 substituents independently selected from R^{10b};
R^{10a} is phenyl optionally substituted with up to 3 substituents independently selected from R^{11a}; or a 5- to 6-membered heterocyclic ring containing ring members selected from carbon atoms and 1 to 4 heteroatoms independently selected from up to 2 O, up to 2 S and up to 4 N atoms, wherein up to 3 carbon atom ring members are independently selected from C(═O) and C(═S), and sulfur atom ring members are independently S(═O)ᵤ(═NR¹²)ᵥ, each ring optionally substituted with up to 3 substituents independently selected from R^{11a} on carbon atom ring members and R^{11b} on nitrogen atom ring members;
each R^{10b} is independently amino, cyano, halogen, hydroxy, nitro, SC≡N, —SH, C₁-C₄ alkyl, C₃-C₄ haloalkyl, C₃-C₆ cycloalkyl, C₃-C₆ halocycloalkyl, C₃-C₄ alkoxy, C₃-C₄ haloalkoxy, C₃-C₄ alkylthio, C₃-C₄ alkylsulfinyl, C₃-C₄ alkylsulfonyl, C₁-C₄ haloalkylsulfonyl, C₁-C₄ alkylamino, C₂-C₄ dialkylamino, C₂-C₄ alkylcarbonyl, C₂-C₄ haloalkylcarbonyl, C₂-C₅ alkoxycarbonyl, C₂-C₅ haloalkoxycarbonyl, C₂-C₅ alkylaminocarbonyl or C₃-C₅ dialkylaminocarbonyl;
each R^{11a} is independently halogen, hydroxy, cyano, amino, nitro, C₁-C₄ alkyl, C₁-C₄ haloalkyl, C₂-C₄ alkenyl, C₂-C₄ alkynyl, C₁-C₄ hydroxyalkyl, C₃-C₆ cycloalkyl, C₄-C₇ cycloalkylalkyl, C₁-C₄ alkoxy, C₁-C₄ haloalkoxy, C₂-C₄ alkenyloxy, C₂-C₄ alkynyloxy, C₂-C₄ alkoxyalkyl, C₂-C₆ alkylcarbonyloxy, C₁-C₄ alkylthio, C₁-C₄ haloalkylthio, C₂-C₆ alkylcarbonylthio, alkylsulfinyl, Q-Q haloalkylsulfinyl, C₁-C₄ alkylsulfonyl, C₁-C₄ haloalkylsulfonyl, C₁-C₄ alkylsulfonyloxy, C₁-C₄ alkylamino, C₂-C₈ dialkylamino, C₃-C₆ cycloalkylamino, C₂-C₄ alkylcarbonyl, C₃-C₅ alkenylcarbonyl, C₃-C₅ alkynylcarbonyl, C₄-C₇ cycloalkylcarbonyl, C₅-C₈ cycloalkylalkylcarbonyl, C₂-C₆ alkoxycarbonyl, C₃-C₇ alkenyloxycarbonyl, C₃-C₇ alkynyloxycarbonyl, C₄-C₇ cycloalkoxylcarbonyl, C₅-C₈ cycloalkylalkoxylcarbonyl, C₂-C₆ alkylaminocarbonyl, C₃-C₆ alkenylaminocarbonyl, C₃-C₆ alkynylaminocarbonyl, C₄-C₇ cycloalkylaminocarbonyl, C₅-C₈ cycloalkylalkylaminocarbonyl, C₃-C₈ dialkylaminocarbonyl or C₃-C₆ trialkylsilyl;
each R^{11b} is independently C(═O)H, C₁-C₃ alkyl, C₁-C₃ alkoxy, C₂-C₃ alkylcarbonyl or C₂-C₃ alkoxycarbonyl;
each R¹² is independently H, cyano, C₁-C₃ alkyl or C₁-C₃ haloalkyl;
each u and v are independently 0, 1 or 2, provided that the sum of u and v are 0, 1 or 2;
E² is G-Z, wherein Z is attached to L;
G is phenyl optionally substituted with up to 3 substituents independently selected from R¹³; or
G is a 5- to 6-membered heteroaromatic ring, each ring containing ring members selected from carbon atoms and 1 to 4 heteroatoms independently selected from up to 2 O, up to 2 S and up to 4 N atoms, each ring optionally substituted with up to 3 substituents independently selected from R¹³; or
G is a 3- to 7-membered nonaromatic ring or an 8- to 11-membered bicyclic ring system, each ring or ring system containing ring members selected from carbon atoms and optionally up to 4 heteroatoms independently selected from up to 2 O, up to 2 S and up to 4 N atoms, wherein up to 2 ring members are independently selected from C(═O), C(═S), S(═O) and S(═O)₂, each ring or ring system optionally substituted with up to 3 substituents independently selected from R¹³;
each R¹³ is independently cyano, halogen, hydroxy, nitro, —SH, SF₅, CH(═O), C(═O)OH, NR^{14a}R^{14b}, C(═O)NR^{14a}R^{14b}, C(═O)C(═O)NR^{14a}R^{14b}, C(═S)NR^{14a}R^{14b}, C(R¹⁵)═NR¹⁶, N═CR¹⁷NR^{18a}R^{18b} or —U—V-Q; or C₁-C₆ alkyl, C₂-C₆ alkenyl, C₂-C₆ alkynyl, C₃-C₇ cycloalkyl, C₃-C₇ cycloalkenyl, C₁-C₆ alkoxy, C₂-C₆ alkenyloxy, C₂-C₆ alkynyloxy, C₃-C₇ cycloalkoxy, C₁-C₆ alkylthio, C₁-C₆ alkylsulfinyl, C₁-C₆ alkylsulfonyl, C₁-C₆ alkylaminosulfinyl, C₂-C₆ dialkylaminosulfinyl, C₁-C₆ alkylsulfonyloxy, C₁-C₆ alkylsulfonylamino, C₂-C₆ alkyl carbonyl, C₄-C₇ cycloalkylcarbonyl, C₂-C₆ alkoxycarbonyl, C₃-C₆ alkenyloxycarbonyl, C₃-C₆ alkynyloxycarbonyl, C₄-C₇ cycloalkoxycarbonyl, C₃-C₆ alkoxycarbonylcarbonyl, C₂-C₆ alkylcarbonyloxy, C₄-C₇ cycloalkylcarbonyloxy, C₂-C₆ alkoxycarbonyloxy, C₄-C₇ cycloalkoxycarbonyloxy, C₂-C₆ alkylaminocarbonyloxy, C₄-C₇ cycloalkylaminocarbonyloxy, C₂-C₆ alkylcarbonylamino, C₄-C₇ cycloalkylcarbonylamino, C₂-C₆ alkoxycarbonylamino, C₄-C₇ cycloalkoxycarbonylamino, C₂-C₆ alkylaminocarbonylamino, C₄-C₇ cycloalkylaminocarbonylamino or C₂-C₆ dialkoxyphosphinyl, each optionally substituted with up to 3 substituents independently selected from R¹⁹;
each R^{14a} is independently H, cyano, hydroxy, C₁-C₄ alkyl, C₁-C₄ haloalkyl, C₂-C₄ alkenyl, C₂-C₄ haloalkenyl, C₂-C₄ alkynyl, C₂-C₄ haloalkynyl, C₃-C₅ alkoxy, C₂-C₄ alkoxyalkyl, C₁-C₄ alkylsulfonyl, C₁-C₄ haloalkylsulfonyl, C₂-C₄ alkylthioalkyl, C₂-C₄ alkylsulfinylalkyl, C₂-C₄ alkylsulfonylalkyl, C₂-C₄ alkylcarbonyl, C₂-C₄ haloalkylcarbonyl, C₄-C₇ cycloalkylcarbonyl, C₂-C₅ alkoxycarbonyl, C₃-C₅ alkoxycarbonylalkyl, C₂-C₅ alkylaminocarbonyl or C₃-C₅ dialkylaminocarbonyl;
each R^{14b} is independently H, C₁-C₆ alkyl, C₁-C₆ haloalkyl, C₂-C₆ alkenyl, C₂-C₆ haloalkenyl, C₂-C₆ alkynyl, C₂-C₆ haloalkynyl, C₁-C₆ hydroxyalkyl, C₂-C₆ cyanoalkyl, C₃-C₈ cycloalkyl, C₃-C₈ halocycloalkyl, C₃-C₈ cycloalkenyl, C₃-C₈ halocycloalkenyl, C₄-C₁₀ alkylcycloalkyl, C₄-C₁₀ cycloalkylalkyl, C₄-C₁₀ halocycloalkylalkyl, C₆-C₁₄ cycloalkylcycloalkyl, C₅-C₁₀ alkylcycloalkylalkyl, C₂-C₆ alkoxyalkyl, C₂-C₆ haloalkoxyalkyl, C₄-C₁₀ cycloalkoxyalkyl, C₃-C₈ alkoxyalkoxyalkyl, C₂-C₆ alkylthioalkyl, C₂-C₆ alkylsulfinylalkyl, C₂-C₆ alkylsulfonylalkyl, C₂-C₆ alkylaminoalkyl, C₂-C₆ haloalkylaminoalkyl, C₃-C₈ dialkylaminoalkyl or C₄-C₁₀ cycloalkylaminoalkyl, each optionally substituted with up to 1 substituent selected from cyano, hydroxy, nitro, C₂-C₄ alkylcarbonyl, C₂-C₄ alkoxycarbonyl, C₃-C₁₅ trialkylsilyl and C₃-C₁₅ halotrialkylsilyl; or
R^{14a} and R^{14b} are taken together to form a 4- to 6-membered fully saturated heterocyclic ring, each ring containing ring members, in addition to the connecting nitrogen atom, selected from carbon atoms and up to 2 heteroatoms independently selected from up to 2 O, up to 2 S and up to 2 N atoms, each ring optionally substituted with up to 3 substituents independently selected from halogen and C₁-C₃ alkyl;
each R¹⁵ is independently H, cyano, halogen, methyl, methoxy, methylthio or methoxy carbonyl;
each R¹⁶ is independently hydroxy or NR^{20a}R^{20b}; or C₁-C₄ alkoxy, C₂-C₄ alkenyloxy, C₂-C₄ alkynyloxy, C₂-C₄ alkylcarbonyloxy, C₂-C₅ alkoxycarbonyloxy, C₂-C₅ alkylaminocarbonyloxy or C₃-C₅ dialkylaminocarbonyloxy, each optionally substituted with up to 1 substituent selected from cyano, halogen, hydroxy and C(═O)OH;
each R¹⁷ is independently H, methyl, methoxy or methylthio;
each R^{18a} and R^{18b} is independently H or C₁-C₄ alkyl; or
R^{18a} and R^{18b} are taken together to form a 5- to 6-membered fully saturated heterocyclic ring, each ring containing ring members, in addition to the connecting nitrogen atom, selected from carbon atoms and up to 2 heteroatoms independently selected from up to 2 O, up to 2 S and up to 2 N atoms, each ring optionally substituted with up to 2 methyl groups;
each R¹⁹ is independently amino, cyano, halogen, hydroxy, nitro, —SH, C₁-C₄ alkyl, C₁-C₄ haloalkyl, C₃-C₆ cycloalkyl, C₃-C₆ halocycloalkyl, C₁-C₄ alkoxy, C₁-C₄ haloalkoxy, C₂-C₄ alkoxyalkoxy, C₁-C₄ alkylthio, alkylsulfinyl, C₁-C₄ alkylsulfonyl, C₁-C₄ haloalkylsulfonyl, C₂-C₄ alkylcarbonyl, C₂-C₄ haloalkylcarbonyl, C₂-C₅ alkoxycarbonyl, C₁-C₆ alkylamino, C₂-C₆ dialkylamino, C₂-C₅ alkylaminocarbonyl, C₃-C₅ dialkylaminocarbonyl, C₃-C₅ alkylthioalkylcarbonyl, C₃-C₁₅ trialkylsily, C₃-C₁₅ halotrialkylsilyl, C(R²¹)═NOR²² or C(R²³)═NR²⁴;
each U is independently a direct bond, C(═O)O, C(═O)N(R²⁵) or C(═S)N(R²⁶), wherein the atom to the left is connected to G, and the atom to the right is connected to V;
each V is independently a direct bond; or C₁-C₆ alkylene, C₂-C₆ alkenylene, C₃-C₆ alkynylene, C₃-C₆ cycloalkylene or C₃-C₆ cycloalkenylene, wherein up to 1 carbon atom is C(═O), each optionally substituted with up to 3 substituents independently selected from halogen, cyano, nitro, hydroxy, alkyl, C₁-C₂ haloalkyl, alkoxy and C₁-C₂ haloalkoxy;
each Q is independently phenyl or phenoxy, each optionally substituted with up to 2 substituents independently selected from R²⁷; or
each Q is independently a 5- to 6-membered heteroaromatic ring, each ring containing ring members selected from carbon atoms and 1 to 4 heteroatoms independently selected from up to 2 O, up to 2 S and up to 4 N atoms, each ring optionally substituted with up to 2 substituents independently selected from R²⁷; or
each Q is independently a 3- to 7-membered nonaromatic heterocyclic ring, each ring containing ring members selected from carbon atoms and 1 to 4 heteroatoms independently selected from up to 2 O, up to 2 S and up to 4 N atoms, wherein up to 2 ring members are independently selected from C(═O), C(═S), S(═O) and S(═O)₂, each ring optionally substituted with up to 2 substituents independently selected from R²⁷;
each R^{20a} is independently H, C₁-C₄ alkyl or C₂-C₄ alkylcarbonyl;
each R^{20b} is independently H, cyano, C₃-C₅ alkyl, C₂-C₅ alkylcarbonyl, C₂-C₅ haloalkylcarbonyl, C₄-C₇ cycloalkylcarbonyl, C₂-C₅ alkoxycarbonyl, C₃-C₅ alkoxycarbonylalkyl, C₂-C₅ alkylaminocarbonyl or C₃-C₅ dialkylaminocarbonyl; or
R^{20a} and R^{20b} are taken together to form a 5- to 6-membered fully saturated heterocyclic ring, each ring containing ring members, in addition to the connecting nitrogen atom, selected from carbon atoms and up to 2 heteroatoms independently selected from up to 2 O, up to 2 S and up to 2 N atoms, each ring optionally substituted with up to 2 methyl groups;
each R²¹ and R²³ is independently H, cyano, halogen, C₁-C₃ alkyl, C₁-C₃ haloalkyl, C₃-C₆ cycloalkyl or C₁-C₃ alkoxy; or phenyl optionally substituted with up to 2 substituents independently selected from halogen and C₁-C₃ alkyl;
each R²² is independently H, C₃-C₅ alkyl, C₃-C₅ haloalkyl, C₂-C₅ alkenyl, C₂-C₅ haloalkenyl, C₂-C₅ alkynyl, C₃-C₆ cycloalkyl, C₃-C₆ halocycloalkyl, C₂-C₅ alkylcarbonyl or C₂-C₅ alkoxycarbonyl; or
each R²² is phenyl optionally substituted with up to 2 substituents independently selected halogen and C₁-C₃ alkyl; or a 5- to 6-membered fully saturated heterocyclic ring, each ring containing ring members selected from carbon atoms and up to 2 heteroatoms independently selected from up to 2 O, up to 2 S and up to 2 N atoms, each ring optionally substituted with up to 2 substituents independently selected from halogen and C₁-C₃ alkyl;
each R²⁴ is independently H, cyano, C₁-C₃ alkyl, C₁-C₃ haloalkyl, alkoxy, C₂-C₄ alkylcarbonyl or C₂-C₄ alkoxycarbonyl;
each R²⁵ and R²⁶ is independently H, cyano, hydroxy, C₁-C₄ alkyl, C₁-C₄ haloalkyl, C₂-C₄ alkylcarbonyl, C₂-C₄ haloalkylcarbonyl, C₂-C₄ alkoxycarbonyl or C₂-C₄ haloalkoxycarbonyl;
each R²⁷ is independently halogen, cyano, hydroxy, nitro, C₁-C₄ alkyl, C₁-C₄ haloalkyl, C₂-C₄ alkenyl, C₁-C₄ alkoxy, C₂-C₄ alkylcarbonyl or C₂-C₄ alkoxycarbonyl;
Z is a direct bond, O, S(═O)ₘ, N(R²⁸), C(═O), C(═O)N(R²⁸), NR²⁸C(═O), N(R²⁸)C(═O)N(R²⁸), N(R²⁸)C(═S)N(R²⁸), OC(═O)N(R²⁸), N(R²⁸)C(═O)O, S(O)₂N(R²⁸), N(R²⁸)S(═O)₂ or N(R²⁸)S(O)₂N(R²⁸), wherein the atom to the right is connected to L;
each R²⁸ is independently H, C₁-C₃ alkyl, C₁-C₃ alkoxy, C₂-C₃ alkylcarbonyl or C₂-C₃ alkoxycarbonyl; and
m is 0, 1 or 2;
provided that:
(c) when A¹ is N(R^{7a}), O or S, then A² is a direct bond or CR^{6e}R^{6f}; and
when A² is N(R^{7b}), O or S; then A¹ is CR^{6c}R^{6d}.

2. A compound claim 1 wherein
R¹ is CF₃, CCl₃ or CF₂Cl;
W is O;
R^{5a} and R^{5b} are each independently H, hydroxy or methyl;
R^{2a} and R^{2b} are each independently H or methyl; or
R^{2a} and R^{2b} are taken together with the atoms X and Y to which they are attached to form a 5- to 6-membered saturated ring containing ring members, in addition to the atoms X and Y, selected from carbon atoms, wherein up to 1 carbon atom ring member is selected from C(═O), the ring optionally substituted with up to 2 substituents independently selected from halogen, cyano, methyl, halomethyl, methoxy and halomethoxy on carbon atom ring members;
R^{2c} is C₁-C₂ alkyl, C₂-C₃ alkenyl or C₂-C₃ alkynyl;
R^{2d} is H or methyl;
A¹ is CR^{6c}R^{6d} or O;
A² is a direct bond, CR^{6e}R^{6f} or O;
R^{6a}, R^{6b}, R^{6c}, R^{6d}, R^{6e} and R^{6f} are each independently H, cyano, hydroxy, Br, Cl, F or methyl;
J is J-1, J-6 or J-14;
each R⁸ is independently F, Cl or methyl;
each R^{9a} and R^{9b} is independently H, halogen or methyl;
n is 0, 1 or 2;
E¹ is C₁-C₆ alkoxy, C₃-C₆ alkylsulfonyl, C₂-C₆ alkylcarbonyl or C₂-C₆ alkoxycarbonyl, wherein each carbon atom is optionally substituted with up to 1 substituent selected from R^{10a} and up to 3 substituents independently selected from R^{10b};
R^{10a} is phenyl optionally substituted with up to 2 substituents independently selected from R^{11a}; or a 5- to 6-membered heterocyclic ring containing ring members selected from carbon atoms and 1 to 4 heteroatoms independently selected from up to 2 O, up to 2 S and up to 4 N atoms, each ring optionally substituted with up to 2 substituents independently selected from R^{11a} on carbon atom ring members and R^{11b} on nitrogen atom ring members;
each R^{10b} is independently halogen, hydroxy, C₁-C₄ alkyl, C₁-C₄ haloalkyl, C₁-C₄ alkoxy, C₁-C₄ haloalkoxy, C₁-C₄ alkylsulfonyl, C₂-C₄ alkylcarbonyl or C₂-C₅ alkoxycarbonyl;
each R^{11a} is independently halogen, C₁-C₂ alkyl, haloalkyl, alkoxy or C₂-C₃ alkoxycarbonyl;
each R^{11b} is independently methyl, methoxy, methylcarbonyl or methoxycarbonyl;
G is selected from the group consisting of: 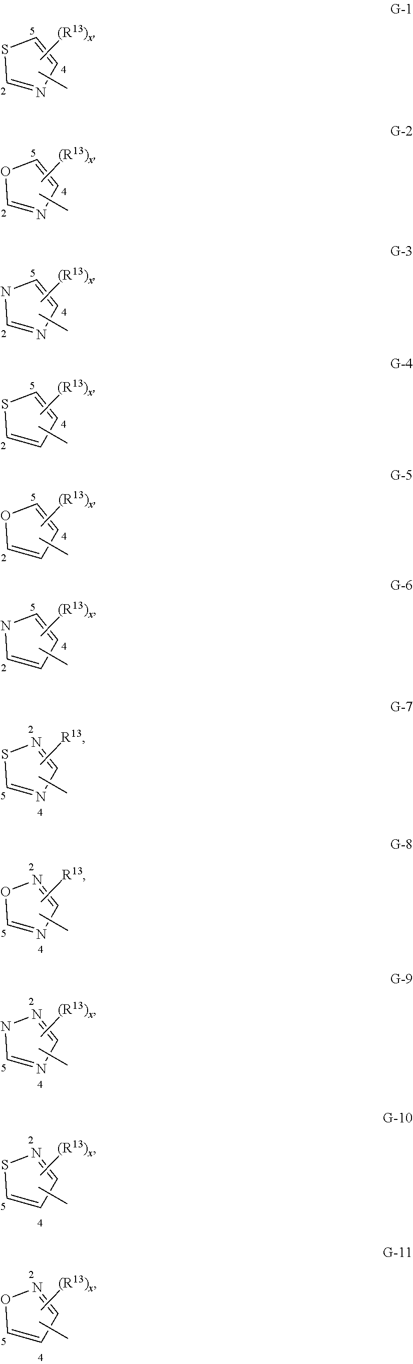 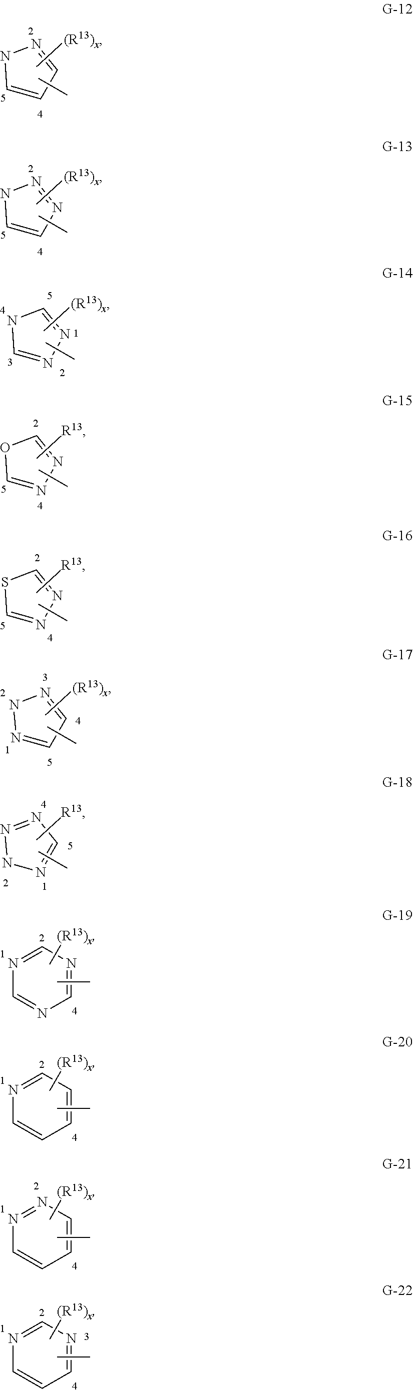 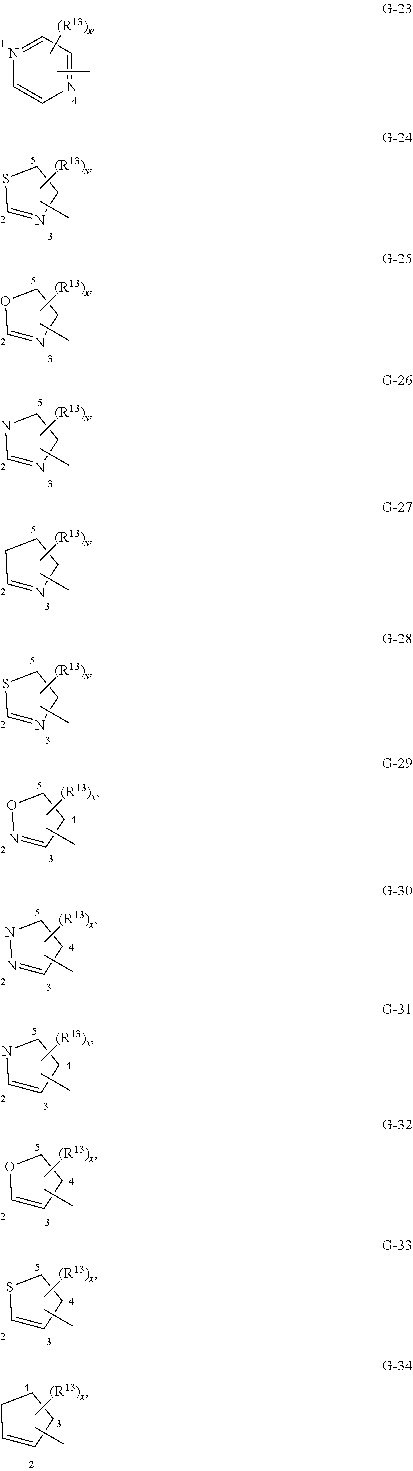 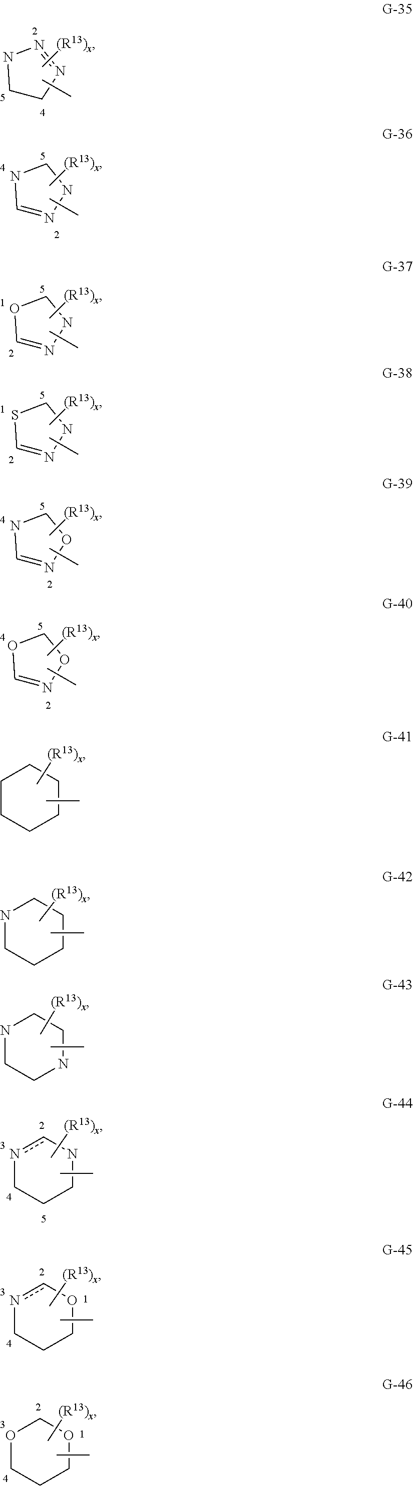 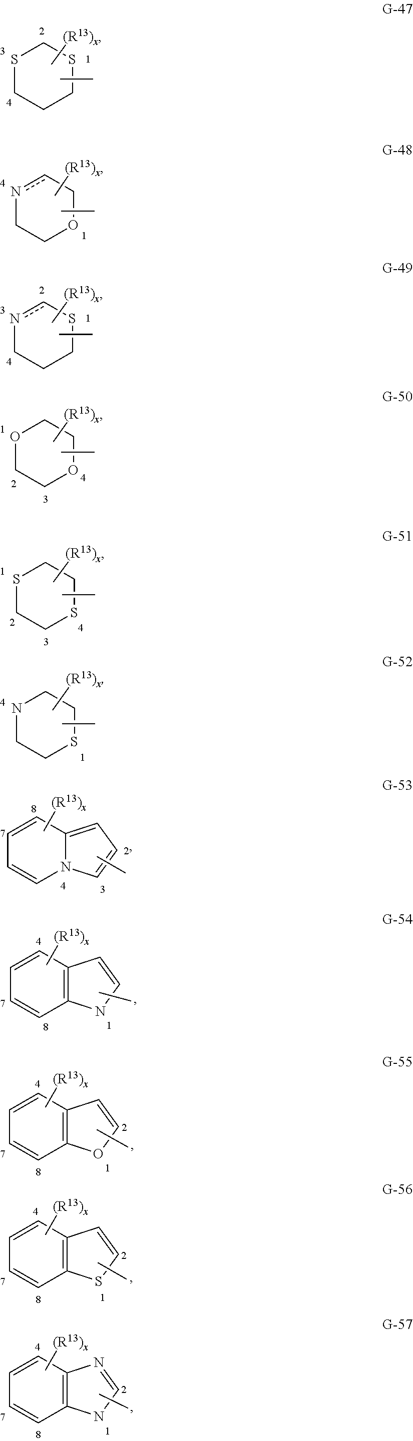 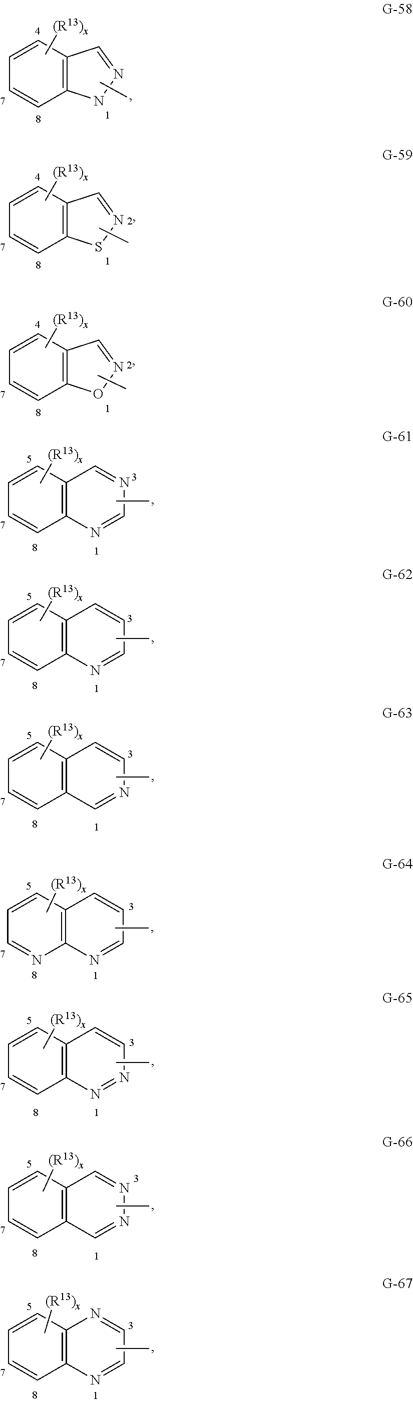 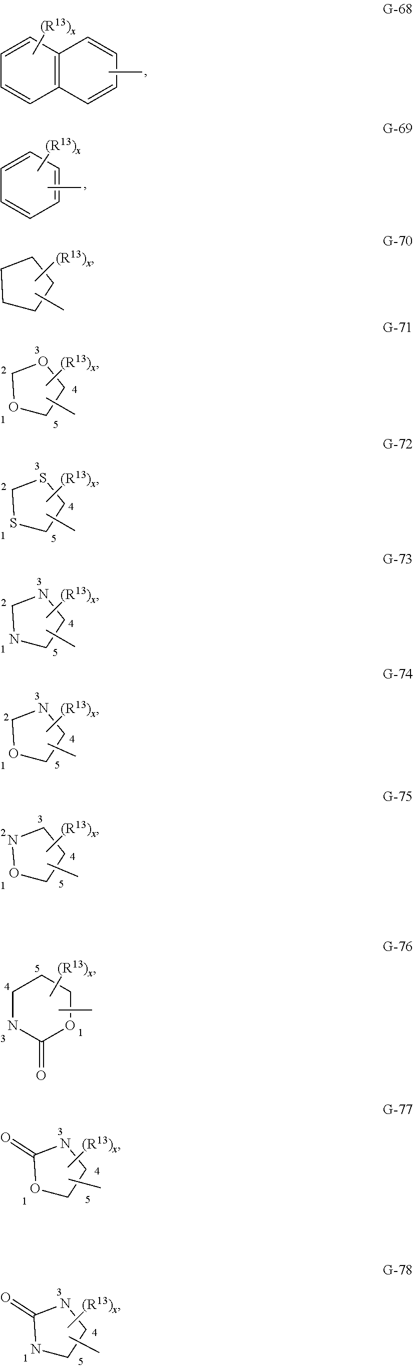 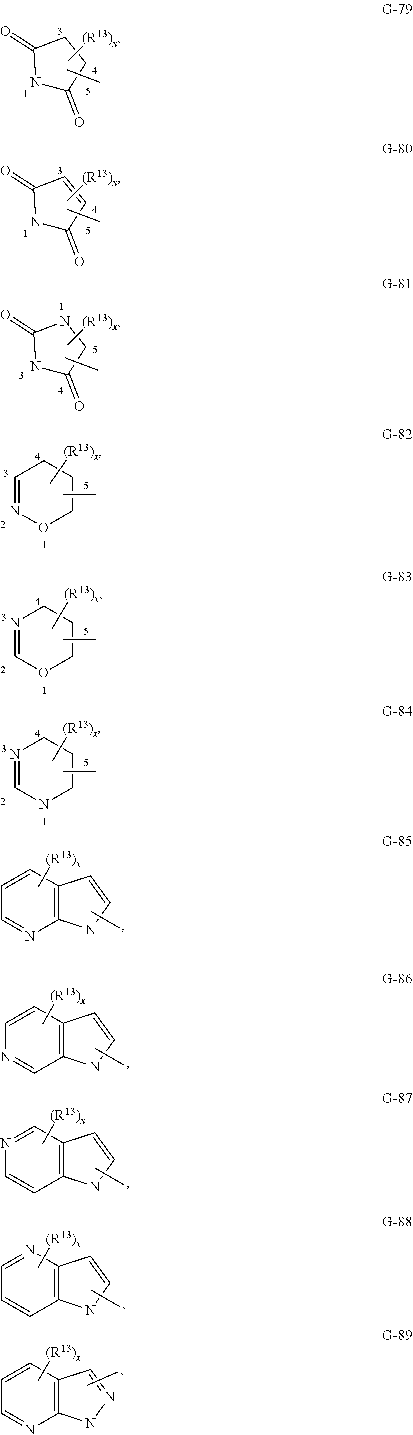 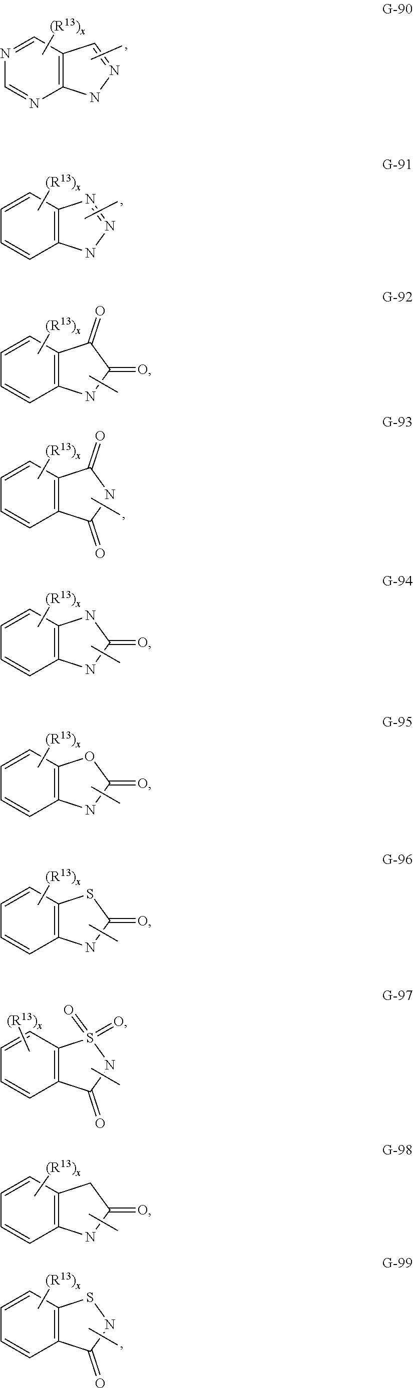 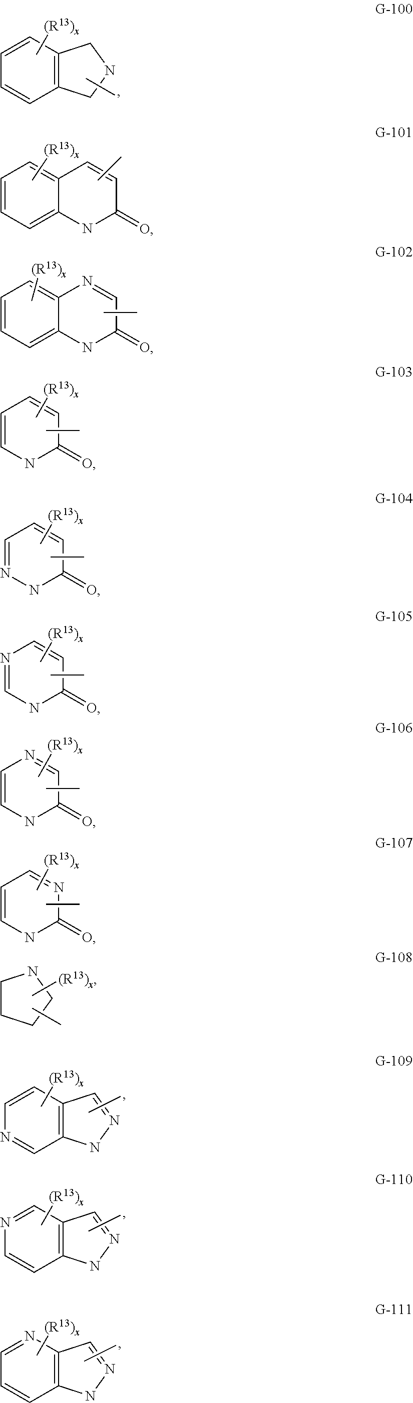 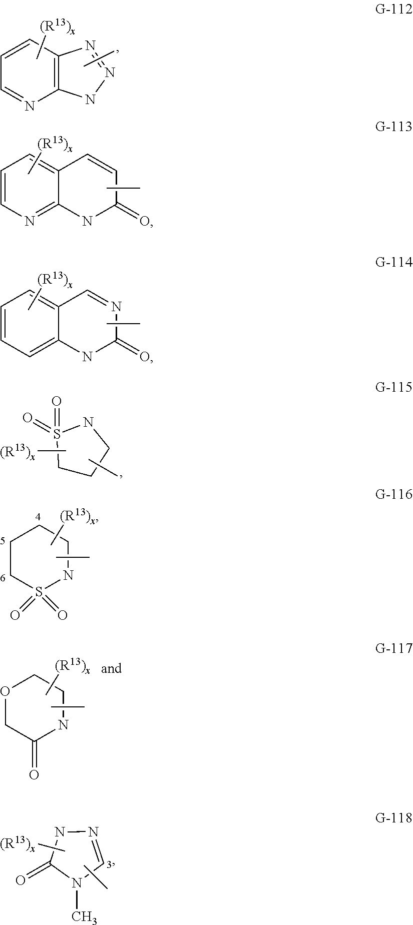
wherein the floating bond is connected to Z in Formula 1 through any available carbon or nitrogen atom of the depicted ring or ring system; and x is 0, 1, 2 or 3;
each R¹³ is independently C(═O)NR^{14a}R^{14b} or —U—V-Q; or C₁-C₆ alkyl, C₂-C₆ alkenyl, C₂-C₆ alkynyl, C₁-C₆ alkoxy, C₂-C₆ alkenyloxy, C₂-C₆ alkynyloxy, C₁-C₆ alkylsulfonyl, C₁-C₆ alkylsulfonyloxy, C₁-C₆ alkylsulfonylamino, C₂-C₆ alkylcarbonyl, C₂-C₆ alkoxycarbonyl, C₃-C₆ alkenyloxycarbonyl, C₃-C₆ alkynyloxycarbonyl, C₄-C₆ cycloalkoxycarbonyl or C₂-C₆ alkoxycarbonyloxy, each optionally substituted with up to 3 substituents independently selected from R¹⁹;
each R^{14a} is independently H, C₁-C₂ alkyl, C₂-C₄ alkenyl, C₂-C₄ alkynyl, C₂-C₄ alkylcarbonyl or C₂-C₄ alkoxycarbonyl;
each R^{14b} is independently H, alkyl, haloalkyl, C₂-C₄ alkenyl, C₂-C₄ haloalkenyl, C₂-C₄ alkynyl, C₃-C₅ cycloalkyl, C₄-C₆ cycloalkylalkyl, C₂-C₄ alkoxyalkyl, C₂-C₄ haloalkoxyalkyl, C₂-C₄ alkylaminoalkyl or C₃-C₅ dialkylaminoalkyl; or
R^{14a} and R^{14b} are taken together to form an azetidinyl, morpholinyl, pyrrolidinyl, piperidinyl, piperazinyl or thiomorpholinyl ring, each ring optionally substituted with up to 2 substituents independently selected from halogen or methyl;
each R¹⁹ is independently cyano, halogen, C₁-C₂ alkyl, C₁-C₂ haloalkyl, C₃-C₆ cycloalkyl, C₁-C₂ alkoxy, C₁-C₂ haloalkoxy, C₂-C₃ alkylcarbonyl, C₂-C₃ haloalkylcarbonyl or C₂-C₃ alkoxycarbonyl;
each U is independently a direct bond, C(═O)O or C(═O)N(R²⁵);
each V is independently a direct bond; or C₁-C₃ alkylene, each optionally substituted with up to 2 substituents independently selected from halogen, hydroxy, C₁-C₂ alkyl, C₁-C₂ alkoxy and C₁-C₂ haloalkoxy;
each Q is independently phenyl optionally substituted with up to 2 substituents independently selected from R²⁷; or pyridinyl, pyrazolyl, imidazolyl, triazolyl, thiazolyl or oxazolyl, each optionally substituted with up to 2 substituents independently selected from R²⁷;
each R²⁵ is independently H, cyano, hydroxy or C₁-C₂ alkyl;
each R²⁷ is independently halogen, cyano, C₁-C₂ alkyl, C₁-C₂ haloalkyl or C₁-C₂ alkoxy; and
Z is a direct bond, O, NH, C(═O), C(═O)NH, NHC(═O), NHC(═O)NH, OC(═O)NH, NHC(═O)O, S(═O)₂NH, NHS(═O)₂ or NHS(═O)₂NH.

3. A compound of claim 2 wherein
T is T-2 or T-3;
R¹ is CF₃;
X is O;
Y is O;
R^{2a} and R^{2b} are each independently H or methyl; or
R^{2a} and R^{2b} are taken together with the atoms X and Y to which they are attached to form a 5-membered saturated ring containing ring members, in addition to the atoms X and Y, selected from carbon atoms, the ring optionally substituted with up to 1 substituent selected from halogen, methyl and halomethyl on a carbon atom ring member;
R^{2c} is methyl or ethyl;
R^{2d} is H;
A¹ is O;
A² is a direct bond, CH₂ or O;
R^{6a} and R^{6b} are each independently H, cyano hydroxy or methyl;
J is J-1 or J-6;
q is 0 or 1;
each R^{9a} and R^{9b} is independently H or methyl;
E¹ is C₁-C₃ alkoxy, C₂-C₃ alkylcarbonyl or C₂-C₃ alkoxycarbonyl, wherein each carbon atom is optionally substituted with up to 1 substituent selected from R^{10a} and up to 3 substituents independently selected from R^{10b};
R^{10a} is pyrazolyl, imidazolyl or triazolyl, each optionally substituted with up to 2 substituents independently selected from R^{11a} on carbon atom ring members;
each R^{10b} is independently halogen, C₁-C₂ alkyl, haloalkyl, alkoxy or C₂-C₄ alkoxycarbonyl;
G is G-1, G-3, G-12 or G-22;
x is 1 or 2;
each R¹³ is independently C(═O)NR^{14a}R^{14b} or —U—V-Q; or C₂-C₅ alkoxycarbonyl, C₃-C₅ alkenyloxycarbonyl, C₃-C₅ alkynyloxycarbonyl or C₄-C₆ cycloalkoxycarbonyl, each optionally substituted with up to 3 substituents independently selected from R¹⁹;
each R^{14a} is independently H or C₁-C₂ alkyl;
each R^{14b} is independently H, C₁-C₃ alkyl, C₁-C₃ haloalkyl, cyclopropylmethyl or C₂-C₄ alkoxy alkyl;
each R¹⁹ is independently cyano, halogen, cyclopropyl, cyclobutyl, methoxy, halomethoxy or methoxycarbonyl;
each U is independently a direct bond or C(═O)O;
each V is independently a direct bond or CH₂;
each Q is independently phenyl or pyridinyl, each optionally substituted with up to 2 substituents independently selected from R²⁷;
each R²⁷ is independently halogen, methyl or methoxy; and
Z is a direct bond, O, NH, C(═O), C(═O)NH or NHC(═O).

4. A compound of claim 3 wherein
R^{2a} and R^{2b} are each H; or
R^{2a} and R^{2b} are taken together with the atoms X and Y to which they are attached to form a 5-membered saturated ring containing ring members, in addition to the atoms X and Y, selected from carbon atoms;
A² is a direct bond;
R^{6a} and R^{6b} are each H;
R⁸ is F or C₁;
Lisa direct bond, CH₂ or CH₂CH₂;
E¹ is C₁-C₂ alkoxy or C₂-C₃ alkoxycarbonyl, wherein each carbon atom is optionally substituted with up to 1 substituent selected from R^{10a};
R^{10a} is pyrazolyl or imidazolyl, each optionally substituted with up to 2 substituents independently selected from R^{11a} on carbon atom ring members;
each R^{11a} is independently methoxy carbonyl or ethoxy carbonyl;
G is G-1 and the 2-position of G-1 is connected to Z and the 4-position is connected to R¹³; or G is G-12 and the 1-position of G-12 is connected to Z and the 4-position is connected to R¹³; or G is G-12 and the 1-position of G-12 is connected to Z and the 3-position is connected to R¹³;
x is 1;
R¹³ is C(═O)NR^{14a}R^{14b} or —U—V-Q; or C₂-C₅ alkoxycarbonyl, C₃-C₅ alkynyloxycarbonyl or C₄-C₀ cycloalkoxycarbonyl, each optionally substituted with up to 1 substituent selected from R¹⁹;
R^{14a} is H;
R^{14b} is H, methyl or cyclopropylmethyl;
R¹⁹ is cyano, halogen, cyclopropyl or methoxy;
U is C(═O)O;
V is CH₂;
Q is phenyl optionally substituted with up to 2 substituents independently selected from R²⁷; and
Z is a direct bond, O, NH or C(═O).

5. A compound of claim 4 wherein
R⁸ is F;
L is a direct bond or CH₂;
E¹ is methoxy substituted with 1 substituent selected from R^{10a};
R^{10a} is pyrazolyl optionally substituted with up to 1 substituent selected from R^{11a} on a carbon atom ring member;
G is G-12 and the 1-position of G-12 is connected to Z and the 4-position is connected to R¹³; or G is G-12 and the 1-position of G-12 is connected to Z and the 3-position is connected to R¹³; and
R¹³ is C₂-C₅ alkoxycarbonyl optionally substituted with up to 1 substituent selected from R¹⁹;
R¹⁹ is cyano, Cl, F, cyclopropyl or methoxy; and
Z is a direct bond.

6. A compound of claim 5 wherein
J is J-1;
q is 0;
L is CH₂;
E is E²;
G is G-12 and the 1-position of G-12 is connected to Z and the 4-position is connected to R¹³; and
R¹³ is methoxycarbonyl or ethoxy carbonyl.

7. A compound of claim 1 which is selected from the group:
ethyl 1-[[4-(3,3,3-trifluoro-2,2-dihydroxypropoxy)phenyl]methyl]-1H-pyrazole-4-carboxylate;
ethyl 1-[[4-[[2-(trifluoromethyl)-1,3-dioxolan-2-yl]methoxy]phenyl]methyl]-1H-pyrazole-4-carboxylate;
ethyl 1-[[4-[[(1Z)-2-ethoxy-3,3,3-trifluoro-1-propen-1-yl]oxy]phenyl]methyl]-1H-pyrazole-4-carboxylate;
ethyl 1-[[4-[[(1Z)-2-ethoxy-3,3,3-trifluoro-1-propen-1-yl]oxy]phenyl]methyl]-1H-pyrazole-3-carboxylate;
ethyl 1-[[4-[[(1Z)-2-ethoxy-3,3,3-trifluoro-1-propen-1-yl]oxy]-3-fluorophenyl]methyl]-1H-pyrazole-4-carboxylate;
ethyl 1-[[3-[[(1Z)-2-ethoxy-3,3,3-trifluoro-1-propen-1-yl]oxy]phenyl]methyl]-1H-pyrazole-4-carboxylate;
ethyl 1-[[4-[[(1Z)-2-ethoxy-3,3,3-trifluoro-1-propen-1-yl]oxy]phenoxy]methyl]-1H-pyrazole-4-carboxylate;
N-(cyclopropylmethyl)-2-[[4-[[2-(trifluoromethyl)-1,3-dioxolan-2-yl]methoxy]phenyl]-methyl]thiazole-4-carboxamide;
2-methylpropyl 1-[[4-[[2-(trifluoromethyl)-1,3-dioxolan-2-yl]methoxy]phenyl]methyl]-1H-pyrazole-4-carboxylate;
cyclopropylmethyl 1-[[4-[[2-(trifluoromethyl)-1,3-dioxolan-2-yl]methoxy]phenyl]methyl]-1H-pyrazole-4-carboxylate;
ethyl 1-[2-[4-[[(1Z)-2-ethoxy-3,3,3-trifluoro-1-propen-1-yl]oxy]phenyl]ethyl]-1H-pyrazole-4-carboxylate;
2-methoxyethyl 1-[[4-[[2-(trifluoromethyl)-1,3-dioxolan-2-yl]methoxy]phenyl]methyl]-1H-pyrazole-4-carboxylate;
2-butyn-1-yl 1-[[4-[[2-(trifluoromethyl)-1,3-dioxolan-2-yl]methoxy]phenyl]methyl]-1H-pyrazole-4-carboxylate;
3-cyanopropyl 1-[[4-[[2-(trifluoromethyl)-1,3-dioxolan-2-yl]methoxy]phenyl]methyl]-1H-pyrazole-4-carboxylate;
phenylmethyl 1-[[4-[[(1Z)-2-ethoxy-3,3,3-trifluoro-1-propen-1-yl]oxy]phenyl]methyl]-1H-pyrazole-4-carboxylate;
butyl 1-[[4-[[(1Z)-2-ethoxy-3,3,3-trifluoro-1-propen-1-yl]oxy]phenyl]methyl]-1H-pyrazole-4-carboxylate;
3-chloropropyl 1-[[4-[[(1Z)-2-ethoxy-3,3,3-trifluoro-1-propen-1-yl]oxy]phenyl]methyl]-1H-pyrazole-4-carboxylate;
methyl 4-(3,3,3-trifluoro-2,2-dihydroxypropoxy)phenylcarboxylate;
ethyl 1-[[3-fluoro-4-[[2-(trifluoromethyl)-1,3-dioxolan-2-yl]methoxy]phenyl]methyl]-1H-pyrazole-4-carboxylate;
ethyl 1-[[4-[[(1Z)-2-ethoxy-3,3,3-trifluoro-1-propen-1-yl]oxy]phenylmethoxy]methyl]-1H-pyrazole-4-carboxylate;
methyl 1-[[3-[[(1Z)-2-ethoxy-3,3,3-trifluoro-1-propen-1-yl]oxy]phenyl]methyl]-1H-pyrazole-4-carboxylate; and
propyl 1-[[3-[[(1Z)-2-ethoxy-3,3,3-trifluoro-1-propen-1-yl]oxy]phenyl]methyl]-1H-pyrazole-4-carboxylate.

8. A fungicidal composition comprising (a) a compound of claim 1; and (b) at least one other fungicide.

9. A fungicidal composition comprising (a) a compound of claim 1; and (b) at least one additional component selected from the group consisting of surfactants, solid diluents and liquid diluents.

10. A method for controlling plant diseases caused by fungal plant pathogens comprising applying to the plant or portion thereof, or to the plant seed, a fungicidally effective amount of a compound of claim 1.

11. A compound selected from Formula 10, A-oxides, and salts thereof, 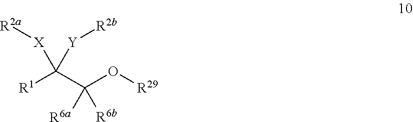
wherein
R¹ is CF₃, CCl₃ or CFCl₂;
X is O;
Y is O;
R^{2a} and R^{2b} are each independently H or methyl; or
R^{2a} and R^{2b} are taken together with the atoms X and Y to which they are attached to form a 5-membered saturated ring containing ring members, in addition to the atoms X and Y, selected from carbon atoms, wherein up to 1 carbon atom ring member is selected from C(═O), the ring optionally substituted with up to 2 substituents independently selected from halogen, cyano, methyl, halomethyl, methoxy and halomethoxy on carbon atom ring members;
R^{6a} and R^{6b} are each independently H, cyano, Br, Cl, F or methyl;
R²⁹ is S(═O)₂R³⁰; and
R³⁰ is C₁-C₄ alkyl, C₁-C₄ haloalkyl, phenyl, 4-methylphenyl 4-bromophenyl or 4-nitrophenyl.

12. A compound of claim 11 wherein
R¹ is CF₃;
R^{2a} and R^{2b} are each H; or
R^{2a} and R^{2b} are taken together with the atoms X and Y to which they are attached to form a 5-membered saturated ring containing ring members, in addition to the atoms X and Y, selected from carbon atoms;
R^{6a} and R^{6b} are each H; and
R³⁰ is CH₃, CF₃, CH₂CF₃, (CF₂)₃CF₃, phenyl or 4-methylphenyl.
